(19) United States
(12) Patent Application Publication
(10) Pub. No.: US 2014/0170168 A1
(43) Pub. Date: Jun. 19, 2014

(54) ANTIBODIES WHICH BIND SOLUBLE T-CELL RECEPTOR LIGANDS

(71) Applicants: Yoram Reiter, Haifa (IL); Rony Dahan, Mazkeret Batia (IL); Arthur A. Vandenbark, Portland, OR (US)

(72) Inventors: Yoram Reiter, Haifa (IL); Rony Dahan, Mazkeret Batia (IL); Arthur A. Vandenbark, Portland, OR (US)

(21) Appl. No.: 13/881,414

(22) PCT Filed: Oct. 26, 2011
(86) PCT No.: PCT/IB2011/054775

§ 371 Date: Apr. 25, 2013

Related U.S. Application Data
(60) Provisional application No. 61/406,609, filed on Oct. 26, 2010.

(51) Int. Cl. C07K 16/28; G01N 33/564

(52) U.S. Cl. CPC C07K 16/2809; G01N 33/564
USPC 424/173.1; 530/389.6; 506/9; 530/388.22; 435/252.1; 435/255.1; 435/7.92

(57) ABSTRACT

Provided are isolated high affinity entities which comprise an antigen binding domain which specifically binds a soluble T-cell receptor ligand comprising a two-domain beta1-alpha1 of a major histocompatibility complex (MHC) class II, wherein said antigen binding domain does not bind a complex comprising a four-domain alpha1-beta1/alpha2-beta2 MHC class II. Also provided are methods and kits using same for detecting and sequestering soluble two-domain T cell receptor ligands in a sample.

### FIELD AND BACKGROUND OF THE INVENTION

The present invention, in some embodiments thereof, relates to isolated high affinity entities (e.g., antibodies) which specifically bind soluble two-domain T-cell receptor ligands, and more particularly, but not exclusively, to methods of using same for detecting presence, level and/or pharmacokinetics of soluble two-domain T-cell receptor ligands and/or sequestering the soluble two-domain T-cell receptor ligands in a subject.

A common basis for several autoimmune diseases, including Multiple Sclerosis (MS), Type 1 Diabetes (T1D) and Rheumatoid Arthritis (RA), is the strong linkage between HLA genotype and susceptibility to the disease (Nepom, 1991; Sawcer, 2005; McDaniel, 1989). While some alleles are tightly linked to certain diseases, others confer protection and are extremely rare in patients. This linkage is not surprising due to the involvement of T-cells in the progression of these diseases. Activation or disregulation of CD4+ T-cells directed to self organ-specific proteins, combined with yet-undefined events, may contribute to the pathogenesis of a variety of human autoimmune diseases.

Multiple sclerosis is an immune-mediated demyelinating and neurodegenerative disease of the central nervous system (CNS) (Trapp, 2008). Susceptibility to MS is associated with human leukocyte antigen (HLA) class II alleles, mostly the DR2 haplotype that includes the DRB1*1501, DRB5*0101, and DQB1*0602 genes (Olerup, 1991). DRB1*1501 is a well-studied risk factor of MS that occurs in about 60% of Caucasian MS patients vs. 25% of healthy controls. Contribution of these risk factors to disease process likely involves presentation of self antigens by disease-associated MHC expressed on antigen presenting cells (APC) that activate T-cell-mediated central nervous system (CNS) inflammation. Suspected MS autoantigens include myelin proteins such as myelin basic protein (MBP), proteolipid protein (PLP), and myelin oligodendrocyte glycoprotein (MOG). T-cells from MS patients were found to predominantly recognize MOG (Kerlero de rosbo, 1993; Kerlero de rosbo, 1998) as well as other myelin proteins, and the MOG-35-55 peptide was found to be highly encephalitogenic in rodents and monkeys (Mendel, 1995; Johns, 1995) and induces severe chronic experimental autoimmune encephalomyelitis (EAE) in HLA-DRB1*1501-Tg mice (Rich, 2004).

Type 1 Diabetes (T1D) involves progressive destruction of pancreatic beta-cells by autoreactive T-cells specific for antigens expressed in the pancreatic islets, including glutamic acid decarboxylase (GAD65) (Karslen, 1991). GAD65 is a suspected islet autoantigen in T1D, stimulating both humoral and cellular self reactivity in at-risk and diseased subjects. Antibodies to GAD65 in combination with antibodies directed at two additional islet autoantigens are predictive markers of T1D in at-risk subjects (Verge, 1996), and GAD-555-567 peptide has identical sequence in all GAD isoforms in human and mouse. This highly immunogenic determinant was found to be a naturally processed T-cell epitope both in disease-associated-HLA-DR4(*0401)-Tg-mice (Patel, 1997) and human T1D subjects (Reijonen, 2002; Nepom, 2001).

Celiac (Coeliac) is an autoimmune disorder of the small intestine that occurs in genetically predisposed people of all ages from middle infancy onward. Celiac is caused by a reaction to gliadin, a prolamin (gluten protein) found in wheat, and similar proteins found in the crops of the tribe Triticeae (e.g., barley and rye). Upon exposure to gliadin, and specifically to two peptides found in prolamins (Gliadin-61-71 and Gliadin-3-24) the immune system cross-reacts with the small-bowel tissue, causing an inflammatory reaction.

Cerebral ischemia, stroke, is associated with the breakdown of the blood-brain barrier, which allows infiltration of lymphocytes into the brain and leakage of antigens from the injured neurons and glial cells into the peripheral circulation, leading to development of auto-immune response to these antigens. Thus, antibodies to brain antigens such as myelin basic protein, neurofilaments and the NR2A/2B subtype of the N-methyl-D-aspartate receptor are documented in persons after stroke [Becker K J. Sensitization and Tolerization to Brain Antigens in Stroke. Neuroscience. 2009, 158(3):1090-7. Review; Subramanian S, et al., Stroke. 2009, 40(7): 2539-45. Recombinant T cell receptor ligand treats experimental stroke].

Antigen-specific activation or regulation of CD4 T-cells is a multistep process where co-ligation of the T-cell receptor (TCR) with complexes of MHC II/peptide on the surface of APC plays a central role. Full activation through the TCR of CD4+ T-cells requires co-stimulation of additional T-cell surface molecules such as CD4, CD28 and CD40, whereas absence of co-stimulation may lead to anergy, a state of unresponsiveness of the T-cells to their presented antigen (Schwartz, 1996; Quill and Schwartz, 1987).

Thus, antigen presenting cell-associated four-domain MHC class-II molecules play a central role in activating autoreactive CD4+ T-cells involved in autoimmune diseases such as multiple Sclerosis, type 1 Diabetes, Rheumatoid Arthritis and celiac.

Recombinant T-cell receptor Ligands (RTLs) are soluble two-domain MHC class II constructs with or without covalently attached antigenic peptides that can selectively bind to the T-cell receptor (TCR) in the absence of co-stimulation (Burrows et al., 1999; Burrows et al., 2001; Chang et al., 2001) and induce specific immunological tolerance in pathogenic CD4+ inflammatory T-cells (Burrows, 2001; Wang, 2003; U.S. Patent Application No. 20050142142 to Burrows, Gregory G. et al.). RTLs constructed with different combinations of MHC class β1α1 domains and pathogenic peptides can reverse clinical and histopathological signs of disease in animal models of multiple sclerosis (Sinha, 2009; Link, 2007), uveitis (Admus, 2006), arthritis (Huan, 2008) and stroke (Subramanian, 2009), and multiple sclerosis (RTL1000; Yadav et al., 2010, Neurology, 74:S2; A293-294). Thus, two-domain MHC-II structures with the covalently-attached self peptide (RTLs) can regulate pathogenic CD4+ T-cells and reverse clinical signs of experimental autoimmune diseases.

RTL1000, comprised of the β1α1 domains of HLA-DR2 linked to the encephalitogenic human MOG-35-55 peptide, was shown to be safe and well-tolerated in a Phase I clinical trial in MS (Yadav et al., 2010, Neurology, 74:S2; A293-294).

Pawelec G, et al., 1985 (Hum. Immunol. 12(3):165-176) and Ziegler A, et al., 1986 (Immunobiology, 171(1-2):77-92) describe the isolation of the TU39 anti-DR/DP/DQ human MHC class II antibody which also binds human RTLs.

Additional background art describe generation of a family of recombinant Fabs with peptide-specific, MHC class I allele-restricted specificity for a wide panel of tumor and viral derived T-cell epitopes, isolated by screening large Ab phage libraries [Lev, 2002; Denkberg, 2002; Cohen, 2002; Denkberg, 2003; Epel, 2008; Michaeli, 2009].

### SUMMARY OF THE INVENTION

According to an aspect of some embodiments of the present invention there is provided an isolated high affinity entity comprising an antigen binding domain which specifically binds a soluble T-cell receptor ligand comprising a two-domain β1-α1 of a major histocompatibility complex (MHC) class II, wherein the antigen binding domain does not bind a complex comprising a four-domain α1-β1/α2-β2 MHC class II.

According to an aspect of some embodiments of the present invention there is provided a method of isolating a high affinity entity which specifically binds to a recombinant T-cell receptor ligand (RTL), comprising: (a) screening a library comprising a plurality of high affinity entities with an isolated complex comprising a major histocompatibility complex (MHC) class II antigenic peptide being covalently linked to a two-domain β1-α1 of the MHC class II; and (b) isolating at least one high affinity entity comprising an antigen binding domain which specifically binds the isolated complex, wherein the at least one high affinity entity does not bind to a complex comprising a four-domain α1-β1/α2-β2 MHC class II and the MHC class II antigenic peptide, thereby isolating the high affinity entities which specifically binds to the recombinant T-cell ligand (RTL).

According to an aspect of some embodiments of the present invention there is provided a method of determining a presence and/or level of a soluble T cell receptor ligand in a sample, comprising contacting the sample with the high affinity entity of some embodiments of the invention under conditions which allow immunocomplex formation, wherein a presence or a level above a predetermined threshold of the immunocomplex is indicative of the presence and/or level of the soluble T cell receptor ligand in the sample, thereby determining the presence and/or the level of the soluble T cell receptor ligand in the sample.

According to an aspect of some embodiments of the present invention there is provided a method of determining pharmacokinetic of a soluble T cell receptor ligand in a blood of a subject, comprising: (a) administering the soluble T cell receptor ligand to the subject, and (b) determining at predetermined time points a presence and/or level of the soluble T cell receptor ligand in a blood sample of the subject according to the method of some embodiments of the invention, thereby determining the pharmacokinetic of the soluble T cell receptor ligand in the blood of a subject

According to an aspect of some embodiments of the present invention there is provided a kit for detecting presence of a soluble T cell receptor ligand in a sample, comprising the high affinity entity of some embodiments of the invention and instructions for use in detecting the presence of the soluble T cell receptor ligand in the sample.

According to an aspect of some embodiments of the present invention there is provided a method of sequestering soluble T cell receptor ligand in a subject, comprising administering the high affinity entity of any of some embodiments of the invention to the subject, thereby sequestering soluble T cell receptor ligand.

According to some embodiments of the invention, the two-domain β1-α1 of the MHC class II is in complex with an MHC class II antigenic peptide.

According to some embodiments of the invention, the four-domain α1-β1/α2-β2 MHC class II is in complex with the MHC class II antigenic peptide.

According to some embodiments of the invention, the antigen binding domain does not bind the two-domain β1-α1 MHC class II in an absence of the MHC class II antigenic peptide, and wherein the antigen binding domain does not bind to the MHC class II antigenic peptide in an absence of the two-domain β1-α1 MHC class II.

According to some embodiments of the invention, the two-domain β1-α1 of the MHC class II is covalently linked to the MHC class II antigenic peptide.

According to some embodiments of the invention, the antigen binding domain comprising complementarity determining regions (CDRs) set forth by SEQ ID NOs:1-3 and 7-9 (CDRs 1-3 of light chain and heavy chain, respectively, of 2E4); SEQ ID NOs:17-19 and 23-25 (CDRs 1-3 of light chain and heavy chain, respectively, of 1F11); SEQ ID NOs:33-35 and 39-41 (CDRs 1-3 of light chain and heavy chain, respectively, of 3A3); SEQ ID NOs:49-51 and 55-57 (CDRs 1-3 of light chain and heavy chain, respectively, of 3H5); SEQ ID NOs:65-67 and 71-73 (CDRs 1-3 of light chain and heavy chain, respectively, of 2C3); SEQ ID NOs:97-99 and 103-105 (CDRs 1-3 of light chain and heavy chain, respectively, of D2);

According to some embodiments of the invention, the antigen binding domain binds the two-domain β1-α1 of MHC class II when in complex with an MHC class II antigenic peptide or in an absence of the MHC class II antigenic peptide.

According to some embodiments of the invention, the antigen binding domain comprising complementarity determining regions (CDRs) set forth by SEQ ID NOs:81-83 and 87-89 (CDRs 1-3 of light and heavy chain, respectively of 1B11).

According to some embodiments of the invention, the at least one high affinity entity does not bind the MHC class II in an absence of the MHC class II antigenic peptide, and wherein the at least one high affinity entity does not bind to the MHC class II antigenic peptide in an absence of the MHC class II.

According to some embodiments of the invention, the isolated complex further comprising a peptide for site specific biotinylation.

According to some embodiments of the invention, the antigen binding domain does not bind a complex of the MHC class II and the MHC class II antigenic peptide when presented on an antigen presenting cell (APC).

According to some embodiments of the invention, the high affinity entity is selected from the group consisting of an antibody, an antibody fragment, a phage displaying an antibody, a peptibody, a bacteria displaying an antibody, a yeast displaying an antibody, and a ribosome displaying an antibody.

According to some embodiments of the invention, the high affinity entity comprises a monoclonal antibody.

According to some embodiments of the invention, the antibody comprises a human antibody.

According to some embodiments of the invention, the MHC class II is selected from the group consisting of HLA-DM, HLA-DO, HLA-DP, HLA-DQ, and HLA-DR.

According to some embodiments of the invention, the MHC class II antigenic peptide is an autoantigenic peptide associated with a disease selected from the group consisting of diabetes, multiple sclerosis, rheumatoid arthritis, celiac uveitis and stroke.

According to some embodiments of the invention, the autoantigenic peptide associated with the diabetes is derived from a polypeptide selected from the group consisting of preproinsulin (SEQ ID NO:113), proinsulin (SEQ ID NO:114), Glutamic acid decarboxylase (GAD (SEQ ID NO:115), Insulinoma Associated protein 2 (IA-2; SEQ ID NO:116), IA-213 (SEQ ID NOs:117, 133 and 134), Islet-specific Glucose-6-phosphatase catalytic subunit-Related Protein (IGRP isoform 1 (SEQ ID NO:118), and Islet-specific Glucose-6-phosphatase catalytic subunit-Related Protein (IGRP isoform 2 (SEQ ID NO:119), chromogranin A (ChgA) (SEQ ID NO:120), Zinc Transporter 8 (ZnT8 (SEQ ID NO:121), Heat Shock Protein-60 (HSP-60; SEQ ID NO:122), Heat Shock Protein-70 (HSP-70; SEQ ID NO:123 and 124).

According to some embodiments of the invention, the GAD autoantigenic peptide comprises a core amino acid sequence set forth by SEQ ID NO:125 (GAD556-565, FFRMVISNPA).

According to some embodiments of the invention, the GAD autoantigenic peptide comprises a core amino acid sequence set forth by SEQ ID NO:125 (GAD₅₅₆₋₅₆₅, FFRMVISNPA) and no more than 30 amino acids.

According to some embodiments of the invention, the GAD autoantigenic peptide is GAD₅₅₅₋₅₆₇ (NFFRMVISNPAAT; SEQ ID NO:126).

According to some embodiments of the invention, the autoantigenic peptide associated with the multiple sclerosis is derived from a polypeptide selected from the group consisting of myelin oligodendrocyte glycoprotein (MOG; SEQ ID NOs:135-143), myelin basic protein (MBP; SEQ ID NOs:127 and 144-148), and proteolipid protein (PLP; SEQ ID NOs:128, 149 and 150).

According to some embodiments of the invention, the MOG autoantigenic peptide is MOG-35-55 (SEQ ID NO:129).

According to some embodiments of the invention, the MBP autoantigenic peptide is MBP-85-99 (SEQ ID NO:130).

According to some embodiments of the invention, the autoantigenic peptide associated with the celiac is derived from an alpha Gliadin polypeptide (SEQ ID NO:131 or 199).

According to some embodiments of the invention, the autoantigenic peptide associated with the rheumatoid arthritis is derived from Collagen II polypeptide (SEQ ID NO:132).

According to some embodiments of the invention, the method further comprising performing a calibration curve using known amounts of the soluble T cell receptor ligand.

According to some embodiments of the invention, the kit further comprising reagents for detecting presence of an immunocomplex comprising the high affinity entity and the recombinant T cell receptor ligand.

According to some embodiments of the invention, the kit further comprising the recombinant T cell receptor ligand.

According to some embodiments of the invention, the soluble T cell receptor ligand exhibits an excessive inhibitory activity.

According to some embodiments of the invention, the excessive inhibitory activity of the soluble T cell receptor ligand is associated with cancer or an infectious disease.

According to some embodiments of the invention, the antigen presenting cells comprise macrophages, dendritic cells or B cells.

According to some embodiments of the invention, the soluble T-cell receptor ligand comprises a recombinant T-cell receptor ligand.

According to some embodiments of the invention, the soluble T-cell receptor ligand comprises a native T-cell receptor ligand.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-E depict purification of RTL1000. FIG. 1A is a graph depicting the purification of RTL1000 and analysis by Size Exclusion Chromatography. RTL1000 is isolated and purified as a monodisperse molecule. Elution volumes of known molecular weight proteins (43, 29, 13.7, 6.5 kD) are marked as *, respectively, with increasing retention volume. FIG. 1B—SDS-polyacrylamide gel electrophoresis (SDS-PAGE) depicting the purified RTL1000 protein. Lane 1—RTL1000; Lane 2—molecular weight (MW) size marker. Note the high purity RTL1000 band of 25 kDa. FIG. 1C—Samples of RTLs with or without β-mercaptoethanol (βME), analyzed by SDS-polyacrylamide gel, showed an increase in apparent MW after reduction of the conserved internal disulfide bond. FIG. 1D—Biotinylated RTL1000 and 100% biotinylated Myelin Basic Protein (MBP) standard were separated by SDS-PAGE, blotted and stained with horse radish peroxidase (HRP)-conjugated sterptavidin. Identical band intensity of the compared proteins was observed. FIG. 1E-A histogram depicting IL-2 dependent CTLL cell line proliferation by DR*1501 antigen presenting cells (APCs) pulsed with MOG-35-55 peptide in the presence of RTL1000, RTL340 or medium. H2-1 cells were pre-incubated with RTL1000, RTL340 or medium alone before their Ag-specific activation with DR*1501 APCs pulsed with MOG-35-55 peptide. Note the inhibition of MOG-35-55-specific response of H2-1 T-cell hybridoma by RTL1000. Altogether, these results demonstrate that RTL1000 is highly purified, monomeric, biotinylated and biologically active. The data presented in FIGS. 1A-E are representative of at least three independent experiments.

FIGS. 2A-E are histograms (FIGS. 2A and 2E) and graphs (FIGS. 2B, 2C and 2D) demonstrating the specificity of recombinant Fab Ab phage clones selected on β1α1DR2/MOG-35-55 complexes (RTL1000). FIG. 2A—a histogram depicting a representative supernatant ELISA of Fab clones selected against RTL1000. Fabs 2B4, 2C3, 2C10, 2E2, 2E4, 2F5, 2F9 (marked by arrows) specifically bind the DR2/MOG-35-55 complex but not the control DR2 complex containing the DR2-restricted MBP peptide (RTL340) and were selected for further characterization. FIGS. 2B-D are graphs depicting the binding of soluble purified Fabs 2E4 (FIG. 2B), 2C3 (FIG. 2C) and 2B4 (FIG. 2D) to immobilized α1β1DR2/MOG-35-55 complex in the presence of various concentrations of the following competitors: β1α1DR2/MOG-35-55 (squares), β1α1DR2/MBP-85-99 (triangles), MOG-35-55 peptide (diamonds), or MBP-85-99 peptide (X). Y axis in each of FIGS. 2B, 2C and 2D—% of maximal binding; X axis in each of FIGS. 2B, 2C and 2D—Log competitor in micromolar (μM). Data are representative of three independent experiments. FIG. 2E—a histogram depicting the binding of soluble purified Fabs or an anti-MHC II mAb TU39 (BD) to immobilized β1α1DR2/MOG-35-55 (DR2/MOG-35-55), control complexes (DR2/MBP-85-99), empty DR2 or MOG-35-55 peptide. Data are representative of four independent experiments. Note the specific binding of soluble purified Fab 2C3, 3A3, 1F11, 2E4, and 3H5 to the DR2/MOG-35-55 complex but not to the control complex DR2/MBP-85-99, empty DR2 (in the absence of the restricted peptide) or MOG-35-55 peptide in the absence of the DR2 molecule, demonstrating that Fabs 2C3, 3A3, 1F11, 2E4, and 3H5 bind to the two-domain MHC class II in a TCRL manner, i.e., only when in complex with the specific peptide (i.e., the DR2/MOG-35-55 two-domain-peptide complex) but not to the two-domain MHC class II when in complex with a non-specific peptide (e.g., the DR2/MBP-85-95 complex) or to an empty two-domain MHC class II molecule (DR2 molecule). Also note that Fab 1B11 binds to DR2/MOG-35-55, DR2/MBP-85-99 and empty DR2 but not to MOG-35-55, indicating that Fab 1B11 recognizes the two-domain MHC class II in a non-peptide specific manner.

FIGS. 3A-B are histograms depicting ELISA assays with the purified soluble Fabs of some embodiments of the invention, demonstrating fine specificity of anti-RTL1000 TCRLs. FIG. 3A—ELISA assay depicting the binding of the soluble Fabs (TCRLs) selected against the DR2/hMOG-35-55 complex (e.g., Fabs 2E4, 1F11, 3A3, 2C3 and 3H5) to various to complexes. Note the specific binding of the Fabs to the RTL1000 (DR2/hMOG-35-55) complex as compared to the low or no binding of the Fabs to RTL342m (DR2/mMOG-35-55) or RTL551 (I-Ab/mMOG-35-55) complexes. These results demonstrate that the Fabs can distinguish between β1α1DR2/hMOG-35-55 complex (RTL1000) and the β1α1DR2/mMOG-35-55 complex (RTL342m). Data are representative of three independent experiments. FIG. 3B—ELISA assay depicting binding assay of the soluble Fabs (e.g., 2E4, 1F11, 3H5, 3A3 or 2C3) to empty β1α1DR2-RTL alone or empty β1α1DR2-RTL loaded with MOG-35-55 peptide. Data are representative of one independent experiment out of two.

FIGS. 4A-G are FACS analyses (FIGS. 4A-F) and a histogram (FIG. 4G) depicting binding assays of the isolated soluble Fabs of some embodiments of the invention to DR2 APCs (L466.1 DR*1501 L cell transfectants) which were pulsed with MOG-35-55 or MBP-85-99 peptides. FIG. 4A—Anti-DR antibody; FIG. 4B—1F11 Fab; FIG. 4C—2C3 Fab; FIG. 4D—2E4 Fab; FIG. 4E—3A3 Fab; FIG. 4F—3H5 Fab. Note that while the anti-DR antibody bound to cells pulsed with either MOG-35-55 peptide or MBP-85-99 peptide, none of the RTL1000 specific Fabs (e.g., 1F11, 2C3, 2E4, 3A3, 3H5) bound to cells loaded with either the MOG-35-55 peptide or the MBP-85-99 peptide, thus demonstrating their specificity to the two-domain MHC class II molecule being in complex with the specific antigenic peptide but not to a native (four-extracellular domain) MHC class II being in complex with the specific peptide. FIG. 4G—a histogram depicting CTLL (an IL-2-dependent murine cell line) proliferation following incubation with APCs pulsed with MOG-35-55 or MBP-85-99 peptides. A portion of the APCs loaded with the MOG-35-55, MBP-85-99 or unloaded cells for the binding assay described in FIGS. 4A-F was tested for efficient peptide loading and therefore for their ability to activate IL-2 dependent CTLL proliferation. Note that while APCs which were loaded with the specific MOG-35-55 peptide activated the proliferation of the specific T-cell hybridoma, APCs which were not loaded with any peptide (Medium alone) or APCs which were loaded with the MBP-85-99 peptide caused no activation of the T-cell hybridoma, thus demonstrating the functionality of the MOG35-55 loaded-APCs in activating the specific T-cell hybridoma. The results presented in FIG. 4G demonstrate that the APCs used in FIGS. 4A-F were actually pulsed with the peptide. Data presented in FIGS. 4A-G is representative of at least three independent experiments.

FIGS. 5A-B are histograms depicting functionality of the isolated Fab antibodies according to some embodiments of the invention. FIG. 5A-A histogram depicting IL-2 dependent CTLL proliferation by the MOG-35-55 loaded-APCs in the presence or absence of the isolated RTL1000-specific Fabs of some embodiments of the invention. Note that the specific Ag-specific activation (by DR2*1501 APC) of H2-1 MOG-35-55 specific T-cell hybridoma was not affected by the anti-β1α1DR2/MOG Fabs 2C3, 3H5, 3A3, 1F11 and 2E4 as demonstrated by IL-2 secretion from the H2-1 T-cell hybridoma as compared to the inhibitory anti-MHC II Ab (TU39, BD). D2 is a control antibody directed against DR4/GAD-555-56 derived RTL. FIG. 5B-A histogram depicting ELISA assay testing the binding of the anti-two-domain β1α1DR2/MOG-35-55 Fabs and anti-MHC II (TU39, BD) to immobilized RTL1000 and full length recombinant DR2/MOG-35-55 complexes. Note lack of reactivity of the anti-two-domain β1α1DR2/MOG-35-55 Fabs to MOG peptide-loaded four-domain DR2 complexes. Data presented in FIGS. 5A-B is representative of at least three independent experiments. These results demonstrate that the anti-RTL1000 TCRLs distinguish between the two idiotopes: two- vs. four-domain DR2/MOG-35-55 complexes.

FIGS. 6A-B are a graph (FIG. 6A) and a histogram (FIG. 6B) demonstrating the functionality of the isolated antibodies according to some embodiments of the invention in neutralizing the effect of the recombinant T cell receptor ligand. FIG. 6A—Fab 2E4 or control Fab D2 were incubated in vitro for 2 hours at room temperature at 2:1 and 1:1 molar ratios with 20 μg RTL342m [β1α1DR2/mMOG-35-55; mMOG=mouse MOG)] and injected subcutaneously daily for 3 days (arrows) into DR2 mice with clinical signs of EAE (scores≧2.0) which were induced by mMOG-35-55 peptide/CFA/Ptx. Note the reduced EAE severity in positive control mice receiving RTL342m+buffer or mice receiving RTL342m+Fab D2 compared to negative control mice receiving TRIS-D5W (buffer). In contrast, incubation of the RTL342m with Fab 2E4 resulted in a significant neutralization of therapeutic effects of RTL342m on EAE in DR2*1501 mice in a dose dependent manner. FIG. 6B—Differences between the Cumulative Disease Indices of the experimental groups over the 14 day observation period were evaluated using the Mann-Whitney test. *p≦0.05; **p≦0.01; ***p≦0.001.

FIGS. 7A-D are histograms (FIGS. 7A-B) and graphs (FIGS. 7C-D) demonstrating that the isolated soluble Fabs according to some embodiments of the invention detect natural RTL-like two-domain MHC class II molecules and infused RTL1000 in serum and plasma samples of subjects having multiple sclerosis (MS) or pool of sera from control subjects. FIG. 7A—A histogram depicting quantitated results of ELISA assay using the Fab 1B11 antibody. Serum or plasma was collected prior to infusion of RTL1000 from MS subjects reference numbers 03-302, 04-402, 24, 40, 42 and 44 at time 0 (0 minutes; prior to infusion with the RTL1000), from MS subject reference No. 42 at 30 minutes after initiating infusion of 200 mg RTL1000; and MS subject reference No. 44 at 120 minutes after initiating infusion of 100 mg RTL1000; and from 3 healthy controls (pooled human sera). Differences between the samples and background were evaluated using two-tailed unpaired t-test. Note that Fab 1B11 detected variable amounts of RTL-like MHC class II material (antigens) in serum and plasma samples from MS subjects (MS subjects Nos. 03-302, 24 and 40 with significant amounts) and a pool of 3 healthy controls prior to administering the RTL1000 molecule, demonstrating that Fab 1B11 can bind to native RTL-like structures (molecules) and not only to RTL1000. FIG. 7B—A histogram depicting quantitated results of ELISA assay using the 2E4 Fab antibody (specific to RTL1000) or the 1B11 Fab antibody (specific to any RTL having the DR α1/β1). Note that Fab 2E4 detected RTL1000 in plasma samples from MS subjects only after infusion of the drug (MS subjects reference No. 42, at 30 minutes after infusion of RTL1000; and MS subject reference No. 44, at 120 minutes after infusion of RTL1000) and not prior to infusion of the drug, thus demonstrating its high specificity of Fab 2E4 to the RTL1000 molecule and not to RTL-like antigens present in blood. In contrast, Fab 1B11 detected RTL-like antigens prior to infusion of the RTL1000 drug and also the RTL1000 after infusion of the drug. These results demonstrate that while Fab 2E4 can discriminate between circulating RTL1000 and native RTL-like material present in the blood, Fab 1B11 binds to both RTL1000 and RTL-like material. Differences between pre- and post-infusion samples of each subject were evaluated using two-tailed paired t-test. FIG. 7C—Standard curves of various concentrations of RTL1000 and RTL340 that were used for calculating the concentration in serum and plasma samples of RTL and RTL-like material. The minimal thresholds for RTL detection were 12 ng/ml for Fab2E4 and 0.1 ng/ml for Fab 1B11. Data in FIGS. 7A-C are representative mean+/−SD of at least three independent experiments. Differences between pre- and post-infusion samples of each subject were evaluated using two-tailed paired t-test. *p≦0.05; **p≦0.01; ***p≦0.001. FIG. 7D—A graph depicting levels of RTL1000 in plasma of an MS patient (No. 42) during and following infusion with RTL1000. RTL1000 was infused for 120 minutes, and the presence and level of RTL1000 was monitored using the Fab 2E4 during infusion and for 60 minutes after RTL1000 infusion. Time from the beginning of RTL1000 infusion and the time after completion of the infusion are indicated by brackets. The completion of RTL1000 infusion is indicated by a dashed line. These results demonstrate the use of the RTL-specific antibodies for pharmacokinetics analysis of RTL drugs.

FIGS. 8A-C are histograms (FIGS. 8A and 8C) and a graph (FIG. 8B) showing binding characterization of G3H8 and D2 Fabs. FIG. 8A—ELISA of purified anti G3H8 (directed against the four-domain DR4/GAD-555-567 complex) or L243 (directed against DR molecule) with immobilized DR4/GAD-555-567 complex, control complex DR4/HA-307-319, GAD-555-567 peptide (SEQ ID NO:126), and HA-307-319 peptide (SEQ ID NO:196). Anti-DR mAb (L243) was used to determine the correct conformation and stability of the bound complexes during the binding assay. Note that while the G3H8 antibody specifically recognized the DR/GAD-555-567 complex, it did not bind to the DR/HA-307-319 control complex, or to the GAD-555-567 or HA-307-319 peptides. FIG. 8B—Flow cytometry analysis of Fab G3H8 binding to Preiss APCs pulsed with GAD-555-567 peptide (SEQ ID NO:126) or the control peptides: InsA-1-15 (SEQ ID NO:158), CII-261-273 (SEQ ID NO:195), and Ha-307-319 (SEQ ID NO:196). Note that the G3H8 binds specifically to APC loaded with the GAD-555-567 but not to the same cells loaded with any of the control peptides. FIG. 8C—Quantitation of ELISA assays showing conformational differences between RTL and full length MHC/peptide complex. Binding of anti-β1α1DR4/GAD-555-567 TCRL (D2), anti-full-length DR4/GAD-555-567 TCRL (G3H8) or anti-MHC II (TU39, BD) to immobilized β1α1DR4/GAD-555-567 RTL and full length DR4/GAD-555-567 complexes. Note that while the G3H8 binds to the four-domain MHC-peptide complex and not to the two-domain RTL, the D2 antibody binds to the two-domain RTL and not to the four-domain MHC-peptide complex. Data in FIGS. 8A-C are representative of at least three independent experiments.

FIGS. 9A-B are images of Western blot analyses using the 2E4 (FIG. 9A) and TU39 (FIG. 9B) antibodies demonstrating that TCRL Fabs against RTL1000 are conformationally-sensitive. RTL1000 were denatured by 2% SDS, 5% beta 2-mercaptoethanol and 10 minutes boiling, or treated in mild detergent conditions of 0.1% SDS (without beta 2-mercaptoethanol or boiling). Treated RTLs were analyzed by Western Blot for reactivity with Fab 2E4 or anti-DR-DP-DQ mAb (clone TU39). Note the low reactivity of 2E4 for RTL1000 at 0.1% SDS compared to TU39. TCRL Fab Clones 1F11, 3A3, 3H5, and 2C3 completely lost their ability to bind RTL1000 in 0.1% SDS (data not shown).

FIG. 10 is a histogram depicting quantitated results of ELISA assays using the isolated soluble Fab 1B11. Binding of purified 1B11 Fab to immobilized RTLs (RTL101, RTL200, RTL400, RTL450, RTL550, RTL600, RTL800, RTL1000, RTL340, RTL302, RTL350 and RTL2010) and four-domain recombinant MHC complexes (DR4/GAD-555-567 and DR2/MBP-85-99). 1B11 Fab binds to the HLA-DR derived two-domain MHC complexes [DR2/MOG-35-55, DR2/MBP-85-99, DR2 (empty), DR4/GAD-555-567 and DR3 (empty)], while no binding to non-HLA-DR derived two-domain [Rat-RT1.B (empty), RT1.B/MBP-72-89; mouse-I-As (empty), I-Ag7(empty), I-Ab (empty); human-DQ2(empty) and DP2 (empty)] or four-domain MHC complexes (DR4/GAD-555-567 and DR2/MBP-85-99) was obtained. *—Two-domain (RTL2010) and four-domain DR4/GAD-555-567 complexes were compared only to RTL1000. Data are representative of three independent experiments

FIGS. 11A-B are flow cytometry analyses depicting binding characterization of Fab D2. FIG. 11A—Flow cytometry analysis of Fab D2 binding to Preiss APCs pulsed with GAD-555-567 peptide or the control HA-307-319 peptide. Control=Secondary Ab alone (no Fab). Note the lack of binding of Fab D2 to APCs presenting the HLA-DR4-GAD555-567 complex. FIG. 11B—Preiss APCs cells pulsed with GAD-555-567 peptide or the control HA-307-319 peptide were simultaneously stained with anti-HLA-DR (TU39). Note the binding of the TU39 antibody to APCs presenting the DR4/GAD555-567 complex. The result show that while the APCs express high level of HLA-DR as detected by binding with the TU39 antibody (FIG. 11B), Fab D2 does not recognize native DR4/GAD-555-567 complexes presented by APC.

FIGS. 12A-C are schematic illustrations depicting a recombinant T cell receptor ligand (FIG. 12A) and three-dimensional model of the MHC class II molecule (FIG. 12B) and the recombinant T cell receptor ligand (FIG. 12C). FIG. 12 A—Recombinant T cell receptor ligand according to some embodiments of the invention in which the antigenic peptide is covalently linked (e.g., via a linker peptide) upstream of the β1 domain of an MHC class II; and the β1 domain is covalently linked upstream of the α1 domain of the MHC class II; and the α1 domain is covalently linked upstream to peptide for directing site-specific biotinylation (e.g., BirA tag). FIG. 12B—a three dimensional schematic illustration [Burrows G G, Chang J W, Bächinger H P, Bourdette D N, Offner H, Vandenbark A A. Design, engineering and production of functional single-chain T cell receptor ligands. Protein Eng. 1999 September; 12(9):771-8] depicting an MHC class II complex composed of two chains: α1-α2 MHC class II (red) and β1-β2 MHC class II (blue), of which the α1 and β1 domains are extracellular. FIG. 12C—a three dimensional illustration depicting the recombinant T cell receptor ligand comprising the β1 (blue) and α1 (red) domains of MHC class II conjugated to a BirA tag (grey) at the C-Terminus of α1; the antigenic peptide is linked to the N-terminus of the β1 (black). Note that the β1 and α1 domains are covalently-linked.

FIGS. 13A-D depict the sequences of RTL1000-BirA (FIGS. 13A-B; DR2 RTL with MOG-35-55 peptide) and RTL 340 BirA (FIGS. 13C-D; DR2 RTL with MBP-85-99 peptide). FIG. 13A—amino acid sequence of RTL1000-BirA (SEQ ID NO:151); FIG. 13B—nucleic acid sequence of RTL1000-BirA (SEQ ID NO:170); FIG. 13C—amino acid sequence of RTL340-BirA (SEQ ID NO:152); FIG. 13D—nucleic acid sequence of RTL340-BirA (SEQ ID NO:193). Color index: Blue—antigenic peptide: MHC class II-restricted MOG-35-55 antigenic peptide [MEVGWYRPPFSRVVHLYRNGK; SEQ ID NO:129; or the nucleic acid sequence encoding same (SEQ ID NO:171) in FIG. 13A-B] or the MHC class II-restricted MBP-85-99 antigenic peptide [MENPVVHFFKNIVTPR; SEQ ID NO:130; or the nucleic acid encoding same (SEQ ID NO:192)]. Black—linker between antigenic peptide and β1 domain [GGGGSLVPRGSGGGG; SEQ ID NO:153) or the nucleic acid encoding same (SEQ ID NO:172)]; Grey—the sequence of the β1 domain (PRFLWQPKRECHFFNGTERVRFLDRYFYNQEESVRFDSDVGEFRAVTELGRPD AEYWNSQKDILEQARAAVDTYCRHNYGVVESFTVQRRV; SEQ ID NO:154) or the nucleic acid encoding same (SEQ ID NO:173)]; Red—the sequence of the α1 domain (IKEEHDIDQDEDYDNPDQSGEFMFDFDGDEIFHVDMAKKETVWRLEEFGRFAS FEAQGALANIAVDKANLEIMTKRSNYTPITN; SEQ ID NO:155) or the nucleic acid encoding same (SEQ ID NO:174) in red; Highlighted in yellow—the sequence of the linker peptide connecting the α1 domain with the BirA tag (GGGGSGGGGSGGGGSGGGGS; SEQ ID NO:156) or the nucleic acid encoding same (SEQ ID NO:175); Highlighted in purple—the sequence of the BirA tag (LHHILDAQKMVWNHR; SEQ ID NO:157) or the nucleic acid encoding same (SEQ ID NO:176)].

FIGS. 14A-D depict the sequences of RTL2011 (FIGS. 14A-B; DR4 RTL with Insulin A-1-15 peptide) and RTL2010 (FIGS. 14C-D; DR4 RTL with GAD-555-567 peptide). FIG. 14A—amino acid sequence of RTL2011-BirA (SEQ ID NO:168); FIG. 14B—nucleic acid sequence encoding RTL2011-BirA (SEQ ID NO:181); FIG. 14C—amino acid sequence of RTL2010-BirA (SEQ ID NO:169); FIG. 14D—nucleic acid sequence encoding RTL2010-BirA (SEQ ID NO:182). Color index: Blue: MHC class II insulin A1 antigenic peptide [GIVEQCCTSICSLYQ (SEQ ID NO:158, FIG. 14A) or the nucleic acid encoding same (SEQ ID NO:177, FIG. 14B); the MHC class II GAD-555-567 antigenic peptide [MFFRMVISNPAAT (SEQ ID NO:126, FIG. 14C) or the nucleic acid encoding same (SEQ ID NO:183, FIG. 14D); Black—the linker connecting the antigenic peptide and the β1-domain [GSGSGSGS (SEQ ID NO:165) or the nucleic acid encoding same (SEQ ID NO:178); Grey—the β1 domain DR4 [GDTRPRFLEQVKHECHFFNGTERVRFLDRYFYHQEEYVRFDSDVGEYRAVTEL GRPDAEYWNSQKDLLEQKRAAVDTYCRHNYGVGESFTVQRRV (SEQ ID NO:166) or the nucleic acid encoding same (SEQ ID NO:179); Red—the α1 domain DR4 [IKEEHVIIQAEFYLNPDQSGEFMFDFDGDEIFHVDMAKKETVWRLEEFGRFASF EAQGALANIAVDKANLEIMTKRSNYTPITN (SEQ ID NO:167)] or the nucleic acid encoding same (SEQ ID NO:180); Highlighted in yellow—the linker connecting the α1 domain and the BirA tag [GGGGSGGGGSGGGGSGGGGS (SEQ ID NO:156)] or the nucleic acid encoding same (SEQ ID NO:175); Highlighted in purple—the BirA tag [LHHILDAQKMVWNHR (SEQ ID NO:157)] or the nucleic acid encoding same (SEQ ID NO:176) highlighted in purple.

FIGS. 15A-D depict the light chain (FIGS. 15A-B) and heavy chain (FIGS. 15C-D) sequences of Fab 2E4. FIG. 15A—amino acid sequence light chain (SEQ ID NO:13). CDRs 1-3 are marked in yellow (SEQ ID NOs:1-3, respectively). FIG. 15B nucleic acid sequence encoding the light chain (SEQ ID NO:14). CDRs 1-3 are marked in yellow (SEQ ID NOs:4-6, respectively). FIG. 15C—amino acid sequence heavy chain (SEQ ID NO:15). CDRs 1-3 are marked in yellow (SEQ ID NOs:7-9, respectively). Blue (CH1); red (connector); purple (His tag); green (Myc tag). FIG. 15D—nucleic acid sequence encoding the heavy chain (SEQ ID NO:16). CDRs 1-3 are marked in yellow (SEQ ID NOs:10-12, respectively). Blue (CH1); red (connector); purple (His tag); green (Myc tag).

FIGS. 16A-D depict the light chain (FIGS. 16A-B) and heavy chain (FIGS. 16C-D) sequences of Fab 1F11. FIG. 16A—amino acid sequence light chain (SEQ ID NO:29). CDRs 1-3 are marked in yellow (SEQ ID NOs:17-19, respectively). FIG. 16B—nucleic acid sequence encoding the light chain (SEQ ID NO:30). CDRs 1-3 are marked in yellow (SEQ ID NOs:20-22, respectively). FIG. 16C—amino acid sequence heavy chain (SEQ ID NO:31). CDRs 1-3 are marked in yellow (SEQ ID NOs:23-25, respectively). Blue (CH1); red (connector); purple (His tag); green (Myc tag). FIG. 16D—nucleic acid sequence encoding the heavy chain (SEQ ID NO:32). CDRs 1-3 are marked in yellow (SEQ ID NOs:26-28, respectively). Blue (CH1); red (connector); purple (His tag); green (Myc tag).

FIGS. 17A-D depict the light chain (FIGS. 17A-B) and heavy chain (FIGS. 17C-D) sequences of Fab 3A3. FIG. 17A—amino acid sequence light chain (SEQ ID NO:45). CDRs 1-3 are marked in yellow (SEQ ID NOs:33-35, respectively). FIG. 17B—nucleic acid sequence encoding the light chain (SEQ ID NO:46). CDRs 1-3 are marked in yellow (SEQ ID NOs:36-38, respectively). FIG. 17C—amino acid sequence heavy chain (SEQ ID NO:47). CDRs 1-3 are marked in yellow (SEQ ID NOs:39-41, respectively). Blue (CH1); red (connector); purple (His tag); green (Myc tag). FIG. 17D—nucleic acid sequence encoding the heavy chain (SEQ ID NO:48). CDRs 1-3 are marked in yellow (SEQ ID NOs:42-44, respectively). Blue (CH1); red (connector); purple (His tag); green (Myc tag).

FIGS. 18A-D depict the light chain (FIGS. 18A-B) and heavy chain (FIGS. 18C-D) sequences of Fab 3H5. FIG. 18A—amino acid sequence light chain (SEQ ID NO:61). CDRs 1-3 are marked in yellow (SEQ ID NOs:49-51, respectively). FIG. 18B—nucleic acid sequence encoding the light chain (SEQ ID NO:62). CDRs 1-3 are marked in yellow (SEQ ID NOs:52-54, respectively). FIG. 18C—amino acid sequence heavy chain (SEQ ID NO:63). CDRs 1-3 are marked in yellow (SEQ ID NOs:55-57, respectively). Blue (CH1); red (connector); purple (His tag); green (Myc tag). FIG. 18D—nucleic acid sequence encoding the heavy chain (SEQ ID NO:64). CDRs 1-3 are marked in yellow (SEQ ID NOs:58-60, respectively). Blue (CH1); red (connector); purple (His tag); green (Myc tag).

FIGS. 19A-D depict the light chain (FIGS. 19A-B) and heavy chain (FIGS. 19C-D) sequences of Fab 2C3. FIG. 19A—amino acid sequence light chain (SEQ ID NO:77). CDRs 1-3 are marked in yellow (SEQ ID NOs:65-67, respectively). FIG. 19B—nucleic acid sequence encoding the light chain (SEQ ID NO:78). CDRs 1-3 are marked in yellow (SEQ ID NOs:68-70, respectively). FIG. 19C—amino acid sequence heavy chain (SEQ ID NO:79). CDRs 1-3 are marked in yellow (SEQ ID NOs:71-73, respectively). Blue (CH1); red (connector); purple (His tag); green (Myc tag). FIG. 19D—nucleic acid sequence encoding the heavy chain (SEQ ID NO:80). CDRs 1-3 are marked in yellow (SEQ ID NOs:74-76, respectively). Blue (CH1); red (connector); purple (His tag); green (Myc tag).

FIGS. 20A-D depict the light chain (FIGS. 20A-B) and heavy chain (FIGS. 20C-D) sequences of Fab 1B11. FIG. 20A—amino acid sequence light chain (SEQ ID NO:93). CDRs 1-3 are marked in yellow (SEQ ID NOs:81-83, respectively). FIG. 20B—nucleic acid sequence encoding the light chain (SEQ ID NO:94). CDRs 1-3 are marked in yellow (SEQ ID NOs:84-86, respectively). FIG. 20C—amino acid sequence heavy chain (SEQ ID NO:95). CDRs 1-3 are marked in yellow (SEQ ID NOs:87-89, respectively). Blue (CH1); red (connector); purple (His tag); green (Myc tag). FIG. 20D—nucleic acid sequence encoding the heavy chain (SEQ ID NO:96). CDRs 1-3 are marked in yellow (SEQ ID NOs:90-92, respectively). Blue (CH1); red (connector); purple (His tag); green (Myc tag).

FIGS. 21A-D depict the light chain (FIGS. 21A-B) and heavy chain (FIGS. 21C-D) sequences of Fab D2. FIG. 21A—amino acid sequence light chain (SEQ ID NO:109). CDRs 1-3 are marked in yellow (SEQ ID NOs:97-99, respectively). FIG. 21B—nucleic acid sequence encoding the light chain (SEQ ID NO:110). CDRs 1-3 are marked in yellow (SEQ ID NOs:100-102, respectively). FIG. 21C—amino acid sequence heavy chain (SEQ ID NO:111). CDRs 1-3 are marked in yellow (SEQ ID NOs:103-105, respectively). Blue (CH1); red (connector); purple (His tag); green (Myc tag). FIG. 21D—nucleic acid sequence encoding the heavy chain (SEQ ID NO:112). CDRs 1-3 are marked in yellow (SEQ ID NOs:106-108, respectively). Blue (CH1); red (connector); purple (His tag); green (Myc tag).

FIGS. 22A-B depict the amino acid (FIG. 22A; SEQ ID NO:159) and nucleic acid (FIG. 22B; SEQ ID NO:160) of the empty RTL800 which comprises the 2-domain HLA-DQ2 MHC class II. The β1 domain (SEQ ID NO:184) and the DNA encoding the β1 domain (SEQ ID NO:186) is marked in blue; the α1 domain (SEQ ID NO:185) and the DNA encoding the α1 domain (SEQ ID NO:187) is in black.

FIGS. 23A-B depict the amino acid (FIG. 23A; SEQ ID NO:161) and nucleic acid (FIG. 23B; SEQ ID NO:162) of the empty RTL600 which comprises the 2-domain HLA-DP2 MHC class II. The β1 domain (SEQ ID NO:188) and the DNA encoding the β1 domain (SEQ ID NO:190) is marked in blue; the α1 domain (SEQ ID NO:189) and the DNA encoding the α1 domain (SEQ ID NO:191) is in black.

FIGS. 24A-B depict the amino acid (FIG. 24A; SEQ ID NO:163) and nucleic acid (FIG. 24B; SEQ ID NO:164) of the empty RTL302 which comprises the 2-domain HLA-DR2 MHC class II. Shown are the β1 domain in grey [(SEQ ID NO:154) and the DNA encoding the β1 domain (SEQ ID NO:173)], the α1 domain in red [(SEQ ID NO:155) and the DNA encoding the α1 domain (SEQ ID NO:174)], the linker connecting the α1 domain and the BirA tag highlighted in yellow [SEQ ID NO:157; and the DNA encoding same (SEQ ID NO:175)] and the BirA tag highlighted in purple [SEQ ID NO:157 and the DNA encoding same (SEQ ID NO:176)].

### DESCRIPTION OF SPECIFIC EMBODIMENTS OF THE INVENTION

The present invention, in some embodiments thereof, relates to high affinity entities which specifically bind soluble T cell receptor ligands (e.g., recombinant T cell ligands) in an either peptide specific or peptide non-specific manner, but which do not bind complexes of MHC class II-antigenic peptides (four-domain complex) or native four-domain MHC class II/peptide complexes when displayed on antigen presenting cells, and, more particularly, but not exclusively, to methods of generating same and using same for detecting presence/level of soluble T cell receptor ligands in a biological sample such as for determining a pharmacokinetic of a recombinant T cell receptor ligand; and to methods of sequestering soluble two domain T cell receptor ligands using specific high affinity entities (e.g., antibodies) and thus preventing/inhibiting their binding to T cell receptors or to RTL-like receptor on antigen presenting cells.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

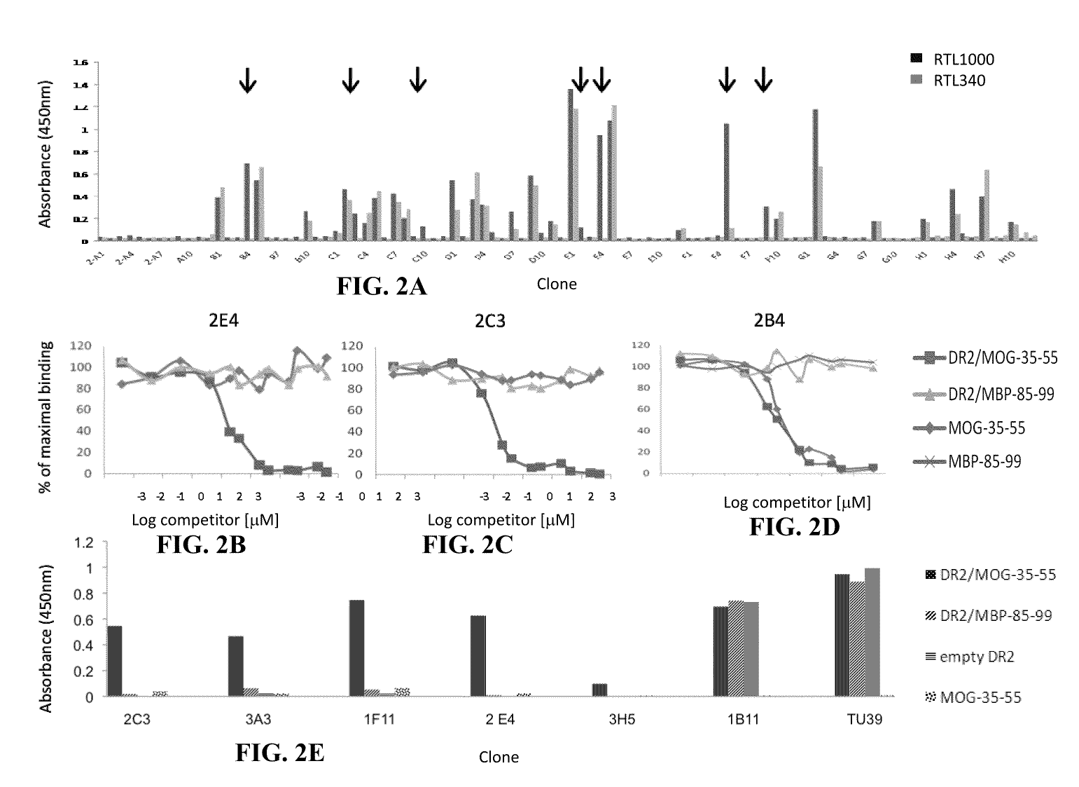

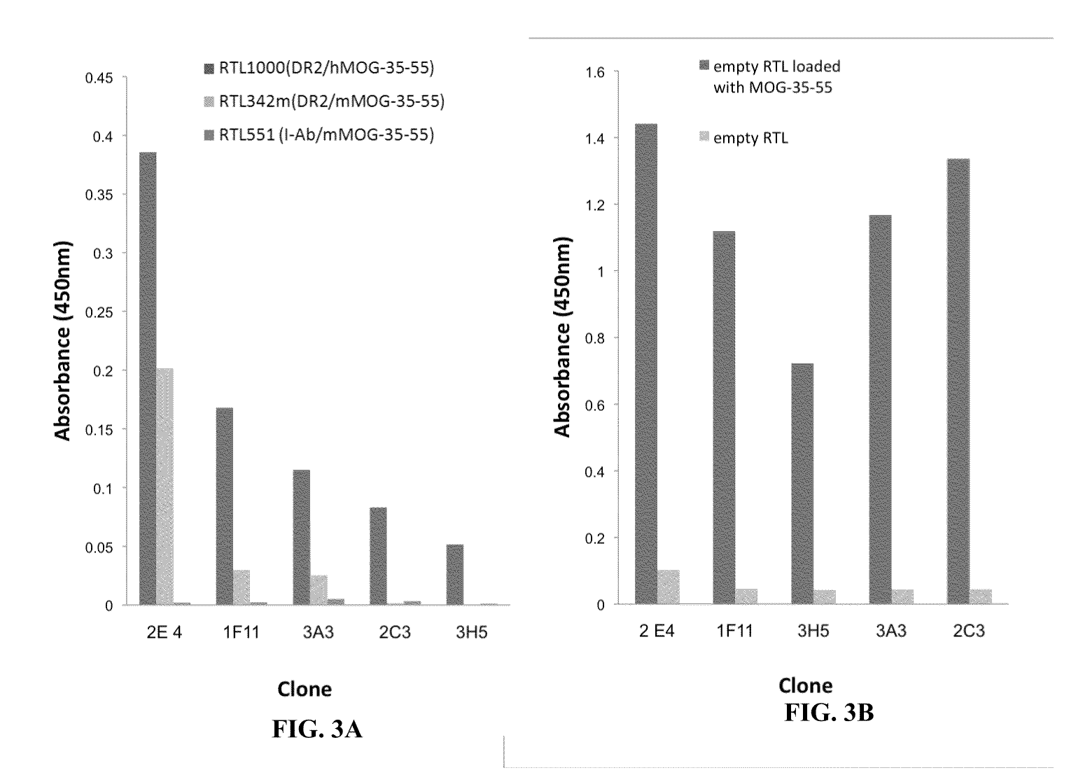

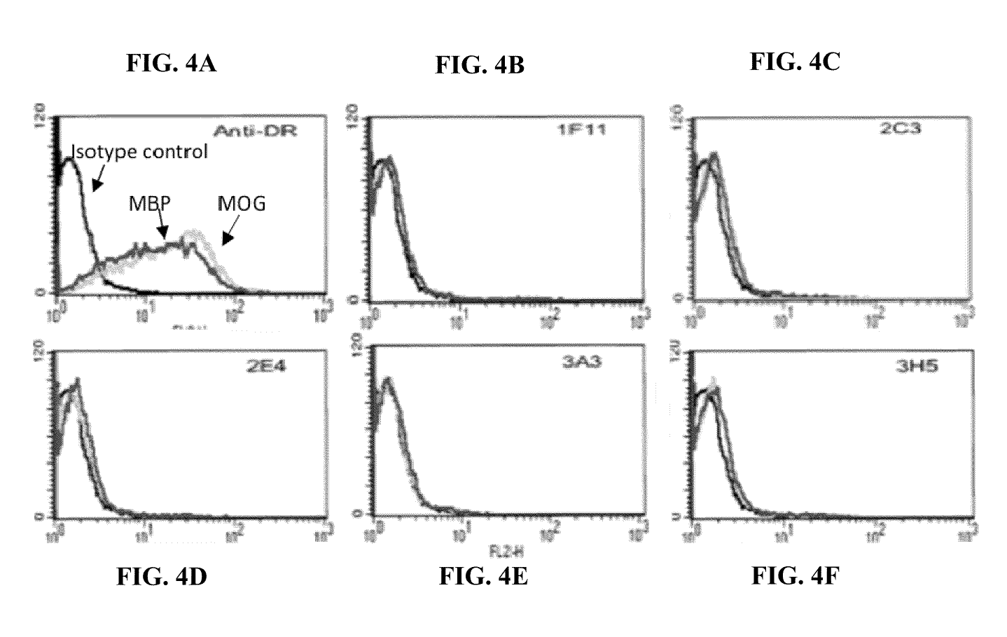

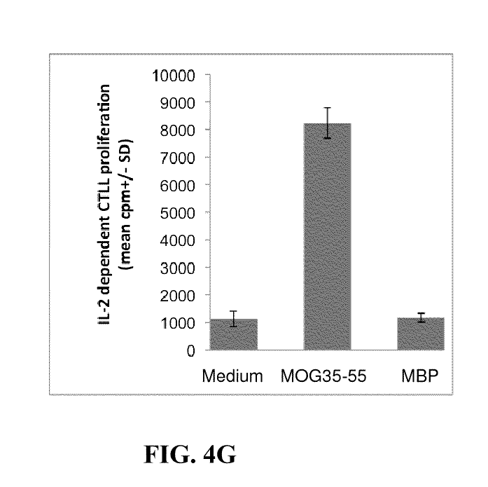

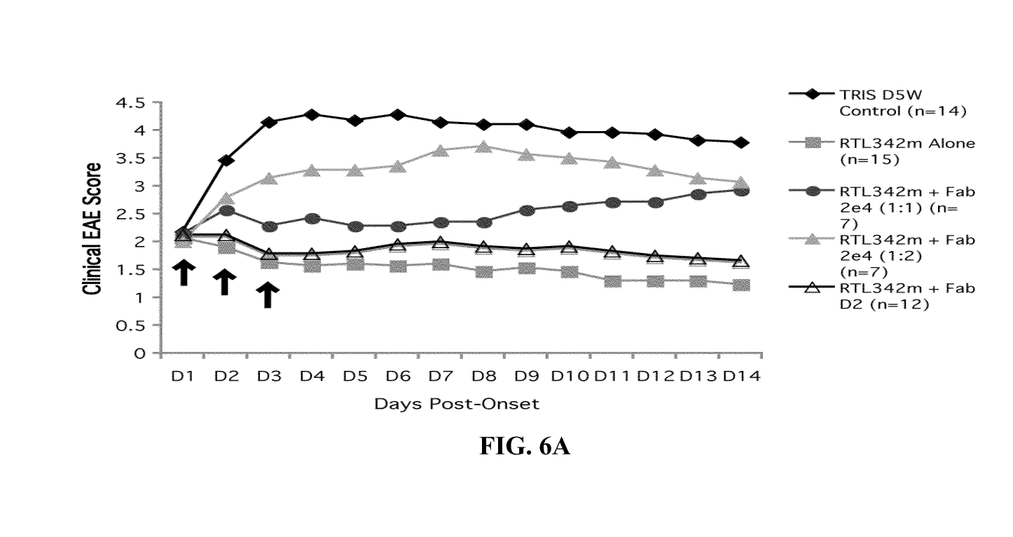

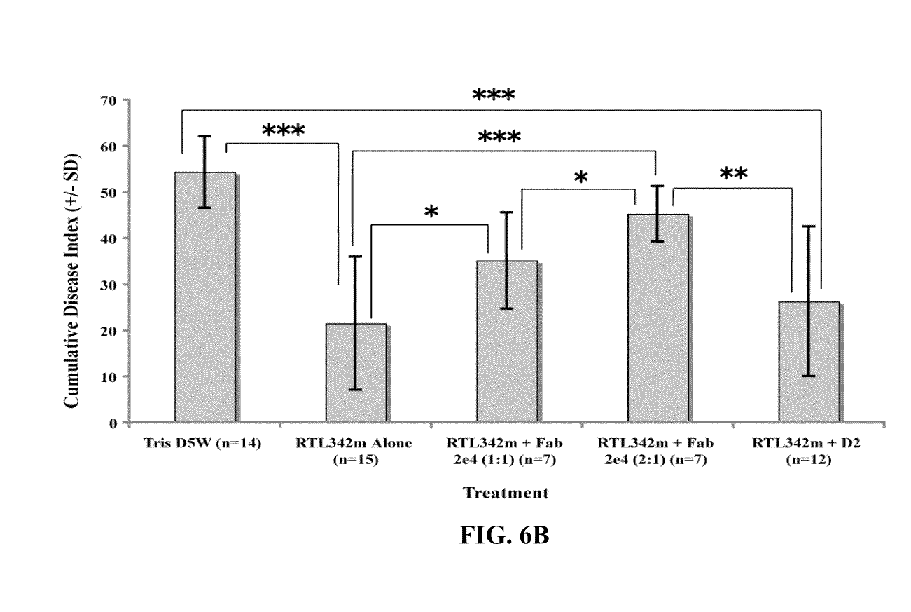

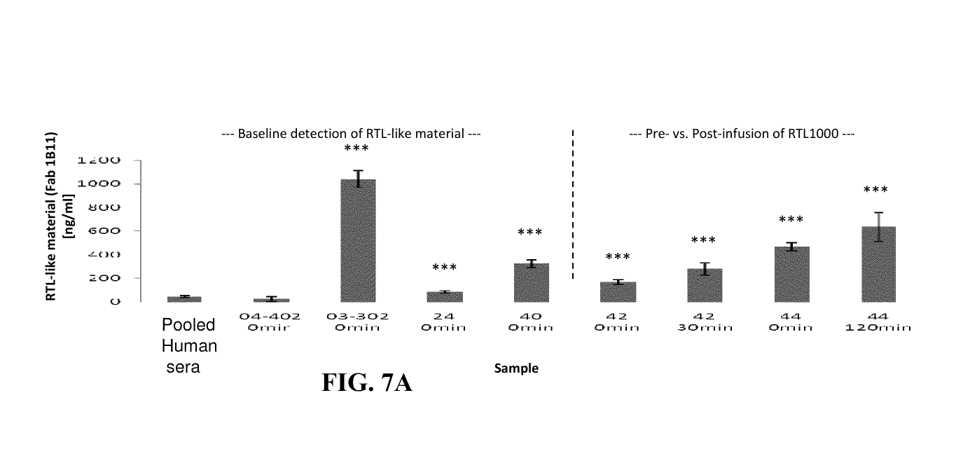

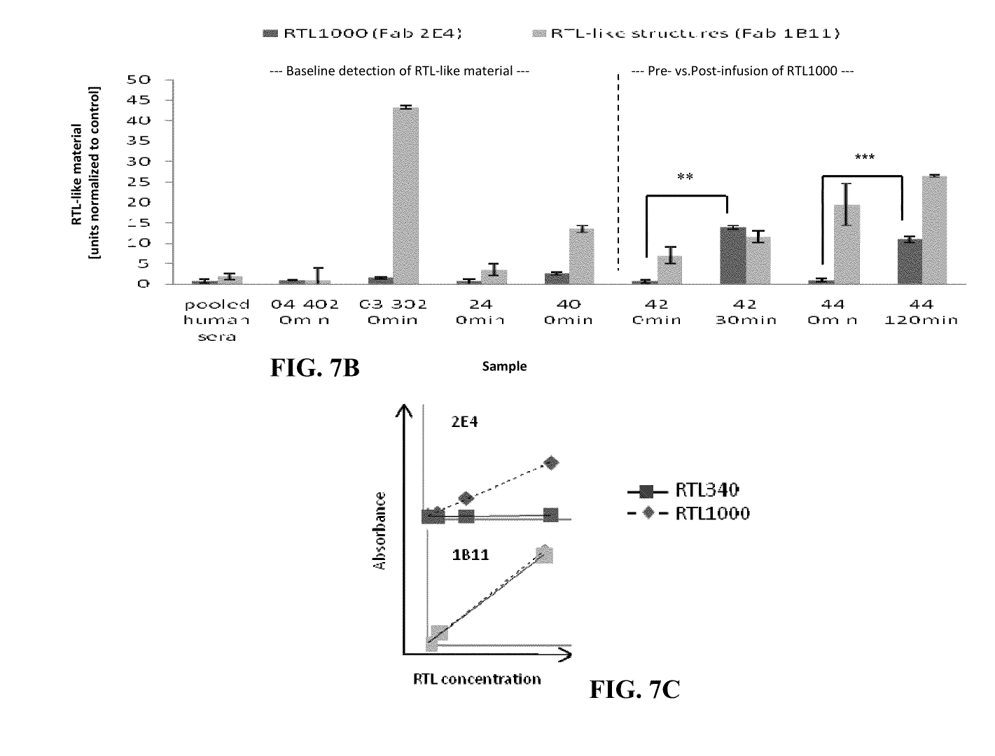

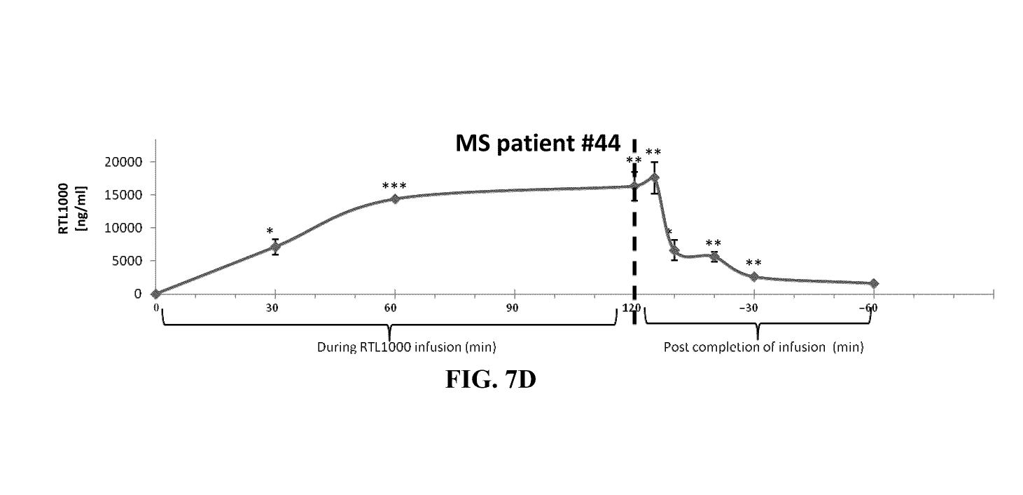

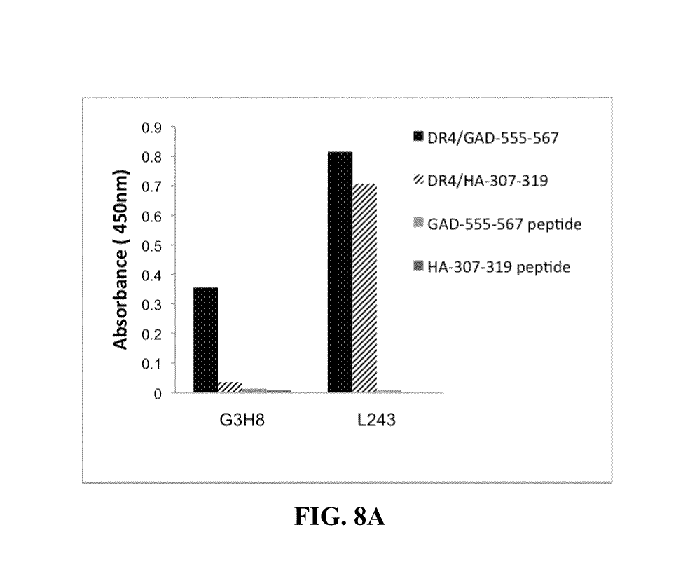

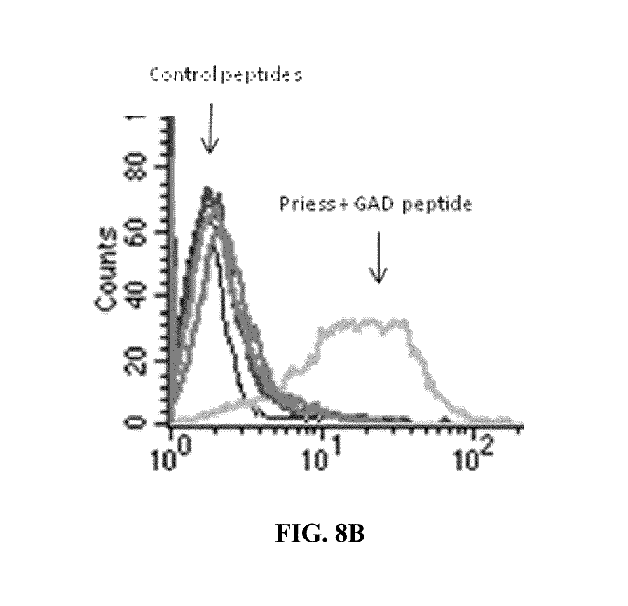

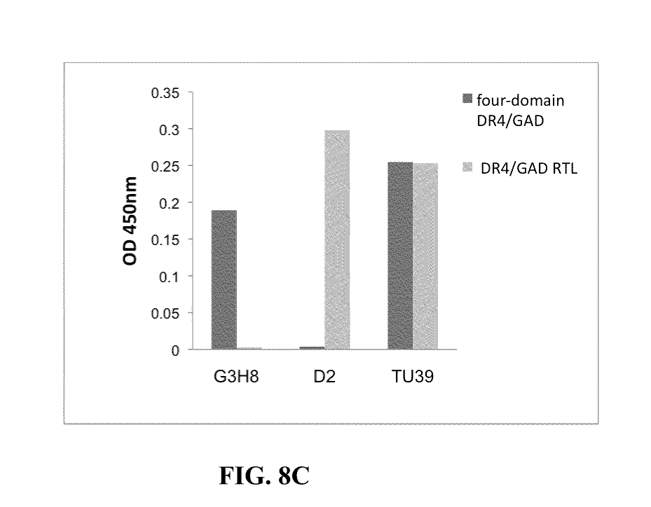

The present inventors isolated high affinity entities which bind soluble T cell receptor ligands comprising a two-domain β1-α1 MHC class II in complex with an MHC class II autoantigenic peptide. As shown in the Examples section which follows, the isolated human high affinity entities (e.g., Fabs 2C3, 3A3, 1F11, 2E4, 3H5 and D2) can distinguish between two-domain β1-α1 MHC class II and four-domain β1-β2/α1-α2 MHC class II complexes in a T-cell receptor like (TCRL) specificity, i.e., binding to the two-domain molecules only when in complex with the specific autoantigenic peptide against which the high affinity entity was selected, but not in the absence of an antigenic peptide (i.e., an empty two-domain molecule), nor when the two-domain molecule is in complex with another (e.g., not the specific) antigenic peptide (FIGS. 2A-E, Example 2; FIGS. 3A-B, Example 3; FIGS. 8A-C and 11A-B, Example 8). The human recombinant Fabs bound to and neutralized activity of the two-domain DR2/MOG-35-55 idiotope present in RTL1000 (FIGS. 6A-B, Example 5), but none of these Fabs recognized the four-domain DR2/MOG-35-55 idiotope present on native MHC (FIGS. 4A-F, 5A-B, Example 4). Thus, the TCRL antibodies could distinguish two-versus (vs.) four-domain idiotopes of the T1D-associated HLA-DR4/GAD-555-567 T-cell determinant (e.g., Fab D2); and a panel of Fabs selected against the DR2/MOG-35-55 idiotope of RTL1000 (e.g., Fabs 2C3, 3A3, 1F11, 2E4 and 3H5) distinguished RTL1000 from the native conformation of DR2/MOG-35-55 complexes presented by antigen presenting cells (APCs). Thus, Fabs directed at either two-domain RTLs (e.g., Fab D2) or native four-domain DR4/GAD-555-567 complexes (e.g., Fab G3H8) recognized the cognate structures but failed to react with the non-cognate idiotopes (FIGS. 8A-C, Example 8). These two novel groups of TCRL-Fabs demonstrate for the first time distinct conformational determinants characteristic of activating four-domain form of MHC class II vs. tolerogenic two-domain form of MHC class II idiotopes coupled to the same antigenic peptide involved in human autoimmune diseases. As is further shown in FIG. 7B-D and described in Examples 6-8 of the Examples section which follows, the isolated TCRL-Fabs (e.g., Fab 2E4) were capable of detecting the cognate RTLs in the plasma of a subject following administration of the specific RTL (e.g., RTL1000) in a manner correlating with the level of RTL1000, thus following the pharmacokinetics of the RTL1000 drug in the plasma. In addition, as shown in FIGS. 6A-B, the isolated Fabs (e.g., Fab 2E4) were able to neutralize the RTL1000 treatment of EAE animal models, thus demonstrating the in vivo functionality of the TCRL-Fabs directed at the two-domain RTL structure. Therefore, the TCRL-Fabs directed at the two-domain RTL structure represent a valuable tool to study Ag-specific therapeutic mechanisms.

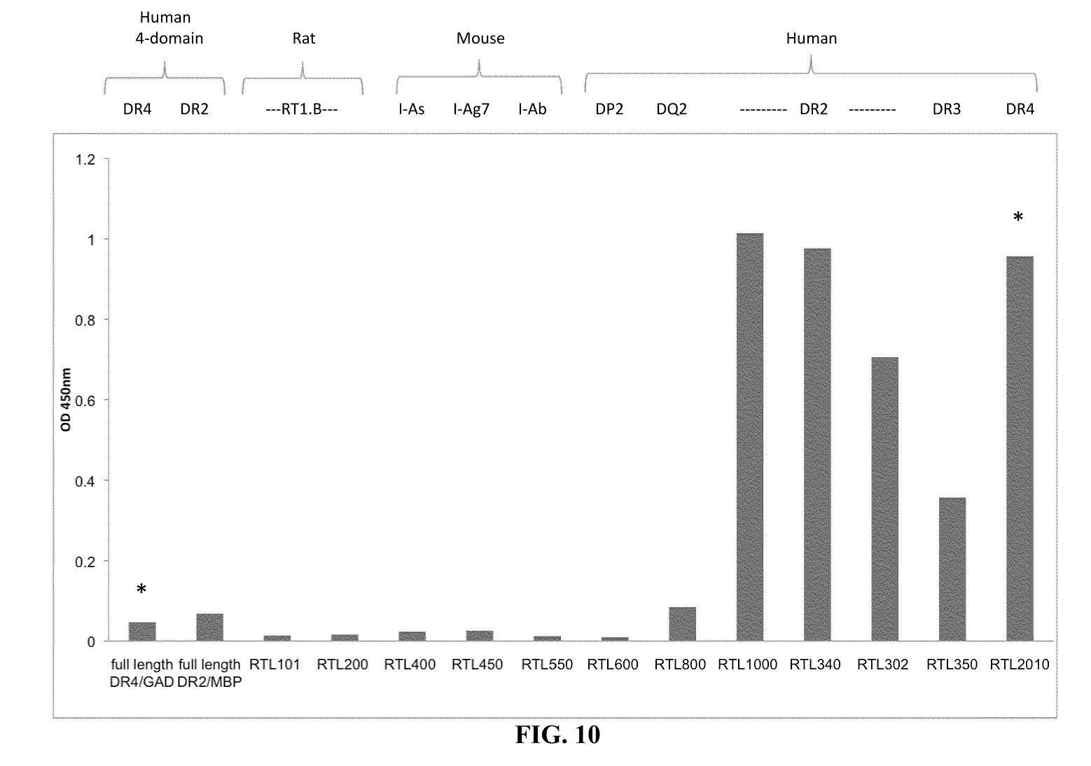

The present inventors have further uncovered Fabs which specifically bind the two-domain conformation of MHC class II (e.g., HLA-DR) in a manner which is specific to the MHC class II (i.e., to the specific HLA allele) but which is not-dependent on the presence or absence of the MHC class II specific antigen peptide. These Fabs (e.g., Fab 1B11) detect recombinant T cell receptor ligand like (RTL-like) structures in human sera/plasma even before administration of the recombinant T cell receptor ligand to a subject (FIG. 7A, Example 6 of the Examples section which follows), but only when the two-domain structure comprises the specific MHC class II allele (e.g., HLA-DR; FIG. 10, Example 6). The detection of native two-domain HLA-DR structures in human plasma implicates naturally-occurring regulatory idiotopes. This type of antibodies can be used to study the appearance of the yet-uncharacterized partial MHC class II structures in human serum and plasma.

According to an aspect of some embodiments of the invention, there is provided an isolated high affinity entity comprising an antigen binding domain which specifically binds a soluble T-cell ligand (RTL) comprising a two-domain β1-α1 of major histocompatibility complex (MHC) class II, wherein the antigen binding domain does not bind a complex comprising a four-domain α1-β1/α2-β2 MHC class II.

As used herein the phrase “major histocompatibility complex (MHC)” refers to a complex of antigens encoded by a group of linked loci, which are collectively termed H-2 in the mouse and human leukocyte antigen (HLA) in humans. The two principal classes of the MHC antigens, class I and class II, each comprise a set of cell surface glycoproteins which play a role in determining tissue type and transplant compatibility. In transplantation reactions, cytotoxic T-cells (CTLs) respond mainly against foreign class I glycoproteins, while helper T-cells respond mainly against foreign class II glycoproteins.

MHC class II molecules are expressed in professional antigen presenting cells (APCs) such as macrophages, dendritic cells and B cells. Each MHC class II molecule is a heterodimer composed of two homologous subunits, alpha chain (with α1 and α2 extracellular domains, transmembrane domain and short cytoplasmic tail) and beta chain (with β1 and β2 extracellular domains, transmembrane domain and short cytoplasmic tail). Peptides, which are derived from extracellular proteins, enter the cells via endocytosis, are digested in the lysosomes and further bind to MHC class II molecules for presentation on the membrane.

Various MHC class II molecules are found in humans. Examples include, but are not limited to HLA-DM, HLA-DO, HLA-DP, HLA-DQ (e.g., DQ2, DQ4, DQ5, DQ6, DQ7, DQ8, DQ9), HLA-DR (e.g., DR1, DR2, DR3, DR4, DR5, DR7, DR8, DR9, DR10, DR11, DR12, DR13, DR14, DR15, and DR16).

Non-limiting examples of DQ A1 alleles include 0501, 0201, 0302, 0301, 0401, 0101, 0102, 0104, 0102, 0103, 0104, 0103, 0102, 0303, 0505 and 0601.

Non-limiting examples of DQ B1 alleles include 0201, 0202, 0402, 0501, 0502, 0503, 0504, 0601, 0602, 0603, 0604, 0609, 0301, 0304, 0302 and 0303.

Non-limiting examples of DPA1 alleles include 01, e.g., 0103, 0104, 0105, 0106, 0107, 0108, 0109; 02, e.g., 0201, 0202, 0203; 03 e.g., 0301, 0302, 0303, 0401.

Non-limiting examples of DPB1 alleles include 01, e.g., 0101, 0102; 02 e.g., 0201, 0202, 0203; 03; 04, e.g., 0401, 0402, 0403; 05, e.g., 0501, 0502; 06; 08, e.g., 0801, 0802; 09, e.g., 0901, 0902; 10, e.g., 1001, 1002; 11 e.g., 1101, 1102; 13, e.g., 1301, 1302; 14, e.g., 1401, 1402; 15, e.g., 1501, 1502; 16, e.g., 1601, 1602; 17, e.g., 1701, 1702; 18, e.g., 1801, 1802; 19, e.g., 1901, 1902; 20, e.g., 2001, 2002; 21; 22; 23; 24; 25; 26, e.g., 2601, 2602; and 27.

Non-limiting examples of DP haplotypes include HLA-DPA1*0103/DPB1*0401 (DP401); and HLA-DPA1*0103/DPB1*0402 (DP402).

Non-limiting examples of DR B1 alleles include 0101, 0102, 0103, 0301, 0401, 0407, 0402, 0403, 0404, 0405, 0701, 0701, 0801, 0803, 0901, 1001, 1101, 1103, 1104, 1201, 1301, 1302, 1302, 1303, 1401, 1501, 1502, 1601 alleles.

Non-limiting examples of DR-DQ haplotypes include DR1-DQ5, DR3-DQ2, DR4-DQ7, DR4-DQ8, DR7-DQ2, DR7-DQ9, DR8-DQ4, DR8-DQ7, DR9-DQ9, DR10-DQ5, DR11-DQ7, DR12-DQ7, DR13-DQ6, DR13-DQ7, DR14-DQ5, DR15-DQ6, and DR16-DQ5.

As used herein the phrase “soluble T-cell receptor ligand” or “soluble two-domain T-cell receptor ligand”, which is interchangeably used herein, refers to a soluble (i.e., not membrane bound) polypeptide comprising the beta 1 (β1) and alpha 1 (α1) domains of an MHC class II beta and alpha chains, respectively, but being devoid of the β2 and α2 domains of the beta and alpha chains, respectively.

The soluble T-cell receptor ligand can be a recombinant polypeptide [recombinant T-cell receptor ligand (RTL)] or a native polypeptide [a native RTL-like structure].

As used herein the phrase “recombinant T-cell receptor ligand (RTL)” refers to a single chain polypeptide comprising the beta 1 (β1) and alpha 1 (α1) domains of an MHC class II beta and alpha chains, respectively, but being devoid of the β2 and α2 domains of the beta and alpha chains, respectively.

As used herein the phrase “native RTL-like structure” refers to a polypeptide or a polypeptide complex naturally present in body fluids (e.g., blood, plasma) of a subject and which exhibits a sequence and structural similarity to a recombinant T-cell receptor ligand such that an antigen binding domain of an antibody which specifically binds to the RTL is capable of binding to the native RTL-like structure with a comparable binding affinity.

It should be noted that while the α1 and β1 domains of the MHC class II are extracellular and form the antigen binding domain of the antigenic peptide, the α2 and β2 domains are membrane anchored domain(s).

According to some embodiments of the invention, the β1 and α1 domains are sufficient for forming the antigen binding domain which binds the MHC class II antigenic peptide.

According to some embodiments of the invention, the beta 1 domain comprises at least the amino acids at positions 1-90 of a HLA-DRB1*0401 beta chain (i.e., amino acids 1-90 of SEQ ID NO:201 which includes amino acids 1-192) of an MHC class II, but being devoid of the beta 2 domain (e.g., the amino acids at positions 91-192 of the beta chain of an MHC class II).

According to some embodiments of the invention, the alpha 1 domain comprises at least the amino acids at positions 1-81 of an HLA-DRA1*0101 alpha chain (i.e., amino acids 1-81 of SEQ ID NO:202) of an MHC class II, but being devoid of the alpha 2 domain (e.g., the amino acids at positions 82-181 of the alpha chain of an MHC class II).

The soluble T cell receptor ligand (e.g., the RTL) can bind to the antigenic peptide to form a complex of soluble two-domain T cell receptor ligand—peptide (e.g., RTL-peptide), which imitates the four-domain complex formed naturally on antigen presenting cells in which the MHC class II molecules bind the antigenic peptide.

According to some embodiments of the invention, the complex is non-covalently.

According to some embodiments of the invention the RTL is covalently bound to the MHC class II antigenic peptide.

According to some embodiments of the invention, the C-terminus of the antigenic peptide is covalently bound to the N-terminus of the β1 domain of the MHC class II beta chain.

According to some embodiments of the invention, the antigenic peptide is covalently embedded between amino acids 1-6 of the beta 1 domain of the MHC class II beta chain.

According to some embodiments of the invention, the C-terminus of the antigenic peptide is flanked by a linker peptide. Such a linker peptide connects between the antigenic peptide and the β1 domain.

According to some embodiments of the invention, the antigenic peptide is translationally fused to the β1 domain (i.e., form a single open reading frame).

The RTL can be produced by means of recombinant DNA technology by expressing in a host cell [e.g., Escherichia coli strain BL21(DE3) cells] a nucleic acid construct comprising a polynucleotide encoding the β1-α1 domains, with or without a nucleotide sequence encoding the antigenic peptide, under the transcriptional regulation of a promoter sequence. The recombinant polypeptide is further purified and isolated, essentially as described in the Examples section which follows and in Burrows et al., 1999; Burrows et al., 2001; Chang et al., 2001, each of which is incorporated herein by reference in its entirety.

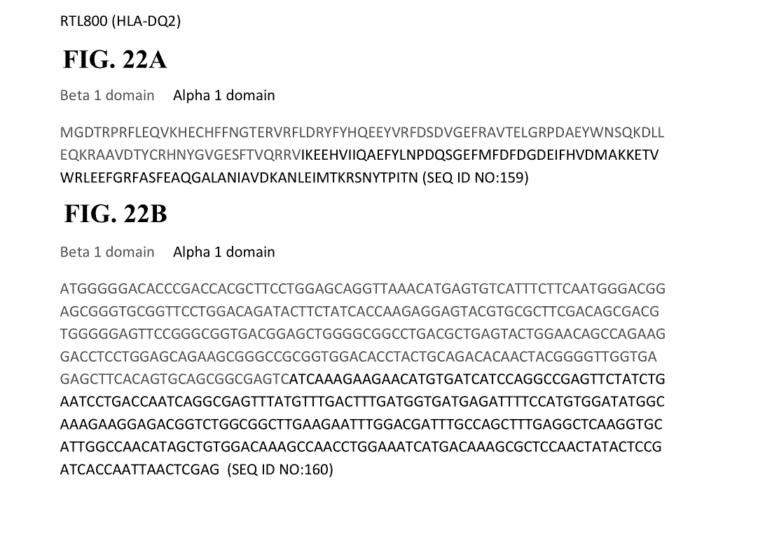

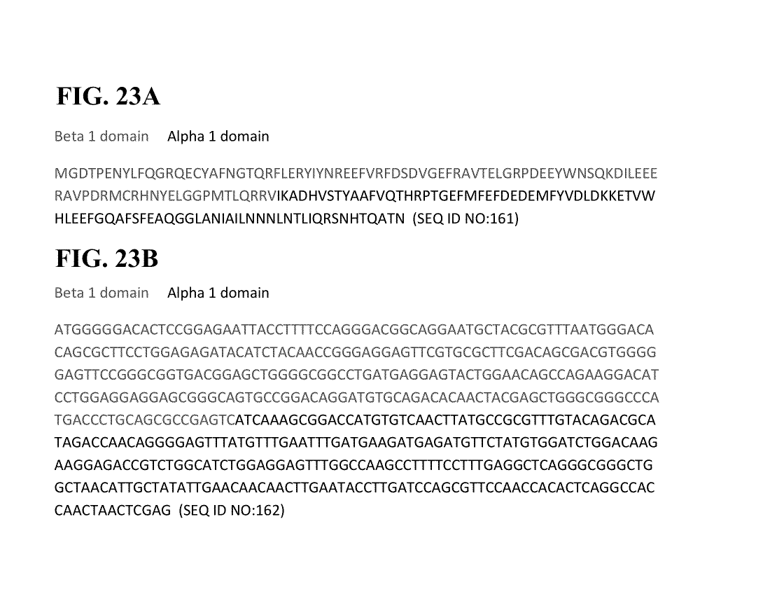

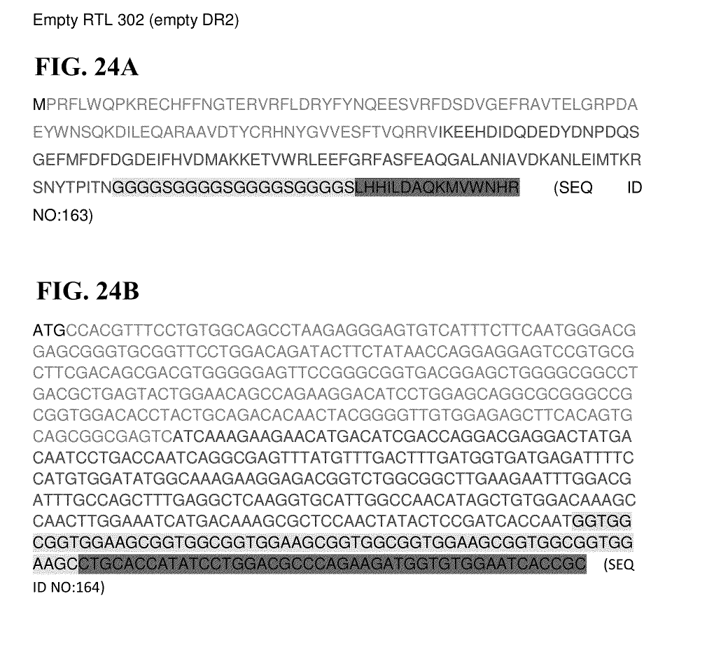

Following are non-limiting examples of empty RTL molecules which can be generated and used according to some embodiments of the invention: RTL302 (empty HLA-DR2-RTL as set forth by SEQ ID NO:163; FIG. 24A); RTL600 (empty HLA-DP2-RTL as set forth by SEQ ID NO: 161; FIG. 23A); RTL800 (empty HLA-DQ2-RTL as set forth by SEQ ID NO:159; FIG. 22A).

Non-limiting examples of coding sequences encoding the empty RTLs are provided in SEQ ID NOs: 160 (RTL800; FIG. 22B); 162 (RTL600; FIG. 23B) and 164 (RTL302; FIG. 24B).

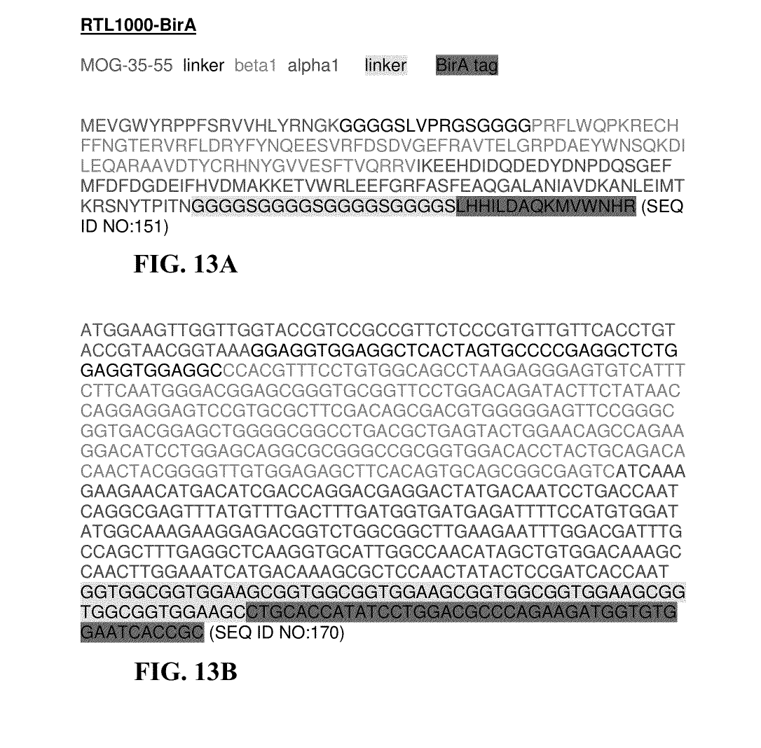

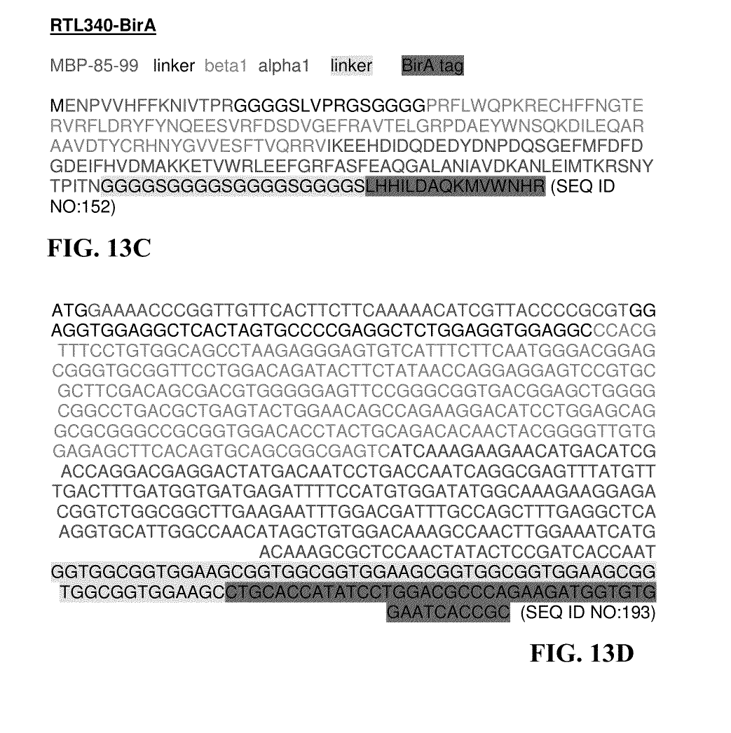

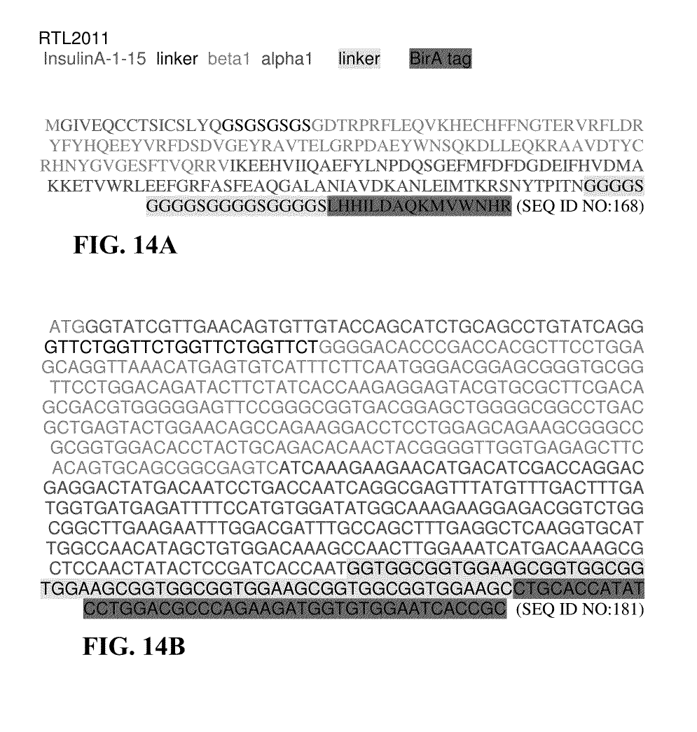

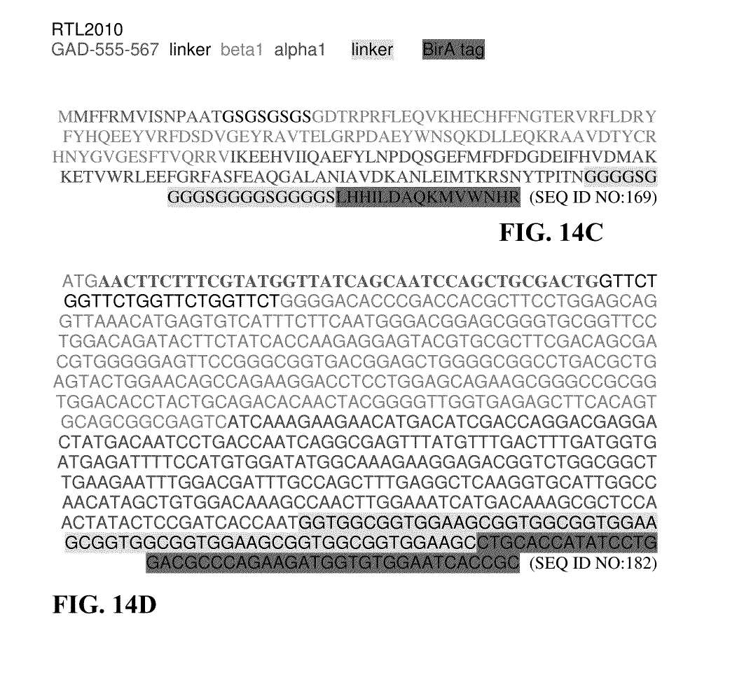

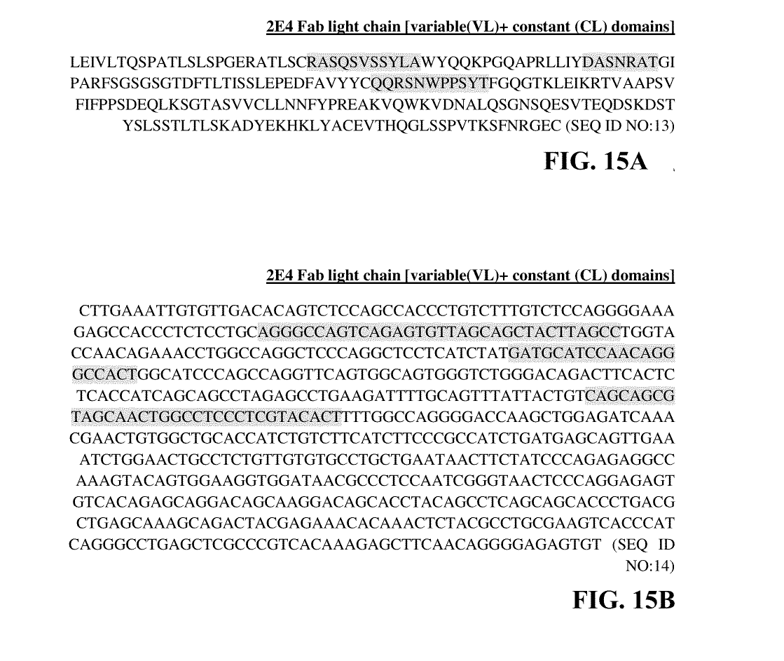

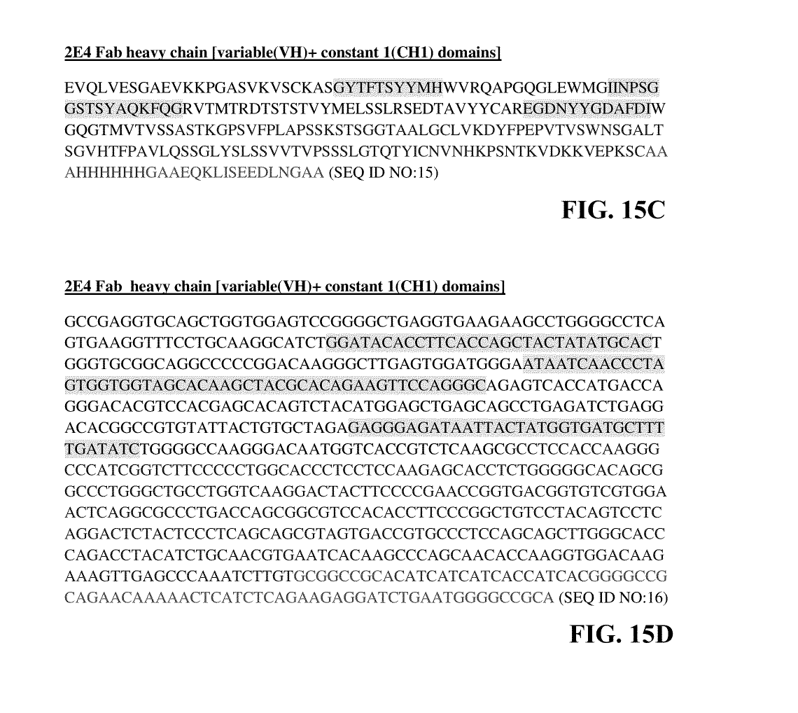

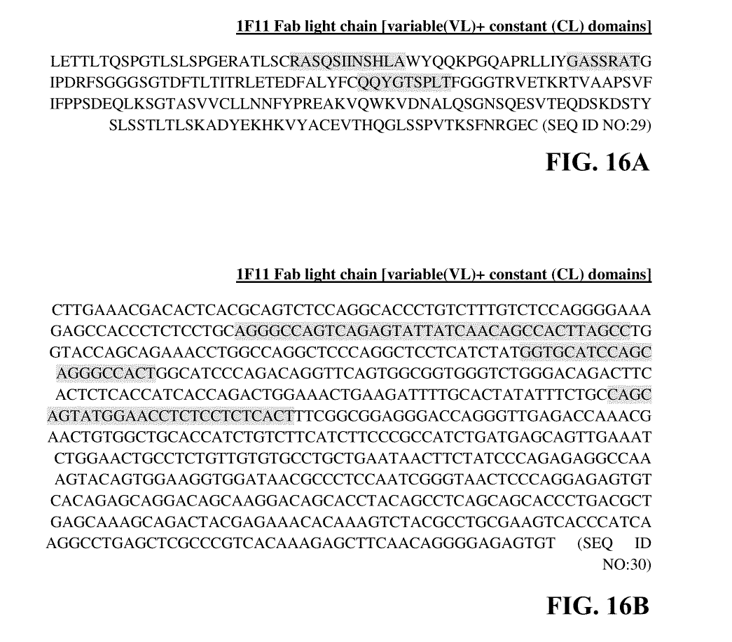

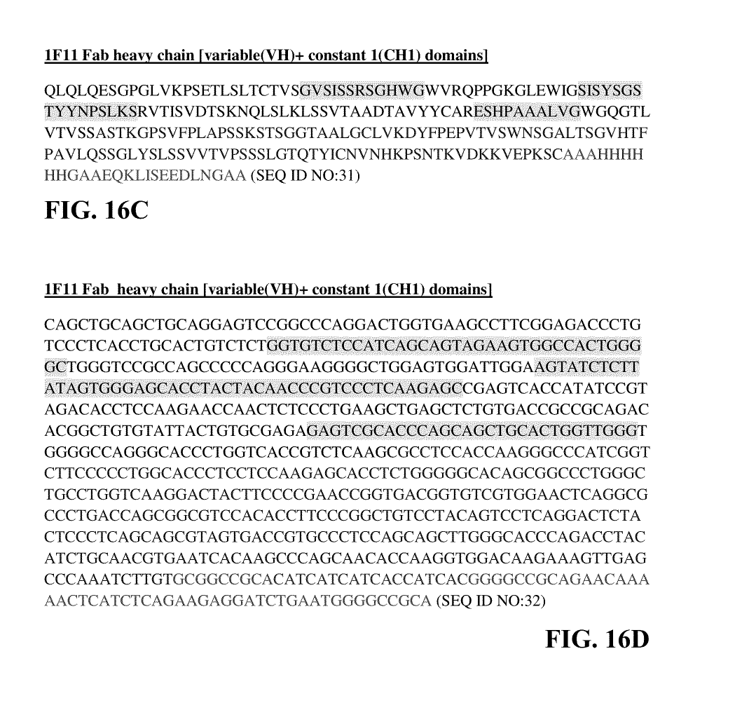

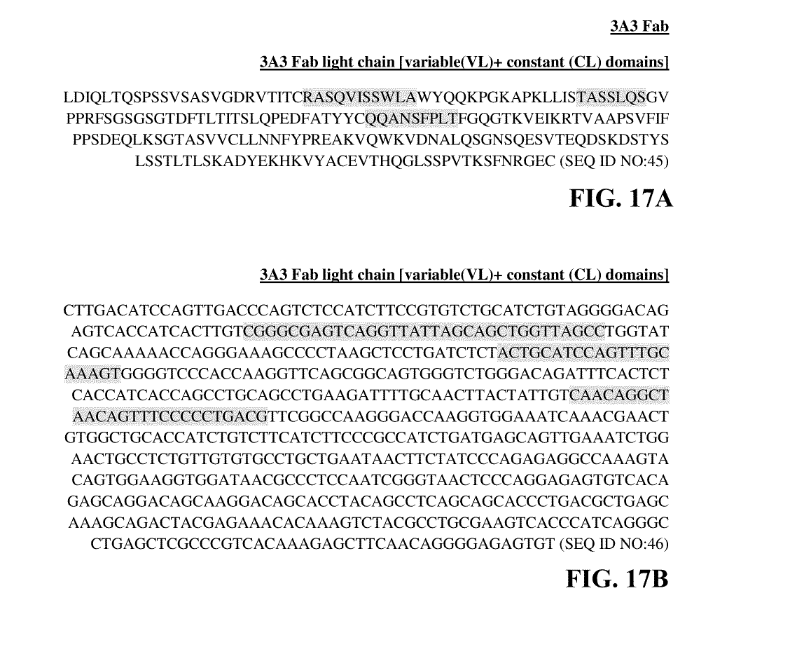

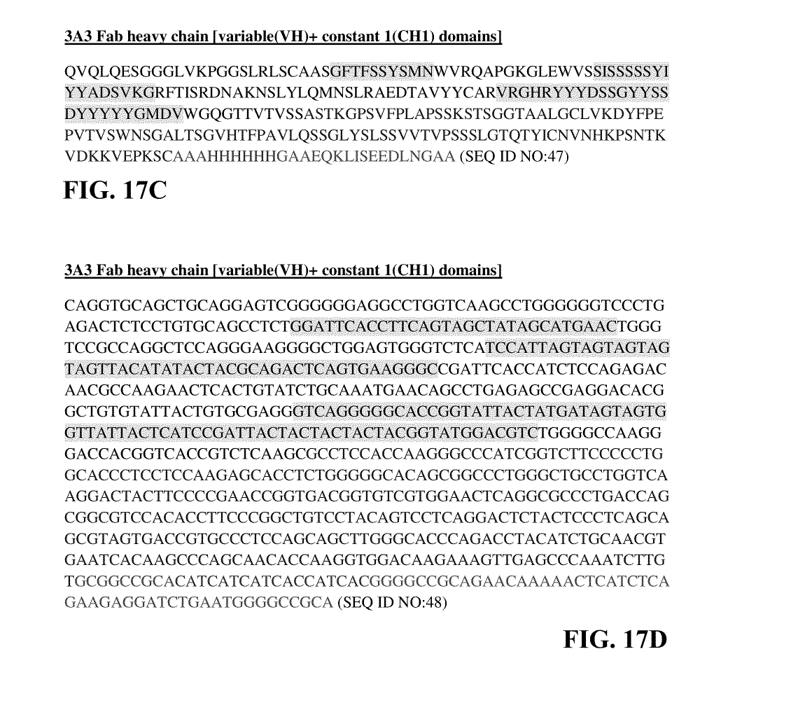

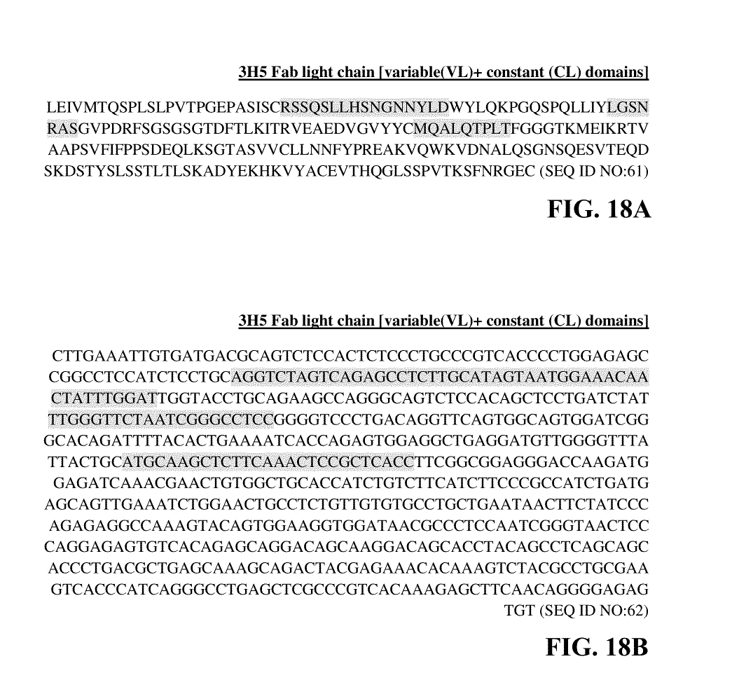

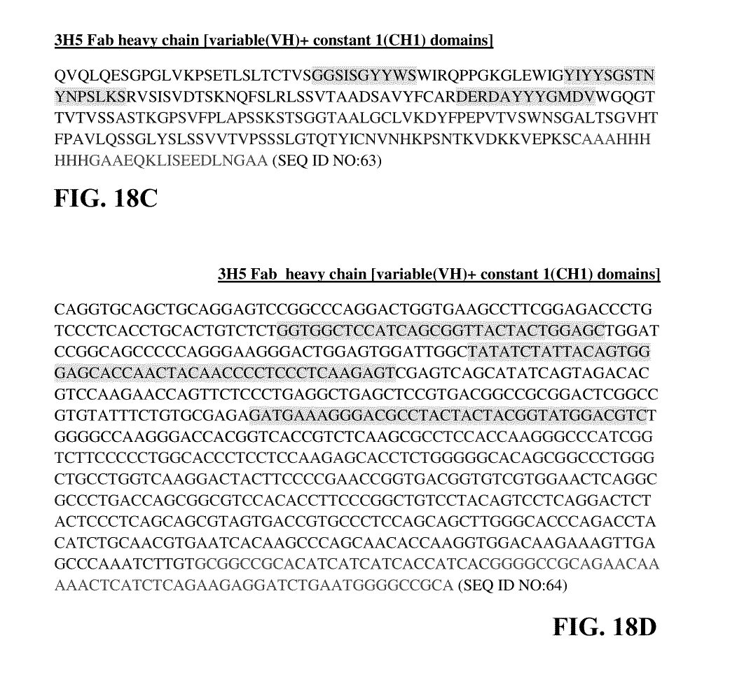

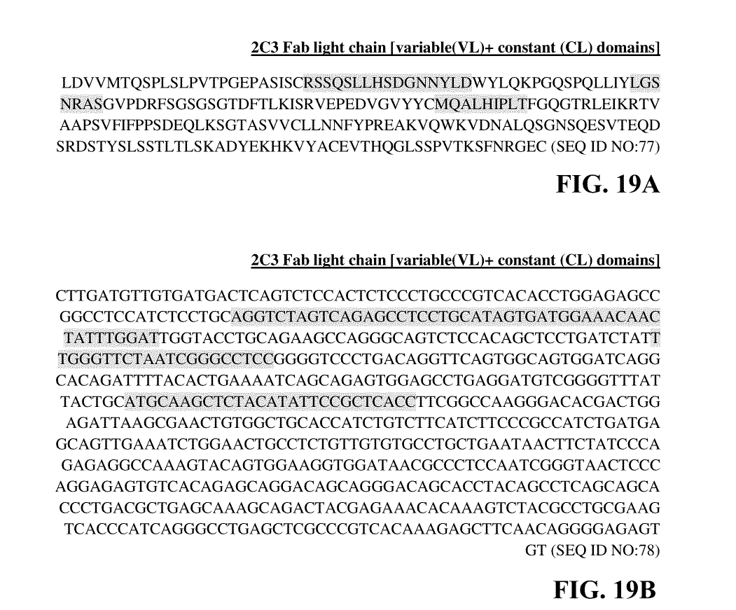

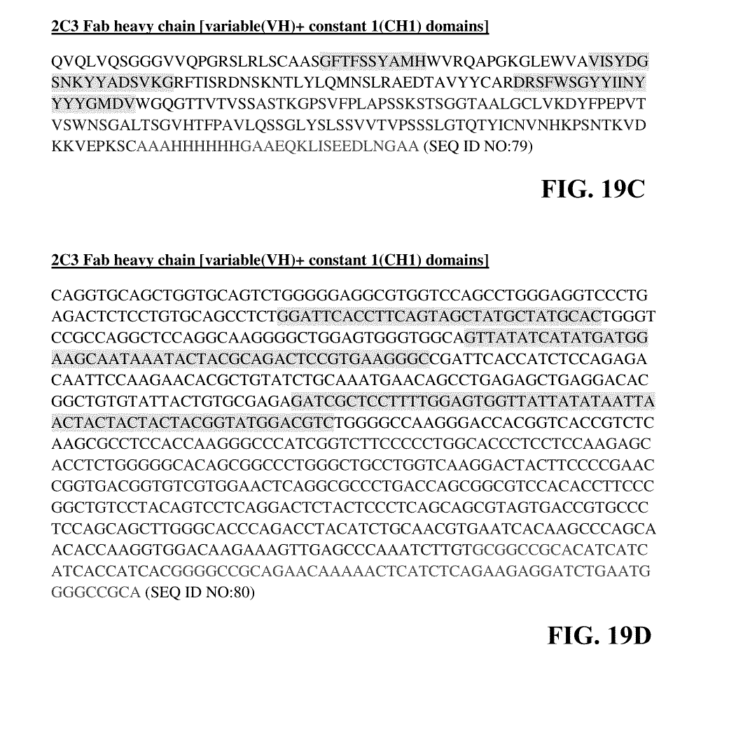

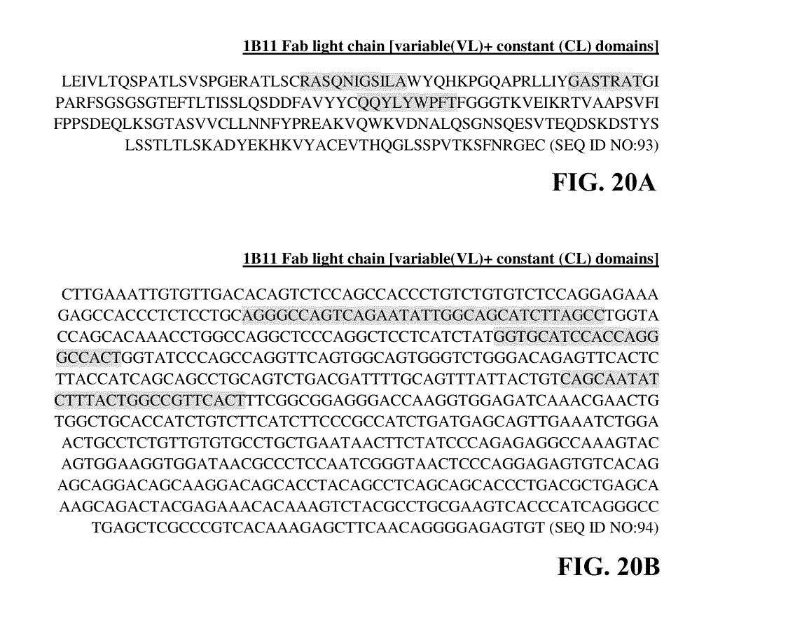

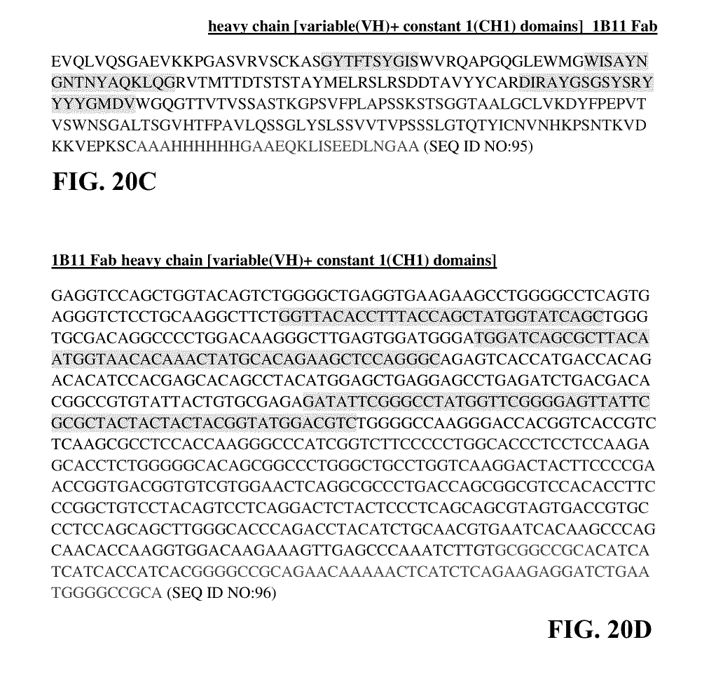

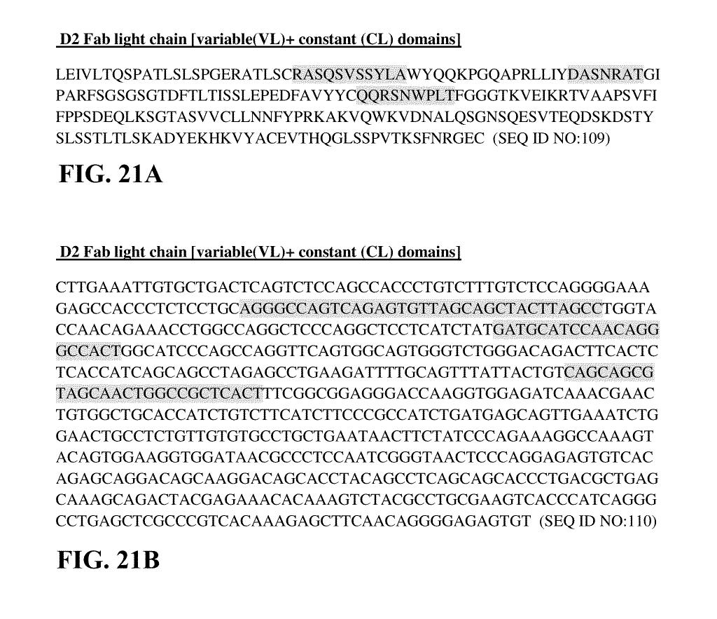

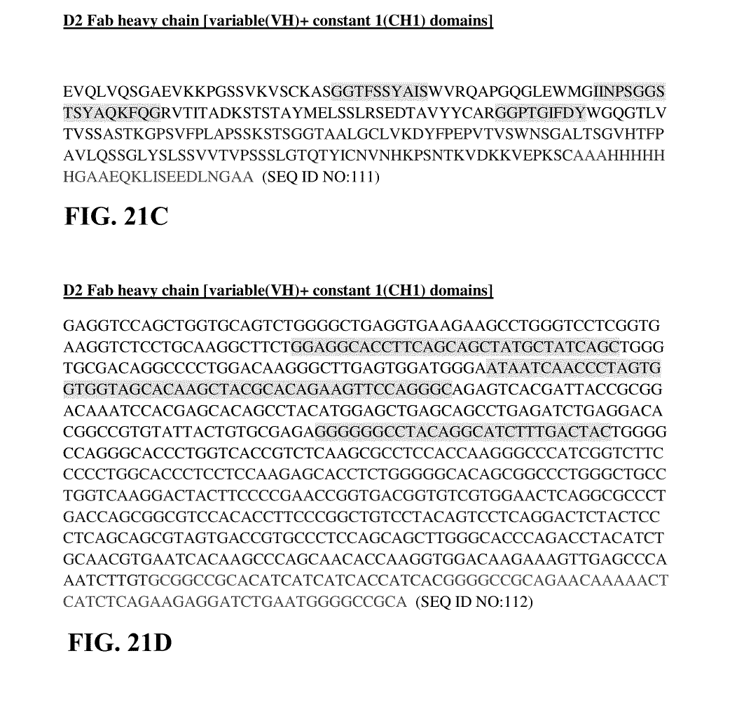

Non-limiting examples of RTLs which include the antigenic peptides are illustrated in SEQ ID NO:151 (RTL1000; MOG-35-55 DR2 RTL; FIG. 13A); SEQ ID NO:152 (RTL340; MBP-85-99 DR2 RTL; FIG. 13BC); SEQ ID NO:168 (RTL2011; Insulin A1-1-15-DR4 RTL; FIG. 14A); and SEQ ID NO:169 (RTL2010; GAD-555-567-DR4 RTL; FIG. 14C).

Non-limiting examples of nucleic acid sequences encoding RTLs which include the antigenic peptides are provided in SEQ ID NO:170 (RTL1000; MOG-35-55 DR2 RTL; FIG. 13B); SEQ ID NO:193 (RTL-340-BirA; MBP-85-99 DR2 RTL, FIG. 13D) (RTL340; MBP-85-99 DR2 RTL; FIG. 13D); SEQ ID NO:181 (RTL2011; Insulin A1-1-15-DR4 RTL; FIG. 14B) and SEQ ID NO:182 (RTL2010; GAD-555-567-DR4 RTL; FIG. 14D).

The antigenic peptide according to some embodiments of the invention is an autoantigenic peptide.

As used herein the phrase “autoantigenic peptide” refers to an antigen derived from an endogenous (i.e., self protein) or a consumed protein (e.g., by food) against which an inflammatory response is elicited as part of an autoimmune inflammatory response.

It should be noted that the phrases “endogenous”, “self” are relative expressions referring to the individual in which the autoimmune response is elicited.

It should be noted that presentation of an autoantigenic peptide on antigen presenting cells (APCs) can result in recognition of the MHC-autoantigenic peptides by specific T cells, and consequently generation of an inflammatory response that can activate and recruit T cell and B cell responses against the APCs cells.

According to some embodiments of the invention the autoantigenic peptide is associated with a disease selected from the group consisting of diabetes, multiple sclerosis, rheumatoid arthritis, celiac disease and stroke.

According to some embodiments of the invention, the diabetes-associated autoantigenic peptide is a beta-cell autoantigenic peptide.

According to some embodiments of the invention, the diabetes-associated autoantigenic peptide is derived from a polypeptide selected from the group consisting of preproinsulin (amino acids 1-110 of GenBank Accession No. NP_{—}000198, SEQ ID NO:113), proinsulin (amino acids 25-110 of GenBank Accession No. NP_{—}000198, SEQ ID NO:114), Glutamic acid decarboxylase (GAD, GenBank Accession No. NP_{—}000809.1, SEQ ID NO:115), Insulinoma Associated protein 2 (IA-2, GenBank accession No. NP_{—}115983) SEQ ID NO:116), IA-2β [also referred to as phogrin, GenBank Accession No. NP_{—}570857.2 (SEQ ID NO:117), NP_{—}570858.2 (SEQ ID NO:133), NP_{—}002838.2 (SEQ ID NO:134)], Islet-specific Glucose-6-phosphatase catalytic subunit-Related Protein [IGRP; GeneID: 57818, GenBank Accession No. NP_{—}066999.1, glucose-6-phosphatase 2 isoform 1 (SEQ ID NO:118) and GenBank Accession No. NP_{—}001075155.1, glucose-6-phosphatase 2 isoform 2 (SEQ ID NO:119)], chromogranin A (GenBank Accession No. NP_{—}001266 (SEQ ID NO:120), Zinc Transporter 8 (ZnT8 (GenBank Accession NO. NP_{—}776250.2, SEQ ID NO:121), Heat Shock Protein-60 (GenBank Accession No. NP_{—}955472.1; SEQ ID NO:122), and Heat Shock Protein-70 (GenBank Accession No. NP_{—}005337.2 (SEQ ID NO:123) and NP_{—}005336.3 (SEQ ID NO:124).

Tables 1, 2 and 3, hereinbelow, provide non-limiting examples of MHC class II restricted diabetes associated autoantigens which can form a complex with the β1-α1 two-domain of an MHC class II allele according to some embodiments of the invention.

TABLE 1
Provided are the diabetes-associated autoantigenic peptides
(with their sequence identifiers, SEQ ID NO:) and the MHC class
II molecules which bind thereto.
GAD, ZnT8 and IA-2 derived autoantigenic peptides

| SEQ | | | SEQ | | | SEQ | | |
|---|---|---|---|---|---|---|---|---|
| ID | | | ID | | | ID | | |
| NO: | GAD | MHC | NO: | ZnT8 | MHC | NO: | IA-2 | MHC |
| 203 | MNILLQYV | DR4 | 251 | LTIQIES | DQ8 | 259 | VSSVSSQ | DR4 |
| | VKSFD | | | AADQDP | | | FSDAAQA | |
| | | | | S | | | SPSSFSD | |
| 204 | IAPVFVLLE | DR4 | 252 | RTGIAQ | DQ8 | 260 | LAKEWQ | DR4 |
| | | | | ALSSFD | | | ALCAYQ | |
| | | | | LH | | | AEPNTCA | |
| | | | | | | | TAQGE | |
| 205 | LPRLIAFTS | DR4 | 253 | LYPDYQ | DQ8 | 261 | KLKVESS | DR4 |
| | EHSHF | | | IQAGIMI | | | PSRSDYIN | |
| | | | | T | | | ASPIIEHD | |
| | | | | | | | P | |
| 206 | IAFTSEHSH | DR4 | 254 | ILSVHV | DQ8 | 262 | IKLKVESS | DR4 |
| | FSLK | | | ATAASQ | | | PSRSDYIN | |
| | | | | DS | | | ASPI | |
| 207 | TVYGAFDP | DR4 | 255 | SKRLTF | DQ8 | 263 | MVWESG | DR4 |
| | LLAVAD | | | GWYRA | | | CTVIVML | |
| | | | | EIL | | | TPLVEDG | |
| | | | | | | | V | |
| 208 | KYKIWMH | DR4 | 256 | AILTDA | DQ8 | 264 | RQHARQ | DQ8 |
| | VDAAWGGG | | | AHLLID | | | QDKERLA | |
| | | | | LT | | | ALGPE | |
| 209 | KHKWKLS | DR4 | 257 | KATGNR | DQ8 | 265 | GPEGAHG | DQ8 |
| | GVERANSV | | | SSKQAH | | | DTTFEYQ | |
| | | | | AK | | | DLCR | |
| 210 | LYNIIKNRE | DR4 | 258 | AVDGVI | DQ8 | 266 | EGPPEPSR | DQ8 |
| | GYEMVF | | | SVHSLHI | | | VSSVSSQ | |
| | | | | W | | | FSD | |
| 211 | PSLRTLED | DR4 | | | | 267 | FSDAAQA | DQ8 |
| | NEERMSR | | | | | | SPSSHSST | |
| | | | | | | | PSW | |
| 212 | RMMEYGT | DR4 | | | | 268 | AEPNTCA | DQ8 |
| | TMVSYQPL | | | | | | TAQGEGN | |
| | | | | | | | IKKN | |
| 213 | SYQPLGDK | DR4 | | | | 269 | NASPIIEH | DQ8 |
| | VNFFRMV | | | | | | DPRMPAY | |
| | | | | | | | IAT | |
| 214 | NFFRMVIS | DR4 | | | | 270 | DEGSALY | DQ8 |
| | NPAAT | | | | | | HVYEVNL | |
| | | | | | | | VSEH | |
| 215 | ATHQDIDF | DR4 | | | | 271 | KGVKEID | DQ8 |
| | LIEEIER | | | | | | IAATLEH | |
| | | | | | | | VRDO | |
| 216 | ATDLLPAC | DQ8 | | | | 272 | FALTAVA | DQ8 |
| | D | | | | | | EEVNAIL | |
| | | | | | | | KALPQ | |
| 217 | FDRSTKVI | DQ8 | | | | 273 | KNRSLAV | DQ8 |
| | DFHYPNE | | | | | | LTYDHSR | |
| | | | | | | | I | |
| 218 | ELLQEYN | DQ8 | | | | 274 | GADPSAD | DQ8 |
| | WE | | | | | | ATEAYQE | |
| | | | | | | | L | |
| 219 | EYNWELA | DQ8 | | | | 275 | EIDIAATL | DQ8 |
| | DQ | | | | | | E | |
| 220 | DIDFLIEEI | DQ8 | | | | 276 | NTCATAQ | DQ8 |
| | | | | | | | GE | |
| 221 | TGHPRYFN | DQ8 | | | | 277 | EPNTCAT | DQ8 |
| | QLSTGLD | | | | | | AQ | |
| 222 | TYEIAPVF | DQ8 | | | | 278 | ERLAALG | DQ8 |
| | VLLEYVT | | | | | | PE | |
| 223 | YVTLKKM | DQ8 | | | | 279 | QHARQQ | DQ8 |
| | RE | | | | | | DKE | |
| 224 | PGGSGDGI | DQ8 | | | | 280 | YEVNLVS | DQ8 |
| | FSPGGAISN | | | | | | EH | |
| | MYA | | | | | | | |
| 225 | NMYAMMI | DQ8 | | | | 281 | GASLYHV | DQ8 |
| | ARFKMFPE | | | | | | YE | |
| | VKEKG | | | | | | | |
| 226 | PEVKEKG | DQ8 | | | | 282 | FALTAVA | DQ8 |
| | MAALPRLI | | | | | | EE | |
| | AFTSE | | | | | | | |
| 227 | DSVILIKCD | DQ8 | | | | 283 | GAHGDTT | DQ8 |
| | | | | | | | FE | |
| 228 | GKMIPSDL | DQ8 | | | | 284 | GDTTFEY | DQ8 |
| | E | | | | | | QD | |
| 229 | ERRILEAK | DQ8 | | | | 285 | AAQASPS | DQ8 |
| | Q | | | | | | SH | |
| 230 | ERANSVT | DQ8 | | | | 286 | SRVSSVS | DQ8 |
| | WN | | | | | | SQ | |
| 231 | QCSALLVR | DQ8 | | | | 287 | TQFHFLS | DQ8 |
| | E | | | | | | WP | |
| 232 | KHYDLSYD | DQ8 | | | | 288 | EEPAQAN | DQ8 |
| | TGDKALQ | | | | | | MD | |
| 233 | AKGTTGFE | DQ8 | | | | 289 | GHMILAY | DQ8 |
| | AHVDKCL | | | | | | ME | |
| 234 | VDKCLELA | DQ8 | | | | 290 | MILAYME | DQ8 |
| | EYLYNIIKN | | | | | | DH | |
| | REG | | | | | | | |
| 235 | IIKNREGYE | DQ8 | | | | 291 | QALCAY | DQ8 |
| | | | | | | | QAE | |
| 236 | MVFDGKP | DQ8 | | | | 292 | EWQALC | DQ8 |
| | QHTNVCF | | | | | | AYQ | |
| | W | | | | | | | |
| 237 | CFWYIPPSL | DQ8 | | | | 293 | LVRSKDQ | DQ8 |
| | RTLEDN | | | | | | FE | |
| 238 | FWYIPPSLR | DQ8 | | | | 294 | VEDGVK | DQ8 |
| | TLED | | | | | | QCD | |
| 239 | SLRTLEDN | DQ8 | | | | 295 | YILIDMV | DQ8 |
| | E | | | | | | LN | |
| 240 | ERMSRLSK | DQ8 | | | | 296 | ESGCTVI | DQ8 |
| | VAPVIKA | | | | | | VM | |
| 241 | IKARMME | DQ8 | | | | 297 | LCAYQAE | DQ8 |
| | YGTTMVS | | | | | | PN | |
| | Y | | | | | | | |
| 242 | RMMEYGT | DQ8 | | | | 298 | ETRTLTQ | DQ8 |
| | TMVSYQPL | | | | | | FH | |
| 243 | VISNPAAT | DQ8 | | | | 299 | VESSPSRS | DQ8 |
| | H | | | | | | D | |
| 244 | IDFLIEEIE | DQ8 | | | | 300 | GPLSHTIA | DQ8 |
| | | | | | | | D | |
| 245 | NWELADQ | DR2 | | | | 301 | SLFNRAE | DQ8 |
| | PQNLEEIL | | | | | | GP | |
| | MHCQT | | | | | | | |
| 246 | GHPRYFNQ | DR2 | | | | 302 | HPDFLPY | DQ8 |
| | LSTG | | | | | | DH | |
| 247 | TYEIAPVF | DR2 | | | | 303 | HFLSWPA | DQ8 |
| | VLLFYVTL | | | | | | EG | |
| | KKMR | | | | | | | |
| 248 | VNFFRMVI | DR4 | | | | 304 | DFRRKVN | DQ8 |
| | SNPAATHQ | | | | | | KC | |
| | D | | | | | | | |
| 249 | DKVNFFR | DR4 | | | | 305 | HCSDGAG | DQ8 |
| | MVISNPAA | | | | | | RT | |
| | THQDID | | | | | | | |
| 250 | FFRMVISN | core | | | | 306 | LVRSFYL | DQ8 |
| | PA | sequence | | | | | KN | |
| | | | | | | 307 | KNRSLAV | DQ8 |
| | | | | | | | LTYDHSR | |
| | | | | | | | I | |
| | | | | | | 308 | GADPSAD | DQ8 |
| | | | | | | | ATEAYQE | |
| | | | | | | | L | |
| | | | | | | 309 | ANMDIST | Unknown |
| | | | | | | | GHMILAY | |
| | | | | | | | ME | |
| | | | | | | 310 | WQALCA | Unknown |
| | | | | | | | YQAEPNT | |
| | | | | | | | CAT | |
| | | | | | | 311 | LSHTIADF | Unknown |
| | | | | | | | WQMVWE | |
| | | | | | | | SG | |
| | | | | | | 312 | DFWQMV | Unknown |
| | | | | | | | WESGCTV | |
| | | | | | | | IVM | |
| | | | | | | 313 | WESGCTV | Unknown |
| | | | | | | | IVMLTPL | |
| | | | | | | | VE | |
| | | | | | | 314 | VIVMLTP | Unknown |
| | | | | | | | LVEDGVK | |
| | | | | | | | QC | |
| | | | | | | 315 | SEHIWCE | Unknown |
| | | | | | | | DFLVRSF | |
| | | | | | | | YL | |
| | | | | | | 316 | WCEDFLV | Unknown |
| | | | | | | | RSFYLKN | |
| | | | | | | | VQ | |
| | | | | | | 317 | EDFLVRS | Unknown |
| | | | | | | | FYLKNVQ | |
| | | | | | | | TQ | |
| | | | | | | 318 | DFRRKVN | Unknown |
| | | | | | | | KCYRGRS | |
| | | | | | | | CP | |
| | | | | | | 319 | YILIDMV | Unknown |
| | | | | | | | LNRMAK | |
| | | | | | | | GVK | |
| | | | | | | 320 | FEFALTA | Unknown |
| | | | | | | | VAEEVNA | |
| | | | | | | | IL | |

TABLE 2
Provided are the diabetes-associated autoantigenic peptides with their
sequence identifiers, SEQ ID NO:) and the MHC class II molecules
which bind thereto.
Preproinsulin and HSP-60 autoantigenic peptide

| SEQ ID | PREPRO- | | SEQ ID | | |
|---|---|---|---|---|---|
| NO: | INSULIN | MHC | NO: | HSP-60 | MHC |
| 321 | EALYLVCGE | DQ8 | 342 | KFGADARALML | Unknown |
| | | | | QGVDLLADA | |
| 322 | SICSLYQLE | DQ8 | 343 | NPVEIRRGVMLA | Unknown |
| | | | | VDAVIAEL | |
| 323 | ALLALWGPD | DQ8 | 344 | QSIVPALEIANAH | Unknown |
| | | | | RKPLVIIA | |
| 324 | GSLQPLALE | DQ8 | 345 | LVLNRLKVGLQV | Unknown |
| | | | | VAVKAPGF | |
| 325 | TPKTRREAE | DQ8 | 346 | IVLGGGCALLRCI | Unknown |
| | | | | PALDSLT | |
| 326 | PAAAFVNQH | DQ8 | 347 | VLGGGCALLRCIP | Unknown |
| | | | | ALDSLTPANED | |
| 327 | DPAAAFVNQ | DQ8 | 348 | EIIKRTLKIPAMTI | Unknown |
| | | | | AKNAGV | |
| 328 | PDPAAAFVN | DQ8 | 349 | VNMVEKGIIDPT | Unknown |
| | | | | KVVRTALL | |
| 329 | QKRGIVEQC | DQ8 | | | |
| 330 | ELGGGPGAG | DQ8 | | | |
| 331 | EAEDLQVGQ | DQ8 | | | |
| 332 | LQVGQVELG | DQ8 | | | |
| 333 | HLCGSHLVE | DQ8 | | | |
| 334 | GIVEQCCTSICS | DR4 | | | |
| 335 | KRGIVEQCCT | DR4 | | | |
| | SICS | | | | |
| 336 | LALLALWGPD | Unknown | | | |
| | PAAAFV | | | | |
| 337 | PAAAFVNQHL | Unknown | | | |
| | CGSHLV | | | | |
| 338 | SHLVEALYLV | Unknown | | | |
| | CGERG | | | | |
| 339 | FFYTPKTRRE | Unknown | | | |
| | AED | | | | |
| 340 | GAGSLQPLAL | Unknown | | | |
| | EGSLQKRG | | | | |
| 341 | SLQKRGIVEQ | Unknown | | | |
| | CCTSICS | | | | |

TABLE 3
Provided are the diabetes-associated autoantigenic peptides (with their
sequence identifiers, SEQ ID NO:) and the MHC class II molecules which
bind thereto.
HSP-70 and IGRP derived autoantigenic peptides

| SEQ ID | | | SEQ ID | | |
|---|---|---|---|---|---|
| NO: | HSP-70 | MHC | NO: | IGRP | MHC |
| 350 | MAKAAAVGIDLGTT | Unknown | 359 | QHLQKDYRAY | DR3 |
| | YSCVGV | | | YTF | |
| 351 | GLNVLRIINEPTAAAI | Unknown | 360 | RVLNIDLLWSV | DR3 |
| | AYGL | | | PI | |
| 352 | TIDDGIFEVKATAGD | Unknown | 361 | YTFLNFMSNV | DR4 |
| | THLGG | | | GDP | |
| 353 | THLGGEDFDNRLVN | Unknown | 362 | DWIHIDTTPFA | DR4 |
| | HFVEEF | | | GL | |
| 354 | KRTLSSSTQASLEIDS | Unknown | | | |
| | LFEG | | | | |
| 355 | LLLLDVAPLSLGLET | Unknown | | | |
| | AGGVM | | | | |
| 356 | PTKQTQIEITYSDNQP | Unknown | | | |
| | GVLI | | | | |
| 357 | KANKITITNDKGRLS | Unknown | | | |
| | KEEIE | | | | |
| 358 | KEEIERMVQEAEKYK | Unknown | | | |

Further description of type I diabetes-associated autoantigenic peptides can be found in Lieberman S M, DiLorenzo T P, 2003. A comprehensive guide to antibody and T-cell responses in type 1 diabetes. Tissue Antigens, 62:359-77; Liu J, Purdy L E, Rabinovitch S, Jevnikar A M, Elliott J F. 1999, Major DQ8-restricted T-cell epitopes for human GAD65 mapped using human CD4, DQA1*0301, DQB1*0302 transgenic IA(null) NOD mice, Diabetes, 48: 469-77; Di Lorenzo T P, Peakman M, Roep B O. 2007, Translational mini-review series on type 1 diabetes: Systematic analysis of T cell epitopes in autoimmune diabetes. Clin Exp Immunol. 148:1-16; Stadinski et α1 Immunity 32:446, 2010; each of which is fully incorporated herein by reference).

According to some embodiments of the invention, the GAD autoantigenic peptide comprises a core amino acid sequence set forth by SEQ ID NO:125 (GAD556-565, FFRMVISNPA).

According to some embodiments of the invention, the GAD autoantigenic peptide comprises a core amino acid sequence set forth by SEQ ID NO:125 (GAD556-565, FFRMVISNPA) and no more than 30 amino acids.

According to some embodiments of the invention, the GAD autoantigenic peptide is GAD₅₅₅₋₅₆₇ (NFFRMVISNPAAT; SEQ ID NO:126).

According to some embodiments of the invention, the multiple sclerosis-associated autoantigenic peptide is derived from a polypeptide selected from the group consisting of myelin oligodendrocyte glycoprotein [MOG; GenBank Accession Nos. NP_{—}001008229.1 (SEQ ID NO:135); NP_{—}001008230.1 (SEQ ID NO:136); NP_{—}001163889 (SEQ ID NO:137); NP_{—}002424.3 (SEQ ID NO:138); NP_{—}996532 (SEQ ID NO:139); NP_{—}996533.2 (SEQ ID NO:140); NP_{—}996534.2 (SEQ ID NO:141); NP_{—}996535.2 (SEQ ID NO:142); NP_{—}996537.3 (SEQ ID NO:143)], myelin basic protein [MBP; GenBank Accession Nos. NP_{—}001020252.1 (SEQ ID NO:127); NP_{—}001020261.1 (SEQ ID NO:144); NP_{—}001020263.1 (SEQ ID NO:145); NP_{—}001020271.1 (SEQ ID NO:146); NP_{—}001020272.1 (SEQ ID NO:147); NP_{—}002376.1 (SEQ ID NO:148)], and proteolipid protein [PLP1; GenBank Accession Nos. NP_{—}000524.3 (SEQ ID NO:128); NP_{—}001122306.1 (SEQ ID NO:149); NP_{—}955772.1 (SEQ ID NO:150)].

Tables 4 and 5, hereinbelow, provide non-limiting examples of MHC class II restricted multiple sclerosis associated autoantigens which can form a complex with the β1-α1 two-domain of an MHC class II allele according to some embodiments of the invention.

TABLE 4
Multiple sclerosis associated autoantigens derived from Myelin
basic protein (MBP) and ProteoLipid Protein (PLP)

| SEQ ID | MBP (Myelin | | SEQ ID | PLP (ProteoLipid | |
|---|---|---|---|---|---|
| NO: | basic protein) | MHC | NO: | Protein) | MHC |
| 363 | RSQPGLCNMYKDSHHP | unknown | 371 | VFACSAVPVYI | unknown |
| | ARTA | | | YFNTWTTCQS | |
| 364 | FKGVDAQGTLSKIFKLG | unknown | 372 | YIYFNTWTTCQ | unknown |
| | GRDS | | | SIAFPSKTSA | |
| 365 | GDRGAPKRGSGKVPWL | DP | 373 | AHSLERVCHCL | DR |
| | KPGRS | | | GKWLGHPDKF | |
| 366 | RSQPGLCNMYKDSHHP | DP | 374 | AVRQIFGDYKT | DR |
| | ARTA | | | TICGKGLSAT | |
| 367 | SDYKSAHKGFKGVDAQ | DR | 375 | FMIAATYNFAV | DR |
| | GTLSK | | | LKLMGRGTKF | |
| 368 | FKGVDAQGTLSKIFKLG | DR | 376 | AHSLERVCHCL | DQ |
| | GRDS | | | GKWLGHPDKF | |
| 369 | SDYKSAHKGFKGVDAQ | DQ | 377 | AVRQIFGDYKT | DQ |
| | GTLSK | | | TICGKGLSAT | |
| 370 | ENPVVHFFKNIVTPR | DR2 | 378 | CQSIAFPSKTSA | DQ |
| | | | | SIGSLCAD | |
| | | | 379 | SKTSASIGSLC | DQ |
| | | | | ADARMYGVL | |
| | | | 380 | GVLPWNAFPG | DQ |
| | | | | KVCGSNLLSI | |
| | | | 381 | FMIAATYNFAV | DQ |
| | | | | LKLMGRGTKF | |

TABLE 5
Multiple sclerosis associated
autoantigens derived
from Myelin Oligodendrocyte Glycoprotein (MOG)

| MHC | SEQ ID NO: | Peptide |
|---|---|---|
| unknown | 382 | ELKVEDPFYWVSPGVLVLLAV |
| unknown | 383 | TFDPHFLRVPCWKITLFVIV |
| unknown | 384 | VIVPVLGPLVALIICYNWLHR |
| unknown | 385 | VALIICYNWLHRRLAGQFLEE |
| DR | 386 | GFTCFFRDHSYQEEAAMELKV |
| DR | 387 | ITVGLVFLCLQYRLRGKLRAE |
| DR | 388 | VALIICYNWLHRRLAGQFLEE |
| DR | 389 | LQYRLRGKLRAEIENLHRTFD |
| DQ | 390 | ELKVEDPFYWVSPGVLVLLAV |
| DQ | 391 | LQYRLRGKLRAEIENLHRTFD |
| DR2 | 129 | MEVGWYRPPFSRVVHLYRNGK |
| DR2 | 393 | PERYGRTELLKDAIGEGKVTLRIRN |
| DR4 | 394 | TCFFRDHSYQEE |
| DR4 | 395 | FVIVPVLGP |
| DR4 | 396 | KITLFVIVPVLGP |

According to some embodiments of the invention, the MOG autoantigenic peptide is MOG-35-55 (SEQ ID NO:129).

According to some embodiments of the invention, the MBP autoantigenic peptide is MBP-85-99 (SEQ ID NO:130).

According to some embodiments of the invention, the rheumatoid arthritis-associated autoantigenic peptide is derived from a polypeptide selected from the group consisting of Collagen II (COL2A1, GenBank Accession NO. NP_{—}001835.3; SEQ ID NO:132).

Tables 6-10, hereinbelow, provide non-limiting examples of MHC class II restricted rheumatoid arthritis associated autoantigens which can form a complex with the β1-α1 two-domain of an MHC class II allele according to some embodiments of the invention.

TABLE 6
Rheumatoid arthritis associated Collagen II and
a Matrix metalloproteinase-1 autoantigens

| | | | | | Matrix |
|---|---|---|---|---|---|
| | | | | | metalloprotein- |
| | SEQ | collagen II | | SEQ | ase-1 auto |
| | ID | autoantigenic | | ID | antigenic |
| MHC | NO: | peptide | MHC | NO: | peptide |
| DR4/ | 397 | AGFKGEQGPKGEP | un- | 399 | GVVSHSFPATLETQE |
| DR1 | | | known | | |
| DR4/ | 398 | EPGIAGFKGEQGPKGEPG | un- | | |
| DR1 | | | known | | |

TABLE 7
Rheumatoid arthritis associated Aggrecan core protein precursor
and Matrix metalloproteinase-3 autoantigens

| | SEQ | AGGRECAN CORE | | SEQ | MATRIX |
|---|---|---|---|---|---|
| | ID | PROTEIN PRECURSOR | | ID | METALLOPROTEINASE-3 |
| MHC | NO: | AUTOANTIGENIC PEPTIDE | MHC | NO: | AUTOANTIGENIC PEPTIDE |
| unknown | 400 | LSGLPSGGEVLEISV | unknown | 402 | FFYFFTGSSQLEFDP |
| unknown | 401 | ISGLPSGGDDLETST | | | |

TABLE 8
Rheumatoid arthritis associated Calpain-2 and Matrix metalloproteinase-10
autoantigens

| | SEQ | Calpain-2 | | SEQ | Matrix |
|---|---|---|---|---|---|
| | ID | autoantigenic | | ID | metalloproteinase-10 |
| MHC | NO: | peptide | MHC | NO: | autoantigenic peptide |
| unknown | 403 | HAYSVTGAEEVESNG | unknown | 404 | SAFWPSLPSGLDAAY |

TABLE 9
Rheumatoid arthritis associated Fibrillin-1 precursor and Matrix
metalloproteinase-16 autoantigens

| | SEQ | Fibrillin-1 precursor | | SEQ | Matrix |
|---|---|---|---|---|---|
| | ID | autoantigenic | | ID | metalloproteinase-16 |
| MHC | NO: | peptide | MHC | NO: | autoantigenic peptide |
| unknown | 405 | CVDTRSGNCYLDIRP | unknown | 406 | VKEGHSPPDDVDIVI |

TABLE 10
Rheumatoid arthritis associated Tenascin and heterogeneous nuclear
ribonucleoprotein A2 autoantigens

| | SEQ | Tenascin | | SEQ | Heterogeneous nuclear |
|---|---|---|---|---|---|
| | ID | autoantigenic | | ID | ribonucleoprotein A2 |
| MHC | NO: | peptide | MHC | NO: | autoantigenic peptide |
| unknown | 407 | EPVSGSFTTALDGPS | DR1/ | 408 | RDYFEEYGKIDTIEIIT |
| | | | DR4 | | |

According to some embodiments of the invention, the celiac-associated autoantigenic peptide is derived from alpha Gliadin [e.g., GenBank Accession Nos. ADM96154 (SEQ ID NO:199), ADD17013.1 (SEQ ID NO:β1)].

Table 11, hereinbelow, provides a non-limiting list of MHC class II restricted celiac associated autoantigens which can form a complex with the β1-α1 two-domain of an MHC class II allele according to some embodiments of the invention.

TABLE 11
Celiac associated gliadin autoantigens

| | SEQ | α-Gliadin | | SEQ | α-gliadin |
|---|---|---|---|---|---|
| | ID | autoantigenic | | ID | autoantigenic |
| MHC | NO: | peptide | MHC | NO: | peptide |
| DQ2/DQ8 | 409 | LGQQQPFPPQQPY | DQ2 | 410 | QLQPFPQPQLPY |
| DQ2/DQ8 | 197 | FPQPELPYPQP | DQ2 | 411 | PQPQLPYPQPQLPY |
| | | (Gliadin-61-71) | | | |
| DQ2/DQ8 | 198 | VPVPQLQPQNPSQ | DQ2 | 412 | PGQQQPFPPQQPY |
| | | QQPQEQVPL | | | |
| | | (Gliadin-3-24) | | | |

TABLE 12
Celiac associated γ-gliadin and heat
shock 20 autoantigens

| | SEQ | γ-Gliadin | | SEQ | Heat shock 20 |
|---|---|---|---|---|---|
| | ID | autoantigenic | | ID | autoantigenic |
| MHC | NO: | peptide | MHC | NO: | peptide |
| DQ2 | 413 | GIIQPQQPAQL | unknown | 418 | ALPTAQVPTDP |
| DQ2 | 414 | FPQQPQQPYPQQP | unknown | 419 | GRLFDQRFGEG |
| DQ2 | 415 | FSQPQQQFPQPQ | | | |
| DQ2 | 416 | PQQPFPQQPQQPY | | | |
| DQ2 | 417 | FLQPQQPFPQQPQQP | | | |
| | | YPQQPQQPFPQ | | | |

According to some embodiments of the invention, the stroke-associated autoantigenic peptide is derived from a brain antigen such as myelin basic protein, neurofilaments and the NR2A/2B subtype of the N-methyl-D-aspartate receptor (MOG-35-55-MEVGWYRPPFSRVVHLYRNGK (SEQ ID NO:129).

Since the amino acid sequence of the autoantigen may vary in length between the same or different MHC class II alleles, the length of the autoantigenic peptides according to some embodiments of the invention may vary from at least 6 amino acids, to autoantigenic peptides having at least 8, 10, 25, or up to 30 amino acids.

According to some embodiments of the invention, the autoantigenic peptide includes a core amino acids of at least 6 amino acids, e.g., at least 7, at least 8, at least 9 and more.

According to some embodiments of the invention, the length of the autoantigenic peptide does not exceed about 100 amino acids, e.g., does not exceed about 50 amino acids, e.g., does not exceed about 30 amino acids.

According to some embodiments of the invention, the length of the autoantigenic peptide includes at least 6 and no more than 30 amino acids.

In addition, it should be noted that although some amino acids in each autoantigenic peptide are conserved between various alleles of MHC class II and cannot be substituted, other amino acids can be substituted with amino acids having essentially equivalent specificity and/or affinity of binding to MHC molecules and resulting in equivalent T cell epitope as the amino acid sequences shown in the exemplary autoantigens described above. Thus, in each autoantigenic peptide there are at least six amino acids constituting a core amino acid which are required for recognition with the respective MHC class II molecule. Identification of the core amino acids for each autoantigenic peptide can be done experimentally, e.g., by mutagenesis of the amino acids constituting the autoantigenic peptide and detection of: (i) binding to the restricted MHC class II molecules; (ii) Stimulating the restricted T cell response. The core amino acid sequence consists of anchor residues and the T-cell receptor (TCR) contact residues. For example, for the GAD autoantigenic peptide the anchor residues in the sequence NFFRMVISNPAAT (SEQ ID NO:126) are the P1 (F557), P4 (V560), P6 (S562), and P9 (A565) MHC pocket-binding residues. TCR contact residues in the sequence NFFRMVISNPAAT (SEQ ID NO:126) are at positions F556, R558, M559, 1561, N563. Accordingly, the core amino acids of the GAD555-567 autoantigenic peptide are GAD556-565 (FFRMVISNPA, SEQ ID NO:125).

The invention according to some embodiments thereof also concerns peptide variants whose sequences do not completely correspond with the aforementioned amino acid sequences but which only have identical or closely related “anchor positions”. The term “anchor position” in this connection denotes an essential amino acid residue for binding to a MHC class II complex (e.g., DR1, DR2, DR3, DR4 or DQ). The anchor position for the DRB1*0401 binding motif are for example stated in Hammer et al., Cell 74 (1993), 197-203. Such anchor positions are conserved in the autoantigenic peptide or are optionally replaced by amino acid residues with chemically very closely related side chains (e.g. alanine by valine, leucine by isoleucine and visa versa). The anchor position in the peptides according to some embodiments of the invention can be determined in a simple manner by testing variants of the aforementioned specific peptides for their binding ability to MHC molecules. Peptides according to some embodiments of the invention are characterized in that they have an essentially equivalent specificity or/and affinity of binding to MHC molecules as the aforementioned peptides. Homologous peptides having at least 50%, e.g., at least 60%, 70%, 80%, 90%, 95% or more identity to the autoantigenic peptides described herein are also contemplated by some embodiments of the invention.

As used herein the phrase “high affinity entity” refers to any naturally occurring or artificially produced molecule, composition, or organism which binds to a specific antigen with a higher affinity than to a non-specific antigen.

As used herein the term “isolated” refers to at least partially separated from the natural environment e.g., the human body.

It should be noted that the affinity can be quantified using known methods such as, Surface Plasmon Resonance (SPR) (described in Scarano S, Mascini M, Turner A P, Minunni M. Surface plasmon resonance imaging for affinity-based biosensors. Biosens Bioelectron. 2010, 25: 957-66), and can be calculated using, e.g., a dissociation constant, Kd, such that a lower Kd reflects a higher affinity.

As described, the antigen binding domain of the high affinity entity binds a soluble T-cell receptor ligand (e.g., an RTL) comprising a two-domain β1-α1 of major histocompatibility complex (MHC) class II, but does not bind a complex comprising a four-domain α1-β1/α2-β2 MHC class II.

As used herein a “four-domain α1-β1/α2-β2 MHC class II” refers to a complex which comprises at least the alpha 1 and 2 domains of MHC class II and beta 1 and 2 domains of MHC class II.

According to some embodiments of the invention, the α1-α2 domains are bound via members of affinity pair to the β1-β2 domains. The members of affinity pairs can be, for example, the leucine zipper dimerization domains of Fos and Jun transcription factors.

According to some embodiments of the invention, the α1-α2 domains are bound via protein-protein interaction to the β1-β2 domains following in vitro refolding of bacterial inclusion bodies.

The four-domain α1-β1/α2-β2 MHC class II can be empty (i.e., devoid of an antigenic peptide) or can include an antigenic peptide.

According to some embodiments of the invention, the four-domain α1-β1/α2-β2 MHC class II is in complex with the MHC class II antigenic peptide.

According to some embodiments of the invention, the four-domain α1-β1/α2-β2 MHC class II and the MHC class II antigenic peptide are covalently linked.

According to some embodiments of the invention, the four-domain complex comprises an artificial complex of α1-β1/α2-β2 to which the peptide is covalently attached. Non-limiting examples of such complexes are described in the Examples section which follows.

According to some embodiments of the invention, the four-domain complex comprises is a native complex (e.g., as presented on an antigen presenting cell) in which the antigenic peptide is not covalently attached to the four-domain complex.

According to some embodiments of the invention, the antigen binding domain of the high affinity entity does not bind a complex of the MHC class II and the MHC class II antigenic peptide when presented on an antigen presenting cell (APC).

It should be noted that the binding or absence of binding (non-binding) of the high affinity entity to an antigen can be expressed in terms of binding affinity.

According to some embodiments of the invention, the binding affinity of the high affinity entity to the two-domain β1-α1 of MHC class II is at least about 10 times higher (i.e., having a Kd at least 10 folds lower) than the binding affinity of the high affinity entity to the four domain α1-β1/α2-β2 MHC class II. According to some embodiments of the invention, the binding affinity of the high affinity entity to the two-domain β1-α1 of MHC class II is at least about 100 times higher, at least about 1000 times higher, e.g., at least about 1×10⁴ higher, e.g., at least about 1×10⁵ higher, e.g., at least about 1×10⁶, higher, e.g., at least about 1×10⁷ higher, e.g., at least about 1×10⁸ higher, e.g., at least about 1×10⁹ higher, e.g., at least about 1×10¹⁰ higher, e.g., at least about 1×10¹¹ higher or more than to the four domain α1-β1/α2-β2 MHC class II.

According to some embodiments of the invention, the dissociation constant of the high affinity entity to the two-domain β1-α1 of MHC class II is about 10⁻⁴ M or less, e.g., about 10⁻⁵ M or less, e.g., about 10⁻⁶ M or less, e.g., about 10⁻⁷ or less, e.g., about 10⁻⁸ or less, e.g., about 10⁻⁹ M or less, e.g., about 10⁻¹⁰ M or less.

According to some embodiments of the invention, the two-domain β1-α1 of the MHC class II is in complex with an MHC class II antigenic peptide.

According to some embodiments of the invention, the two-domain β1-α1 of the MHC class II is covalently linked to the MHC class II antigenic peptide.

According to some embodiments of the invention, the antigen binding domain does not bind the two-domain β1-α1 MHC class II in an absence of the MHC class II antigenic peptide, and wherein the antigen binding domain does not bind to the MHC class II antigenic peptide in an absence of the two-domain β1-α1 MHC class II.

Non-limiting examples of high affinity entities include an antibody, an antibody fragment, a phage displaying an antibody, a peptibody, a cell-based display entity (e.g., a bacterium or yeast displaying an antibody), and cell-free displaying entity (e.g., a ribosome displaying a peptide or antibody).

Bacteriophages which display antibodies and which can be used according to some embodiments of the invention include M13 and fd filamentous phage, T4, T7, and λ phages.

The techniques of using bacteria (e.g., E. Coli) and yeast for displaying antibodies are well (See e.g., Daugherty P S., et al., 1998. Antibody affinity maturation using bacterial surface display. Protein Engineering 11:825-832; Johan Rockberg et al., Epitope mapping of antibodies using bacterial surface display. Nature Methods 5, 1039-1045 (2008); Sachdev S Sidhu, Full-length antibodies on display, Nature Biotechnology 25, 537-538 (2007); each of which is fully incorporated herein by reference).

Cell-free displaying entities include a ribosome displaying a protein (described in Mingyue He and Michael J. Taussig, 2002. Ribosome display: Cell-free protein display technology. Briefings in functional genomics and proteomics. Vol 1: 204-212; Patrick Dufner et al., 2006. Harnessing phage and ribosome display for antibody optimization. Trends in Biotechnology, Vol. 24: 523-529; each of which is fully incorporated herein by reference).

Peptibodies are isolated polypeptide comprising at least one peptide capable of binding to an antigen (e.g., a CDR) attached to an Fc domain of an antibody (e.g., IgG, IgA, IgD, IgE, IgM antibodies) or a fragment of an Fc domain. A peptibody can include more than one peptide capable of binding an antigen (e.g., 2, 3, 4 or 5 peptides) which may be the same as one another or may be different from one another.

The term “antibody” as used in this invention includes intact molecules as well as functional fragments thereof, such as Fab, F(ab′)2, and Fv that are capable of binding to macrophages. These functional antibody fragments are defined as follows: (1) Fab, the fragment which contains a monovalent antigen-binding fragment of an antibody molecule, can be produced by digestion of whole antibody with the enzyme papain to yield an intact light chain and a portion of one heavy chain; (2) Fab′, the fragment of an antibody molecule that can be obtained by treating whole antibody with pepsin, followed by reduction, to yield an intact light chain and a portion of the heavy chain; two Fab′ fragments are obtained per antibody molecule; (3) (Fab′)2, the fragment of the antibody that can be obtained by treating whole antibody with the enzyme pepsin without subsequent reduction; F(ab′)2 is a dimer of two Fab′ fragments held together by two disulfide bonds; (4) Fv, defined as a genetically engineered fragment containing the variable region of the light chain and the variable region of the heavy chain expressed as two chains; (5) Single chain antibody (“SCA”), a genetically engineered molecule containing the variable region of the light chain and the variable region of the heavy chain, linked by a suitable polypeptide linker as a genetically fused single chain molecule; (6) CDR peptide is a peptide coding for a single complementarity-determining region (CDR); and (7) Single domain antibodies (also called nanobodies), a genetically engineered single monomeric variable antibody domain which selectively binds to a specific antigen. Nanobodies have a molecular weight of only 12-15 kDa, which is much smaller than a common antibody (150-160 kDa).

Non-limiting examples of such high affinity entities include the Fab antibodies 2C3, 3A3, 1F11, 2E4 and 3H5 which specifically recognize RTL1000, and Fab D2 which specifically recognizes α1/β1DR4/GAD555-567.

According to some embodiments of the invention, the antigen binding domain comprises complementarity determining regions (CDRs) 1-3 for light chain of 2E4 as set forth by SEQ ID NOs:1-3 (encoded by SEQ ID NOs:4-6, respectively) and CDRs 1-3 for heavy chain of 2E4 as set forth by SEQ ID NOs:7-9 (encoded by SEQ ID NOs:10-12, respectively).

According to some embodiments of the invention, the antigen binding domain comprises complementarity determining regions (CDRs) 1-3 for light chain of 1F11 as set forth by SEQ ID NOs:17-19 (encoded by SEQ ID NOs:20-22, respectively) and CDRs 1-3 for heavy chain of 1F11 as set forth by SEQ ID NOs:23-25 (encoded by SEQ ID NOs:26-28, respectively).

According to some embodiments of the invention, the antigen binding domain comprises complementarity determining regions (CDRs) 1-3 for light chain of 3A3 as set forth by SEQ ID NOs:33-35 (encoded by SEQ ID NOs:36-38, respectively) and CDRs 1-3 for heavy chain of 3A3 as set forth by SEQ ID NOs:39-41 (encoded by SEQ ID NOs:42-44, respectively).

According to some embodiments of the invention, the antigen binding domain comprises complementarity determining regions (CDRs) 1-3 for light chain of 3H5 as set forth by SEQ ID NOs:49-51 (encoded by SEQ ID NOs:52-54, respectively) and CDRs 1-3 for heavy chain of 3H5 as set forth by SEQ ID NOs:55-57 (encoded by SEQ ID NOs:58-60, respectively).

According to some embodiments of the invention, the antigen binding domain comprises complementarity determining regions (CDRs) 1-3 for light chain of 2C3 as set forth by SEQ ID NOs:65-67 (encoded by SEQ ID NOs:68-70, respectively) and CDRs 1-3 for heavy chain of 2C3 as set forth by SEQ ID NOs:71-73 (encoded by SEQ ID NOs:74-76, respectively).

According to some embodiments of the invention, the antigen binding domain comprises complementarity determining regions (CDRs) 1-3 for light chain of D2 as set forth by SEQ ID NOs:97-99 (encoded by SEQ ID NOs:100-102, respectively) and CDRs 1-3 for heavy chain of D2 as set forth by SEQ ID NOs:103-105 (encoded by SEQ ID NOs:106-108, respectively).

According to some embodiments of the invention, the antibody is a monoclonal antibody.

Methods of producing polyclonal and monoclonal antibodies as well as fragments thereof are well known in the art (See for example, Harlow and Lane, Antibodies: A Laboratory Manual, Cold Spring Harbor Laboratory, New York, 1988, incorporated herein by reference).

Antibody fragments according to the present invention can be prepared by proteolytic hydrolysis of the antibody or by expression in E. coli or mammalian cells (e.g. Chinese hamster ovary cell culture or other protein expression systems) of DNA encoding the fragment. Antibody fragments can be obtained by pepsin or papain digestion of whole antibodies by conventional methods. For example, antibody fragments can be produced by enzymatic cleavage of antibodies with pepsin to provide a 5S fragment denoted F(ab′)2. This fragment can be further cleaved using a thiol reducing agent, and optionally a blocking group for the sulfhydryl groups resulting from cleavage of disulfide linkages, to produce 3.5S Fab′ monovalent fragments. Alternatively, an enzymatic cleavage using pepsin produces two monovalent Fab′ fragments and an Fc fragment directly. These methods are described, for example, by Goldenberg, U.S. Pat. Nos. 4,036,945 and 4,331,647, and references contained therein, which patents are hereby incorporated by reference in their entirety. See also Porter, R. R. [Biochem. J. 73: 119-126 (1959)]. Other methods of cleaving antibodies, such as separation of heavy chains to form monovalent light-heavy chain fragments, further cleavage of fragments, or other enzymatic, chemical, or genetic techniques may also be used, so long as the fragments bind to the antigen that is recognized by the intact antibody.

Fv fragments comprise an association of VH and VL chains. This association may be noncovalent, as described in Inbar et al. [Proc. Nat'l Acad. Sci. USA 69:2659-62 (19720]. Alternatively, the variable chains can be linked by an intermolecular disulfide bond or cross-linked by chemicals such as glutaraldehyde. Preferably, the Fv fragments comprise VH and VL chains connected by a peptide linker. These single-chain antigen binding proteins (sFv) are prepared by constructing a structural gene comprising DNA sequences encoding the VH and VL domains connected by an oligonucleotide. The structural gene is inserted into an expression vector, which is subsequently introduced into a host cell such as E. coli. The recombinant host cells synthesize a single polypeptide chain with a linker peptide bridging the two V domains. Methods for producing sFvs are described, for example, by [Whitlow and Filpula, Methods 2: 97-105 (1991); Bird et al., Science 242:423-426 (1988); Pack et al., Bio/Technology 11:1271-77 (1993); and U.S. Pat. No. 4,946,778, which is hereby incorporated by reference in its entirety.

CDR peptides (“minimal recognition units”) can be obtained by constructing genes encoding the CDR of an antibody of interest. Such genes are prepared, for example, by using the polymerase chain reaction to synthesize the variable region from RNA of antibody-producing cells. See, for example, Larrick and Fry [Methods, 2: 106-10 (1991)].

According to some embodiments of the invention, the antibodies are multivalent forms such as tetrameric Fabs, IgM or IgG1 antibodies, thus forming a multivalent composition with higher avidity to the target.

According to some embodiments of the invention, the antibody comprises a human antibody.

Humanized forms of non-human (e.g., murine) antibodies are chimeric molecules of immunoglobulins, immunoglobulin chains or fragments thereof (such as Fv, Fab, Fab′, F(ab′).sub.2 or other antigen-binding subsequences of antibodies) which contain minimal sequence derived from non-human immunoglobulin. Humanized antibodies include human immunoglobulins (recipient antibody) in which residues form a complementary determining region (CDR) of the recipient are replaced by residues from a CDR of a non-human species (donor antibody) such as mouse, rat or rabbit having the desired specificity, affinity and capacity. In some instances, Fv framework residues of the human immunoglobulin are replaced by corresponding non-human residues. Humanized antibodies may also comprise residues which are found neither in the recipient antibody nor in the imported CDR or framework sequences. In general, the humanized antibody will comprise substantially all of at least one, and typically two, variable domains, in which all or substantially all of the CDR regions correspond to those of a non-human immunoglobulin and all or substantially all of the FR regions are those of a human immunoglobulin consensus sequence. The humanized antibody optimally also will comprise at least a portion of an immunoglobulin constant region (Fc), typically that of a human immunoglobulin [Jones et al., Nature, 321:522-525 (1986); Riechmann et al., Nature, 332:323-329 (1988); and Presta, Curr. Op. Struct. Biol., 2:593-596 (1992)].

Methods for humanizing non-human antibodies are well known in the art. Generally, a humanized antibody has one or more amino acid residues introduced into it from a source which is non-human. These non-human amino acid residues are often referred to as import residues, which are typically taken from an import variable domain. Humanization can be essentially performed following the method of Winter and co-workers [Jones et al., Nature, 321:522-525 (1986); Riechmann et al., Nature 332:323-327 (1988); Verhoeyen et al., Science, 239:1534-1536 (1988)], by substituting rodent CDRs or CDR sequences for the corresponding sequences of a human antibody. Accordingly, such humanized antibodies are chimeric antibodies (U.S. Pat. No. 4,816,567), wherein substantially less than an intact human variable domain has been substituted by the corresponding sequence from a non-human species. In practice, humanized antibodies are typically human antibodies in which some CDR residues and possibly some FR residues are substituted by residues from analogous sites in rodent antibodies.

Human antibodies can also be produced using various techniques known in the art, including screening of phage display libraries [Hoogenboom and Winter, J. Mol. Biol., 227:381 (1991); Marks et al., J. Mol. Biol., 222:581 (1991)]. The techniques of Cole et al. and Boerner et al. are also available for the preparation of human monoclonal antibodies (Cole et al., Monoclonal Antibodies and Cancer Therapy, Alan R. Liss, p. 77 (1985) and Boerner et al., J. Immunol., 147(1):86-95 (1991)]. Similarly, human antibodies can be made by introduction of human immunoglobulin loci into transgenic animals, e.g., mice in which the endogenous immunoglobulin genes have been partially or completely inactivated. Upon challenge, human antibody production is observed, which closely resembles that seen in humans in all respects, including gene rearrangement, assembly, and antibody repertoire. This approach is described, for example, in U.S. Pat. Nos. 5,545,807; 5,545,806; 5,569,825; 5,625,126; 5,633,425; 5,661,016, and in the following scientific publications: Marks et al., Bio/Technology 10,: 779-783 (1992); Lonberg et al., Nature 368: 856-859 (1994); Morrison, Nature 368 812-13 (1994); Fishwild et al., Nature Biotechnology 14, 845-51 (1996); Neuberger, Nature Biotechnology 14: 826 (1996); and Lonberg and Huszar, Intern. Rev. Immunol. 13, 65-93 (1995).

For in vivo use (for administering in a subject, e.g., human), the human or humanized antibody will generally tend to be better tolerated immunologically than one of non human origin since non variable portions of non human antibodies will tend to trigger xenogeneic immune responses more potent than the allogeneic immune responses triggered by human antibodies which will typically be allogeneic with the individual. It will be preferable to minimize such immune responses since these will tend to shorten the half-life, and hence the effectiveness, of the antibody in the individual. Furthermore, such immune responses may be pathogenic to the individual, for example by triggering harmful inflammatory reactions.

Alternately, an antibody of a human origin, or a humanized antibody, will also be advantageous for targeting of soluble RTL-like structures in which a functional physiological effect, for example phagocytosis of the soluble RTL-like structures, activated by a constant region of the antibody in the individual is desired. In these cases, an optimal functional interaction occurs when the functional portion of the antibody, such as the Fc region, and the molecule interacting therewith such as the Fc receptor or the Fc-binding complement component are of a similar origin (e.g., human origin).

Depending on the application and purpose, the antibody of the invention, which includes a constant region, or a portion thereof of any of various isotypes, may be employed. According to some embodiments of the invention, the isotype is selected so as to enable or inhibit a desired physiological effect, or to inhibit an undesired specific binding of the antibody via the constant region or portion thereof. For example, for inducing antibody-dependent cell mediated cytotoxicity (ADCC) by a natural killer (NK) cell, the isotype can be IgG; for inducing ADCC by a mast cell/basophil, the isotype can be IgE; and for inducing ADCC by an eosinophil, the isotype can be IgE or IgA. For inducing a complement cascade the antibody may comprise a constant region or portion thereof capable of initiating the cascade. For example, the antibody may advantageously comprise a Cgamma2 domain of IgG or Cmu3 domain of IgM to trigger a C1q-mediated complement cascade.

Conversely, for avoiding an immune response, such as the aforementioned one, or for avoiding a specific binding via the constant region or portion thereof, the antibody of the invention may not comprise a constant region (be devoid of a constant region), a portion thereof or specific glycosylation moieties (required for complement activation) of the relevant isotype.

Once the CDRs of an antibody are identified, using conventional genetic engineering techniques, expressible polynucleotides encoding any of the forms or fragments of antibodies described herein can be synthesized and modified in one of many ways in order to produce a spectrum of related-products.

For example, to generate the high affinity entity of the invention (e.g., the antibody of the invention), an isolated polynucleotide sequence [e.g., a polynucleotide comprising the CDRs 1-3 of the heavy chain and CDRs 1-3 of the light chain] is preferably ligated into a nucleic acid construct (expression vector) suitable for expression in a host cell. Such a nucleic acid construct includes a promoter sequence for directing transcription of the polynucleotide sequence in the cell in a constitutive or inducible manner.

The nucleic acid construct of the invention may also include an enhancer, a transcription and translation initiation sequence, transcription and translation terminator and a polyadenylation signal, a 5′ LTR, a tRNA binding site, a packaging signal, an origin of second-strand DNA synthesis, and a 3′ LTR or a portion thereof; a signal sequence for secretion of the antibody polypeptide from a host cell; additional polynucleotide sequences that allow, for example, the translation of several proteins from a single mRNA such as an internal ribosome entry site (IRES) and sequences for genomic integration of the promoter-chimeric polypeptide; sequences engineered to enhance stability, production, purification, yield or toxicity of the expressed peptide.

Examples for mammalian expression vectors include, but are not limited to, pcDNA3, pcDNA3.1(+/−), pGL3, pZeoSV2(+/−), pSecTag2, pDisplay, pEF/myc/cyto, pCMV/myc/cyto, pCR3.1, pSinRep5, DH26S, DHBB, pNMT1, pNMT41, pNMT81, which are available from Invitrogen, pCI which is available from Promega, pMbac, pPbac, pBK-RSV and pBK-CMV which are available from Strategene, pTRES which is available from Clontech, and their derivatives.

Expression vectors containing regulatory elements from eukaryotic viruses such as retroviruses can be also used. SV40 vectors include pSVT7 and pMT2. Vectors derived from bovine papilloma virus include pBV-1MTHA, and vectors derived from Epstein Bar virus include pHEBO, and p2O5. Other exemplary vectors include pMSG, pAV009/A⁺, pMTO10/A⁺, pMAMneo-5, baculovirus pDSVE, and any other vector allowing expression of proteins under the direction of the SV-40 early promoter, SV-40 later promoter, metallothionein promoter, murine mammary tumor virus promoter, Rous sarcoma virus promoter, polyhedrin promoter, or other promoters shown effective for expression in eukaryotic cells.

Various methods can be used to introduce the nucleic acid construct of the invention into cells. Such methods are generally described in Sambrook et al., Molecular Cloning: A Laboratory Manual, Cold Springs Harbor Laboratory, New York (1989, 1992), in Ausubel et al., Current Protocols in Molecular Biology, John Wiley and Sons, Baltimore, Md. (1989), Chang et al., Somatic Gene Therapy, CRC Press, Ann Arbor, Mich. (1995), Vega et al., Gene Targeting, CRC Press, Ann Arbor Mich. (1995), Vectors: A Survey of Molecular Cloning Vectors and Their Uses, Butterworths, Boston Mass. (1988) and Gilboa et at. [Biotechniques 4 (6): 504-512, 1986] and include, for example, stable or transient transfection, lipofection, electroporation and infection with recombinant viral vectors. In addition, see U.S. Pat. Nos. 5,464,764 and 5,487,992 for positive-negative selection methods.

Recombinant viral vectors are useful for in vivo expression since they offer advantages such as lateral infection and targeting specificity. Introduction of nucleic acids by viral infection offers several advantages over other methods such as lipofection and electroporation, since higher transfection efficiency can be obtained due to the infectious nature of viruses.

Currently preferred in vivo nucleic acid transfer techniques include transfection with viral or non-viral constructs, such as adenovirus, lentivirus, Herpes simplex I virus, or adeno-associated virus (AAV) and lipid-based systems. Useful lipids for lipid-mediated transfer of the gene are, for example, DOTMA, DOPE, and DC-Chol [Tonkinson et al., Cancer Investigation, 14(1): 54-65 (1996)]. The most preferred constructs for use in gene therapy are viruses, most preferably adenoviruses, AAV, lentiviruses, or retroviruses.

As mentioned hereinabove, a variety of prokaryotic or eukaryotic cells can be used as host-expression systems to express the antibody of the invention. These include, but are not limited to, microorganisms, such as bacteria transformed with a recombinant bacteriophage DNA, plasmid DNA or cosmid DNA expression vector containing the coding sequence; yeast transformed with recombinant yeast expression vectors containing the coding sequence; plant cell systems infected with recombinant virus expression vectors (e.g., cauliflower mosaic virus, CaMV; tobacco mosaic virus, TMV) or transformed with recombinant plasmid expression vectors, such as Ti plasmid, containing the coding sequence. Mammalian expression systems can also be used to express the antibody of the invention.

Recovery of the recombinant antibody polypeptide is effected following an appropriate time in culture. The phrase “recovering the recombinant polypeptide” refers to collecting the whole fermentation medium containing the polypeptide and need not imply additional steps of separation or purification. Not withstanding the above, antibody polypeptides of the invention can be purified using a variety of standard protein purification techniques, such as, but not limited to, affinity chromatography, ion exchange chromatography, filtration, electrophoresis, hydrophobic interaction chromatography, gel filtration chromatography, reverse phase chromatography, concanavalin A chromatography, chromatofocusing and differential solubilization.

According to an aspect of some embodiments of the invention, there is provided a molecule comprising the high affinity entity (e.g., the antibody) of the invention being conjugated to a functional moiety (also referred to as an “immunoconjugate”) such as a detectable or a therapeutic moiety. The immunoconjugate molecule can be an isolated molecule such as a soluble or synthetic molecule.

Various types of detectable or reporter moieties may be conjugated to the high affinity entity of the invention (e.g., the antibody of the invention). These include, but not are limited to, a radioactive isotope (such as ^{[125]}iodine), a phosphorescent chemical, a chemiluminescent chemical, a fluorescent chemical (fluorophore), an enzyme, a fluorescent polypeptide, an affinity tag, and molecules (contrast agents) detectable by Positron Emission Tomagraphy (PET) or Magnetic Resonance Imaging (MRI).

Examples of suitable fluorophores include, but are not limited to, phycoerythrin (PE), fluorescein isothiocyanate (FITC), Cy-chrome, rhodamine, green fluorescent protein (GFP), blue fluorescent protein (BFP), Texas red, PE-Cy5, and the like. For additional guidance regarding fluorophore selection, methods of linking fluorophores to various types of molecules see Richard P. Haugland, “Molecular Probes: Handbook of Fluorescent Probes and Research Chemicals 1992-1994”, 5th ed., Molecular Probes, Inc. (1994); U.S. Pat. No. 6,037,137 to Oncoimmunin Inc.; Hermanson, “Bioconjugate Techniques”, Academic Press New York, N.Y. (1995); Kay M. et al., 1995. Biochemistry 34:293; Stubbs et al., 1996. Biochemistry 35:937; Gakamsky D. et al., “Evaluating Receptor Stoichiometry by Fluorescence Resonance Energy Transfer,” in “Receptors: A Practical Approach,” 2nd ed., Stanford C. and Horton R. (eds.), Oxford University Press, UK. (2001); U.S. Pat. No. 6,350,466 to Targesome, Inc.]. Fluorescence detection methods which can be used to detect the high affinity entity (e.g., antibody) when conjugated to a fluorescent detectable moiety include, for example, fluorescence activated flow cytometry (FACS), immunofluorescence confocal microscopy, fluorescence in-situ hybridization (FISH) and fluorescence resonance energy transfer (FRET).

Numerous types of enzymes may be attached to the high affinity entity (e.g., the antibody) of some embodiments of the invention [e.g., horseradish peroxidase (HPR), beta-galactosidase, and alkaline phosphatase (AP)] and detection of enzyme-conjugated antibodies can be performed using ELISA (e.g., in solution), enzyme-linked immunohistochemical assay (e.g., in a fixed tissue), enzyme-linked chemiluminescence assay (e.g., in an electrophoretically separated protein mixture) or other methods known in the art [see e.g., Khatkhatay M I. and Desai M., 1999. J Immunoassay 20:151-83; Wisdom G B., 1994. Methods Mol Biol. 32:433-40; Ishikawa E. et al., 1983. J Immunoassay 4:209-327; Oellerich M., 1980. J Clin Chem Clin Biochem. 18:197-208; Schuurs A H. and van Weemen B K., 1980. J Immunoassay 1:229-49).

The affinity tag (or a member of a binding pair) can be an antigen identifiable by a corresponding antibody [e.g., digoxigenin (DIG) which is identified by an anti-DIG antibody) or a molecule having a high affinity towards the tag [e.g., streptavidin and biotin]. The antibody or the molecule which binds the affinity tag can be fluorescently labeled or conjugated to enzyme as described above.

Various methods, widely practiced in the art, may be employed to attach a streptavidin or biotin molecule to the antibody of the invention. For example, a biotin molecule may be attached to the antibody of the invention via the recognition sequence of a biotin protein ligase (e.g., BirA) as described in the Examples section which follows and in Denkberg, G. et al., 2000. Eur. J. Immunol. 30:3522-3532. Alternatively, a streptavidin molecule may be attached to an antibody fragment, such as a single chain Fv, essentially as described in Cloutier S M. et al., 2000. Molecular Immunology 37:1067-1077; Dubel S. et al., 1995. J Immunol Methods 178:201; Huston J S. et al., 1991. Methods in Enzymology 203:46; Kipriyanov S M. et al., 1995. Hum Antibodies Hybridomas 6:93; Kipriyanov S M. et al., 1996. Protein Engineering 9:203; Pearce L A. et al., 1997. Biochem Molec Biol Intl 42:1179-1188).

Functional moieties, such as fluorophores, conjugated to streptavidin are commercially available from essentially all major suppliers of immunofluorescence flow cytometry reagents (for example, Pharmingen or Becton-Dickinson).

According to some embodiments of the invention, biotin conjugated antibodies are bound to a streptavidin molecule to form a multivalent composition (e.g., a dimer or tetramer form of the antibody).

Table 13 provides non-limiting examples of identifiable moieties which can be conjugated to the antibody of the invention.

TABLE 13

| | Amino Acid sequence | Nucleic Acid sequence |
|---|---|---|
| | (GenBank Accession No.)/ | (GenBank Accession No.)/ |
| Identifiable Moiety | SEQ ID NO: | SEQ ID NO: |
| Green Fluorescent protein | AAL33912/420 | AF435427/427 |
| Alkaline phosphatase | AAK73766/421 | AY042185/428 |
| Peroxidase | CAA00083/422 | A00740/429 |
| Histidine tag | Amino acids 264-269 of | Nucleotides 790-807 of |
| | GenBank Accession No. | GenBank Accession No. |
| | AAK09208/423 | AF329457/430 |
| Myc tag | Amino acids 273-283 of | Nucleotides 817-849 of |
| | GenBank Accession No. | GenBank Accession No. |
| | AAK09208/423 | AF329457/430 |
| Biotin lygase tag | LHHILDAQKMVWNHR/ | |
| | SEQ ID NO: 157 | |
| orange fluorescent protein | AAL33917/424 | AF435432/431 |
| Beta galactosidase | ACH42114/425 | EU626139/432 |
| Streptavidin | AAM49066/426 | AF283893/433 |

According to some embodiments, the high affinity entity (e.g., the antibody) may be conjugated to a therapeutic moiety. The therapeutic moiety can be, for example, a cytotoxic moiety, a toxic moiety, a cytokine moiety and a second antibody moiety comprising a different specificity to the antibodies of the invention.

Non-limiting examples of therapeutic moieties which can be conjugated to the high affinity entity (e.g., the antibody) of the invention are provided in Table 14, hereinbelow.

TABLE 14

| | Amino acid sequence | Nucleic acid sequence |
|---|---|---|
| | (GenBank Accession | (GenBank Accession |
| Therapeutic moiety | No.)/SEQ ID NO: | No.)/SEQ ID NO: |
| Pseudomonas exotoxin | ABU63124/434 | EU090068/443 |
| Diphtheria toxin | AAV70486/435 | AY820132.1/444 |
| interleukin 2 | CAA00227/436 | A02159/445 |
| CD3 | P07766/437 | X03884/446 |
| CD16 | NP_000560.5/438 | NM_000569.6/447 |
| interleukin 4 | NP_000580.1/439 | NM_000589.2/448 |
| HLA-A2 | P01892/440 | K02883/449 |
| interleukin 10 | P22301/441 | M57627/450 |
| Ricin toxin | EEF27734/442 | EQ975183/451 |

According to some embodiments of the invention, the toxic moiety is PE38KDEL [SEQ ID NO:452 for protein and SEQ ID NO:453 for nucleic acid].

The functional moiety (the detectable or therapeutic moiety of the invention) may be attached or conjugated to the high affinity entity (e.g., the antibody) of the invention in various ways, depending on the context, application and purpose.

When the functional moiety is a polypeptide, the immunoconjugate may be produced by recombinant means. For example, the nucleic acid sequence encoding a toxin (e.g., PE38KDEL) or a fluorescent protein [e.g., green fluorescent protein (GFP), red fluorescent protein (RFP) or yellow fluorescent protein (YFP)] may be ligated in-frame with the nucleic acid sequence encoding the high affinity entity (e.g., the antibody) of the invention and be expressed in a host cell to produce a recombinant conjugated antibody. Alternatively, the functional moiety may be chemically synthesized by, for example, the stepwise addition of one or more amino acid residues in defined order such as solid phase peptide synthetic techniques.

A functional moiety may also be attached to the high affinity entity (e.g., the antibody) of the invention using standard chemical synthesis techniques widely practiced in the art [see e.g., hypertext transfer protocol://world wide web (dot) chemistry (dot) org/portal/Chemistry)], such as using any suitable chemical linkage, direct or indirect, as via a peptide bond (when the functional moiety is a polypeptide), or via covalent bonding to an intervening linker element, such as a linker peptide or other chemical moiety, such as an organic polymer. Chimeric peptides may be linked via bonding at the carboxy (C) or amino (N) termini of the peptides, or via bonding to internal chemical groups such as straight, branched or cyclic side chains, internal carbon or nitrogen atoms, and the like. Description of fluorescent labeling of antibodies is provided in details in U.S. Pat. Nos. 3,940,475, 4,289,747, and 4,376,110.

Exemplary methods for conjugating peptide moieties (therapeutic or detectable moieties) to the high affinity entity (e.g., the antibody) of the invention are described herein below:

SPDP Conjugation

A non-limiting example of a method of SPDP conjugation is described in Cumber et al. (1985, Methods of Enzymology 112: 207-224). Briefly, a peptide, such as a detectable or therapeutic moiety (e.g., 1.7 mg/ml) is mixed with a 10-fold excess of SPDP (50 mM in ethanol); the antibody is mixed with a 25-fold excess of SPDP in 20 mM sodium phosphate, 0.10 M NaCl pH 7.2 and each of the reactions is incubated for about 3 hours at room temperature. The reactions are then dialyzed against PBS. The peptide is reduced, e.g., with 50 mM DTT for 1 hour at room temperature. The reduced peptide is desalted by equilibration on G-25 column (up to 5% sample/column volume) with 50 mM KH₂PO₄ pH 6.5. The reduced peptide is combined with the SPDP-antibody in a molar ratio of 1:10 antibody:peptide and incubated at 4° C. overnight to form a peptide-antibody conjugate.

Glutaraldehyde Conjugation

A non-limiting example of a method of glutaraldehyde conjugation is described in G. T. Hermanson (1996, “Antibody Modification and Conjugation, in Bioconjugate Techniques, Academic Press, San Diego). Briefly, the antibody and the peptide (1.1 mg/ml) are mixed at a 10-fold excess with 0.05% glutaraldehyde in 0.1 M phosphate, 0.15 M NaCl pH 6.8, and allowed to react for 2 hours at room temperature. 0.01 M lysine can be added to block excess sites. After-the reaction, the excess glutaraldehyde is removed using a G-25 column equilibrated with PBS (10% v/v sample/column volumes)

Carbodiimide Conjugation

Conjugation of a peptide with an antibody can be accomplished using a dehydrating agent such as a carbodiimide, e.g., in the presence of 4-dimethyl aminopyridine. Carbodiimide conjugation can be used to form a covalent bond between a carboxyl group of peptide and an hydroxyl group of an antibody (resulting in the formation of an ester bond), or an amino group of an antibody (resulting in the formation of an amide bond) or a sulfhydryl group of an antibody (resulting in the formation of a thioester bond). Likewise, carbodiimide coupling can be used to form analogous covalent bonds between a carbon group of an antibody and an hydroxyl, amino or sulfhydryl group of the peptide [see, J. March, Advanced Organic Chemistry: Reaction's, Mechanism, and Structure, pp. 349-50 & 372-74 (3d ed.), 1985]. For example, the peptide can be conjugated to an antibody via a covalent bond using a carbodiimide, such as dicyclohexylcarbodiimide [B. Neises et al. (1978), Angew Chem., Int. Ed. Engl. 17:522; A. Hassner et al. (1978, Tetrahedron Lett. 4475); E. P. Boden et al. (1986, J. Org. Chem. 50:2394) and L. J. Mathias (1979, Synthesis 561)].

According to an aspect of some embodiments of the invention there is provided a method of isolating a high affinity which specifically binds to a recombinant T-cell receptor ligand (RTL). The method is effected by (a) screening a library comprising a plurality of high affinity entities with an isolated complex comprising an MHC class II antigenic peptide being covalently linked to a two-domain β1-α1 of the MHC class II; and (b) isolating at least one high affinity entity comprising an antigen binding domain which specifically binds the isolated complex, wherein the at least one high affinity entity does not bind to a complex comprising a four-domain α1-β1/α2-β2 MHC class II and the MHC class II antigenic peptide, thereby isolating the high affinity entities which specifically binds to the recombinant T-cell receptor ligand (RTL).

According to some embodiments of the invention the at least one high affinity entity does not bind the MHC class II in an absence of the MHC class II antigenic peptide, and wherein the at least one high affinity entity does not bind to the MHC class II antigenic peptide in an absence of the MHC class II.

According to some embodiments of the invention the isolated complex further comprising an in-frame tag, i.e., a peptide capable of being enzymatically modified to include a binding entity. For example, such a peptide can be used for site specific biotinylation using e.g., a biotin protein ligase-Bir A enzyme (AVIDITY). Non-limiting examples of such tags includes the Bir A recognition sequence is set forth by SEQ ID NO:392 (Leu Gly Gly Ile Phe Glu Ala Met Lys Met Glu Leu Arg Asp).

According to some embodiments of the invention, the Bir A recognition sequence for biotinylation is covalently conjugated at the carboxy terminal (C) of the recombinant alpha 1 domain.

It should be noted that an in-frame tag can be used for isolation of antibodies which specifically bind to the specific two-domain β1-α1 MHC class II, such as using streptavidin.

According to some embodiments of the invention, the peptide-bound two-domain β1-α1 MHC class II forms multimers which are bound by a common binding entity.

For example, multimers (e.g., tetramers) of peptide-bound two-domain β1-α1 MHC class II can be formed using a streptavidin which binds to the biotinylated complexes.

As described hereinabove, the present inventors have also isolated antibodies which recognize the two-domain β1-α1 conformation regardless the presence or absence of the antigenic peptide. Such antibodies can detect soluble two-domain T-cell receptor ligands (e.g., RTLs or native RTL-like structures) with a wide variety of antigenic peptides being bound to them, as well as empty RTLs.

Thus, according to an aspect of some embodiments of the invention, there is provided an isolated high affinity entity comprising an antigen binding domain which specifically binds a soluble T-cell receptor ligand comprising a two-domain β1-α1 of a major histocompatibility complex (MHC) class II whether in complex with an MHC class II antigenic peptide or in an absence of the MHC class II antigenic peptide (i.e., when not in complex with the antigenic peptide), wherein the antigen binding domain does not bind a complex comprising a four-domain α1-β1/α2-β2 MHC class II.

According to some embodiments of the invention, the antigen binding domain of the high affinity entity binds with similar binding affinities to soluble T-cell receptor ligands (e.g., RTLs or native RTL-like structures) which are in complex with an antigenic peptide and to soluble T-cell receptor ligands (e.g., RTLs or native RTL-like structures) which are devoid of an antigenic peptide (e.g., an empty RTL devoid of an antigenic peptide). Non-limiting examples of such antibodies include the 1B11 antibody (see FIG. 7B for example).

According to some embodiments of the invention, two binding affinities are considered to be similar if they are within the same order of magnitude, e.g., wherein the difference between the binding affinities does not exceed about 10 times, e.g., does not exceed about 8 times, does not exceed about 6 times, does not exceed about 5 times, does not exceed about 4 times, does not exceed about 3 times, does not exceed about 2 times, e.g., does not exceed about 1.5 times.

It should be noted that an empty RTL can bind to an MHC class II-restricted antigenic peptide to form a two-domain β1-α1 MHC class II—antigen peptide complex.

According to some embodiments of the invention, the antigen binding domain comprising complementarity determining regions (CDRs) 1-3 for the heavy chain as set forth by SEQ ID NOs:87-89 (encoded by SEQ ID NOs:90-92, respectively); and CDRs 1-3 for the light chain as set forth by SEQ ID NOs:81-83 (encoded by SEQ ID NOs:84-86, respectively).

As mentioned above and in the Examples section which follows, the antibodies which bind the two-domain MHC class II (e.g., the 1B11 antibody) can detect naturally occurring soluble two-domain MHC class II structures (RTL-like structures) that may function as inhibitors of T-cell responses. Such MHC class II-derived structures may act as natural analogues of RTL constructs and induce similar regulatory effects on T-cell responses. Antibodies which are directed to the two-domain MHC conformation are valuable tool for isolation and identification of such native structures, while distinguishing it from full-length MHC class II structures.

Reversal of Tolerogenic Activity

Immunosuppressive function of two domain MHC class II structures might contribute to physiological conditions characterized with excessive CD4+ T-cell tolerance such as in cancer and infectious diseases. The isolated antibodies according to some embodiments of the invention, which are directed to two-domain MHC class II structures, have the ability to reverse the tolerogenic activity of these structures and therefore to be used as agents for treatment of cancer and infectious diseases. Pan-two domain MHC II structures antibodies (Abs) such as 1B11 can naturalize general CD4+ T-cells suppression, while TCR-like Abs such as 2E4 can naturalize RTL-like tolerogenic activity in an antigen and dose-specific manner.

The therapeutic effects of RTLs on T-cell mediated autoimmunity may involve several complementary pathways. In addition to direct TCR ligation, RTL regulatory effects on inflammatory CD4+ T-cells might work through manipulation of antigen presenting cells (APCs). Recent studies (Sinha, et al., 2010) demonstrate high avidity binding of RTLs to macrophages, dendritic cells and B cells, and such RTL “armed” myeloid cells (but not B cells) could tolerize T-cells specific for the RTL-bound peptide. Thus, the antibodies according to some embodiments of the invention can naturalize the tolerogenic activity of RTL-like structures by blocking two major interactions leading to RTL-induced immunosuppression: (1) Blocking of RTL-T-cell receptor (TCR) interaction by TCR-like Abs (e.g., using the 2E4 antibody) and (2) blocking of RTL binding to the RTL-receptor on APCs (e.g., using the 1B11 antibody), thus sequestering the soluble T cell receptor ligand in the subject.

Thus, according to an aspect of some embodiments of the invention there is provided a method of sequestering soluble T cell receptor ligand in a subject. The method is effected by administering the high affinity entity of some embodiments of the invention the subject, thereby sequestering soluble T cell receptor ligand.

It should be noted that sequestering the soluble T cell receptor ligand results in inhibition of the binding of the soluble T cell receptor ligand to the T cell receptor or to the RTL-receptor on the antigen presenting cells.

According to some embodiments of the invention, the antigen presenting cells comprise macrophages, dendritic cells or B cells.

According to some embodiments of the invention, the soluble T cell receptor ligand exhibits an excessive inhibitory activity on T cells of the subject.

It should be noted that the excessive inhibitory activity can result from a direct binding of the soluble T cell receptor ligand to the T cell receptor, or can be mediated by binding of the soluble T cell receptor ligand to the RTL-receptor present on antigen presenting cells (APCs), which result in internalization of the soluble T cell receptor (e.g., RTL or native RTL-like structure) into the APCs, and presentation of the antigenic peptide originating from the soluble T cell receptor by the APCs. Such presentation of the antigenic peptide by the APCs inhibits the activity of the T-cells (Sinha, et al., 2010).

According to some embodiments of the invention, the excessive inhibitory activity of the soluble T cell receptor ligand is associated with cancer or an infectious disease.

Thus, the teachings of some embodiments of the invention can be used to treat a subject having pathology characterized by excessive inhibitory activity of soluble T cell receptor ligand such as cancer or an infectious disease.

The term “treating” refers to inhibiting, preventing or arresting the development of a pathology (disease, disorder or condition) and/or causing the reduction, remission, or regression of a pathology. Those of skill in the art will understand that various methodologies and assays can be used to assess the development of a pathology, and similarly, various methodologies and assays may be used to assess the reduction, remission or regression of a pathology.

As used herein, the term “subject” includes mammals, preferably human beings at any age which suffer from the pathology.

The high affinity entity of some embodiments of the invention can be administered to an organism per se, or in a pharmaceutical composition where it is mixed with suitable carriers or excipients.

As used herein a “pharmaceutical composition” refers to a preparation of one or more of the active ingredients described herein with other chemical components such as physiologically suitable carriers and excipients. The purpose of a pharmaceutical composition is to facilitate administration of a compound to an organism.

Herein the term “active ingredient” refers to the high affinity entity of some embodiments of the invention accountable for the biological effect.

Hereinafter, the phrases “physiologically acceptable carrier” and “pharmaceutically acceptable carrier” which may be interchangeably used refer to a carrier or a diluent that does not cause significant irritation to an organism and does not abrogate the biological activity and properties of the administered compound. An adjuvant is included under these phrases.

Herein the term “excipient” refers to an inert substance added to a pharmaceutical composition to further facilitate administration of an active ingredient. Examples, without limitation, of excipients include calcium carbonate, calcium phosphate, various sugars and types of starch, cellulose derivatives, gelatin, vegetable oils and polyethylene glycols.

Techniques for formulation and administration of drugs may be found in “Remington's Pharmaceutical Sciences,” Mack Publishing Co., Easton, Pa., latest edition, which is incorporated herein by reference.

Suitable routes of administration may, for example, include oral, rectal, transmucosal, especially transnasal, intestinal or parenteral delivery, including intramuscular, subcutaneous and intramedullary injections as well as intrathecal, direct intraventricular, intracardiac, e.g., into the right or left ventricular cavity, into the common coronary artery, intravenous, inrtaperitoneal, intranasal, or intraocular injections.

Conventional approaches for drug delivery to the central nervous system (CNS) include: neurosurgical strategies (e.g., intracerebral injection or intracerebroventricular infusion); molecular manipulation of the agent (e.g., production of a chimeric fusion protein that comprises a transport peptide that has an affinity for an endothelial cell surface molecule in combination with an agent that is itself incapable of crossing the BBB) in an attempt to exploit one of the endogenous transport pathways of the BBB; pharmacological strategies designed to increase the lipid solubility of an agent (e.g., conjugation of water-soluble agents to lipid or cholesterol carriers); and the transitory disruption of the integrity of the BBB by hyperosmotic disruption (resulting from the infusion of a mannitol solution into the carotid artery or the use of a biologically active agent such as an angiotensin peptide). However, each of these strategies has limitations, such as the inherent risks associated with an invasive surgical procedure, a size limitation imposed by a limitation inherent in the endogenous transport systems, potentially undesirable biological side effects associated with the systemic administration of a chimeric molecule comprised of a carrier motif that could be active outside of the CNS, and the possible risk of brain damage within regions of the brain where the BBB is disrupted, which renders it a suboptimal delivery method.

Alternately, one may administer the pharmaceutical composition in a local rather than systemic manner, for example, via injection of the pharmaceutical composition directly into a tissue region of a patient.

Pharmaceutical compositions of the present invention may be manufactured by processes well known in the art, e.g., by means of conventional mixing, dissolving, granulating, dragee-making, levigating, emulsifying, encapsulating, entrapping or lyophilizing processes.

Pharmaceutical compositions for use in accordance with the present invention thus may be formulated in conventional manner using one or more physiologically acceptable carriers comprising excipients and auxiliaries, which facilitate processing of the active ingredients into preparations which, can be used pharmaceutically. Proper formulation is dependent upon the route of administration chosen.

For injection, the active ingredients of the pharmaceutical composition may be formulated in aqueous solutions, preferably in physiologically compatible buffers such as Hank's solution, Ringer's solution, or physiological salt buffer. For transmucosal administration, penetrants appropriate to the barrier to be permeated are used in the formulation. Such penetrants are generally known in the art.

For oral administration, the pharmaceutical composition can be formulated readily by combining the active compounds with pharmaceutically acceptable carriers well known in the art. Such carriers enable the pharmaceutical composition to be formulated as tablets, pills, dragees, capsules, liquids, gels, syrups, slurries, suspensions, and the like, for oral ingestion by a patient. Pharmacological preparations for oral use can be made using a solid excipient, optionally grinding the resulting mixture, and processing the mixture of granules, after adding suitable auxiliaries if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carbomethylcellulose; and/or physiologically acceptable polymers such as polyvinylpyrrolidone (PVP). If desired, disintegrating agents may be added, such as cross-linked polyvinyl pyrrolidone, agar, or alginic acid or a salt thereof such as sodium alginate.

Dragee cores are provided with suitable coatings. For this purpose, concentrated sugar solutions may be used which may optionally contain gum arabic, talc, polyvinyl pyrrolidone, carbopol gel, polyethylene glycol, titanium dioxide, lacquer solutions and suitable organic solvents or solvent mixtures. Dyestuffs or pigments may be added to the tablets or dragee coatings for identification or to characterize different combinations of active compound doses.

Pharmaceutical compositions which can be used orally, include push-fit capsules made of gelatin as well as soft, sealed capsules made of gelatin and a plasticizer, such as glycerol or sorbitol. The push-fit capsules may contain the active ingredients in admixture with filler such as lactose, binders such as starches, lubricants such as talc or magnesium stearate and, optionally, stabilizers. In soft capsules, the active ingredients may be dissolved or suspended in suitable liquids, such as fatty oils, liquid paraffin, or liquid polyethylene glycols. In addition, stabilizers may be added. All formulations for oral administration should be in dosages suitable for the chosen route of administration.

For buccal administration, the compositions may take the form of tablets or lozenges formulated in conventional manner.

For administration by nasal inhalation, the active ingredients for use according to the present invention are conveniently delivered in the form of an aerosol spray presentation from a pressurized pack or a nebulizer with the use of a suitable propellant, e.g., dichlorodifluoromethane, trichlorofluoromethane, dichloro-tetrafluoroethane or carbon dioxide. In the case of a pressurized aerosol, the dosage unit may be determined by providing a valve to deliver a metered amount. Capsules and cartridges of, e.g., gelatin for use in a dispenser may be formulated containing a powder mix of the compound and a suitable powder base such as lactose or starch.

The pharmaceutical composition described herein may be formulated for parenteral administration, e.g., by bolus injection or continuous infusion. Formulations for injection may be presented in unit dosage form, e.g., in ampoules or in multidose containers with optionally, an added preservative. The compositions may be suspensions, solutions or emulsions in oily or aqueous vehicles, and may contain formulatory agents such as suspending, stabilizing and/or dispersing agents.

Pharmaceutical compositions for parenteral administration include aqueous solutions of the active preparation in water-soluble form. Additionally, suspensions of the active ingredients may be prepared as appropriate oily or water based injection suspensions. Suitable lipophilic solvents or vehicles include fatty oils such as sesame oil, or synthetic fatty acids esters such as ethyl oleate, triglycerides or liposomes. Aqueous injection suspensions may contain substances, which increase the viscosity of the suspension, such as sodium carboxymethyl cellulose, sorbitol or dextran. Optionally, the suspension may also contain suitable stabilizers or agents which increase the solubility of the active ingredients to allow for the preparation of highly concentrated solutions.

Alternatively, the active ingredient may be in powder form for constitution with a suitable vehicle, e.g., sterile, pyrogen-free water based solution, before use.

The pharmaceutical composition of the present invention may also be formulated in rectal compositions such as suppositories or retention enemas, using, e.g., conventional suppository bases such as cocoa butter or other glycerides.

Pharmaceutical compositions suitable for use in context of the present invention include compositions wherein the active ingredients are contained in an amount effective to achieve the intended purpose. More specifically, a therapeutically effective amount means an amount of active ingredients (the antibody according to some embodiments of the invention) effective to prevent, alleviate or ameliorate symptoms of a disorder (e.g., cancer or an infectious disease) or prolong the survival of the subject being treated.

Determination of a therapeutically effective amount is well within the capability of those skilled in the art, especially in light of the detailed disclosure provided herein.

For any preparation used in the methods of the invention, the therapeutically effective amount or dose can be estimated initially from in vitro and cell culture assays. For example, a dose can be formulated in animal models to achieve a desired concentration or titer. Such information can be used to more accurately determine useful doses in humans.

Toxicity and therapeutic efficacy of the active ingredients described herein can be determined by standard pharmaceutical procedures in vitro, in cell cultures or experimental animals. The data obtained from these in vitro and cell culture assays and animal studies can be used in formulating a range of dosage for use in human. The dosage may vary depending upon the dosage form employed and the route of administration utilized. The exact formulation, route of administration and dosage can be chosen by the individual physician in view of the patient's condition. (See e.g., Fingl, et al., 1975, in “The Pharmacological Basis of Therapeutics”, Ch. 1 p. 1).

Dosage amount and interval may be adjusted individually to provide levels of the active ingredient (e.g., in the blood, plasma) which are sufficient to induce or suppress the biological effect (minimal effective concentration, MEC). The MEC will vary for each preparation, but can be estimated from in vitro data. Dosages necessary to achieve the MEC will depend on individual characteristics and route of administration. Detection assays can be used to determine plasma concentrations.

Depending on the severity and responsiveness of the condition to be treated, dosing can be of a single or a plurality of administrations, with course of treatment lasting from several days to several weeks or until cure is effected or diminution of the disease state is achieved.

The amount of a composition to be administered will, of course, be dependent on the subject being treated, the severity of the affliction, the manner of administration, the judgment of the prescribing physician, etc.

Compositions of the present invention may, if desired, be presented in a pack or dispenser device, such as an FDA approved kit, which may contain one or more unit dosage forms containing the active ingredient. The pack may, for example, comprise metal or plastic foil, such as a blister pack. The pack or dispenser device may be accompanied by instructions for administration. The pack or dispenser may also be accommodated by a notice associated with the container in a form prescribed by a governmental agency regulating the manufacture, use or sale of pharmaceuticals, which notice is reflective of approval by the agency of the form of the compositions or human or veterinary administration. Such notice, for example, may be of labeling approved by the U.S. Food and Drug Administration for prescription drugs or of an approved product insert. Compositions comprising a preparation of the invention formulated in a compatible pharmaceutical carrier may also be prepared, placed in an appropriate container, and labeled for treatment of an indicated condition, as is further detailed above.

In addition, the antibodies which recognize the two-domain structures regardless of the antigenic peptide can be used for pharmacokinetic studies in which the background levels originating from RTL-like structures is normalized; for detection of naturally RTL-like serum structures and for isolation and purification of these structures.

As mentioned above and further illustrated in the Examples section which follows, the isolated high affinity entity (e.g., the antibody) according to some embodiments of the invention can be used to detect the soluble T cell receptor ligand in a sample and thus can be used to monitor the presence and/or level of the soluble T cell receptor ligand in a biological sample obtained from a subject (e.g., blood, serum, plasma). This is of particular importance in cases where the recombinant T cell receptor ligand (a drug) is administered to a subject (e.g., for the treatment of an autoimmune disease such as multiple sclerosis) and the half life of the drug (e.g., pharmacokinetics analysis) can be determined using the specific high affinity entities of some embodiments of the invention. In addition, detection of native RTL-like structures in a sample of a subject is important in order to identify conditions characterized by excessive regulation of T cells by native RTL-like structures.

Thus, according to an aspect of some embodiments of the invention, there is provided a method of determining a presence and/or level of a soluble T cell receptor ligand in a sample, comprising contacting the sample with the high affinity entity of some embodiments of the invention under conditions which allow immunocomplex formation, wherein a presence or a level above a predetermined threshold of the immunocomplex is indicative of the presence and/or level of the soluble T cell receptor ligand in the sample, thereby determining the presence and/or the level of the soluble T cell receptor ligand in the sample.

The sample can be any biological sample obtained from the individual such as body fluids e.g., whole blood, serum, plasma, cerebrospinal fluid, urine, lymph fluids, and various external secretions of the respiratory, intestinal and genitourinary tracts, tears, saliva, milk as well as white blood cells, malignant tissues, amniotic fluid, chorionic villi, and bone marrow sample.

Contacting the sample with the high affinity entity (e.g., the antibody)/molecule or multivalent composition of the invention may be effected in vitro (e.g., in a sample of an individual), ex vivo or in vivo.

As mentioned, the method of the invention is effected under conditions sufficient to form an immunocomplex; such conditions (e.g., appropriate concentrations, buffers, temperatures, reaction times) as well as methods to optimize such conditions are known to those skilled in the art, and examples are disclosed herein.

As used herein the phrase “immunocomplex” refers to a complex which comprises the high affinity entity of some embodiments of the invention (e.g., the antibody) and the soluble T cell receptor ligand (e.g., the RTL or the native RTL-like structure, with or without the antigenic peptide).

Determining a presence or level of the immunocomplex of the invention can be performed using various methods are known in the art (e.g., immunological detection methods such as Western Blot, Immunohistochemistry, immunofluorescence, and the like) and further described hereinabove. For example, when the high affinity entity is conjugated to a detectable moiety, detection can be directly via the detectable moiety. Alternatively or additionally, a secondary labeled high affinity entity (e.g., antibody), directed against the high affinity entity of the invention can be used. For example, a rabbit anti-human antibody, a mouse anti-human antibody, and the like, can be used as is well known and accepted in the art.

The level of the immunocomplex in the tested sample is compared to a predetermined threshold. The threshold may be determined based on a known reference level and/or a level in a control sample (e.g., a sample of a healthy individual, control individual devoid of the disease which require administration of the RTL; or a sample of the same subject obtained prior to administration of the recombinant T cell receptor ligand into the subject). According to some embodiments of the invention, the control sample is of the same subject obtained prior to administration of the recombinant T cell receptor ligand to the subject.

According to some embodiments of the invention, the method further comprising performing a calibration curve using known amounts of the recombinant T cell receptor ligand, such as described in FIG. 7C.

According to an aspect of some embodiments of the invention there is provided a method of determining pharmacokinetic of a recombinant T cell receptor ligand in a blood of a subject. The method is effected by (a) administering the recombinant T cell receptor ligand to the subject, and (b) determining at predetermined time points a presence and/or level of the recombinant T cell receptor ligand in a blood sample of the subject according to the method of some embodiments of the invention, thereby determining the pharmacokinetic of the recombinant T cell receptor ligand in the blood of a subject

According to some embodiments of the invention, determining presence and/or level of the recombinant T cell receptor ligand in a blood sample is performed at least once after administration of the recombinant T cell receptor ligand to the subject.

It should be noted that such determination can be effected after at least 1 minute, 5, 10, 20, 30, 40, 50, 60 minutes, 1 hour, 2, 3, 4, 5, 6, 7, 8, 9, 10, 11, 12, 13, 14, 15, 16, 17, 18, 19, 20, 21, 22, 23, 24 hours, 2, 3, or more days after administration of the recombinant T cell receptor ligand.

In pharmacokinetic (PK) studies of a clinical trial using RTL1000 (Yadav et al., 2010) a short half-life (˜5 minutes) of circulating RTL1000 post infusion was observed. For the detection of RTL1000 in plasma and serum samples of the subjects, polyclonal Abs in sera from mice immunized with RTL1000 were used. The high specificity of Fab 2E4 to RTL1000 in a peptide-restricted manner enables a sensitive detection of circulating RTL1000 in plasma samples with no background (non-specific) binding to native MHC complexes or to other native RTL-like structures. Using Fab 2E4 a new assay was developed for PK studies and measurement of RTL1000 levels in serum. This assay was found to have greater sensitivity (of at least ˜two-fold) compared to the poly-clonal serum Abs used in the clinical study (Yadav et al., 2010) and therefore allows more accurate PK studies.

The high affinity entities of some embodiments of the invention which are described hereinabove for detecting the complexes of soluble T cell receptor ligands (e.g., RTLs or native RTL-like structures) with or without antigenic peptide may be included in a diagnostic kit/article of manufacture preferably along with appropriate instructions for use and labels indicating FDA approval for use in detecting the presence of the recombinant T cell receptor ligand in the sample.

Thus, according to an aspect of some embodiments of the invention there is provided a kit for detecting presence of a soluble T cell receptor ligand (e.g., RTL or native RTL-like structure) in a sample, comprising the high affinity entity of some embodiments of the invention and instructions for use in detecting the presence of the soluble T cell receptor ligand (e.g., RTL or native RTL-like structure) in the sample.

Such a kit can include, for example, at least one container including at least one of the above described diagnostic agents (e.g., the high affinity entity, e.g., the antibody) and reagents for detecting presence of an immunocomplex comprising the high affinity entity and the soluble T cell receptor ligand (e.g., RTL or native RTL-like structure) such as an imaging reagent packed in another container (e.g., enzymes, secondary antibodies, buffers, chromogenic substrates, fluorogenic material). The kit may also include appropriate buffers and preservatives for improving the shelf-life of the kit.

According to some embodiments of the invention, the kit further comprising the recombinant T cell receptor ligand.

According to some embodiments of the invention, the recombinant T cell receptor ligand included in the kit has known amounts of serial dilutions which can be used as reference for detection and quantification of an RTL in a sample.

As used herein the term “about” refers to ±10%.

The terms “comprises”, “comprising”, “includes”, “including”, “having” and their conjugates mean “including but not limited to”.

The term “consisting of means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et α1., (1989); “Current Protocols in Molecular Biology” Volumes I-III Ausubel, R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”, Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”, Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press, San Diego, Calif. (1990); Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### General Materials and Experimental Methods

Generation of Biotinylated RTLs

RTL1000 and RTL340 constructs were modified for a biotinylated version. In these constructs, a Bir-A tag (LHHILDAQKMVWNHR, SEQ ID NO:157) for biotinylation was introduced to the C-terminus of the RTL using a 20-aa flexible linker. RTLs DNA sequences were amplified and modified from PET-21(d+)-RTL1000 and PET-21(d+)-RTL340 DNA plasmid constructs [Chang J W, Mechling D E, Bächinger H P, Burrows G G. J. Biol. Chem. 2001 276(26): 24170-6. Design, engineering, band production of human recombinant t cell receptor ligands derived from human leukocyte antigen DR2) by PCR. The primers used to generate the RTL1000-biotin were 5′-TTAAGCGTTGGCGCATATGGAAGTTGGTTGG-3′ (NdeI RTL1000 Forward primer) (SEQ ID NO: 454) and 5′-TTAAGCGTTGGCGGAATTCTTATCA GCGGTGATTCCACACCATCTTCTGGGCGTCCAGGATATGGTGCAGAGACCC GGGATTGGTGATCGGAGTATAG-3′ (EcoRI Bir-A-Tag reverse primer) (SEQ ID NO: 455) and for RTL340-biotin were 5′-TTAAGCGTTGGCGCATATGGGGGACACCCGAG-3′ (NdeI RTL340 forward primer) (SEQ ID NO: 456) and 5′-TTAAGCGTTGGCGGAATTCTTATCA GCGGTGATTCCACACCATCTTCTGGGCGTCCAGGATATGGTGCAGAGACCC GGGATTGGTGATCGGAGTATAG-3′ (EcoRI Bir-A-Tag reverse primer) (SEQ ID NO:194). The amplification reactions were gel-purified, and the desired bands were isolated (QIAquick gel extraction kit; Qiagen). Each PCR amplification product was digest with NdeI and EcoRI restriction enzymes (New England BioLabs Inc., Beverly, Mass.) and gel-purified, and the RTLs DNA fragments were isolated. The RTLs DNA inserts were ligated with NdeI/EcoRI-digested pRB98 plasmid expression vector and transformed into BL21(DE3)pBirA-competent cells for protein expression.

Production of Biotinylated RTLs

DNA constructs encoding the biotinylated RTLs on the pRB98 plasmid were transformed into BL21(DE3)pBirA-competent cells for protein expression. These cells carry an additional plasmid with exogenous BirA ligase under the lac promoter. Bacteria were grown in 1-liter cultures to mid-logarithmic phase (OD₆₀₀ 0.6-0.8) in Luria-Bertani broth containing ampicillin (100 μg/ml) at 37° C. Recombinant protein production was induced by addition of 1 mM isopropyl-β-D-thiogalactoside. After overnight incubation at 30° C., the cells were centrifuged and stored at −20° C. before processing. Biotinylated inclusion bodies were isolated and solubilized in 20 mM ethanolamine, 6 M urea, pH 10, for 4 hours. After centrifugation, the supernatant containing RTL constructs were purified and concentrated by Fast Protein Liquid Chromatography (FPLC) ion exchange chromatography using Q Sepharose anion exchange media (GE healthcare, UK). Homogeneous peaks of the appropriate size were collected and further purified for homogeneity by size exclusion chromatography on a Sephacryl 5200 column (GE healthcare). The pooled fractions were dialyzed extensively against 20 mM TRIS buffer, pH=8.5 at 4° C., and concentrated to 1 mg/ml. The final yield of purified protein varied between 5 and 10 mg/L of bacterial culture.

Production of DR4 Molecules in S2 Cells

DES TOPO DR-A1*0101/DR-B1*0401(HA-307-319) plasmids for inducible expression in Schneider S2 cells, a gift from Dr. Lars Fugger, were used for cloning of the DR-B1*0401(GAD-555-567) construct, transfection and expression of recombinant four-domain MHC class II as previously reported (Cosson, P., J. S. Bonifacino. 1992. Role of transmembrane domain interactions in the assembly of class II MHC molecules. Science 258:659; Svendsen P, Andersen C B, Willcox N, Coyle A J, Holmdahl R, Kamradt T, Fugger L. 2004. Tracking of proinflammatory collagen-specific T cells in early and late collagen-induced arthritis in humanized mice. J Immunol. 1; 173(11):7037-45). Briefly, in these constructs the intracellular domains of the DR-A and DR-B chains were replaced by leucine-zipper dimerization domains for heterodimer assembly. The antigenic peptide was introduced to the N-terminus of the DR-B chain through a flexible linker. The Bir A recognition sequence for biotinylation was introduced to the C-terminus of the DR-A chain. DR-A and DR-B plasmids were co-transfected with pCoBlast selection vector to S2 cells using cellfectin reagent (Invitrogen, Carlsbad, Calif., US). Stable single-cell line clones were verified for protein expression. Upon induction with CuSO₄, cell supernatants were collected and DR4 complexes were affinity purified by anti-DR LB3.1 mAb (ATCC number HB-298). The purified DR4 complexes were biotinylated by Bir-A ligase (Avidity) and characterized by SDS-PAGE. The correct folding of the complexes were verified by recognition of anti-DR conformation sensitive mAb (L243) in an ELISA binding assay.

Selection of Phage Abs on Biotinylated Complexes

Selection of phage Abs on biotinylated complexes was performed as described before (Denkberg, 2002; Lev, 2002). Briefly, a large human Fab library containing 3.7×10¹⁰ different Fab clones was used for the selection. Phages were first preincubated with streptavidin-coated paramagnetic beads (200 μl; Dynal) to deplete the streptavidin binders. The remaining phages were subsequently used for panning with decreasing amounts of biotinylated MHC-peptide complexes. The streptavidin-depleted library was incubated in solution with soluble biotinylated RTLs or four-domain DR4/GAD (500 nM for the first round, and 100 nM for the following rounds) for 30 minutes at room temperature. Streptavidin-coated magnetic beads (200 μl for the first round of selection and 100 μl for the following rounds) were added to the mixture and incubated for 10-15 minutes at room temperature. The beads were washed extensively 12 times with PBS/0.1% Tween 20 and an additional two washes were with PBS. Bound phages were eluted with triethylamine (100 mM, 5 minutes at room temperature), followed by neutralization with Tris-HCl (1 M, pH 7.4), and used to infect E. coli TG1 cells (OD=0.5) for 30 minutes at 37° C. The diversity of the selected Abs was determined by DNA fingerprinting using a restriction endonuclease (BstNI), which is a frequent cutter of Ab V gene sequences.

Expression and Purification of Soluble Recombinant Fab Abs

Fab Abs were expressed and purified as described before (Denkberg, et al., 2002). TG1 or BL21 cells were grown to OD₆₀₀=0.8-1.0 and induced to express the recombinant Fab Ab by the addition of IPTG for 3-4 hours at 30° C. Periplasmic content was released using the B-PER solution (Pierce), which was applied onto a prewashed TALON column (Clontech). Bound Fabs were eluted using 0.5 ml of 100 mM imidazole in PBS. The eluted Fabs were dialyzed twice against PBS (overnight, 4° C.) to remove residual imidazole.

ELISA with Phage Clone Sups and Purified Fab Antibodies

Binding specificity of individual phage clone supernatants and soluble Fab fragments were determined by ELISA using biotinylated two and four-domain MHC/peptide complexes. ELISA plates (Falcon) were coated overnight with BSA-biotin (1 μg/well). After being washed, the plates were incubated (1 hour at room temperature) with streptavidin (10 μg/ml), washed extensively and further incubated (1 hour at room temperature) with 5 μg/ml of MHC/peptide complexes. The plates were blocked for 30 minutes at room temperature with PBS/2% skim milk and subsequently were incubated for 1 hour at room temperature with phage clone supernatants (induced at OD₆₀₀=0.8-1.0 for overnight expression at 30° C.) or 5 μg/ml soluble purified Fab. After washing, plates were incubated with horseradish peroxidase-conjugated/anti-human-Fab antibody. Detection was performed using TMB reagent (Sigma). For binding of peptide-loaded RTLs, ELISA plates were coated 2 hours at 37° C. with purified Fab, washed extensively and blocked for 30 minutes with PBS/2% skim milk. Loaded complexes were incubated for 1 hour followed by 1 hour incubation with anti-MHC class II mAb (TU39, BD). After washing, plates were incubated with horseradish peroxidase-conjugated/anti-mouse-IgG antibody and detection was performed as described above.

Competition Binding Assays

ELISA plates were coated with BSA-biotin and MHC-peptide complexes were immobilized as described above. Binding of soluble purified Fabs was performed by competitive binding analysis, which examined the ability of varying concentrations of soluble recombinant MHC-peptide complexes to inhibit the binding of the purified Fab to the specific immobilized MHC-peptide complex. Detection of Fabs binding to the immobilized MHC-peptide complexes was performed as described above.

Flow Cytometry

Cells were incubated for 4 hours with medium containing 70 μM MOG-35-55 (MEVGWYRPPFSRVVHLYRNGK, SEQ ID NO:129) or MBP-85-99 (ENPVVHFFKNIVTPR, SEQ ID NO:130) for L-cell DR*1501 transfectants and with GAD-555-567 (NFFRMVISNPAAT, SEQ ID NO:126) or control peptide: HA-307-319 (PKYVKQNTLKLAT, SEQ ID NO:196), InsA-1-15 (GIVEQCCTSICSLYQ, SEQ ID NO:158), and CII-261-273 (AGFKGEQGPKGEP, SEQ ID NO:195)—for DR4-EBV-transformed B lymphoblast Preiss cells. Cells (10⁶) were washed and incubated with 1-2 μg of specific Fab for 1 hour at 4° C., followed by incubation with FITC-labeled anti-human Ab for 45 minutes at 4° C. Cells were finally washed and analyzed by a FACSCalibur flow cytometer (BD Biosciences).

IL-2 Bioassay for the H2-1 T-Cell Hybridoma

H2-1 T-cell hybridoma cells (2×10⁵/well in a 96-well plate) in 100 μl of 10% FBS-containing medium were combined with 2×10⁵ irradiated (4,500 rad) HLA-DRB1*1501-transfected L cells (2) in 100 μl alone or in the presence of 10 μg/ml individual peptides and incubated at 37° C. and 7% CO₂ for 72 hours. Supernatants were collected from the top of the culture, followed by centrifugation for 1 minute at 1,000 rounds per minute (rpm). Hybridoma supernatants were added in triplicate into wells containing 5,000 CTLL-2 cells in 100 μl of 10% FBS culture medium. After 24 hours of culture, the cells were pulsed with 0.5 μCi [³H]thymidine for an additional 5 hr and the net counts per minute (cpm) (mean+/−SD) were calculated.

RTL In Vitro Potency Assay Using H2-1 T-Cell Hybridomas

Human MOG-35-55 peptide-specific H2-1 T-cell hybridoma cells (2×10⁵/well) were co-cultured in triplicate with 2 mM Tris-containing medium alone, 8 μM RTL1000, or 8 μM RTL340 in 2 mM Tris-containing medium for 72 hours. Aliquotted hybridoma cell cultures were thoroughly washed with RPMI and further stimulated with and without 10 μg/ml hMOG-35-55 peptide presented by irradiated (4,500 rad) DRB1*1501-transfected cell lines at a 1:1 ratio in triplicate for 48 hours. Half of the supernatant was collected from the top of each well and transferred into corresponding wells of another culture plate in which 100 μl of 10% FBS-containing medium with 5,000 CTLL cells per well had been seeded. After 24 hours of culture, the CTLL cells were pulsed with [³H]thymidine for additional 4 hours and the net cpm (mean+/−SD) were calculated.

RTL Treatment of EAE in DR2-Tg Mice

HLA-DR2 mice were screened by FACS for the expression of the HLA transgenes. HLA-DR2 positive male and female mice between 8 and 12 weeks of age were immunized subcutaneously (s.c.) at four sites on the flanks with 0.2 ml of an emulsion of 200 μg mouse MOG-35-55 peptide and complete Freund's adjuvant containing 400 μg of Mycobacterium tuberculosis H37RA (Difco, Detroit, Mich.). In addition, mice were given pertussis toxin (Ptx) from List Biological Laboratories (Campbell, Calif.) on days 0 and 2 post-immunization (75 ng and 200 ng per mouse, respectively) Immunized mice were assessed daily for clinical signs of EAE on a 6 point scale of combined hind limb and forelimb paralysis scores. For hind limb scores: 0=normal; 0.5=limp tail or mild hind limb weakness (i.e., a mouse cannot resist inversion after a 90° turn of the base of the tail); 1=limp tail and mild hind limb weakness; 2=limp tail and moderate hind limb weakness (i.e., an inability of the mouse to rapidly right itself after inversion); 3=limp tail and moderately severe hind limb weakness (i.e., inability of the mouse to right itself after inversion and clear tilting of hind quarters to either side while walking); 4=limp tail and severe hind limb weakness (hind feet can move but drag more frequently than face forward); 5=limp tail and paraplegia (no movement of hind limbs). Front limb paralysis scores are either 0.5 for clear restriction in normal movement or 1 for complete forelimb paralysis. The combined score is the sum of the hind limb score and the forelimb score. Rarely, there is mortality of HLA-DR2 mice with severe EAE, and in these cases, mice are scored as a 6 for the remainder of the experiment.

HLA-DR2 mice were treated with vehicle, RTL342m alone, or RTL342m pre-incubated with one of the FAbs. Treatment began on the first day that the combined clinical EAE score for each individual mouse reached 2 or higher. Once-daily treatments were administered to each mouse subcutaneously in the interscapular region for three days. RTL342m and RTL342m+FAb were prepared in 100 μl of 20 mM Tris-HCl pH 8.0 with 5% weight per volume (w/v) D-glucose (Sigma-Aldrich, St. Louis, Mo.). Vehicle treatments consisted of only Tris-HCl pH 8.0 with 5% w/v D-glucose. Mean EAE scores and standard deviations for mice grouped according to initiation of RTL or vehicle treatment were calculated for each day. The Cumulative Disease Index (CDI) was determined for each mouse by summing the daily EAE scores. Group CDI scores were calculated by determining the mean+SD of the individual mice in the group.

Serum ELISA with Fabs

Detection of RTL-like material in human serum or plasma was determined by ELISA using Fab 1B11. ELISA plates (Falcon) were coated for 2 hours with anti-MHC mAb TU39 (10 μg/well). The plates were blocked for 30 minutes at room temperature with PBS/2% skim milk and subsequently were incubated for 2 hours at room temperature with serial dilutions of RTL1000 (for standard curve) and 1:10 serum dilutions. After being washed, the plates were incubated (1 hour at room temperature) with 1B11 Fab (10 μg/ml), washed extensively and further incubated (1 hour at room temperature) with anti-myc-biotin Ab (9E10 clone, Covance). The plates were washed and incubated for 30 minutes with horseradish peroxidase-conjugated streptavidin. Further amplification steps were performed using the ELAST ELISA amplification system (PerkinElmer), according to the manufacturer's protocol. Detection was performed using TMB reagent (Sigma). Detection of RTL1000 in human serum or plasma was determined by ELISA using biotinylated Fab 2E4. ELISA plates (Falcon) were coated overnight with BSA-biotin (1 μg/well). After being washed, the plates were incubated (1 hour at room temperature) with streptavidin (10 μg/ml), washed extensively and further incubated (1 hour at room temperature) with 5 μg/ml of biotinylated Fab 2E4. The plates were blocked for 30 minutes at room temperature with PBS/2% skim milk and subsequently were incubated for 2 hours at room temperature with serial dilutions of RTL1000 and RTL340 (for standard curve) and 1:10 serum dilutions. After washing, plates were incubated with anti-DR/DP/DQ mAb (Tu39 clone, BD) followed by horseradish peroxidase-conjugated/anti-mouse antibody. Detection was performed using TMB reagent (Sigma).

Surface Plasmon Resonance

Immobilization of goat anti-human IgG Fab-specific Fab (Jackson ImmunoResearch Cat #109006097) was performed on a GLM (General Layer Medium) chip (Bio-Rad Laboratories, Hercules, Calif., USA) at 25° C. in the vertical orientation, and the continuous running buffer was PBST (10 mM Na-phosphate, 150 mM NaCl, and 0.005% Tween 20, pH 7.4). Six channels were activated with 50 μl of a mixture of 0.04 M N-ethyl-N-(3-dimethylaminopropyl) carbodiimide (EDC) and 0.01 M sulfo-N-hydroxysuccinimide (Sulfo-NHS) at a flow rate of 30 μl/min. The anti-Fab specific Fab was diluted in 10 mM sodium acetate buffer pH 4.5 to a final concentration of 25 μg/ml, and 150 μl were injected followed by an injection of 150 μl of 1 M ethanolamine-HCl pH 8.5. The immobilization levels were about 4,000 RU. Next, 150 μl of five different supernatants were injected in the vertical orientation in five different channels to allow their capture by the immobilized Fab anti Fab. The sixth channel remained empty to serve as a reference. The RTL1000 antigen was injected (75 μl at 50 μl/minute) in the horizontal orientation of the ProteOn XPR36 system using five different concentrations (1000, 500, 250, 125 and 62.5 nM). Running buffer was injected simultaneously in the sixth channel for double referencing to correct for loss of the captured supernatant from the chip sensor surface during the experiment. All binding sensorgrams were collected, processed and analyzed using the integrated ProteOn XPR36 system Manager (Bio-Rad Laboratories, Hercules, USA) software. Binding curves were fitted using the Langmuir model describing 1:1 binding stoichiometry, or with the Langmuir and mass transfer limitation model. Each individually captured antibody interacting with the five concentrations of antigen was fitted using a global ka, kd and Rmax. Global fitting is used when the same ka, kd and Rmax values describe a specific biological model like five antigen concentrations interacting with a certain antibody.

Production of an Recombinant Four Domain β1-α1/β2-α2 Complex with a Covalently Bound Peptide

DES TOPO DR-A1*0101/DR-B1*0401(HA-307-319) plasmids for inducible expression in Schneider S2 cell were used for cloning of DR-B1*0401(GAD₅₅₅₋₅₆₇) construct, transfection and expression of recombinant four-domain MHC class II as previously reported (Svendsen, P., et al., 2004). Briefly, in these constructs the intracellular domains of the DR-A and DR-B chains were replaced by leucine-zipper dimerization domains of Fos and Jun transcription factors, respectively, for heterodimer assembly. The antigenic peptide was introduced to the N-terminus of the DR-B chain through a flexible linker. Bir A recognition sequence for biotinylation was introduced to the C-terminus of the DR-A chain. DR-A and DR-B plasmids were co-transfected with pCoBlast selection vector to S2 cells using cellfectin reagent (invitrogen). Stable single-cell line clones were verified for protein expression. Upon induction with CuSO₄, cells supernatant were collected and DR4 complexes were affinity purified by anti-DR LB3.1 (ATCC number HB-298) monoclonal antibody (mAb). The purified DR4 complexes were biotinylated by Bir-A ligase (Avidity) and characterized by SDS-PAGE. The right folding of the complexes was verified by recognition of anti-DR conformation sensitive mAb (L243) in ELISA binding assay.

Statistics

All experiments performed under this study are presented as independent assays which are representative of three to nine independent experiments. IL-2 bioassays were performed in triplicates with SD bars indicated. For neutralization of RTL treatment of DR2-mice by Fabs, a two-tailed Mann-Whitney test for nonparametric comparisons was used to gauge the significance of difference between the mean daily and CDI scores of vehicle vs. RTL treatment groups. A one sided Fisher's exact test was used to gauge the significance of the number of “treated” mice between groups. A Kruskal-Wallis nonparametric analysis of variance test was also performed with a Dunn's multiple-comparison post-test to confirm significance between all groups. A two-tailed unpaired t-test was used to confirm significance of signal in 1B11 serum ELISA, while two-tailed paired t-test was used to gauge the significance between pre- vs. post-infusion samples. All statistical tests were computed using GraphPad Prism 4 (GraphPad software, Inc.).

### Example 1

### Generation and Characterization of Biotinylated RTLS

Human RTLs were found to have a secondary structure composition similar to the TCR recognition/peptide-binding α1β1 domain of native human MHC class II molecule (Burrows, 1999; Burrows 2001). In order to select for T-cell receptor like (TCR-like, or TCRL) antibodies (Abs) the present inventors have generated biotinylated versions of HLA-DR2 derived RTLs, RTL1000 (DR2/MOG-35-55) and RTL340 (DR2/MBP-85-99) and used them to select for antibodies having specificity to the RTLs but not to the four-domain MHC-peptide complexes.

Experimental Results

Characterization of Biotinylated RTLs

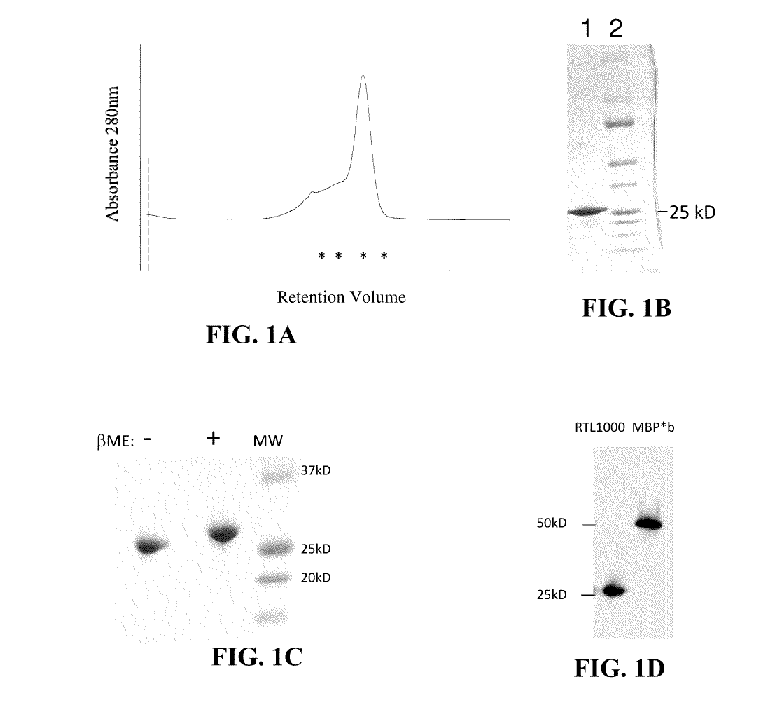

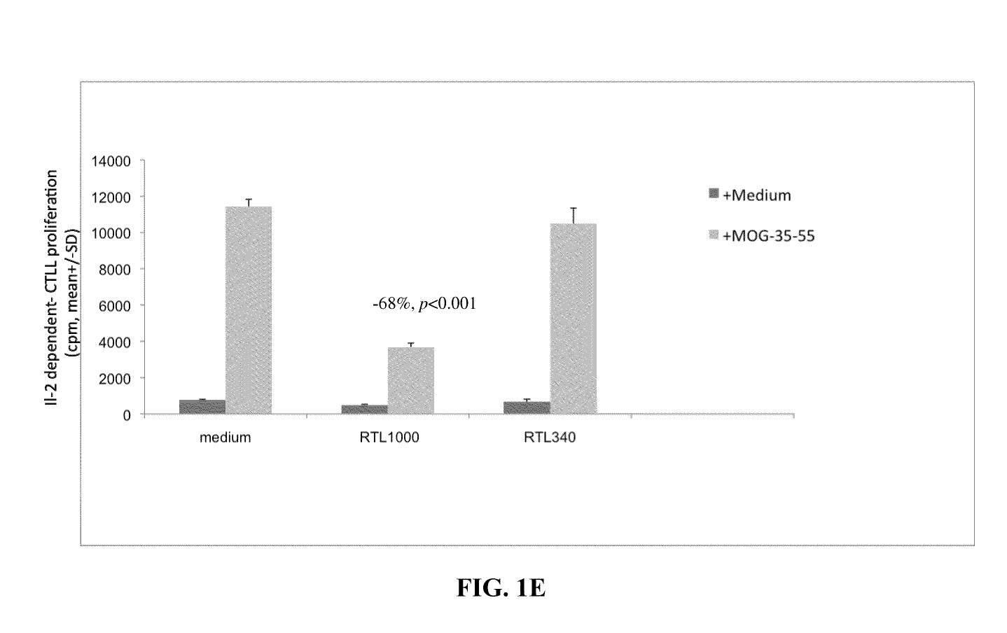

The RTL constructs were produced in bacteria and were isolated by in vitro refolding of purified inclusion bodies. The RTLs were found to be very pure, homogenous, and monomeric by SDS-PAGE and size exclusion chromatography analyses (FIGS. 1A-B). HLA-DR2 (DRA1*0101, DRB1*1501) contains a disulfide bond between conserved cysteines in the β1 domain (residues 15 and 79 of the DR-B chain) (Smith, 1998). The formation of this native conserved disulfide bond within the RTL molecule was verified by gel-shift assay (FIG. 1C). SDS-PAGE analyzes of reduced and non-reduced RTL1000 samples revealed that the non reduced sample has a smaller apparent molecular weight, indicating the presence of internal disulfide bond leading to a more compact structure. High biotinylation levels are essential for a successful screening of the desired Abs using the phage display screening strategy. The RTL constructs were found to have high biotinylation levels, identical to the compared 100% biotinylated MBP standard (FIG. 1D).

RTLs Mimic the Specific Interaction of the MHC Class II Peptide Complex with the T-Cell Receptor

In previous reports, RTLs were found to deliver peptide-specific rudimentary signals through the TCR of human Th1 cells (Burrows, 2001) and a murine T-cell hybridoma (Wang, 2003). The present inventors have verified the interaction of biotinylated RTL1000 with the cognate TCR of H2-1 T-cell hybridoma specific for the DR2/MOG-35-55 epitope. As shown in FIG. 1E, MOG-35-55 specific activation of H2-1 hybridoma was inhibited by pre-incubation of H2-1 with RTL1000. Control RTL340 (DR2/MBP-85-99) did not inhibit this antigen-specific response, indicating selective RTL1000 ligation of the TCR leading to inhibitory signaling. These results demonstrate that the RTL1000 construct mimics the minimal MHC II domains necessary for specific interaction with the TCR. Accordingly, the recombinant RTL1000 was used as a soluble recombinant protein for the selection of Abs directed to the α1β1 DR2/MOG-35-55 idiotope in a TCR-like fashion.

### Example 2

### Isolation of Recombinant Antibodies which Specifically Bind RTLS

Experimental Results

Isolation of Recombinant Abs with TCR-Like Specificity Toward RTL1000

For selection of TCRL Abs directed to MHC class II, the present inventors screened a large Ab phage library consisting of a repertoire of 3.7×10¹⁰ human recombinant Fab fragments (de Haard, 1999). RTL1000 was used as a minimal DR2/MOG-35-55 epitope recognized by autoreactive T-cells. The library was applied to panning on soluble RTL1000. Seven hundred-fold enrichment in phage titer was observed following four rounds of panning. The specificity of the selected phage Abs was determined by ELISA comparison of streptavidin-coated wells incubated with biotinylated RTL1000 (DR2/MOG-35-55) or RTL340 (DR2/MBP-85-99) (FIG. 2A). Fab clones with peptide-dependent, MHC-restricted binding were picked for further characterization. DNA fingerprinting, by BstNI restriction reaction, revealed 23 different restriction patterns of MOG peptide-dependent DR2 specific Fabs, indicating the selection of several different Fabs with this unique specificity.

Specificity and Affinity of TCR-Like Fabs Specific for RTL1000

Bacteria E. coli cells were used to produce a soluble Fab form of a representative clone of each DNA restriction pattern. The specificity of the selected clones was characterized in a competition ELISA binding assay. Binding of the Fabs to the immobilized RTL1000 complex was competed with a soluble RTL1000 (DR2/MOG-35-55), control RTL340 (DR2/MBP-85-99), with free MOG-35-55 peptide (SEQ ID NO:129) alone or with free MBP-85-99 alone. By this assay the present inventors were able to verify the binding of the Fabs to soluble DR/peptide complexes and to exclude a conformational distortion by direct binding to plastic. As shown in FIGS. 2B-C for two representative Fabs (2E4 and 2C3), neither RTL340 (DR2/MBP-85-99) nor MOG-35-55 peptide alone could compete the Fab binding to immobilized RTL1000. By performing this assay the present inventors were able to discriminate between Fabs that bind soluble MOG-35-55 peptide (represented by 2B4, FIG. 2D) and those that bind a portion of this peptide when bound to the two-domain DR2 molecules in a TCR-like fashion (such as 2E4 and 2C3, FIGS. 2B-C). FIG. 2E shows five different Fabs that were found to have a MOG-35-55 specific, DR2 restricted TCR-like specificity to the α1β1 DR2/MOG complex. These Fabs were tested in an ELISA binding assay and were found to bind only to the two-domain DR2/MOG-35-55 complex (RTL1000) and not to a two-domain DR2 complex containing a control peptide (RTL340; marked as DR2/MBP-85-99 in FIG. 2E), an empty two-domain DR2 complex (marked as empty DR2 in FIG. 2E), or MOG-35-55 peptide. Fab 1B11 was isolated and found to bind all HLA-DR-derived RTLs with no peptide-specificity and dependency (FIG. 2E). Commercially available TU39 anti-MHC class II mAb [described in Pawelec G, et al., 1985, Hum. Immunol. 1985; 12(3):165-176; and Ziegler A, et al., 1986, Immunobiology, 171(1-2):77-92] was used to verify identical quantities of the different complexes that were compared (FIG. 2E).

Determination of Complementarity Determining Regions (CDRs) of the Isolated Fabs

DNA sequencing confirmed the selection of five different clones directed specifically to the α1β1 DR2/MOG-35-55 complex (Table 15, hereinbelow). The affinities of the Fabs to RTL1000 were measured and analyzed by a Surface Plasmon Resonance (SPR) biosensor (ProteOn XPR36, Bio-Rad Laboratories) and found to be in the range of 30-150 nM.

TABLE 15
CDR (Complementarity Determining Region) sequences and binding
affinity of the anti-RTL1000 TCRL Fab Abs.
Isolated antibodies which specifically recognize RTL1000 CDR

| Variable H chain | | | Variable L chain | | | Affinity | Antibody |
|---|---|---|---|---|---|---|---|
| CDR3 | CDR2 | CDR1 | CDR3 | CDR2 | CDR1 | (nM) | Name |
| EGDNYYG | IINPSGGST | GYTFT | QQRSN | DASNR | RASQSV | 33 | 2E4 |
| DAFDI | SYAQKFQ | SYYMH | WPPSYT | AT (SEQ | SSYLA | | |
| (SEQ ID | (SEQ ID | (SEQ ID | (SEQ ID | ID NO: 2) | (SEQ ID | | |
| NO: 9) | NO: 8) | NO: 7) | NO: 3) | | NO: 1) | | |
| ESHPAAAL | SISYSGSTY | GVSISS | QQYGTS | GASSR | RASQSII | 60 | 1F11 |
| VG (SEQ ID | YNPSLKS | RSGH | PLT | AT (SEQ | NSHLA | | |
| NO: 25) | (SEQ ID | WG | (SEQ ID | ID | (SEQ ID | | |
| | NO: 24) | (SEQ ID | NO: 19) | NO: 18) | NO: 17) | | |
| | | NO: 23 | | | | | |
| VRGHRYY | SISSSSSYIY | GETFS | QQANSF | TASSLQ | RASQVIS | 58 | 3A3 |
| YDSSGYYS | YADSVKG | SYSMN | PLT | S (SEQ | SWLA | | |
| SDYYYYY | (SEQ ID | (SEQ ID | (SEQ ID | ID | (SEQ ID | | |
| GMDV | NO: 40) | NO: 39) | NO: 35) | NO: 34) | NO: 33) | | |
| (SEQ ID | | | | | | | |
| NO: 41) | | | | | | | |
| DERDAYY | YIYYSGST | GGSIS | MQALQ | LGSNR | RSSQSLL | 129 | 3H5 |
| YGMDV | NYNPSLKS | GYYW | TPLT | AS (SEQ | HSNGNN | | |
| (SEQ ID | (SEQ ID | S (SEQ | (SEQ ID | ID | YLD | | |
| NO: 57) | NO: 56) | ID | NO: 51) | NO: 50) | (SEQ ID | | |
| | | NO: 55) | | | NO: 49) | | |
| DRSFWSGY | VISYDGSN | GFTFS | MQALHI | LGSNR | RSSQSLL | 153 | 2C3 |
| YIINYYYY | KYYADSV | SYAM | PLT | AS (SEQ | HSDGNN | | |
| GMDV | KG (SEQ ID | H (SEQ | (SEQ ID | ID | YLD | | |
| (SEQ ID | NO: 72) | ID | NO:67) | NO: 66) | (SEQ ID | | |
| NO: 73) | | NO: 71) | | | NO: 65) | | |

### Example 3

### Fine Specificity of the Isolated Fabs

Experimental Results

Fine Specificity of Anti-Two-Domain DR2/MOG-35-55 TCRL Fabs

To analyze the fine specificity of the isolated Fabs the present inventors tested the recognition of the Fabs to RTL342m, a two-domain DR2 complex with mouse MOG-35-55 peptide. Mouse (m)MOG-35-55 peptide (MEVGWYRSPFSRVVHLYRNGK) (SEQ ID NO:200) carries a Pro→Ser substitution at position 42 of the MOG polypeptide as compared to human (h)MOG-35-55 (SEQ ID NO:129). This single amino-acid substitution altered the recognition of all the 5 anti-RTL1000 Fabs (2E4, 1F11, 3A3, 2C3 and 3H5; FIG. 3A) as detected by ELISA binding. Fabs 2C3 and 3H5 completely lost their ability to bind the RTL when the peptide was of a mouse origin (the altered complex). Reduction in the binding of the Fabs to RTL342m compared to RTL1000 was obtained for 1F11 and 3A3 (5-fold) and 2E4 (2 fold). The dependence of reactivity of these selected Fabs on this 42-Pro anchor residue implies a unique peptide conformation in the context of the HLA-DR2 α1β1 domains. In addition, none of the Fabs reacted with the mMOG-35-55 in the context of the murine allele I-A^{b} (RTL551) (FIG. 3A), emphasizing the TCR-like requirement of the Fabs to the cognate peptide within the MHC allele.

To exclude the possibility of reactivity of the Fabs with the linker attaching the MOG-35-55 peptide to the RTL construct, the present inventors tested the binding of the isolated Fabs to empty DR2 derived RTL (RTL302) loaded with free MOG-35-55 peptide. All the Fabs kept their peptide-specific, MHC restricted binding to the MOG-35-55 loaded empty RTL302 (FIG. 3B) excluding any binding-dependence to non-native sequences of RTL1000.

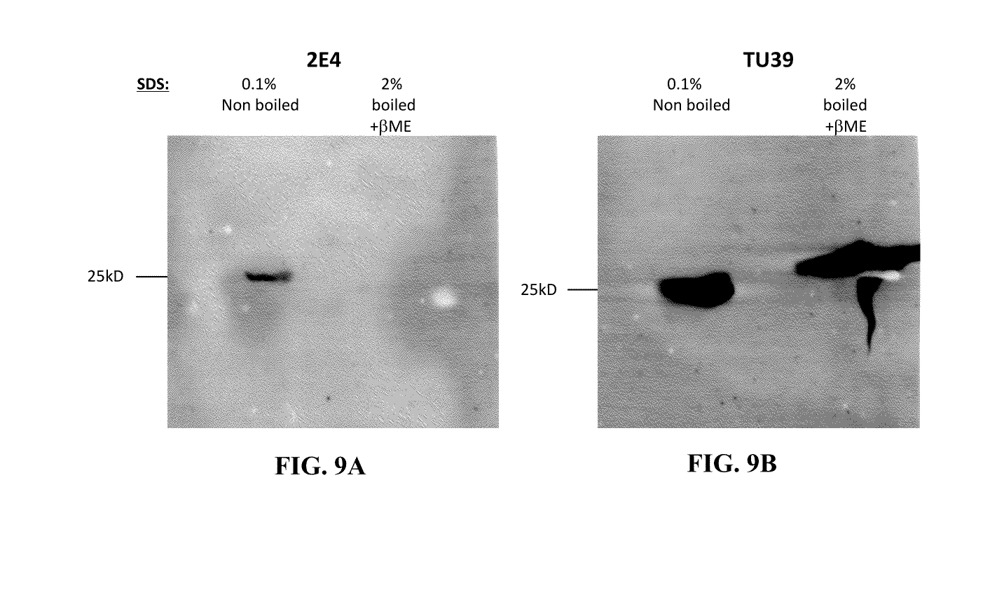

Additionally, the present inventors tested Fab binding to RTL1000 in different buffer conditions and found the Fabs to be conformational sensitive, losing their ability to react with denatured RTL1000 (FIGS. 9A-B).

Taken together, these data indicate selective Fab binding to the α1β1 DR2/MOG-35-55 native sequence of the folded RTL1000.

### Example 4

### Conformational Differences Between RTL and Full Length MHC Class II Molecule

Experimental Results

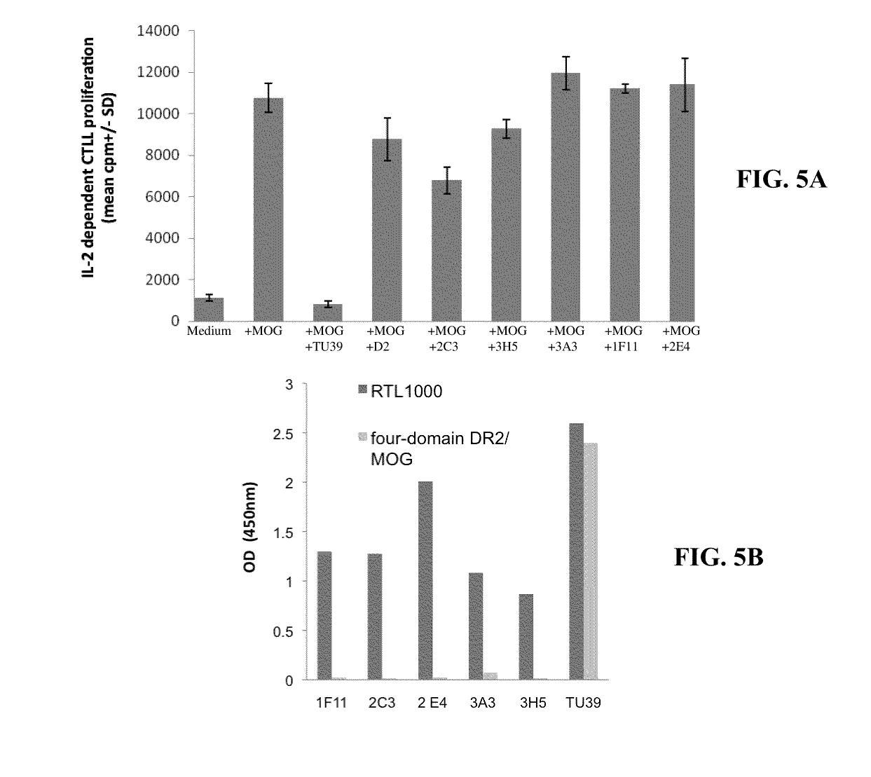

The isolated anti RTL1000 Fabs do not bind to the four-domain MHC class II-antigenic peptide complex when loaded on antigen presenting cells (APCs)—The present inventors have tested the ability of the anti-two-domain DR2/MOG-35-55 Fabs to bind the native full length four-domain form of MHC II complexes as expressed on APCs. L-cell DR*1501 transfectants (L466.1 cells) were loaded with MOG-35-55 or control peptide. The loaded cells were incubated with the purified Fabs following anti-Fab-FITC incubation. No specific binding of the 1F11, 2C3, 2E4, 3A3 and 3H5 Fabs was observed for MOG-35-55 loaded cells (FIGS. 4B-F). MOG-35-55 and control peptide loaded cells produced the same fluorescence intensity as background. MHC expression on the APC surface was confirmed by anti-DR mAb (L243, BD, FIG. 4A). A portion of the loaded cells that were used for the FACS analysis was incubated with H2-1 T-cell hybridoma specific for the DR2/MOG-35-55 idiotope. Following 72 hours of incubation, cell supernatants were transferred to IL-2-dependent CTLL cells (Chou Y K, Culbertson N, Rich C, LaTocha D, Buenafe A C, Huan J, Link J, Wands J M, Born W K, Offner H, Bourdette D N, Burrows G G, Vandenbark A A. T-cell hybridoma specific for myelin oligodendrocyte glycoprotein-35-55 peptide produced from HLA-DRB1*1501-transgenic mice. J Neurosci Res. 2004 Sep. 1; 77(5):670-80) for detection of IL-2 levels secreted from H2-1 hybridoma (FIG. 5A). H2-1 cells were activated only by the MOG-35-55 pulsed cells, secreting 8-fold higher levels of IL-2 compared to non-pulsed or control peptide-pulsed APCs. Peptide-specific H2-1 activation confirmed a successful loading of MOG-35-55 peptide to the native MHC on the APCs used for the FACS analysis.

Despite the presence of a biologically active determinant in the form of DR2/MOG-35-55 molecules presented by the APCs, no staining of such complex was obtained by any of the isolated anti RTL1000 Fabs. Considering the high affinity of the selected Fabs and the permissive conditions used for this experiment, it is conclude that the Fabs do not bind the native DR2/MOG-35-55 complex presented by APCs.

Further support for this finding came from blocking experiments which tested the Fabs ability to inhibit peptide-specific activation of the H2-1 hybridoma by DR2 APCs pulsed with MOG-35-55 peptide (FIG. 5B). None of the selected Fabs (2C3, 3H5, 3A3, 1F11 and 2E4) were able to block this peptide-specific, MHC restricted activation of the H2-1 hybridoma, as compared to a control TCRL Fab specific for DR4/GAD-555-567 (D2). Complete blocking was achieved by the control anti-MHC class II Mab (TU39, BD). The failure of the Fabs to interfere with MHC presentation to TCR implies the inability to bind native four domain DR2/MOG-35-55 complexes. This was indeed the case, as demonstrated by ELISA (FIG. 5C). Altogether, these data strongly suggest that there are conformational differences between two- vs. four-domain forms of HLA-DR2 loaded with the MOG-35-55 peptide

### Example 5

### The Isolated Anti-RTL Fabs can Reverse the Effect Achieved by the Specific RTL

Reversal of RTL342m Treatment of EAE in DR2 Tg Mice

To further test the functional attributes of Fab specific for the two-domain RTL1000 idiotope, the present inventors utilized a Fab specific for the RTL1000 idiotope that was also cross-reactive with a similar idiotope on RTL342m (α1β1 domains of DR2 linked to mouse (m)MOG-35-55 peptide). DR2 Tg mice were immunized with mMOG-35-55 peptide/CFA/Ptx to induce EAE and were treated with pre-formed complexes of 2E4 Fab:RTL342m, the control D2 Fab:RTL342m (specific for the DR4/GAD-555-567 RTL idiotope described below) or TRIS buffer. As is shown in FIG. 6A, mice receiving RTL342m plus TRIS buffer (RTL342m alone) were effectively treated, whereas a 2:1 ratio of 2E4 Fab:RTL342m almost completely neutralized the RTL therapeutic effect on EAE. In contrast, a 1:1 ratio of 2E4 Fab:RTL342m had less neutralizing activity as assessed by daily EAE scores (FIG. 6A) and by the entire experimental effect on EAE for each group as assessed by the Cumulative Disease Index (CDI) (FIG. 6B) Importantly, D2 Fab (also used at a 2:1 ratio) did not neutralize the therapeutic effect of RTL342m on EAE, indicating specificity of the 2E4 Fab for the two-domain RTL342m idiotope.

### Example 6

### Detection of Natural RTL-Like Two Domain MHC Class H Molecules in Human Plasma

Experimental Results

Detection of Natural RTL-Like Two-Domain MHC Class II Molecules in Human Plasma

In a recent Phase I safety study (Yadav, et al., 2010) using serum of mice immunized with RTL (polyclonal antibodies against RTL) baseline plasma levels of two-domain RTL-like structures were observed in 4 of 13 donors (31%) DR2+ MS subjects which were about to be treated with RTL1000 or placebo. This observation suggested the natural occurrence of two-domain structures that could be derived from four-domain intermediates possibly shed from class II expressing APC upon immunization. Using the power of the isolated conformational sensitive Fabs of some embodiments of the invention, the present inventors have evaluated the appearance and persistence of naturally occurring two domain MHC class II structures in human MS subjects. Fab 1B11 is specific for two-domain HLA-DR-conformation. It was found to bind to all HLA-DR-derived RTLs (with no peptide specificity), but not to other human and murine allele-derived RTLs or four-domain HLA-DR molecules (FIG. 10). Serum or plasma samples were diluted 1:10 and adsorbed onto plastic wells pre-coated with the TU39 mAb (that detects all forms of MHC), washed and reacted with 1B11 Fab specific for HLA-DR-derived RTLs, followed by addition of enzyme-labeled anti-Fab and substrate for ELISA detection. As is shown in FIG. 7A, the 1B11 Fab detected RTL-like material in serum or plasma from the healthy control pool as well as all six MS subjects tested at baseline, with detected levels of protein ranging from 13 ng/ml to 1,100 ng/ml. Increased signal for two-domain class II was also observed in MS Subject No. 42 after 30 minutes of infusion of 200 mg RTL1000 and in MS Subject No. 44 after 2 hours of infusion of 100 mg RTL1000, consistent with increased levels of injected RTL1000.

### Example 7

### Detection of RTL1000 in Plasma of Treated MS Subjects

Experimental Results

In order to detect injected RTL1000 in serum and plasma samples of MS subjects treated with RTL1000 and to discriminate it from the native RTL-like structures obtained by Fab 1B11, the present inventors have used Fab 2E4 which binds RTL1000 in a MOG-35-55 peptide-specific, DR2-restricted manner. As shown in FIG. 7B, 2E4 Fab successfully detected RTL1000 in plasma samples of MS subjects post RTL1000 infusion (MS samples No. 42 after 30 minutes and MS subject No. 44 120 minutes) while the pre-infusion samples (MS samples Nos. 04-402, 03-302, 24, 40, 42, and 44 at 0 minutes) and the pooled healthy human serum kept low background signal levels. The increase of the 1B11 Fab signal in the post- vs. pre-RTL1000 infusion samples is consistent with the detection of serum RTL1000 in the post-infusion samples by Fab 2E4. The combined Fabs data strongly support the presence of other peptide-specificities of native two-domain structures in the serum/plasma samples and the high utility of the isolated Fabs for such a sensitive and specific detection. FIG. 7D demonstrates the utility of 2E4 Fab for pharmacokinetic (PK) studies of RTL1000 infusion. RTL1000 levels in plasma of DR2+ MS subject No. 42 were measured during 120 minutes of RTL1000 infusion and during the following 60 minutes. Results from this PK study elegantly confined a previously determined half-life of RTL1000 in plasma as ˜5 minutes (Yadav, 2010).

### Example 8

### Isolation of Recombinant Abs with TCR-Like Specificity Toward RTL and Native DR4/GAD-555-567 Complex

Experimental Results

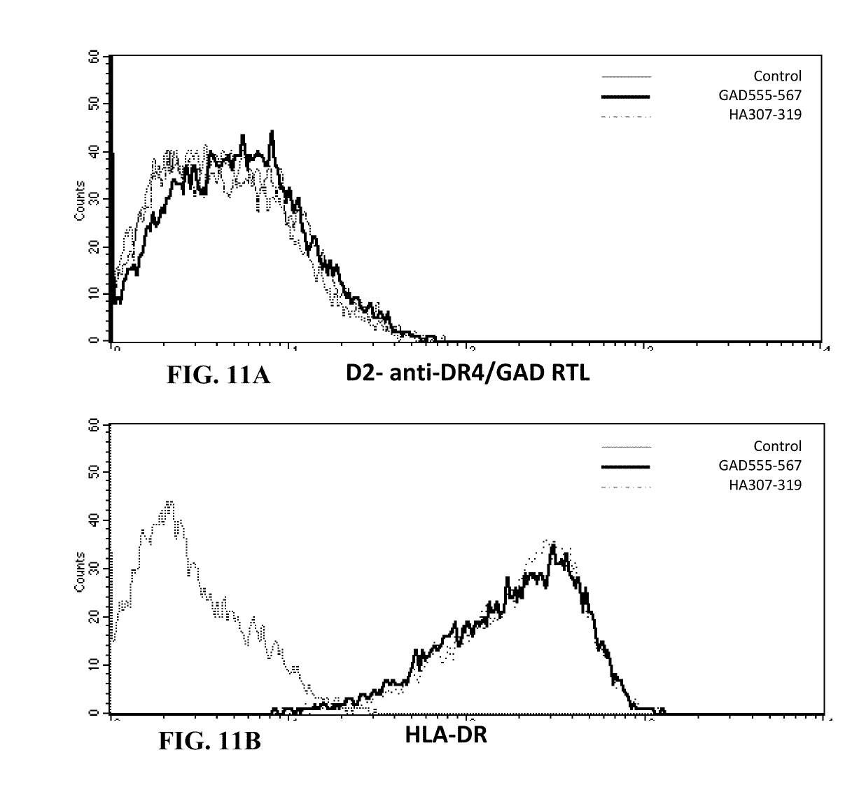

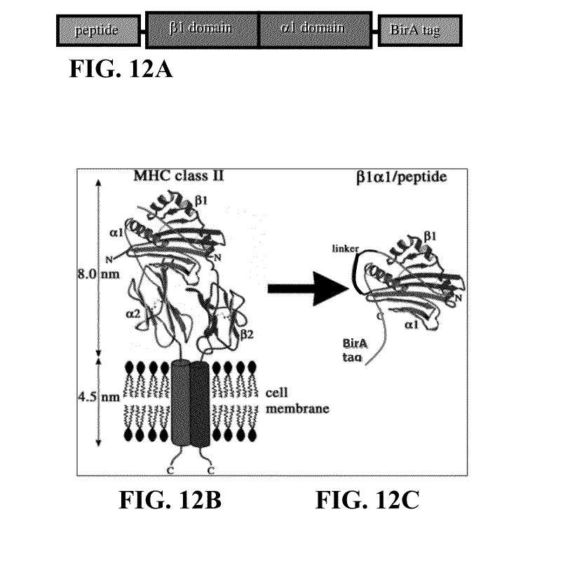

The present inventors have constructed DR4/GAD-555-567 RTL molecules and isolated a TCRL Fab, named D2, which is specific for the DR4/GAD RTL in a GAD-peptide dependent, DR4-restricted manner. D2 failed to react with four-domain DR4/GAD-555-567 complexes, both as recombinant protein (FIG. 8C) and as native complexes present by APCs (FIGS. 11A-B). Thus, similar to anti-RTL1000 TCRLs, D2 identified a distinct conformational difference between the two domain RTL structure vs. the four domain native MHC/peptide.

For the isolation of TCRLs directed to the native MHC/peptide complexes the present inventors applied the phage display strategy directed to recombinant full-length DR4/GAD-555-567 peptide. Four different TCRL Fab Abs were isolated and found to bind solely to recombinant full length DR4/GAD-555-567 complexes and not to DR4 complexes with control peptides, or to the GAD-555-567 peptide alone (FIG. 8A, for representative G3H8 Fab). Additionally, these TCRLs successfully detected native DR4/GAD-555-567 complexes presented by EBV transformed DR4+B cells (FIG. 7B for representative G3H8 Fab) and a variety of APC populations in PBMCs from a DR4+ donor (Manuscript in preparation). Of importance, G3H8 Fab did not recognize the DR4/GAD-555-567 derived RTL in an ELISA binding assay (FIG. 7D). By using these two novel distinct TCRL Fab groups, unique conformational differences were detected between the two- and four-domain MHC versions of the DR4/GAD idiotope.

### Example 9

### Increasing Avidity of the Isolated Fabs by Generating Whole IgG Antibodies

The avidity of the isolated 1B11 Fab is increased by expressing the Fab as a whole IgG. This allows to use the antibody for immunoprecipitation of the novel serum structures which are RTL-like. For affinity column with specificities described above, the relevant Fab fragments are constructed into whole IgG Abs. The H and L Fab genes are cloned for expression as human IgG1 Ab into the eukaryotic expression vector pCMV/myc/ER. For the H chain, the multiple cloning site, the myc epitope tag, and the endoplasmic reticulum (ER) retention signal of pCMV/myc/ER were replaced by a cloning site containing recognition sites for BssHI and NheI followed by the human IgG1 constant H chain region cDNA isolated by RT-PCR from human lymphocyte total RNA. A similar construct was generated for the L chain. Each such expression vector carries a different antibiotic resistance gene. Expression is facilitated by cotransfection of the two constructs into the human embryonic kidney HEK293 cell by using the FuGENE 6 Transfection Reagent (Roche). After cotransfection, cells are grown on selective medium and clones are tested for obtaining the same specificity as the original Fab fragment. Positive clones are adapted to grow in 0.5% serum and are further purified using protein A (Sigma) affinity chromatography. SDS-PAGE analysis of the purified protein tests the existence of homogenous, pure IgG with the expected molecular mass of ˜150 kDa. The IgG Ab is crossed-linked to protein A beads using a standard protocol. Plasma and culture supernatants are loaded to the affinity column in neutralized pH and bound proteins are eluted by 0.1N acetic acid, pH=3 and are immediately neutralized.

Discussion and Analysis

In this study the present inventors have shown the ability to select, from a large non-immune repertoire of human Fab fragments, a panel of recombinant antibodies with TCR-like specificity directed to auto-reactive T-cell epitopes in the form of self peptide presented by MHC class II. Abs directed to MHC II/peptide complexes have been generated before, using epitope-specific immunization (MHC+peptide) as the initial step for further conventional hybridoma technology or construction of a phage display library (Stang, 1998; Rudensky, 1994; Krogsgaard, 2000; Zhong 1997; Eastman, 1996). The present inventors show here, for the first time, the generation of MHC II/peptide TCRL Fabs from a naïve human Ab library. Moreover, due to the large size of the phage display library, the present inventors were able to isolate several different Fabs directed to each targeted epitope. This method can be employed for generating of TCRL Fabs directed to other MHC II/peptide complexes.

Five different TCRL Fab clones directed to the minimal two-domain DR2/MOG-35-55 epitope of RTL1000 were isolated. Characterization of these Fabs indicated a requirement for both DR2 and MOG-35-55 peptide for recognition. The Fabs could further discern conformational differences in the P42S variant of DR2-bound MOG-35-55 peptide present in RTL342m, demonstrating individual variation in binding to specific contact residues within the DR2/MOG-35-55 complex. Moreover, cross-recognition of RTL342m by the 2E4 Fab allowed neutralization of RTL treatment of mMOG-35-55 induced EAE, illustrating the functional activity of this highly characterized Fab in vivo. These Abs therefore mimic the fine specificity of TCRs with the advantages of high-affinity and stable characteristics of the recombinant Fab fragment.

The TCRLs antibodies described herein exhibit high structural sensitivity while firmly distinguishing two- vs. four-domain MHC II/peptide idiotopes. None of the anti-RTL1000 TCRL Fabs were able to recognize four-domain DR2/MOG-35-55 presented by APC or in a recombinant form. Similarly, two panels of TCRL Fabs directed to two- or four-domain DR4/GAD-555-567 complexes clearly distinguished these two conformational idiotopes. The possibility that the Fabs directed to two-domain MHC are reacting with unique epitopes specific for bacterial derived products and not with conformational-specific epitopes is not likely, mainly due to the fact that several Fabs specificities were characterized to different RTL constructs made in the same bacterial system.

While the previous bio-physical and biochemical data suggest a similar secondary structure content for the RTL constructs and the peptide binding domains of native MHC (Burrows 1999), the novel TCRL Fabs have identified distinct conformational differences between MHC II/peptide and RTL/peptide complexes. Moreover, the present inventors have characterized specific interactions of both RTL1000 and four-domain DR2/MOG-35-55 with the cognate TCR present on the H2-1 T-cell hybridoma. The ability of defined TCR to bind these two distinct conformational idiotopes highlights the permissive nature of the TCR as compared to the TCRL Fabs. This characteristic is the basis for the design of TCR agonist and antagonist ligands such as RTLs.

It is conceivable that the RTL constructs are representative of naturally occurring soluble two-domain MHC class II structures that may function as inhibitors of T-cell responses. In recent Phase I safety study of RTL1000 in DR2+ MS subjects discussed above, detectable pre-infusion plasma levels of two-domain RTL-like structure were observed in 4 of 13 donors (31%). To verify these intriguing results, we re-evaluated pre- and post-infusion serum or plasma samples from 6 MS subjects from our trial and serum from a pool of 3 healthy donors using the 1B11 Fab specific for two-domain MHC class II structures (with no specificity for bound peptide). Diverse quantities of such structures (ranged from 13 ng/ml to 1038 ng/ml) were found in all evaluated subjects. These novel results suggest the natural occurrence of two-domain structures that could be derived from four-domain intermediates possibly shed from class II expressing APC upon immunization (MacKay, 2006). Such MHC class II-derived structures may act as natural analogues of RTL constructs and induce similar regulatory effects on T-cell responses. Most importantly, the appearance of natural two-domain class II molecules in human plasma would provide support for the biological relevance of the RTL constructs. The Abs directed to the two-domain MHC conformation are valuable tool for isolation and identification of such native structures. The comparison between the signal levels detected by Fab 1B11 (pan DR two domain structures) and Fab 2E4 (DR2/MOG-35-55 two-domain structure of RTL1000) in the plasma of subjects after infusion of RTL1000 demonstrate the high sensitivity of the novel Fabs isolated herein.

This study presents novel finding that autoreactive four vs. two domain MHC class II TCR ligands have distinct conformational shapes that can be distinguished by human Fab molecules and that apparently confer opposing immunological functions (peptide-specific T cell activation vs. tolerance). This concept is of fundamental importance for understanding immunological tolerance, since it implies that the distinct shape of class II idiotopes formed by truncated two-domain structures may provide a natural tolerogen for regulating inflammatory T cells selected originally on four-domain structures.

In PK studies of the clinical trial discussed above the present inventors observed a short half-life (˜5 minutes) of circulating RTL1000 post infusion (personal communication, Vandenbark AA). For the detection of RTL1000 in plasma and serum samples of the subjects, the present inventors used polyclonal Abs in sera from mice immunized with RTL1000. The high specificity of Fab 2E4 to RTL1000 in a peptide-restricted manner enabled its sensitive detection of circulating RTL1000 in plasma samples with no background of native MHC and other-peptide specificities of RTL-like structures. Using Fab 2E4 the present inventors developed a new assay for PK studies and measurement of RTL1000 levels in serum. This assay was found to have greater sensitivity (˜two-fold) compared to the use of poly-clonal serum Abs in the original assay and therefore allows more accurate PK studies (manuscript in preparation).

The therapeutic effects of RTLs on T-cell mediated autoimmunity may involve several complementary pathways. In addition to direct TCR ligation, RTL regulatory effects on inflammatory CD4+ T-cells might work through manipulation of APCs. Recent studies (Sinha et al., 2010) demonstrated high avidity binding of RTLs to macrophages, dendritic cells and B cells, and such RTL “armed” myeloid cells (but not B cells) could tolerize T-cells specific for the RTL-bound peptide. The current study clearly demonstrates that two-domain idiotope embodied by RTLs are distinct from the corresponding four-domain idiotopes, and these two-domain structures deliver tolerogenic rather than activating signals through the cognate TCR. The TCRL Fabs will be used to further elucidate the in-vivo therapeutic pathways of RTL1000 in the humanized DR2-Tg EAE model. RTL342m idiotype-specific TCRLs can be used to both inhibit RTL binding to APC and block RTL association with the TCR, as would be predicted for Fab 2E4.

In recent years, with the advantage of fluorochrome-labeled MHC class II multimers, there is increased knowledge about specific CD4+ T-cells in various inflammatory autoimmune conditions (Reijonen, 2002; Reijonen, 2004; Svendsen, 2004; Korn, 2007; Macaubas 2006). T1D patients and at-risk subjects were found to have a significantly higher prevalence of GAD-555-567 specific CD4 T-cells than control subjects (Oling, 2005). The novel TCRL to four vs. two-domain idiotopes have the potential to selectively recognize APCs presenting disease-inducing or regulatory idiotopes, respectively, to islet cell-responsive CD4+ T-cells during T1D. Similarly, Fabs to four vs. two domain DR2/MOG-35-55 idiotopes may be invaluable in localizing and quantifying encephalitogenic vs. tolerogenic APC in subjects with MS.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

### REFERENCES

### Additional References are Cited in Text

Adamus, G., G. G. Burrows, A. A. Vandenbark, and H. Offner. 2006. Treatment of Autoimmune Anterior Uveitis with Recombinant TCR Ligands. Invest. Ophthalmol. Vis. Sci. 47:2555-2561.
Burrows, G. G., J. W. Chang, H. P. Bachinger, D. N. Bourdette, H. Offner, and A. A. Vandenbark. 1999. Design, engineering and production of functional single-chain T− cell receptor ligands. Protein Eng. 12:771-778.
Burrows, G. G., Y. K. Chou, C. Wang, J. W. Chang, T. P. Finn, N. E. Culbertson, J. Kim, D. N. Bourdette, D. A. Lewinsohn, D. M. Lewinsohn, M. Ikeda, T. Yoshioka, C. N. Allen, H. Offner, and A. A. Vandenbark. Rudimentary TCR Signaling Triggers Default IL-10 Secretion by Human Th1 Cells. J Immunol 167:4386-4395, 2001.
Chang J W, Mechling D E, Bächinger H P, Burrows G G. Design, engineering, and production of human recombinant t cell receptor ligands derived from human leukocyte antigen DR2. J Biol Chem. 2001 Jun 29; 276(26):24170-6.
Cohen, C. J., N. Hoffmann, M. Farago, H. R. Hoogenboom, L. Eisenbach, and Y. Reiter. 2002. Direct Detection and Quantitation of a Distinct T-Cell Epitope Derived from Tumor-specific Epithelial Cell-associated Mucin Using Human Recombinant Antibodies Endowed with the Antigen-specific, Major Histocompatibility Complex-restricted Specificity of T-Cells. Cancer Res 62:5835-5844.
Denkberg, G., C. J. Cohen, A. Lev, P. Chames, H. R. Hoogenboom, and Y. Reiter. 2002. Direct visualization of distinct T-cell epitopes derived from a melanoma tumor-associated antigen by using human recombinant antibodies with MHC-restricted T-cell receptor-like specificity. Proc Natl Acad Sci USA. 99:9421-9426.
Denkberg, G., A. Lev, L. Eisenbach, I. Benhar, and Y. Reiter. 2003. Selective Targeting of Melanoma and APCs Using a Recombinant Antibody with TCR-Like Specificity Directed Toward a Melanoma Differentiation Antigen. J Immunol 171:2197-2207.
Eastman S, M Deftos, P. C. DeRoos, D. H. Hsu, L Teyton, N. S. Braunstein, C. J. Hackett, and A. Rudensky. 1996. A study of complexes of class II invariant chain peptide: major histocompatibility complex class II molecules using a new complex-specific monoclonal antibody. Eur J Immunol 26(2):385-393.
Epel M, I Carmi, S Soueid-Baumgarten, S. K. Oh, T Bera, I Pastan, J Berzofsky, and Reiter Y. 2008. Targeting TARP, a novel breast and prostate tumor-associated antigen, with T-cell receptor-like human recombinant antibodies. Eur J Immunol 38(6): 1706-20.
de Haard, H. J., N. van Neer, A. Reurs, S. E. Hufton, R. C. Roovers, P. Henderikx, A. P. de Brune, J. W. Arends, and H. R. Hoogenboom. 1999. A Large Non-immunized Human Fab Fragment Phage Library That Permits Rapid Isolation and Kinetic Analysis of High Affinity Antibodies. J Biol Chem 274:18218-18230.
Huan, J., L. J. Kaler, J. L. Mooney, S. Subramanian, C. Hopke, A. A. Vandenbark, E. F. Rosloniec, G. G. Burrows, and H. Offner. 2008. MHC Class II Derived Recombinant T-Cell Receptor Ligands Protect DBA/1LacJ Mice from Collagen-Induced Arthritis. J Immunol 180:1249-1257.
Johns TG, K. N., Menon, K K, Abo S, Gonzales M. F., Bernard C. C. 1995. Myelin oligodendrocyte glycoprotein induces a demyelinating encephalomyelitis resembling multiple sclerosis. J Immunol 154:5536-5541.
Karlsen, A. E., W. A. Hagopian, C. E. Grubin, S. Dube, C. M. et al. 1991. Cloning and primary structure of a human islet isoform of glutamic acid decarboxylase from chromosome 10. Proc Natl Acad Sci USA 88:8337-8341.
Kerlero de Rosbo N, and A. Ben-Nun. 1998. T-cell responses to myelin antigens in multiple sclerosis: relevance of the predominant autoimmune reactivity to myelin oligodendrocyte glycoprotein. J Autoimmun 11:287-295.
Kerlero de Rosbo N, R Milo, M. B. Lees, D Burger, C. C Bernard, and A. Ben-Nun. 1993. Reactivity to myelin antigens in multiple sclerosis: peripheral blood lymphocytes respond predominantly to myelin oligodendrocyte glycoprotein. J Clin Invest 92:2602-2608
Korn, T., J. Reddy, W. Gao, E. Bettelli, A. Awasthi, T. R. Petersen, B. T. Backstrom, R. A. Sobel, K. W. Wucherpfennig, T. B. Strom, M. Oukka, and V. K. Kuchroo. 2007. Myelin-specific regulatory T-cells accumulate in the CNS but fail to control autoimmune inflammation. Nat Med 13:423-431.
Krogsgaard M, K. W. Wucherpfennig, B Cannella, B. E. Hansen, A. Svejgaard, J Pyrdol, H Ditzel, C Raine, and L Fugger. 2000. Visualization of Myelin Basic Protein (Mbp) T-Cell Epitopes in Multiple Sclerosis Lesions Using a Monoclonal Antibody Specific for the Human Histocompatibility Leukocyte Antigen (Hla)-Dr2-Mbp 85-99 Complex. J Exp Med 191:1395-1412.
Lev, A, G. Denkberg, C J. Cohen, M. Tzukerman, K. L. Skorecki, P. Chames, H. R. Hoogenboom, and Y. Reiter. 2002. Isolation and Characterization of Human Recombinant Antibodies Endowed with the Antigen-specific, Major Histocompatibility Complex-restricted Specificity of T-Cells Directed toward the Widely Expressed Tumor T-cell Epitopes of the Telomerase Catalytic Subunit. Cancer Res 62:3184-3194.
Link, J. M., C. M. Rich, M. Korat, G. G. Burrows, H. Offner, and A. A. Vandenbark. 2007. Monomeric DR2/MOG-35-55 recombinant TCR ligand treats relapses of experimental encephalomyelitis in DR2 transgenic mice. Clin Immunol 123:95-104.
Macaubas C, J. Wahlstrom, A. P. Galvao da Silva, T. G. Forsthuber, G. Sonderstrup, W. W. Kwok, R. H. DeKruyff, and D. T. Umetsu. 2006. Allergen-Specific MHC Class II Tetramer+ Cells Are Detectable in Allergic, but Not in Nonallergic, Individuals. J Immunol 176:5069-5077.
MacKay P. A., S. Leibundgut-Landmann, N Koch, A. C. Dunn., W Reith, R. W. and A. D. McLellan. 2006. Circulating, soluble forms of major histocompatability complex antigens are not exosome-associated. Eur J Immunol 36:2875-2884.
McDaniel D. O., B. O. Barger, R. T. Acton, W. J. Koopman, and G. S. Alarcn. 1989. Molecular analysis of HLA-D region genes in seropositive rheumatoid arthritis. Tissue Antigens 34:299-308.
Mendel I, N Kerlero de Rosbo, and A. Ben-Nun. 1995. A myelin oligodendrocyte glycoprotein peptide induces typical chronic experimental autoimmune encephalomyelitis in H-2 mice: Fine specificity and T-cell receptor Vbeta expression of encephalitogenic T-cells. Eur J Immunol 25:1951-1959.
Michaeli, Y., G. Denkberg, K. Sinik, L. Lantzy, C. Chih-Sheng, C. Beauverd, T. Ziv, P. Romero, and Y. Reiter. 2009. Expression Hierarchy of T-Cell Epitopes from Melanoma Differentiation Antigens: Unexpected High Level Presentation of Tyrosinase-HLA-A2 Complexes Revealed by Peptide-Specific, MHC-Restricted, TCR-Like Antibodies. J Immunol 182:6328-6341.
Nepom, G. T., and H. Erlich. 1991. MHC Class-II Molecules and Autoimmunity. Annu Rev Immunol 9:493-525.
Nepom, G. T., J. D. Lippolis, F. M. White, S. Masewicz, J. A. Marto, A. Herman, C. J. Luckey, B. Falk, J. Shabanowitz, D. F. Hunt, V. H. Engelhard, and B. S. Nepom. 2001. Identification and modulation of a naturally processed T-cell epitope from the diabetes-associated autoantigen human glutamic acid decarboxylase 65 (hGAD65). Proc Natl Acad Sci USA 98:1763-1768.
Olerup O, and J Hilert. 1991. HLA class II-associated genetic susceptibility in multiple sclerosis: a critical evaluation. Tissue Antigens. 38:1-15.
Oling V, J Marttila, J Ilonen, W. W. Kwok, G Nepom, M Knip, O Simell, and H Reijonen. 2005. GAD65− and proinsulin-specific CD4+ T-cells detected by MHC class II tetramers in peripheral blood of type 1 diabetes patients and at-risk subjects. J Autoimmun 25:235-243.
Patel, S. D., A. P. Cope, M. Congia, T. T. Chen, E. Kim, L. Fugger, D. Wherrett, and G. Sonderstrup-McDevitt. 1997. Identification of immunodominant T-cell epitopes of human glutamic acid decarboxylase 65 by using HLA-DR (alpha1*0101,beta1*0401) transgenic mice. Proc Natl Acad Sci USA 94:8082-8087.
Pawelec G, Ziegler A, Wernet P. Dissection of human allostimulatory determinants with cloned T cells: stimulation inhibition by monoclonal antibodies TU22, 34, 35, 36, 37, 39, 43, and 58 against distinct human MHC class II molecules. Hum. Immunol. 1985; 12(3):165-176;
Quill, H., and R. H. Schwartz. 1987. Stimulation of normal inducer T-cell clones with antigen presented by purified Ia molecules in planar lipid membranes: specific induction of a long-lived state of proliferative nonresponsiveness. J Immunol 138:3704-3712.
Reijonen, H., R. Mallone, A. K. Heninger, E. M. Laughlin, S. A. Kochik, B. Falk, W. W. Kwok, C. Greenbaum, and G. T. Nepom. 2004. GAD65-Specific CD4+ T-Cells with High Antigen Avidity Are Prevalent in Peripheral Blood of Patients With Type 1 Diabetes. Diabetes 53:1987-1994.
Reijonen, H, E. J. Novak., S. Kochik, A. Heninger, A. Liu and W. Kwok. 2002. Detection of GAD65-Specific T-Cells by Major Histocompatibility Complex Class II Tetramers in Type 1 Diabetic Patients and At-Risk Subjects. Diabetes 51:1375-1382
Rich C, J. M Link., A Zamora, H Jacobsen, R Meza-Romero, H Offner, R Jones, G. G Burrows, L Fugger, and A. A Vandenbark. 2004. Myelin oligodendrocyte glycoprotein-35-55 peptide induces severe chronic experimental autoimmune encephalomyelitis in HLA-DR2-transgenic mice. Eur J Immunol34:1251-1261.
Rudensky, A. Y., M. Maric, S. Eastman, L. Shoemaker, P. C. DeRoos, and J. S. Blum. 1994. Intracellular assembly and transport of endogenous peptide-MHC class II complexes. Immunity 1:585-594.
Sawcer S, M Ban, M Maranian, T. W. Yeo, A Compston, A. Kirby, M. J. Daly, P. L. De Jager, E Walsh, E. S. Lander, J. D. Rioux, D. A. Hafler, A Ivinson, J Rimmler, S. G. Gregory, S Schmidt, M. A. Pericak-Vance, E Akesson, J Hillert, P Datta, A Oturai, L. P. Ryder, H. F. Harbo, A Spurkland, K. M. Myhr, M Laaksonen, D Booth, R Heard, G Stewart, R Lincoln, L. F. Barcellos, S. L. Hauser, J. R. Oksenberg, S. J. Kenealy, J. L. Haines; International Multiple Sclerosis Genetics Consortium. 2005. A High-Density Screen for Linkage in Multiple Sclerosis. Am J Hum Genet 77:454-467.
Schwartz R. H., Models of T-cell anergy: is there a common molecular mechanism? 1996. J Exp Med 1:19-29.
Sinha S, L Miller, S Subramanian, O McCarty, T Proctor, R Meza-Romero, G. G Burrows, A. A Vandenbark, and H Offner. 2010. Binding of recombinant T-cell receptor ligands (RTL) to antigen presenting cells prevents upregulation of CD11b and inhibits T-cell activation and transfer of experimental autoimmune encephalomyelitis. J Neuroimmunol.
Sinha, S., S. Subramanian, L. Miller, T. M. Proctor, C. Roberts, G. G. Burrows, A. A. Vandenbark, and H. Offner. 2009. Cytokine Switch and Bystander Suppression of Autoimmune Responses to Multiple Antigens in Experimental Autoimmune Encephalomyelitis by a Single Recombinant T-Cell Receptor Ligand. J. Neurosci 29:3816-3823.
Sinha S, S Subramanian, T. M. Proctor, L. J Kaler, M Grafe, R Dahan, J Huan, A. A. Vandenbark, G. G Burrows, and H Offner. 2007. A promising therapeutic approach for multiple sclerosis: recombinant T-cell receptor ligands modulate experimental autoimmune encephalomyelitis by reducing interleukin-17 production and inhibiting migration of encephalitogenic cells into the CNS. J Neurosci 14; 27(46):12531-9.
Smith, K. J., J. Pyrdol, L. Gauthier, D. C. Wiley, and K. W. Wucherpfennig. 1998. Crystal Structure of HLA-DR2 (DRA*0101, DRB1*1501) Complexed with a Peptide from Human Myelin Basic Protein. J Exp Med 188:1511-1520.
Stang, E., C. B. Guerra, M. Amaya, Y. Paterson, O. Bakke, and E. D. Mellins 1998. DR/CLIP (Class II-Associated Invariant Chain Peptides) and DR/Peptide Complexes Colocalize in Prelysosomes in Human B Lymphoblastoid Cells. J Immunol 160:4696-4707.
Subramanian, S., B. Zhang, Y. Kosaka, G. G. Burrows, M. R. Grafe, A. A. Vandenbark, P. D. Hum, and H. Offner. 2009. Recombinant T-Cell Receptor Ligand Treats Experimental Stroke. Stroke 40:2539-2545.
Trapp B. D, and K. A. Nave. 2008. Multiple Sclerosis: An Immune or Neurodegenerative Disorder? Annu Rev Neurosci 31:247-269.
Verge, C. F., R Gianani, E Kawasaki, L Yu, M Pietropaolo, R. A. Jackson, H. P. Chase, G. S. Eisenbarth. 1996. Prediction of type I diabetes in first-degree relatives using a combination of insulin, GAD, and ICA512bdc/IA-2 autoantibodies Diabetes 45:926-933
Wang C., J. L. Mooney, R. Meza-Romero, Y. K. Chou, J. Huan, A. A. Vandenbark, H. Offner, and G. G. Burrows. 2003. Recombinant TCR Ligand Induces Early TCR Signaling and a Unique Pattern of Downstream Activation. J Immunol 171:1934-1940.
Yadav Rudensky V, D Bourdette, J. D. Bowen, S. G. Lynch, D Mattson, J Preinigerova, C Rose, R. B Stead, A. J. Ferro, A. S. Goldstein, G. G. Burrows, H Offner, and A. A. Vandenbark. (2010). Recombinant T-Cell Receptor Ligand (RTL) for the Treatment of Multiple Sclerosis: Report of a Phase I Clinical Trial. Neurology, 74:S2; A293-294.
Zhong G, C Reis e Sousa, R. N. Germain. 1997. Production, specificity, and functionality of monoclonal antibodies to specific peptide-major histocompatibility complex class II complexes formed by processing of exogenous protein. Proc Natl Acad Sci USA 94(25):13856-β861.
Ziegler A, Heinig J, Muller C, et al. Analysis by sequential immunoprecipitations of the specificities of the monoclonal antibodies TU22, 34, 35, 36, 37, 39, 43, 58 and YD1/63. HLK directed against human HLA class II antigens. Immunobiology. 1986; 171(1-2):77-92.

1. An isolated high affinity entity comprising an antigen binding domain which specifically binds a soluble T-cell receptor ligand comprising a two-domain β1-α1 of a major histocompatibility complex (MHC) class II, wherein said antigen binding domain does not bind a complex comprising a four-domain α1-β1/α2-β2 MHC class II.

2. The isolated high affinity entity of claim 1, wherein said two-domain β1-α1 of said MHC class II is in complex with an MHC class II antigenic peptide.

3. The isolated high affinity entity of claim 1, wherein said four-domain α1-β1/α2-β2 MHC class II is in complex with said MHC class II antigenic peptide.

4. The isolated high affinity entity of claim 2, wherein said antigen binding domain does not bind said two-domain β1-α1 MHC class II in an absence of said MHC class II antigenic peptide, and wherein said antigen binding domain does not bind to said MHC class II antigenic peptide in an absence of said two-domain β1-α1 MHC class II.

5. The isolated high affinity entity of claim 2, wherein said two-domain β1-α1 of said MHC class II is covalently linked to said MHC class II antigenic peptide.

6. The isolated high affinity entity of claim 1, wherein said antigen binding domain comprising complementarity determining regions (CDRs) set forth by SEQ ID NOs:1-3 and 7-9 (CDRs 1-3 of light chain and heavy chain, respectively, of 2E4); SEQ ID NOs:17-19 and 23-25 (CDRs 1-3 of light chain and heavy chain, respectively, of 1F11); SEQ ID NOs:33-35 and 39-41 (CDRs 1-3 of light chain and heavy chain, respectively, of 3A3); SEQ ID NOs:49-51 and 55-57 (CDRs 1-3 of light chain and heavy chain, respectively, of 3H5); SEQ ID NOs:65-67 and 71-73 (CDRs 1-3 of light chain and heavy chain, respectively, of 2C3); SEQ ID NOs:97-99 and 103-105 (CDRs 1-3 of light chain and heavy chain, respectively, of D2).

7. The isolated high affinity entity of claim 1, wherein said antigen binding domain binds said two-domain β1-α1 of MHC class II when in complex with an MHC class II antigenic peptide or in an absence of said MHC class II antigenic peptide.

8. The isolated high affinity entity of claim 7, wherein said antigen binding domain comprising complementarity determining regions (CDRs) set forth by SEQ ID NOs:81-83 and 87-89 (CDRs 1-3 of light and heavy chain, respectively of 1B11).

9. A method of isolating a high affinity entity which specifically binds to a recombinant T-cell receptor ligand (RTL), comprising:
(a) screening a library comprising a plurality of high affinity entities with an isolated complex comprising a major histocompatibility complex (MHC) class II antigenic peptide being covalently linked to a two-domain β1-α1 of said MHC class II; and
(b) isolating at least one high affinity entity comprising an antigen binding domain which specifically binds said isolated complex, wherein said at least one high affinity entity does not bind to a complex comprising a four-domain α1-β1/α2-β2 MHC class II and said MHC class II antigenic peptide,
thereby isolating the high affinity entities which specifically binds to the recombinant T-cell ligand (RTL).

10. The method of claim 9, wherein said at least one high affinity entity does not bind said MHC class II in an absence of said MHC class II antigenic peptide, and wherein said at least one high affinity entity does not bind to said MHC class II antigenic peptide in an absence of said MHC class II.

11. The method of claim 9, wherein said isolated complex further comprising a peptide for site specific biotinylation.

12. The isolated high affinity entity of claim 1, wherein said antigen binding domain does not bind a complex of said MHC class II and said MHC class II antigenic peptide when presented on an antigen presenting cell (APC).

13. The isolated high affinity entity of claim 1, wherein said high affinity entity is selected from the group consisting of an antibody, an antibody fragment, a phage displaying an antibody, a peptibody, a bacteria displaying an antibody, a yeast displaying an antibody, and a ribosome displaying an antibody.

14. The isolated high affinity entity of claim 1, wherein the high affinity entity comprises a monoclonal antibody.

15. The isolated high affinity entity or the method of claim 13, wherein said antibody comprises a human antibody.

16. The isolated high affinity entity of claim 1, wherein said MHC class II is selected from the group consisting of HLA-DM, HLA-DO, HLA-DP, HLA-DQ, and HLA-DR.

17. The isolated high affinity entity of claim 1, wherein said MHC class II antigenic peptide is an autoantigenic peptide associated with a disease selected from the group consisting of diabetes, multiple sclerosis, rheumatoid arthritis, celiac uveitis and stroke.

18. The isolated high affinity entity of claim 17, wherein said autoantigenic peptide associated with said diabetes is derived from a polypeptide selected from the group consisting of preproinsulin (SEQ ID NO:113), proinsulin (SEQ ID NO:114), Glutamic acid decarboxylase (GAD (SEQ ID NO:115), Insulinoma Associated protein 2 (IA-2; SEQ ID NO:116), IA-213 (SEQ ID NOs:117, 133 and 134), Islet-specific Glucose-6-phosphatase catalytic subunit-Related Protein (IGRP isoform 1 (SEQ ID NO:118), and Islet-specific Glucose-6-phosphatase catalytic subunit-Related Protein (IGRP isoform 2 (SEQ ID NO:119), chromogranin A (ChgA) (SEQ ID NO:120), Zinc Transporter 8 (ZnT8 (SEQ ID NO:121), Heat Shock Protein-60 (HSP-60; SEQ ID NO:122), Heat Shock Protein-70 (HSP-70; SEQ ID NO:123 and 124).

19. The isolated high affinity entity or the method of claim 18, wherein said GAD autoantigenic peptide comprises a core amino acid sequence set forth by SEQ ID NO:125 (GAD556-565, FFRMVISNPA).

20.-21. (canceled)

22. The isolated high affinity entity or the method of claim 17, wherein said autoantigenic peptide associated with said multiple sclerosis is derived from a polypeptide selected from the group consisting of myelin oligodendrocyte glycoprotein (MOG; SEQ ID NOs:135-143), myelin basic protein (MBP; SEQ ID NOs:127 and 144-148), and proteolipid protein (PLP; SEQ ID NOs:128, 149 and 150).

23.-26. (canceled)

27. A method of determining a presence and/or level of a soluble T cell receptor ligand in a sample, comprising contacting the sample with the isolated high affinity entity of claim 1, under conditions which allow immunocomplex formation, wherein a presence or a level above a predetermined threshold of said immunocomplex is indicative of the presence and/or level of the soluble T cell receptor ligand in the sample, thereby determining the presence and/or the level of the soluble T cell receptor ligand in the sample.

28. The method of claim 27, further comprising performing a calibration curve using known amounts of the soluble T cell receptor ligand.

29. A method of determining pharmacokinetic of a soluble T cell receptor ligand in a blood of a subject, comprising:
(a) administering the soluble T cell receptor ligand to the subject, and
(b) determining at predetermined time points a presence and/or level of the soluble T cell receptor ligand in a blood sample of the subject according to the method of claim 27,
thereby determining the pharmacokinetic of the soluble T cell receptor ligand in the blood of a subject.

30. A kit for detecting presence of a soluble T cell receptor ligand in a sample, comprising the isolated high affinity entity of claim 1 and instructions for use in detecting the presence of the soluble T cell receptor ligand in the sample.

31.-32. (canceled)

33. A method of sequestering soluble T cell receptor ligand in a subject, comprising administering the isolated high affinity entity of claim 1 to the subject, thereby sequestering soluble T cell receptor ligand.

34.-36. (canceled)

37. The isolated high affinity entity of claim 1, wherein said soluble T-cell receptor ligand comprises a recombinant T-cell receptor ligand.

38. The isolated high affinity entity of claim 1, wherein said soluble T-cell receptor ligand comprises a native T-cell receptor ligand.

39. The isolated high affinity entity of claim 1, wherein said four-domain α1-β1/α2-β2 MHC class II is a native MHC class II molecule presented on a cell.


SEQUENCE LISTING (456 sequences)

SEQ ID NO: 1 - protein, 11 aa, Artificial sequence
  2E4 light chain CDR 1
RASQSVSSYLA

SEQ ID NO: 2 - protein, 7 aa, Artificial sequence
  2E4 light chain CDR 2
DASNRAT

SEQ ID NO: 3 - protein, 11 aa, Artificial sequence
  2E4 light chain CDR 3
QQRSNWPPSYT

SEQ ID NO: 4 - DNA, 33 bp, Artificial sequence
  2E4 light chain CDR1
agggccagtcagagtgttagcagctacttagcc

SEQ ID NO: 5 - DNA, 21 bp, Artificial sequence
  2E4 light chain CDR2
gatgcatccaacagggccact

SEQ ID NO: 6 - DNA, 33 bp, Artificial sequence
  2E4 light chain CDR3
cagcagcgtagcaactggcctccctcgtacact

SEQ ID NO: 7 - protein, 10 aa, Artificial sequence
  2E4 heavy chain CDR 1
GYTFTSYYMH

SEQ ID NO: 8 - protein, 16 aa, Artificial sequence
  2E4 heavy chain CDR 2
IINPSGGSTSYAQKFQ

SEQ ID NO: 9 - protein, 12 aa, Artificial sequence
  2E4 heavy chain CDR 3
EGDNYYGDAFDI

SEQ ID NO: 10 - DNA, 30 bp, Artificial sequence
  2E4 Heavy chain CDR1
ggatacaccttcaccagctactatatgcac

SEQ ID NO: 11 - DNA, 51 bp, Artificial sequence
  2E4 Heavy chain CDR2
ataatcaaccctagtggtggtagcacaagctacgcacagaagttccagggc

SEQ ID NO: 12 - DNA, 36 bp, Artificial sequence
  2E4 Heavy chain CDR3
gagggagataattactatggtgatgcttttgatatc

SEQ ID NO: 13 - protein, 217 aa, Artificial sequence
  2E4 Fab light chain
LEIVLTQSPATLSLSPGERATLSCRASQSVSSYLAWYQQKPGQAPRLLIYDASNRATGIPARFSGSGSGTDFTLTISSLEPEDFAVYYCQQRSNWPPSYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKLYACEVTHQGLSSPVTKSFNRGEC

SEQ ID NO: 14 - DNA, 651 bp, Artificial sequence
  2E4 Fab light chain
cttgaaattgtgttgacacagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggcctccctcgtacacttttggccaggggaccaagctggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaactctacgcctgcgaagtcacccatcagggcctgagctcgcccgtcacaaagagcttcaacaggggagagtgt

SEQ ID NO: 15 - protein, 250 aa, Artificial sequence
  2E4 Fab heavy chain
EVQLVESGAEVKKPGASVKVSCKASGYTFTSYYMHWVRQAPGQGLEWMGIINPSGGSTSYAQKFQGRVTMTRDTSTSTVYMELSSLRSEDTAVYYCAREGDNYYGDAFDIWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 16 - DNA, 753 bp, Artificial sequence
  2E4 Fab heavy chain
gccgaggtgcagctggtggagtccggggctgaggtgaagaagcctggggcctcagtgaaggtttcctgcaaggcatctggatacaccttcaccagctactatatgcactgggtgcggcaggcccccggacaagggcttgagtggatgggaataatcaaccctagtggtggtagcacaagctacgcacagaagttccagggcagagtcaccatgaccagggacacgtccacgagcacagtctacatggagctgagcagcctgagatctgaggacacggccgtgtattactgtgctagagagggagataattactatggtgatgcttttgatatctggggccaagggacaatggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgca

SEQ ID NO: 17 - protein, 12 aa, Artificial sequence
  1F11 light chain CDR 1
RASQSIINSHLA

SEQ ID NO: 18 - protein, 7 aa, Artificial sequence
  1F11 light chain CDR 2
GASSRAT

SEQ ID NO: 19 - protein, 9 aa, Artificial sequence
  1F11 light chain CDR 3
QQYGTSPLT

SEQ ID NO: 20 - DNA, 36 bp, Artificial sequence
  1F11 light chain CDR1
agggccagtcagagtattatcaacagccacttagcc

SEQ ID NO: 21 - DNA, 21 bp, Artificial sequence
  1F11 light chain CDR2
ggtgcatccagcagggccact

SEQ ID NO: 22 - DNA, 27 bp, Artificial sequence
  1F11 light chain CDR3
cagcagtatggaacctctcctctcact

SEQ ID NO: 23 - protein, 12 aa, Artificial sequence
  1F11 heavy chain CDR 1
GVSISSRSGHWG

SEQ ID NO: 24 - protein, 16 aa, Artificial sequence
  1F11 heavy chain CDR 2
SISYSGSTYYNPSLKS

SEQ ID NO: 25 - protein, 10 aa, Artificial sequence
  1F11 heavy chain CDR 3
ESHPAAALVG

SEQ ID NO: 26 - DNA, 36 bp, Artificial sequence
  1F11 heavy chain  CDR1
ggtgtctccatcagcagtagaagtggccactggggc

SEQ ID NO: 27 - DNA, 48 bp, Artificial sequence
  1F11 heavy chain  CDR2
agtatctcttatagtgggagcacctactacaacccgtccctcaagagc

SEQ ID NO: 28 - DNA, 30 bp, Artificial sequence
  1F11 heavy chain  CDR3
gagtcgcacccagcagctgcactggttggg

SEQ ID NO: 29 - protein, 216 aa, Artificial sequence
  1F11 Fab light chain
LETTLTQSPGTLSLSPGERATLSCRASQSIINSHLAWYQQKPGQAPRLLIYGASSRATGIPDRFSGGGSGTDFTLTITRLETEDFALYFCQQYGTSPLTFGGGTRVETKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 30 - DNA, 648 bp, Artificial sequence
  1F11 Fab light chain
cttgaaacgacactcacgcagtctccaggcaccctgtctttgtctccaggggaaagagccaccctctcctgcagggccagtcagagtattatcaacagccacttagcctggtaccagcagaaacctggccaggctcccaggctcctcatctatggtgcatccagcagggccactggcatcccagacaggttcagtggcggtgggtctgggacagacttcactctcaccatcaccagactggaaactgaagattttgcactatatttctgccagcagtatggaacctctcctctcactttcggcggagggaccagggttgagaccaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcaaggcctgagctcgcccgtcacaaagagcttcaacaggggagagtgt

SEQ ID NO: 31 - protein, 249 aa, Artificial sequence
  1F11 Fab  heavy chain
QLQLQESGPGLVKPSETLSLTCTVSGVSISSRSGHWGWVRQPPGKGLEWIGSISYSGSTYYNPSLKSRVTISVDTSKNQLSLKLSSVTAADTAVYYCARESHPAAALVGWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 32 - DNA, 747 bp, Artificial sequence
  1F11 Fab  heavy chain
cagctgcagctgcaggagtccggcccaggactggtgaagccttcggagaccctgtccctcacctgcactgtctctggtgtctccatcagcagtagaagtggccactggggctgggtccgccagcccccagggaaggggctggagtggattggaagtatctcttatagtgggagcacctactacaacccgtccctcaagagccgagtcaccatatccgtagacacctccaagaaccaactctccctgaagctgagctctgtgaccgccgcagacacggctgtgtattactgtgcgagagagtcgcacccagcagctgcactggttgggtggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgca

SEQ ID NO: 33 - protein, 11 aa, Artificial sequence
  3A3 light chain CDR 1
RASQVISSWLA

SEQ ID NO: 34 - protein, 7 aa, Artificial sequence
  3A3 light chain CDR 2
TASSLQS

SEQ ID NO: 35 - protein, 9 aa, Artificial sequence
  3A3 light chain CDR 3
QQANSFPLT

SEQ ID NO: 36 - DNA, 33 bp, Artificial sequence
  3A3 light chain CDR1
cgggcgagtcaggttattagcagctggttagcc

SEQ ID NO: 37 - DNA, 21 bp, Artificial sequence
  3A3 light chain CDR2
actgcatccagtttgcaaagt

SEQ ID NO: 38 - DNA, 27 bp, Artificial sequence
  3A3 light chain CDR3
caacaggctaacagtttccccctgacg

SEQ ID NO: 39 - protein, 10 aa, Artificial sequence
  3A3 heavy chain CDR 1
GFTFSSYSMN

SEQ ID NO: 40 - protein, 17 aa, Artificial sequence
  3A3 heavy chain CDR 2
SISSSSSYIYYADSVKG

SEQ ID NO: 41 - protein, 26 aa, Artificial sequence
  3A3 heavy chain CDR 3
VRGHRYYYDSSGYYSSDYYYYYGMDV

SEQ ID NO: 42 - DNA, 30 bp, Artificial sequence
  3A3 heavy chain CDR1
ggattcaccttcagtagctatagcatgaac

SEQ ID NO: 43 - DNA, 51 bp, Artificial sequence
  3A3 heavy chain CDR2
tccattagtagtagtagtagttacatatactacgcagactcagtgaagggc

SEQ ID NO: 44 - DNA, 78 bp, Artificial sequence
  3A3 heavy chain CDR3
gtcagggggcaccggtattactatgatagtagtggttattactcatccgattactactactactacggtatggacgtc

SEQ ID NO: 45 - protein, 215 aa, Artificial sequence
  3A3 Fab light chain
LDIQLTQSPSSVSASVGDRVTITCRASQVISSWLAWYQQKPGKAPKLLISTASSLQSGVPPRFSGSGSGTDFTLTITSLQPEDFATYYCQQANSFPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - DNA, 645 bp, Artificial sequence
  3A3 Fab light chain
cttgacatccagttgacccagtctccatcttccgtgtctgcatctgtaggggacagagtcaccatcacttgtcgggcgagtcaggttattagcagctggttagcctggtatcagcaaaaaccagggaaagcccctaagctcctgatctctactgcatccagtttgcaaagtggggtcccaccaaggttcagcggcagtgggtctgggacagatttcactctcaccatcaccagcctgcagcctgaagattttgcaacttactattgtcaacaggctaacagtttccccctgacgttcggccaagggaccaaggtggaaatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 47 - protein, 264 aa, Artificial sequence
  3A3 Fab heavy chain
QVQLQESGGGLVKPGGSLRLSCAASGFTFSSYSMNWVRQAPGKGLEWVSSISSSSSYIYYADSVKGRFTISRDNAKNSLYLQMNSLRAEDTAVYYCARVRGHRYYYDSSGYYSSDYYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 48 - DNA, 792 bp, Artificial sequence
  3A3 Fab heavy chain
caggtgcagctgcaggagtcggggggaggcctggtcaagcctggggggtccctgagactctcctgtgcagcctctggattcaccttcagtagctatagcatgaactgggtccgccaggctccagggaaggggctggagtgggtctcatccattagtagtagtagtagttacatatactacgcagactcagtgaagggccgattcaccatctccagagacaacgccaagaactcactgtatctgcaaatgaacagcctgagagccgaggacacggctgtgtattactgtgcgagggtcagggggcaccggtattactatgatagtagtggttattactcatccgattactactactactacggtatggacgtctggggccaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgca

SEQ ID NO: 49 - protein, 16 aa, Artificial sequence
  3H5 light chain CDR 1
RSSQSLLHSNGNNYLD

SEQ ID NO: 50 - protein, 7 aa, Artificial sequence
  3H5 light chain CDR 2
LGSNRAS

SEQ ID NO: 51 - protein, 9 aa, Artificial sequence
  3H5 light chain CDR 3
MQALQTPLT

SEQ ID NO: 52 - DNA, 48 bp, Artificial sequence
  3H5 light chain CDR1
aggtctagtcagagcctcttgcatagtaatggaaacaactatttggat

SEQ ID NO: 53 - DNA, 21 bp, Artificial sequence
  3H5 light chain CDR2
ttgggttctaatcgggcctcc

SEQ ID NO: 54 - DNA, 27 bp, Artificial sequence
  3H5 light chain CDR3
atgcaagctcttcaaactccgctcacc

SEQ ID NO: 55 - protein, 10 aa, Artificial sequence
  3H5 heavy chain CDR 1
GGSISGYYWS

SEQ ID NO: 56 - protein, 16 aa, Artificial sequence
  3H5 heavy chain CDR 2
YIYYSGSTNYNPSLKS

SEQ ID NO: 57 - protein, 12 aa, Artificial sequence
  3H5 heavy chain CDR 3
DERDAYYYGMDV

SEQ ID NO: 58 - DNA, 30 bp, Artificial sequence
  3H5 heavy chain CDR1
ggtggctccatcagcggttactactggagc

SEQ ID NO: 59 - DNA, 48 bp, Artificial sequence
  3H5 heavy chain CDR2
tatatctattacagtgggagcaccaactacaacccctccctcaagagt

SEQ ID NO: 60 - DNA, 36 bp, Artificial sequence
  3H5 heavy chain CDR3
gatgaaagggacgcctactactacggtatggacgtc

SEQ ID NO: 61 - protein, 220 aa, Artificial sequence
  3H5 Fab light chain
LEIVMTQSPLSLPVTPGEPASISCRSSQSLLHSNGNNYLDWYLQKPGQSPQLLIYLGSNRASGVPDRFSGSGSGTDFTLKITRVEAEDVGVYYCMQALQTPLTFGGGTKMEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 62 - DNA, 660 bp, Artificial sequence
  3H5 Fab light chain
cttgaaattgtgatgacgcagtctccactctccctgcccgtcacccctggagagccggcctccatctcctgcaggtctagtcagagcctcttgcatagtaatggaaacaactatttggattggtacctgcagaagccagggcagtctccacagctcctgatctatttgggttctaatcgggcctccggggtccctgacaggttcagtggcagtggatcgggcacagattttacactgaaaatcaccagagtggaggctgaggatgttggggtttattactgcatgcaagctcttcaaactccgctcaccttcggcggagggaccaagatggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 63 - protein, 249 aa, Artificial sequence
  3H5 Fab  heavy chain
QVQLQESGPGLVKPSETLSLTCTVSGGSISGYYWSWIRQPPGKGLEWIGYIYYSGSTNYNPSLKSRVSISVDTSKNQFSLRLSSVTAADSAVYFCARDERDAYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 64 - DNA, 747 bp, Artificial sequence
  3H5 Fab  heavy chain
caggtgcagctgcaggagtccggcccaggactggtgaagccttcggagaccctgtccctcacctgcactgtctctggtggctccatcagcggttactactggagctggatccggcagcccccagggaagggactggagtggattggctatatctattacagtgggagcaccaactacaacccctccctcaagagtcgagtcagcatatcagtagacacgtccaagaaccagttctccctgaggctgagctccgtgacggccgcggactcggccgtgtatttctgtgcgagagatgaaagggacgcctactactacggtatggacgtctggggccaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgca

SEQ ID NO: 65 - protein, 16 aa, Artificial sequence
  2C3 light chain CDR 1
RSSQSLLHSDGNNYLD

SEQ ID NO: 66 - protein, 7 aa, Artificial sequence
  2C3 light chain CDR 2
LGSNRAS

SEQ ID NO: 67 - protein, 9 aa, Artificial sequence
  2C3 light chain CDR 3
MQALHIPLT

SEQ ID NO: 68 - DNA, 48 bp, Artificial sequence
  2C3 light chain CDR1
aggtctagtcagagcctcctgcatagtgatggaaacaactatttggat

SEQ ID NO: 69 - DNA, 21 bp, Artificial sequence
  2C3 light chain CDR2
ttgggttctaatcgggcctcc

SEQ ID NO: 70 - DNA, 27 bp, Artificial sequence
  2C3 light chain CDR3
atgcaagctctacatattccgctcacc

SEQ ID NO: 71 - protein, 10 aa, Artificial sequence
  2C3 heavy chain CDR 1
GFTFSSYAMH

SEQ ID NO: 72 - protein, 17 aa, Artificial sequence
  2C3 heavy chain CDR 2
VISYDGSNKYYADSVKG

SEQ ID NO: 73 - protein, 20 aa, Artificial sequence
  2C3 heavy chain CDR 3
DRSFWSGYYIINYYYYGMDV

SEQ ID NO: 74 - DNA, 30 bp, Artificial sequence
  2C3 heavy chain CDR1
ggattcaccttcagtagctatgctatgcac

SEQ ID NO: 75 - DNA, 51 bp, Artificial sequence
  2C3 heavy chain CDR2
gttatatcatatgatggaagcaataaatactacgcagactccgtgaagggc

SEQ ID NO: 76 - DNA, 60 bp, Artificial sequence
  2C3 heavy chain CDR3
gatcgctccttttggagtggttattatataattaactactactactacggtatggacgtc

SEQ ID NO: 77 - protein, 220 aa, Artificial sequence
  2C3 Fab light chain
LDVVMTQSPLSLPVTPGEPASISCRSSQSLLHSDGNNYLDWYLQKPGQSPQLLIYLGSNRASGVPDRFSGSGSGTDFTLKISRVEPEDVGVYYCMQALHIPLTFGQGTRLEIKRTVAAPSVFIFPPSDEQLKSGTASVVCLLNNFYPREAKVQWKVDNALQSGNSQESVTEQDSRDSTYSLSSTLTLSKADYEKHKVYACEVTHQGLSSPVTKSFNRGEC

SEQ ID NO: 78 - DNA, 660 bp, Artificial sequence
  2C3 Fab light chain
cttgatgttgtgatgactcagtctccactctccctgcccgtcacacctggagagccggcctccatctcctgcaggtctagtcagagcctcctgcatagtgatggaaacaactatttggattggtacctgcagaagccagggcagtctccacagctcctgatctatttgggttctaatcgggcctccggggtccctgacaggttcagtggcagtggatcaggcacagattttacactgaaaatcagcagagtggagcctgaggatgtcggggtttattactgcatgcaagctctacatattccgctcaccttcggccaagggacacgactggagattaagcgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcagggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 79 - protein, 258 aa, Artificial sequence
  2C3 Fab heavy chain
QVQLVQSGGGVVQPGRSLRLSCAASGFTFSSYAMHWVRQAPGKGLEWVAVISYDGSNKYYADSVKGRFTISRDNSKNTLYLQMNSLRAEDTAVYYCARDRSFWSGYYIINYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 80 - DNA, 774 bp, Artificial sequence
  2C3 Fab heavy chain
caggtgcagctggtgcagtctgggggaggcgtggtccagcctgggaggtccctgagactctcctgtgcagcctctggattcaccttcagtagctatgctatgcactgggtccgccaggctccaggcaaggggctggagtgggtggcagttatatcatatgatggaagcaataaatactacgcagactccgtgaagggccgattcaccatctccagagacaattccaagaacacgctgtatctgcaaatgaacagcctgagagctgaggacacggctgtgtattactgtgcgagagatcgctccttttggagtggttattatataattaactactactactacggtatggacgtctggggccaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgca

SEQ ID NO: 81 - protein, 11 aa, Artificial sequence
  1B11 light chain CDR1
RASQNIGSILA

SEQ ID NO: 82 - protein, 7 aa, Artificial sequence
  1B11 light chain CDR2
GASTRAT

SEQ ID NO: 83 - protein, 9 aa, Artificial sequence
  1B11 light chain CDR3
QQYLYWPFT

SEQ ID NO: 84 - DNA, 33 bp, Artificial sequence
  1B11 light chain CDR1
agggccagtcagaatattggcagcatcttagcc

SEQ ID NO: 85 - DNA, 21 bp, Artificial sequence
  1B11 light chain CDR2
ggtgcatccaccagggccact

SEQ ID NO: 86 - DNA, 27 bp, Artificial sequence
  1B11 light chain CDR3
cagcaatatctttactggccgttcact

SEQ ID NO: 87 - protein, 10 aa, Artificial sequence
  1B11 heavy chain CDR1
GYTFTSYGIS

SEQ ID NO: 88 - protein, 17 aa, Artificial sequence
  1B11 heavy chain CDR2
WISAYNGNTNYAQKLQG

SEQ ID NO: 89 - protein, 20 aa, Artificial sequence
  1B11 heavy chain CDR3
DIRAYGSGSYSRYYYYGMDV

SEQ ID NO: 90 - DNA, 30 bp, Artificial sequence
  1B11 heavy chain CDR1
ggttacacctttaccagctatggtatcagc

SEQ ID NO: 91 - DNA, 51 bp, Artificial sequence
  1B11 heavy chain CDR2
tggatcagcgcttacaatggtaacacaaactatgcacagaagctccagggc

SEQ ID NO: 92 - DNA, 60 bp, Artificial sequence
  1B11 heavy chain CDR3
gatattcgggcctatggttcggggagttattcgcgctactactactacggtatggacgtc

SEQ ID NO: 93 - protein, 215 aa, Artificial sequence
  1B11 Fab light chain
LEIVLTQSPATLSVSPGERATLSCRASQNIGSILAWYQHKPGQAPRLLIYGASTRATGIPARFSGSGSGTEFTLTISSLQSDDFAVYYCQQYLYWPFTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 94 - DNA, 645 bp, Artificial sequence
  1B11 Fab light chain
cttgaaattgtgttgacacagtctccagccaccctgtctgtgtctccaggagaaagagccaccctctcctgcagggccagtcagaatattggcagcatcttagcctggtaccagcacaaacctggccaggctcccaggctcctcatctatggtgcatccaccagggccactggtatcccagccaggttcagtggcagtgggtctgggacagagttcactcttaccatcagcagcctgcagtctgacgattttgcagtttattactgtcagcaatatctttactggccgttcactttcggcggagggaccaaggtggagatcaaacgaactgtggctgcaccatctgtcttcatcttcccgccatctgatgagcagttgaaatctggaactgcctctgttgtgtgcctgctgaataacttctatcccagagaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 95 - protein, 258 aa, Artificial sequence
  1B11 Fab heavy chain
EVQLVQSGAEVKKPGASVRVSCKASGYTFTSYGISWVRQAPGQGLEWMGWISAYNGNTNYAQKLQGRVTMTTDTSTSTAYMELRSLRSDDTAVYYCARDIRAYGSGSYSRYYYYGMDVWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 96 - DNA, 774 bp, Artificial sequence
  1B11 Fab heavy chain
gaggtccagctggtacagtctggggctgaggtgaagaagcctggggcctcagtgagggtctcctgcaaggcttctggttacacctttaccagctatggtatcagctgggtgcgacaggcccctggacaagggcttgagtggatgggatggatcagcgcttacaatggtaacacaaactatgcacagaagctccagggcagagtcaccatgaccacagacacatccacgagcacagcctacatggagctgaggagcctgagatctgacgacacggccgtgtattactgtgcgagagatattcgggcctatggttcggggagttattcgcgctactactactacggtatggacgtctggggccaagggaccacggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgca

SEQ ID NO: 97 - protein, 11 aa, Artificial sequence
  D2 light chain CDR1
RASQSVSSYLA

SEQ ID NO: 98 - protein, 7 aa, Artificial sequence
  D2 light chain CDR2
DASNRAT

SEQ ID NO: 99 - protein, 9 aa, Artificial sequence
  D2 light chain CDR3
QQRSNWPLT

SEQ ID NO: 100 - DNA, 33 bp, Artificial sequence; identical to SEQ ID NO: 4
  D2 light chain CDR1

SEQ ID NO: 101 - DNA, 21 bp, Artificial sequence
  D2 light chain CDR2
gatgcatccaacagggccact

SEQ ID NO: 102 - DNA, 27 bp, Artificial sequence
  D2 light chain CDR3
cagcagcgtagcaactggccgctcact

SEQ ID NO: 103 - protein, 10 aa, Artificial sequence
  D2 heavy chain CDR1
GGTFSSYAIS

SEQ ID NO: 104 - protein, 17 aa, Artificial sequence
  D2 heavy chain CDR2
IINPSGGSTSYAQKFQG

SEQ ID NO: 105 - protein, 9 aa, Artificial sequence
  D2 heavy chain CDR3
GGPTGIFDY

SEQ ID NO: 106 - DNA, 30 bp, Artificial sequence
  D2 heavy chain CDR1
ggaggcaccttcagcagctatgctatcagc

SEQ ID NO: 107 - DNA, 51 bp, Artificial sequence; identical to SEQ ID NO: 11
  D2 heavy chain CDR2

SEQ ID NO: 108 - DNA, 27 bp, Artificial sequence
  D2 heavy chain CDR3
ggggggcctacaggcatctttgactac

SEQ ID NO: 109 - protein, 215 aa, Artificial sequence
  D2 Fab light chain
LEIVLTQSPATLSLSPGERATLSCRASQSVSSYLAWYQQKPGQAPRLLIYDASNRATGIPARFSGSGSGTDFTLTISSLEPEDFAVYYCQQRSNWPLTFGGGTKVEIKRTVAAPSVFIFPPSDEQLKSGTASVVCLLNNFYPRKAKVQWKVDNALQSGNSQESVTEQDSKDSTYSLSSTLTLSKADYEKHKVYACEVTHQGLSSPVTKSFNRGEC

SEQ ID NO: 110 - DNA, 645 bp, Artificial sequence
  D2 Fab light chain
cttgaaattgtgctgactcagtctccagccaccctgtctttgtctccaggggaaagagccaccctctcctgcagggccagtcagagtgttagcagctacttagcctggtaccaacagaaacctggccaggctcccaggctcctcatctatgatgcatccaacagggccactggcatcccagccaggttcagtggcagtgggtctgggacagacttcactctcaccatcagcagcctagagcctgaagattttgcagtttattactgtcagcagcgtagcaactggccgctcactttcggcggagggaccaaggtggagatcaaacgaactgtggctgcaccatctgtcttcatcttcccgccatctgatgagcagttgaaatctggaactgcctctgttgtgtgcctgctgaataacttctatcccagaaaggccaaagtacagtggaaggtggataacgccctccaatcgggtaactcccaggagagtgtcacagagcaggacagcaaggacagcacctacagcctcagcagcaccctgacgctgagcaaagcagactacgagaaacacaaagtctacgcctgcgaagtcacccatcagggcctgagctcgcccgtcacaaagagcttcaacaggggagagtgt

SEQ ID NO: 111 - protein, 247 aa, Artificial sequence
  D2 Fab heavy chain
EVQLVQSGAEVKKPGSSVKVSCKASGGTFSSYAISWVRQAPGQGLEWMGIINPSGGSTSYAQKFQGRVTITADKSTSTAYMELSSLRSEDTAVYYCARGGPTGIFDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCAAAHHHHHHGAAEQKLISEEDLNGAA

SEQ ID NO: 112 - DNA, 741 bp, Artificial sequence
  D2 Fab heavy chain
gaggtccagctggtgcagtctggggctgaggtgaagaagcctgggtcctcggtgaaggtctcctgcaaggcttctggaggcaccttcagcagctatgctatcagctgggtgcgacaggcccctggacaagggcttgagtggatgggaataatcaaccctagtggtggtagcacaagctacgcacagaagttccagggcagagtcacgattaccgcggacaaatccacgagcacagcctacatggagctgagcagcctgagatctgaggacacggccgtgtattactgtgcgagaggggggcctacaggcatctttgactactggggccagggcaccctggtcaccgtctcaagcgcctccaccaagggcccatcggtcttccccctggcaccctcctccaagagcacctctgggggcacagcggccctgggctgcctggtcaaggactacttccccgaaccggtgacggtgtcgtggaactcaggcgccctgaccagcggcgtccacaccttcccggctgtcctacagtcctcaggactctactccctcagcagcgtagtgaccgtgccctccagcagcttgggcacccagacctacatctgcaacgtgaatcacaagcccagcaacaccaaggtggacaagaaagttgagcccaaatcttgtgcggccgcacatcatcatcaccatcacggggccgcagaacaaaaactcatctcagaagaggatctgaatggggccgca

SEQ ID NO: 113 - protein, 110 aa, Homo sapiens
MALWMRLLPLLALLALWGPDPAAAFVNQHLCGSHLVEALYLVCGERGFFYTPKTRREAEDLQVGQVELGGGPGAGSLQPLALEGSLQKRGIVEQCCTSICSLYQLENYCN

SEQ ID NO: 114 - protein, 86 aa, Homo sapiens
FVNQHLCGSHLVEALYLVCGERGFFYTPKTRREAEDLQVGQVELGGGPGAGSLQPLALEGSLQKRGIVEQCCTSICSLYQLENYCN

SEQ ID NO: 115 - protein, 585 aa, Homo sapiens
MASPGSGFWSFGSEDGSGDSENPGTARAWCQVAQKFTGGIGNKLCALLYGDAEKPAESGGSQPPRAAARKAACACDQKPCSCSKVDVNYAFLHATDLLPACDGERPTLAFLQDVMNILLQYVVKSFDRSTKVIDFHYPNELLQEYNWELADQPQNLEEILMHCQTTLKYAIKTGHPRYFNQLSTGLDMVGLAADWLTSTANTNMFTYEIAPVFVLLEYVTLKKMREIIGWPGGSGDGIFSPGGAISNMYAMMIARFKMFPEVKEKGMAALPRLIAFTSEHSHFSLKKGAAALGIGTDSVILIKCDERGKMIPSDLERRILEAKQKGFVPFLVSATAGTTVYGAFDPLLAVADICKKYKIWMHVDAAWGGGLLMSRKHKWKLSGVERANSVTWNPHKMMGVPLQCSALLVREEGLMQNCNQMHASYLFQQDKHYDLSYDTGDKALQCGRHVDVFKLWLMWRAKGTTGFEAHVDKCLELAEYLYNIIKNREGYEMVFDGKPQHTNVCFWYIPPSLRTLEDNEERMSRLSKVAPVIKARMMEYGTTMVSYQPLGDKVNFFRMVISNPAATHQDIDFLIEEIERLGQDL

SEQ ID NO: 116 - protein, 566 aa, Homo sapiens
MPRGFLVKRTKRTGGLYRVRLAERVFPLLGPQGAPPFLEEAPSASLPGAERATPPTREEPGKGLTAEAAREQSGSPCRAAGVSPGTGGREGAEWRAGGREGPGPSPSPSPSPAKPAGAELRRAFLERCLSSPVSAESFPGGAAAVAAFSCSVAPAAAPTPGEQFLLPLRAPFPEPALQPDPAPLSAALQSLKRAAGGERRGKAPTDCASGPAAAGIKKPKAMRKLSFADEVTTSPVLGLKIKEEEPGAPSRGLGGSRTPLGEFICQLCKEQYADPFALAQHRCSRIVRVEYRCPECDKVFSCPANLASHRRWHKPRPAAANAATVSSADGKPPSSSSSSSRDSGAIASFLAEGKENSRIERTADQHPQARDSSGADQHPDSAPRQGLQVLTHPEPPLPQGPYTEGVLGRRVPVPGSTSGGRGSEIFVCPYCHKKFRRQAYLRKHLSTHEAGSARALAPGFGSERGAPLAFACPLCGAHFPTADIREKHRLWHAVREELLLPALAGAPPETSGPSGPSDGSAQQIFSCKHCPSTFFSSPGLTRHINKCHPSESRQVLLLQMPLRPGC

SEQ ID NO: 117 - protein, 986 aa, Homo sapiens
MGPPLPLLLLLLLLLPPRVLPAAPSSVPRGRQLPGRLGCLLEEGLCGASEACVNDGVFGRCQKVPAMDFYRYEVSPVALQRLRVALQKLSGTGFTWQDDYTQYVMDQELADLPKTYLRRPEASSPARPSKHSVGSERRYSREGGAALANALRRHLPFLEALSQAPASDVLARTHTAQDRPPAEGDDRFSESILTYVAHTSALTYPPGSRTQLREDLLPRTLGQLQPDELSPKVDSGVDRHHLMAALSAYAAQRPPAPPGEGSLEPQYLLRAPSRMPRPLLAPAAPQKWPSPLGDSEDPSSTGDGARIHTLLKDLQRQPAEVRGLSGLELDGMAELMAGLMQGVDHGVARGSPGRAALGESGEQADGPKATLRGDSFPDDGVQDDDDRLYQEVHRLSATLGGLLQDHGSRLLPGALPFARPLDMERKKSEHPESSLSSEEETAGVENVKSQTYSKDLLGQQPHSEPGAAAFGELQNQMPGPSKEEQSLPAGAQEALSDGLQLEVQPSEEEARGYIVTDREVLGPAVTFKVSANVQNVTTEDVEKATVDNKDKLEETSGLKILQTGVGSKSKLKFLPPQAEQEDSTKFIALTLVSLACILGVLLASGLIYCLRHSSQHRLKEKLSGLGGDPGADATAAYQELCRQRMATRPPDRPEGPHTSRISSVSSQFSDGPIPSPSARSSASSWSEEPVQSNMDISTGHMILSYMEDHLKNKNRLEKEWEALCAYQAEPNSSFVAQREENVPKNRSLAVLTYDHSRVLLKAENSHSHSDYINASPIMDHDPRNPAYIATQGPLPATVADFWQMVWESGCVVIVMLTPLAENGVRQCYHYWPDEGSNLYHIYEVNLVSEHIWCEDFLVRSFYLKNLQTNETRTVTQFHFLSWYDRGVPSSSRSLLDFRRKVNKCYRGRSCPIIVHCSDGAGRSGTYVLIDMVLNKMAKGAKEIDIAATLEHLRDQRPGMVQTKEQFEFALTAVAEEVNAILKALPQ

SEQ ID NO: 118 - protein, 355 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRLTWSFLWSVFWLIQISVCISRVFIATHFPHQVILGVIGGMLVAEAFEHTPGIQTASLGTYLKTNLFLFLFAVGFYLLLRVLNIDLLWSVPIAKKWCANPDWIHIDTTPFAGLVRNLGVLFGLGFAINSEMFLLSCRGGNNYTLSFRLLCALTSLTILQLYHFLQIPTHEEHLFYVLSFCKSASIPLTVVAFIPYSVHMLMKQSGKKSQ

SEQ ID NO: 119 - protein, 154 aa, Homo sapiens
MDFLHRNGVLIIQHLQKDYRAYYTFLNFMSNVGDPRNIFFIYFPLCFQFNQTVGTKMIWVAVIGDWLNLIFKWILFGHRPYWWVQETQIYPNHSSPCLEQFPTTCETGPGSPSGHAMGASCVWYVMVTAALSHTVCGMDKFSITLHRHAGGRGL

SEQ ID NO: 120 - protein, 457 aa, Homo sapiens
MRSAAVLALLLCAGQVTALPVNSPMNKGDTEVMKCIVEVISDTLSKPSPMPVSQECFETLRGDERILSILRHQNLLKELQDLALQGAKERAHQQKKHSGFEDELSEVLENQSSQAELKEAVEEPSSKDVMEKREDSKEAEKSGEATDGARPQALPEPMQESKAEGNNQAPGEEEEEEEEATNTHPPASLPSQKYPGPQAEGDSEGLSQGLVDREKGLSAEPGWQAKREEEEEEEEEAEAGEEAVPEEEGPTVVLNPHPSLGYKEIRKGESRSEALAVDGAGKPGAEEAQDPEGKGEQEHSQQKEEEEEMAVVPQGLFRGGKSGELEQEEERLSKEWEDSKRWSKMDQLAKELTAEKRLEGQEEEEDNRDSSMKLSFRARAYGFRGPGPQLRRGWRPSSREDSLEAGLPLQVRGYPEEKKEEEGSANRRPEDQELESLSAIEAELEKVAHQLQALRRG

SEQ ID NO: 121 - protein, 369 aa, Homo sapiens
MEFLERTYLVNDKAAKMYAFTLESVELQQKPVNKDQCPRERPEELESGGMYHCHSGSKPTEKGANEYAYAKWKLCSASAICFIFMIAEVVGGHIAGSLAVVTDAAHLLIDLTSFLLSLFSLWLSSKPPSKRLTFGWHRAEILGALLSILCIWVVTGVLVYLACERLLYPDYQIQATVMIIVSSCAVAANIVLTVVLHQRCLGHNHKEVQANASVRAAFVHALGDLFQSISVLISALIIYFKPEYKIADPICTFIFSILVLASTITILKDFSILLMEGVPKSLNYSGVKELILAVDGVLSVHSLHIWSLTMNQVILSAHVATAASRDSQVVRREIAKALSKSFTMHSLTIQMESPVDQDPDCLFCEDPCD

SEQ ID NO: 122 - protein, 573 aa, Homo sapiens
MLRLPTVFRQMRPVSRVLAPHLTRAYAKDVKFGADARALMLQGVDLLADAVAVTMGPKGRTVIIEQSWGSPKVTKDGVTVAKSIDLKDKYKNIGAKLVQDVANNTNEEAGDGTTTATVLARSIAKEGFEKISKGANPVEIRRGVMLAVDAVIAELKKQSKPVTTPEEIAQVATISANGDKEIGNIISDAMKKVGRKGVITVKDGKTLNDELEIIEGMKFDRGYISPYFINTSKGQKCEFQDAYVLLSEKKISSIQSIVPALEIANAHRKPLVIIAEDVDGEALSTLVLNRLKVGLQVVAVKAPGFGDNRKNQLKDMAIATGGAVFGEEGLTLNLEDVQPHDLGKVGEVIVTKDDAMLLKGKGDKAQIEKRIQEIIEQLDVTTSEYEKEKLNERLAKLSDGVAVLKVGGTSDVEVNEKKDRVTDALNATRAAVEEGIVLGGGCALLRCIPALDSLTPANEDQKIGIEIIKRTLKIPAMTIAKNAGVEGSLIVEKIMQSSSEVGYDAMAGDFVNMVEKGIIDPTKVVRTALLDAAGVASLLTTAEVVVTEIPKEEKDPGMGAMGGMGGGMGGGMF

SEQ ID NO: 123 - protein, 641 aa, Homo sapiens
MAKAAAIGIDLGTTYSCVGVFQHGKVEIIANDQGNRTTPSYVAFTDTERLIGDAAKNQVALNPQNTVFDAKRLIGRKFGDPVVQSDMKHWPFQVINDGDKPKVQVSYKGETKAFYPEEISSMVLTKMKEIAEAYLGYPVTNAVITVPAYFNDSQRQATKDAGVIAGLNVLRIINEPTAAAIAYGLDRTGKGERNVLIFDLGGGTFDVSILTIDDGIFEVKATAGDTHLGGEDFDNRLVNHFVEEFKRKHKKDISQNKRAVRRLRTACERAKRTLSSSTQASLEIDSLFEGIDFYTSITRARFEELCSDLFRSTLEPVEKALRDAKLDKAQIHDLVLVGGSTRIPKVQKLLQDFFNGRDLNKSINPDEAVAYGAAVQAAILMGDKSENVQDLLLLDVAPLSLGLETAGGVMTALIKRNSTIPTKQTQIFTTYSDNQPGVLIQVYEGERAMTKDNNLLGRFELSGIPPAPRGVPQIEVTFDIDANGILNVTATDKSTGKANKITITNDKGRLSKEEIERMVQEAEKYKAEDEVQRERVSAKNALESYAFNMKSAVEDEGLKGKISEADKKKVLDKCQEVISWLDANTLAEKDEFEHKRKELEQVCNPIISGLYQGAGGPGPGGFGAQGPKGGSGSGPTIEEVD

SEQ ID NO: 124 - protein, 641 aa, Homo sapiens; identical to SEQ ID NO: 123

SEQ ID NO: 125 - protein, 10 aa, Artificial sequence
  GAD556-565 peptide
FFRMVISNPA

SEQ ID NO: 126 - protein, 13 aa, Artificial sequence
  Diabetes-associated autoantigenic peptide
MFFRMVISNPAAT

SEQ ID NO: 127 - protein, 197 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 128 - protein, 277 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATVTGGQKGRGSRGQHQAHSLERVCHCLGKWLGHPDKFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 129 - protein, 21 aa, Artificial sequence
  Autoantigenic peptide
MEVGWYRPPFSRVVHLYRNGK

SEQ ID NO: 130 - protein, 16 aa, Artificial sequence
  MHC class II-restricted MBP-85-99 antigenic peptide
MENPVVHFFKNIVTPR

SEQ ID NO: 131 - protein, 280 aa, Aegilops tauschii
MKTFLILALLAIVATTATIAVRVPVPQLQPQNPSQQQPQEQVPLVQQQQFPGQQQPFPPQQPYPQPQPFPSQQPYLQLQPFPQPQLPYPQPQPFRPQQPYPQPQPQYSQPQQPISQQQQQQQQQQQQQQQILQQILQQQLIPCRDVVLQQHNVAHGSSQVLQQSTYQLVQQLCCQQLWQIPEQSRCQAIHNVVHAIILHQQQQQPLSQVSFQQPQQQYPSGQGSFQPSQQNPQAQGSVQPQQLPQFEEIRNLALETLPAMCNVYIPPYCTIAPGGIFGTN

SEQ ID NO: 132 - protein, 1487 aa, Homo sapiens
MIRLGAPQTLVLLTLLVAAVLRCQGQDVQEAGSCVQDGQRYNDKDVWKPEPCRICVCDTGTVLCDDIICEDVKDCLSPEIPFGECCPICPTDLATASGQPGPKGQKGEPGDIKDIVGPKGPPGPQGPAGEQGPRGDRGDKGEKGAPGPRGRDGEPGTPGNPGPPGPPGPPGPPGLGGNFAAQMAGGFDEKAGGAQLGVMQGPMGPMGPRGPPGPAGAPGPQGFQGNPGEPGEPGVSGPMGPRGPPGPPGKPGDDGEAGKPGKAGERGPPGPQGARGFPGTPGLPGVKGHRGYPGLDGAKGEAGAPGVKGESGSPGENGSPGPMGPRGLPGERGRTGPAGAAGARGNDGQPGPAGPPGPVGPAGGPGFPGAPGAKGEAGPTGARGPEGAQGPRGEPGTPGSPGPAGASGNPGTDGIPGAKGSAGAPGIAGAPGFPGPRGPPGPQGATGPLGPKGQTGEPGIAGFKGEQGPKGEPGPAGPQGAPGPAGEEGKRGARGEPGGVGPIGPPGERGAPGNRGFPGQDGLAGPKGAPGERGPSGLAGPKGANGDPGRPGEPGLPGARGLTGRPGDAGPQGKVGPSGAPGEDGRPGPPGPQGARGQPGVMGFPGPKGANGEPGKAGEKGLPGAPGLRGLPGKDGETGAAGPPGPAGPAGERGEQGAPGPSGFQGLPGPPGPPGEGGKPGDQGVPGEAGAPGLVGPRGERGFPGERGSPGAQGLQGPRGLPGTPGTDGPKGASGPAGPPGAQGPPGLQGMPGERGAAGIAGPKGDRGDVGEKGPEGAPGKDGGRGLTGPIGPPGPAGANGEKGEVGPPGPAGSAGARGAPGERGETGPPGPAGFAGPPGADGQPGAKGEQGEAGQKGDAGAPGPQGPSGAPGPQGPTGVTGPKGARGAQGPPGATGFPGAAGRVGPPGSNGNPGPPGPPGPSGKDGPKGARGDSGPPGRAGEPGLQGPAGPPGEKGEPGDDGPSGAEGPPGPQGLAGQRGIVGLPGQRGERGFPGLPGPSGEPGKQGAPGASGDRGPPGPVGPPGLTGPAGEPGREGSPGADGPPGRDGAAGVKGDRGETGAVGAPGAPGPPGSPGPAGPTGKQGDRGEAGAQGPMGPSGPAGARGIQGPQGPRGDKGEAGEPGERGLKGHRGFTGLQGLPGPPGPSGDQGASGPAGPSGPRGPPGPVGPSGKDGANGIPGPIGPPGPRGRSGETGPAGPPGNPGPPGPPGPPGPGIDMSAFAGLGPREKGPDPLQYMRADQAAGGLRQHDAEVDATLKSLNNQIESIRSPEGSRKNPARTCRDLKLCHPEWKSGDYWIDPNQGCTLDAMKVFCNMETGETCVYPNPANVPKKNWWSSKSKEKKHIWFGETINGGFHFSYGDDNLAPNTANVQMTFLRLLSTEGSQNITYHCKNSIAYLDEAAGNLKKALLIQGSNDVEIRAEGNSRFTYTALKDGCTKHTGKWGKTVIEYRSQKTSRLPIIDIAPMDIGGPEQEFGVDIGPVCFL

SEQ ID NO: 133 - protein, 998 aa, Homo sapiens
MGPPLPLLLLLLLLLPPRVLPAAPSSVPRGRQLPGRLDGVFGRCQKVPAMDFYRYEVSPVALQRLRVALQKLSGTGFTWQDDYTQYVMDQELADLPKTYLRRPEASSPARPSKHSVGSERRYSREGGAALANALRRHLPFLEALSQAPASDVLARTHTAQDRPPAEGDDRFSESILTYVAHTSALTYPPGSRTQLREDLLPRTLGQLQPDELSPKVDSGVDRHHLMAALSAYAAQRPPAPPGEGSLEPQYLLRAPSRMPRPLLAPAAPQKWPSPLGDSEDPSSTGDGARIHTLLKDLQRQPAEVRGLSGLELDGMAELMAGLMQGVDHGVARGSPGRAALGESGEQADGPKATLRGDSFPDDGVQDDDDRLYQEVHRLSATLGGLLQDHGSRLLPGALPFARPLDMERKKSEHPESSLSSEEETAGVENVKSQTYSKDLLGQQPHSEPGAAAFGELQNQMPGPSKEEQSLPAGAQEALSDGLQLEVQPSEEEARGYIVTDRDPLRPEEGRRLVEDVARLLQVPSSAFADVEVLGPAVTFKVSANVQNVTTEDVEKATVDNKDKLEETSGLKILQTGVGSKSKLKFLPPQAEQEDSTKFIALTLVSLACILGVLLASGLIYCLRHSSQHRLKEKLSGLGGDPGADATAAYQELCRQRMATRPPDRPEGPHTSRISSVSSQFSDGPIPSPSARSSASSWSEEPVQSNMDISTGHMILSYMEDHLKNKNRLEKEWEALCAYQAEPNSSFVAQREENVPKNRSLAVLTYDHSRVLLKAENSHSHSDYINASPIMDHDPRNPAYIATQGPLPATVADFWQMVWESGCVVIVMLTPLAENGVRQCYHYWPDEGSNLYHIYEVNLVSEHIWCEDFLVRSFYLKNLQTNETRTVTQFHFLSWYDRGVPSSSRSLLDFRRKVNKCYRGRSCPIIVHCSDGAGRSGTYVLIDMVLNKMAKGAKEIDIAATLEHLRDQRPGMVQTKEQFEFALTAVAEEVNAILKALPQ

SEQ ID NO: 134 - protein, 1015 aa, Homo sapiens
MGPPLPLLLLLLLLLPPRVLPAAPSSVPRGRQLPGRLGCLLEEGLCGASEACVNDGVFGRCQKVPAMDFYRYEVSPVALQRLRVALQKLSGTGFTWQDDYTQYVMDQELADLPKTYLRRPEASSPARPSKHSVGSERRYSREGGAALANALRRHLPFLEALSQAPASDVLARTHTAQDRPPAEGDDRFSESILTYVAHTSALTYPPGSRTQLREDLLPRTLGQLQPDELSPKVDSGVDRHHLMAALSAYAAQRPPAPPGEGSLEPQYLLRAPSRMPRPLLAPAAPQKWPSPLGDSEDPSSTGDGARIHTLLKDLQRQPAEVRGLSGLELDGMAELMAGLMQGVDHGVARGSPGRAALGESGEQADGPKATLRGDSFPDDGVQDDDDRLYQEVHRLSATLGGLLQDHGSRLLPGALPFARPLDMERKKSEHPESSLSSEEETAGVENVKSQTYSKDLLGQQPHSEPGAAAFGELQNQMPGPSKEEQSLPAGAQEALSDGLQLEVQPSEEEARGYIVTDRDPLRPEEGRRLVEDVARLLQVPSSAFADVEVLGPAVTFKVSANVQNVTTEDVEKATVDNKDKLEETSGLKILQTGVGSKSKLKFLPPQAEQEDSTKFIALTLVSLACILGVLLASGLIYCLRHSSQHRLKEKLSGLGGDPGADATAAYQELCRQRMATRPPDRPEGPHTSRISSVSSQFSDGPIPSPSARSSASSWSEEPVQSNMDISTGHMILSYMEDHLKNKNRLEKEWEALCAYQAEPNSSFVAQREENVPKNRSLAVLTYDHSRVLLKAENSHSHSDYINASPIMDHDPRNPAYIATQGPLPATVADFWQMVWESGCVVIVMLTPLAENGVRQCYHYWPDEGSNLYHIYEVNLVSEHIWCEDFLVRSFYLKNLQTNETRTVTQFHFLSWYDRGVPSSSRSLLDFRRKVNKCYRGRSCPIIVHCSDGAGRSGTYVLIDMVLNKMAKGAKEIDIAATLEHLRDQRPGMVQTKEQFEFALTAVAEEVNAILKALPQ

SEQ ID NO: 135 - protein, 224 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFESFGVLGPQVKEPKKTGQFLEELRNPF

SEQ ID NO: 136 - protein, 206 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFVFHLEALSG

SEQ ID NO: 137 - protein, 108 aa, Homo sapiens
MASLSRPSLPSCLCSFLLLLLLQVSSSYADPFYWVSPGVLVLLAVLPVLLLQITVGLIFLCLQYRLRGKLRAEIENLHRTFESFGVLGPQVKEPKKTGQFLEELRNPF

SEQ ID NO: 138 - protein, 252 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELLFHLEALSG

SEQ ID NO: 139 - protein, 247 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 140 - protein, 213 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFGQFLEELLFHLEALSG

SEQ ID NO: 141 - protein, 229 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFESFGVLGPQVKEPKKTGQFLEELLFHLEALSG

SEQ ID NO: 142 - protein, 208 aa, Homo sapiens
MASLSRPSLPSCLCSFLLLLLLQVSSSYAGQFRVIGPRHPIRALVGDEVELPCRISPGKNATGMEVGWYRPPFSRVVHLYRNGKDQDGDQAPEYRGRTELLKDAIGEGKVTLRIRNVRFSDEGGFTCFFRDHSYQEEAAMELKVEDPFYWVSPGVLVLLAVLPVLLLQITVGLIFLCLQYRLRGKLRAEIENLHRTFGQFLEELRNPF

SEQ ID NO: 143 - protein, 131 aa, Homo sapiens
MASLSRPSLPSCLCSFLLLLLLQVSSSYADPFYWVSPGVLVLLAVLPVLLLQITVGLIFLCLQYRLRGKLRAEIENLHRTFDPHFLRVPCWKITLFVIVPVLGPLVALIICYNWLHRRLAGQFLEELRNPF

SEQ ID NO: 144 - protein, 171 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 145 - protein, 160 aa, Homo sapiens
MASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 146 - protein, 197 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKVSSEE

SEQ ID NO: 147 - protein, 304 aa, Homo sapiens
MGNHAGKRELNAEKASTNSETNRGESEKKRNLGELSRTTSEDNEVFGEADANQNNGTSSQDTAVTDSKRTADPKNAWQDAHPADPGSRPHLIRLFSRDAPGREDNTFKDRPSESDELQTIQEDSAATSESLDVMASQKRPSQRHGSKYLATASTMDHARHGFLPRHRDTGILDSIGRFFGGDRGAPKRGSGKDSHHPARTAHYGSLPQKSHGRTQDENPVVHFFKNIVTPRTPPPSQGKGRGLSLSRFSWGAEGQRPGFGYGGRASDYKSAHKGFKGVDAQGTLSKIFKLGGRDSRSGSPMARR

SEQ ID NO: 148 - protein, 186 aa, Homo sapiens
MASQKRPSQRHGSKYLATASTMDHARHGFLPRHRDTGILDSIGRFFGGDRGAPKRGSGKVPWLKPGRSPLPSHARSQPGLCNMYKDSHHPARTAHYGSLPQKSHGRTQDENPVVHFFKNIVTPRTPPPSQGKGAEGQRPGFGYGGRASDYKSAHKGFKGVDAQGTLSKIFKLGGRDSRSGSPMARR

SEQ ID NO: 149 - protein, 277 aa, Homo sapiens; identical to SEQ ID NO: 128

SEQ ID NO: 150 - protein, 242 aa, Homo sapiens
MGLLECCARCLVGAPFASLVATGLCFFGVALFCGCGHEALTGTEKLIETYFSKNYQDYEYLINVIHAFQYVIYGTASFFFLYGALLLAEGFYTTGAVRQIFGDYKTTICGKGLSATFVGITYALTVVWLLVFACSAVPVYIYFNTWTTCQSIAFPSKTSASIGSLCADARMYGVLPWNAFPGKVCGSNLLSICKTAEFQMTFHLFIAAFVGAAATLVSLLTFMIAATYNFAVLKLMGRGTKF

SEQ ID NO: 151 - protein, 246 aa, Artificial sequence
  RTL1000-BirA
MEVGWYRPPFSRVVHLYRNGKGGGGSLVPRGSGGGGPRFLWQPKRECHFFNGTERVRFLDRYFYNQEESVRFDSDVGEFRAVTELGRPDAEYWNSQKDILEQARAAVDTYCRHNYGVVESFTVQRRVIKEEHDIDQDEDYDNPDQSGEFMFDFDGDEIFHVDMAKKETVWRLEEFGRFASFEAQGALANIAVDKANLEIMTKRSNYTPITNGGGGSGGGGSGGGGSGGGGSLHHILDAQKMVWNHR

SEQ ID NO: 152 - protein, 209 aa, Artificial sequence
  RTL340-BirA
MENPVVHFFKNIVTPRGGGGSLVPRGSGGGGPRFLWQPKRECHFFNGTERVRFLDRYFYNQEESVRFDSDVGEFRAVTELGRPDAEYWNSQKDILEQARAAVDTYCRHNYGVVESFTVQRRVIKEEHDIDQDEDYDNPDQSGEFMFDFDGDEIFHVDMAKKETVWRLEEFGRFASFEAQGALANIAVDKANLEIMTKRSNYTPITNGGG

SEQ ID NO: 153 - protein, 15 aa, Artificial sequence
  A linker between antigenic peptide and Beta-1 domain
GGGGSLVPRGSGGGG

SEQ ID NO: 154 - protein, 91 aa, Artificial sequence
  Beta-1 domain of DR2
PRFLWQPKRECHFFNGTERVRFLDRYFYNQEESVRFDSDVGEFRAVTELGRPDAEYWNSQKDILEQARAAVDTYCRHNYGVVESFTVQRRV

SEQ ID NO: 155 - protein, 84 aa, Artificial sequence
  Alpha-1 domain of DR2
IKEEHDIDQDEDYDNPDQSGEFMFDFDGDEIFHVDMAKKETVWRLEEFGRFASFEAQGALANIAVDKANLEIMTKRSNYTPITN

SEQ ID NO: 156 - protein, 20 aa, Artificial sequence
  A linker peptide connecting the Alpha-1 domain with the BirA tag
GGGGSGGGGSGGGGSGGGGS

SEQ ID NO: 157 - protein, 15 aa, Artificial sequence
  BirA tag
LHHILDAQKMVWNHR

SEQ ID NO: 158 - protein, 15 aa, Artificial sequence
  MHC class II insulin A1 antigenic peptide
GIVEQCCTSICSLYQ

SEQ ID NO: 159 - protein, 180 aa, Artificial sequence
  RTL800 (HLA-DQ2)
MGDTRPRFLEQVKHECHFFNGTERVRFLDRYFYHQEEYVRFDSDVGEFRAVTELGRPDAEYWNSQKDLLEQKRAAVDTYCRHNYGVGESFTVQRRVIKEEHVIIQAEFYLNPDQSGEFMFDFDGDEIFHVDMAKKETVWRLEEFGRFASFEAQGALANIAVDKANLEIMTKRSNYTPITN

SEQ ID NO: 160 - DNA, 549 bp, Artificial sequence
  RTL800 (HLA-DQ2)
atgggggacacccgaccacgcttcctggagcaggttaaacatgagtgtcatttcttcaatgggacggagcgggtgcggttcctggacagatacttctatcaccaagaggagtacgtgcgcttcgacagcgacgtgggggagttccgggcggtgacggagctggggcggcctgacgctgagtactggaacagccagaaggacctcctggagcagaagcgggccgcggtggacacctactgcagacacaactacggggttggtgagagcttcacagtgcagcggcgagtcatcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaactcgag

SEQ ID NO: 161 - protein, 178 aa, Artificial sequence
  RTL600 (HLA-DP2)
MGDTPENYLFQGRQECYAFNGTQRFLERYIYNREEFVRFDSDVGEFRAVTELGRPDEEYWNSQKDILEEERAVPDRMCRHNYELGGPMTLQRRVIKADHVSTYAAFVQTHRPTGEFMFEFDEDEMFYVDLDKKETVWHLEEFGQAFSFEAQGGLANIAILNNNLNTLIQRSNHTQATN

SEQ ID NO: 162 - DNA, 543 bp, Artificial sequence
  RTL600 (HLA-DP2)
atgggggacactccggagaattaccttttccagggacggcaggaatgctacgcgtttaatgggacacagcgcttcctggagagatacatctacaaccgggaggagttcgtgcgcttcgacagcgacgtgggggagttccgggcggtgacggagctggggcggcctgatgaggagtactggaacagccagaaggacatcctggaggaggagcgggcagtgccggacaggatgtgcagacacaactacgagctgggcgggcccatgaccctgcagcgccgagtcatcaaagcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaactaactcgag

SEQ ID NO: 163 - protein, 211 aa, Artificial sequence
  Empty RTL 302 (empty DR2)
MPRFLWQPKRECHFFNGTERVRFLDRYFYNQEESVRFDSDVGEFRAVTELGRPDAEYWNSQKDILEQARAAVDTYCRHNYGVVESFTVQRRVIKEEHDIDQDEDYDNPDQSGEFMFDFDGDEIFHVDMAKKETVWRLEEFGRFASFEAQGALANIAVDKANLEIMTKRSNYTPITNGGGGSGGGGSGGGGSGGGGSLHHILDAQKMVWNHR

SEQ ID NO: 164 - DNA, 633 bp, Artificial sequence
  Empty RTL 302 (empty DR2)
atgccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtcatcaaagaagaacatgacatcgaccaggacgaggactatgacaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatggtggcggtggaagcggtggcggtggaagcggtggcggtggaagcggtggcggtggaagcctgcaccatatcctggacgcccagaagatggtgtggaatcaccgc

SEQ ID NO: 165 - protein, 8 aa, Artificial sequence
  A linker between antigenic peptide and the Beta-1 domain of DR4
GSGSGSGS

SEQ ID NO: 166 - protein, 95 aa, Artificial sequence
  Beta-1 domain DR4
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRV

SEQ ID NO: 167 - protein, 84 aa, Artificial sequence
  Alpha-1 domain DR4
IKEEHVIIQAEFYLNPDQSGEFMFDFDGDEIFHVDMAKKETVWRLEEFGRFASFEAQGALANIAVDKANLEIMTKRSNYTPITN

SEQ ID NO: 168 - protein, 238 aa, Artificial sequence
  RTL2011
MGIVEQCCTSICSLYQGSGSGSGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVIKEEHVIIQAEFYLNPDQSGEFMFDFDGDEIFHVDMAKKETVWRLEEFGRFASFEAQGALANIAVDKANLEIMTKRSNYTPITNGGGGSGGGGSGGGGSGGGGSLHHILDAQKMVWNHR

SEQ ID NO: 169 - protein, 236 aa, Artificial sequence
  RTL2010
MMFFRMVISNPAATGSGSGSGSGDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVIKEEHVIIQAEFYLNPDQSGEFMFDFDGDEIFHVDMAKKETVWRLEEFGRFASFEAQGALANIAVDKANLEIMTKRSNYTPITNGGGGSGGGGSGGGGSGGGGSLHHILDAQKMVWNHR

SEQ ID NO: 170 - DNA, 738 bp, Artificial sequence
  RTL1000-BirA
atggaagttggttggtaccgtccgccgttctcccgtgttgttcacctgtaccgtaacggtaaaggaggtggaggctcactagtgccccgaggctctggaggtggaggcccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtcatcaaagaagaacatgacatcgaccaggacgaggactatgacaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatggtggcggtggaagcggtggcggtggaagcggtggcggtggaagcggtggcggtggaagcctgcaccatatcctggacgcccagaagatggtgtggaatcaccgc

SEQ ID NO: 171 - DNA, 63 bp, Artificial sequence
  sequence encoding peptide MOG-35-55
atggaagttggttggtaccgtccgccgttctcccgtgttgttcacctgtaccgtaacggtaaa

SEQ ID NO: 172 - DNA, 45 bp, Artificial sequence
  Sequence encoding a linker between antigenic peptide and the Beta1 domain in RTL
ggaggtggaggctcactagtgccccgaggctctggaggtggaggc

SEQ ID NO: 173 - DNA, 273 bp, Artificial sequence
  Sequence encoding  Beta-1 domain of DR2
ccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtc

SEQ ID NO: 174 - DNA, 252 bp, Artificial sequence
  Sequence encoding  Alpha-1 domain of DR2
atcaaagaagaacatgacatcgaccaggacgaggactatgacaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaat

SEQ ID NO: 175 - DNA, 60 bp, Artificial sequence
  Sequence encoding a linker between  Alpha-1 domain and the BirA tag in RTL
ggtggcggtggaagcggtggcggtggaagcggtggcggtggaagcggtggcggtggaagc

SEQ ID NO: 176 - DNA, 45 bp, Artificial sequence
  A sequence encoding for the BirA tag
ctgcaccatatcctggacgcccagaagatggtgtggaatcaccgc

SEQ ID NO: 177 - DNA, 46 bp, Artificial sequence
  A sequence encoding insulin A-1-15 antigenic peptide
ggtatcgttgaacagtgttgtaccagcatctgcagcctgtatcagg

SEQ ID NO: 178 - DNA, 23 bp, Artificial sequence
  sequence encoding a linker between an antigenic peptide and the Beta-1 domain in RTL
gttctggttctggttctggttct

SEQ ID NO: 179 - DNA, 285 bp, Artificial sequence
  A sequence encoding  the  Beta-1 domain DR4
ggggacacccgaccacgcttcctggagcaggttaaacatgagtgtcatttcttcaatgggacggagcgggtgcggttcctggacagatacttctatcaccaagaggagtacgtgcgcttcgacagcgacgtgggggagttccgggcggtgacggagctggggcggcctgacgctgagtactggaacagccagaaggacctcctggagcagaagcgggccgcggtggacacctactgcagacacaactacggggttggtgagagcttcacagtgcagcggcgagtc

SEQ ID NO: 180 - DNA, 252 bp, Artificial sequence; identical to SEQ ID NO: 174
  A sequence encoding  the  Alpha-1 domain DR4

SEQ ID NO: 181 - DNA, 714 bp, Artificial sequence
  RTL2011
atgggtatcgttgaacagtgttgtaccagcatctgcagcctgtatcagggttctggttctggttctggttctggggacacccgaccacgcttcctggagcaggttaaacatgagtgtcatttcttcaatgggacggagcgggtgcggttcctggacagatacttctatcaccaagaggagtacgtgcgcttcgacagcgacgtgggggagttccgggcggtgacggagctggggcggcctgacgctgagtactggaacagccagaaggacctcctggagcagaagcgggccgcggtggacacctactgcagacacaactacggggttggtgagagcttcacagtgcagcggcgagtcatcaaagaagaacatgacatcgaccaggacgaggactatgacaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatggtggcggtggaagcggtggcggtggaagcggtggcggtggaagcggtggcggtggaagcctgcaccatatcctggacgcccagaagatggtgtggaatcaccgc

SEQ ID NO: 182 - DNA, 708 bp, Artificial sequence
  RTL2010
atgaacttctttcgtatggttatcagcaatccagctgcgactggttctggttctggttctggttctggggacacccgaccacgcttcctggagcaggttaaacatgagtgtcatttcttcaatgggacggagcgggtgcggttcctggacagatacttctatcaccaagaggagtacgtgcgcttcgacagcgacgtgggggagttccgggcggtgacggagctggggcggcctgacgctgagtactggaacagccagaaggacctcctggagcagaagcgggccgcggtggacacctactgcagacacaactacggggttggtgagagcttcacagtgcagcggcgagtcatcaaagaagaacatgacatcgaccaggacgaggactatgacaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatggtggcggtggaagcggtggcggtggaagcggtggcggtggaagcggtggcggtggaagcctgcaccatatcctggacgcccagaagatggtgtggaatcaccgc

SEQ ID NO: 183 - DNA, 40 bp, Artificial sequence
  A sequence encoding GAD-555-567 antigenic peptide
aacttctttcgtatggttatcagcaatccagctgcgactg

SEQ ID NO: 184 - protein, 96 aa, Artificial sequence
  Beta-1 domain HLA-DQ2
MGDTRPRFLEQVKHECHFFNGTERVRFLDRYFYHQEEYVRFDSDVGEFRAVTELGRPDAEYWNSQKDLLEQKRAAVDTYCRHNYGVGESFTVQRRV

SEQ ID NO: 185 - protein, 84 aa, Artificial sequence; identical to SEQ ID NO: 167
  Alpha-1 domain HLA-DQ2

SEQ ID NO: 186 - DNA, 288 bp, Artificial sequence
  Beta-1 domain HLA-DQ2
atgggggacacccgaccacgcttcctggagcaggttaaacatgagtgtcatttcttcaatgggacggagcgggtgcggttcctggacagatacttctatcaccaagaggagtacgtgcgcttcgacagcgacgtgggggagttccgggcggtgacggagctggggcggcctgacgctgagtactggaacagccagaaggacctcctggagcagaagcgggccgcggtggacacctactgcagacacaactacggggttggtgagagcttcacagtgcagcggcgagtc

SEQ ID NO: 187 - DNA, 261 bp, Artificial sequence
  Alpha-1 domain HLA-DQ2
atcaaagaagaacatgtgatcatccaggccgagttctatctgaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacctggaaatcatgacaaagcgctccaactatactccgatcaccaattaactcgag

SEQ ID NO: 188 - protein, 94 aa, Artificial sequence
  Beta-1 domain of the empty RTL800
MGDTPENYLFQGRQECYAFNGTQRFLERYIYNREEFVRFDSDVGEFRAVTELGRPDEEYWNSQKDILEEERAVPDRMCRHNYELGGPMTLQRRV

SEQ ID NO: 189 - protein, 84 aa, Artificial sequence
  Alpha-1 domain of the empty RTL800
IKADHVSTYAAFVQTHRPTGEFMFEFDEDEMFYVDLDKKETVWHLEEFGQAFSFEAQGGLANIAILNNNLNTLIQRSNHTQATN

SEQ ID NO: 190 - DNA, 282 bp, Artificial sequence
  Beta-1 domain of the empty RTL800
atgggggacactccggagaattaccttttccagggacggcaggaatgctacgcgtttaatgggacacagcgcttcctggagagatacatctacaaccgggaggagttcgtgcgcttcgacagcgacgtgggggagttccgggcggtgacggagctggggcggcctgatgaggagtactggaacagccagaaggacatcctggaggaggagcgggcagtgccggacaggatgtgcagacacaactacgagctgggcgggcccatgaccctgcagcgccgagtc

SEQ ID NO: 191 - DNA, 261 bp, Artificial sequence
  Alpha-1 domain of the empty RTL800
atcaaagcggaccatgtgtcaacttatgccgcgtttgtacagacgcatagaccaacaggggagtttatgtttgaatttgatgaagatgagatgttctatgtggatctggacaagaaggagaccgtctggcatctggaggagtttggccaagccttttcctttgaggctcagggcgggctggctaacattgctatattgaacaacaacttgaataccttgatccagcgttccaaccacactcaggccaccaactaactcgag

SEQ ID NO: 192 - DNA, 45 bp, Artificial sequence
  DNA encoding the MBP-85-99 antigenic-peptide
gaaaacccggttgttcacttcttcaaaaacatcgttaccccgcgt

SEQ ID NO: 193 - DNA, 723 bp, Artificial sequence
  RTL340-BirA
atggaaaacccggttgttcacttcttcaaaaacatcgttaccccgcgtggaggtggaggctcactagtgccccgaggctctggaggtggaggcccacgtttcctgtggcagcctaagagggagtgtcatttcttcaatgggacggagcgggtgcggttcctggacagatacttctataaccaggaggagtccgtgcgcttcgacagcgacgtgggggagttccgggcggtgacggagctggggcggcctgacgctgagtactggaacagccagaaggacatcctggagcaggcgcgggccgcggtggacacctactgcagacacaactacggggttgtggagagcttcacagtgcagcggcgagtcatcaaagaagaacatgacatcgaccaggacgaggactatgacaatcctgaccaatcaggcgagtttatgtttgactttgatggtgatgagattttccatgtggatatggcaaagaaggagacggtctggcggcttgaagaatttggacgatttgccagctttgaggctcaaggtgcattggccaacatagctgtggacaaagccaacttggaaatcatgacaaagcgctccaactatactccgatcaccaatggtggcggtggaagcggtggcggtggaagcggtggcggtggaagcggtggcggtggaagcctgcaccatatcctggacgcccagaagatggtgtggaatcaccgc

SEQ ID NO: 194 - DNA, 98 bp, Artificial sequence
  Single strand DNA oligonucleotide
ttaagcgttggcggaattcttatcagcggtgattccacaccatcttctgggcgtccaggatatggtgcagagacccgggattggtgatcggagtatag

SEQ ID NO: 195 - protein, 13 aa, Artificial sequence
  CII-261-273 peptide
AGFKGEQGPKGEP

SEQ ID NO: 196 - protein, 13 aa, Artificial sequence
  HA-307-319 peptide
PKYVKQNTLKLAT

SEQ ID NO: 197 - protein, 11 aa, Artificial sequence
  Autoantigenic peptide
FPQPELPYPQP

SEQ ID NO: 198 - protein, 22 aa, Artificial sequence
  Autoantigenic peptide
VPVPQLQPQNPSQQQPQEQVPL

SEQ ID NO: 199 - protein, 279 aa, Aegilops tauschii
MKTFLILALLAIVATTATTAVRVPVPQLQPQNPSQQQPQEQVPLVQQQQFLGQQQPFPPQQPYPQPQPFPSQQPYLQLQPFPQPQLPYSQPQPFRPQQPYPQPQPQYSQPQEPISQQQQQQQQQQQILQQILQQQLIPCMDVVLQQHNIAHGRSQVLQQSTYQLLQELCCQHLWQIPEQSQCQAIQNVVHAIILHQQQKQQQQPSSQVSFQQPLQQYPLGQGSFRPSQQNPQDQGSVQPQQLPQFEEIRNLALQTLPAMCNVYIPPYCTIAPFGIFGTN

SEQ ID NO: 200 - protein, 21 aa, Artificial sequence
  Mouse (m)MOG-35-55 peptide
MEVGWYRSPFSRVVHLYRNGK

SEQ ID NO: 201 - protein, 192 aa, Artificial sequence
  Extracellular domain of HLA-DRB1*0401 beta chain
GDTRPRFLEQVKHECHFFNGTERVRFLDRYFYHQEEYVRFDSDVGEYRAVTELGRPDAEYWNSQKDLLEQKRAAVDTYCRHNYGVGESFTVQRRVYPEVTVYPAKTQPLQHHNLLVCSVNGFYPGSIEVRWFRNGQEEKTGVVSTGLIQNGDWTFQTLVMLETVPRSGEVYTCQVEHPSLTSPLTVEWRARS

SEQ ID NO: 202 - protein, 181 aa, Artificial sequence
  Extracellular domain of HLA-DRA1*0101 alpha chain
IKEEHVIIQAEFYLNPDQSGEFMFDFDGDEIFHVDMAKKETVWRLEEFGRFASFEAQGALANIAVDKANLEIMTKRSNYTPITNVPPEVTVLTNSPVELREPNVLICFIDKFTPPVVNVTWLRNGKPVTTGVSETVFLPREDHLFRKFHYLPFLPSTEDVYDCRVEHWGLDEPLLKHWEFD

SEQ ID NO: 203 - protein, 13 aa, Artificial sequence
  Autoantigenic peptide
MNILLQYVVKSFD

SEQ ID NO: 204 - protein, 9 aa, Artificial sequence
  Autoantigenic peptide
IAPVFVLLE

SEQ ID NO: 205 - protein, 14 aa, Artificial sequence
  Autoantigenic peptide
LPRLIAFTSEHSHF

SEQ ID NO: 206 - protein, 13 aa, Artificial sequence
  Autoantigenic peptide
IAFTSEHSHFSLK

SEQ ID NO: 207 - protein, 14 aa, Artificial sequence
  Autoantigenic peptide
TVYGAFDPLLAVAD

SEQ ID NO: 208 - protein, 15 aa, Artificial sequence
  Autoantigenic peptide
KYKIWMHVDAAWGGG

SEQ ID NO: 209 - protein, 15 aa, Artificial sequence
  Autoantigenic peptide
KHKWKLSGVERANSV

SEQ ID NO: 210 - protein, 15 aa, Artificial sequence
  Autoantigenic peptide
LYNIIKNREGYEMVF

SEQ ID NO: 211 - protein, 15 aa, Artificial sequence
  Autoantigenic peptide
PSLRTLEDNEERMSR

SEQ ID NO: 212 - protein, 15 aa, Artificial sequence
  Autoantigenic peptide
RMMEYGTTMVSYQPL

SEQ ID NO: 213 - protein, 15 aa, Artificial sequence
  Autoantigenic peptide
SYQPLGDKVNFFRMV

SEQ ID NO: 214 - protein, 13 aa, Artificial sequence
  Autoantigenic peptide
NFFRMVISNPAAT

SEQ ID NO: 215 - protein, 15 aa, Artificial sequence
  Autoantigenic peptide
ATHQDIDFLIEEIER

SEQ ID NO: 216 - protein, 9 aa, Artificial sequence
  Autoantigenic peptide
ATDLLPACD

SEQ ID NO: 217 - protein, 15 aa, Artificial sequence
  Autoantigenic peptide
FDRSTKVIDFHYPNE

SEQ ID NO: 218 - protein, 9 aa, Artificial sequence
  Autoantigenic peptide
ELLQEYNWE

SEQ ID NO: 219 - protein, 9 aa, Artificial sequence
  Autoantigenic peptide
EYNWELADQ

SEQ ID NO: 220 - protein, 9 aa, Artificial sequence
  Autoantigenic peptide
DIDFLIEEI

SEQ ID NO: 221 - protein, 15 aa, Artificial sequence
  Autoantigenic peptide
TGHPRYFNQLSTGLD

SEQ ID NO: 222 - protein, 15 aa, Artificial sequence
  Autoantigenic peptide
TYEIAPVFVLLEYVT

SEQ ID NO: 223 - protein, 9 aa, Artificial sequence
  Autoantigenic peptide
YVTLKKMRE

SEQ ID NO: 224 - protein, 20 aa, Artificial sequence
  Autoantigenic peptide
PGGSGDGIFSPGGAISNMYA

SEQ ID NO: 225 - protein, 20 aa, Artificial sequence
  Autoantigenic peptide
NMYAMMIARFKMFPEVKEKG

SEQ ID NO: 226 - protein, 20 aa, Artificial sequence
  Autoantigenic peptide
PEVKEKGMAALPRLIAFTSE

SEQ ID NO: 227 - protein, 9 aa, Artificial sequence
  Autoantigenic peptide
DSVILIKCD

SEQ ID NO: 228 - protein, 9 aa, Artificial sequence
  Autoantigenic peptide
GKMIPSDLE

SEQ ID NO: 229 - protein, 9 aa, Artificial sequence
  Autoantigenic peptide
ERRILEAKQ

SEQ ID NO: 230 - protein, 9 aa, Artificial sequence
  Autoantigenic peptide
ERANSVTWN

SEQ ID NO: 231 - protein, 9 aa, Artificial sequence
  Autoantigenic peptide
QCSALLVRE

SEQ ID NO: 232 - protein, 15 aa, Artificial sequence
  Autoantigenic peptide
KHYDLSYDTGDKALQ

SEQ ID NO: 233 - protein, 15 aa, Artificial sequence
  Autoantigenic peptide
AKGTTGFEAHVDKCL

SEQ ID NO: 234 - protein, 20 aa, Artificial sequence
  Autoantigenic peptide
VDKCLELAEYLYNIIKNREG

SEQ ID NO: 235 - protein, 9 aa, Artificial sequence
  Autoantigenic peptide
IIKNREGYE

SEQ ID NO: 236 - protein, 15 aa, Artificial sequence
  Autoantigenic peptide
MVFDGKPQHTNVCFW

SEQ ID NO: 237 - protein, 15 aa, Artificial sequence
  Autoantigenic peptide
CFWYIPPSLRTLEDN

SEQ ID NO: 238 - protein, 13 aa, Artificial sequence
  Autoantigenic peptide
FWYIPPSLRTLED

SEQ ID NO: 239 - protein, 9 aa, Artificial sequence
  Autoantigenic peptide
SLRTLEDNE

SEQ ID NO: 240 - protein, 15 aa, Artificial sequence
  Autoantigenic peptide
ERMSRLSKVAPVIKA

SEQ ID NO: 241 - protein, 15 aa, Artificial sequence
  Autoantigenic peptide
IKARMMEYGTTMVSY

SEQ ID NO: 242 - protein, 15 aa, Artificial sequence
  Autoantigenic peptide
RMMEYGTTMVSYQPL

SEQ ID NO: 243 - protein, 9 aa, Artificial sequence
  Autoantigenic peptide
VISNPAATH

SEQ ID NO: 244 - protein, 9 aa, Artificial sequence
  Autoantigenic peptide
IDFLIEEIE

SEQ ID NO: 245 - protein, 20 aa, Artificial sequence
  Autoantigenic peptide
NWELADQPQNLEEILMHCQT

SEQ ID NO: 246 - protein, 12 aa, Artificial sequence
  Autoantigenic peptide
GHPRYFNQLSTG

SEQ ID NO: 247 - protein, 20 aa, Artificial sequence [sequence omitted]

SEQ ID NO: 248 - protein, 17 aa, Artificial sequence
  Autoantigenic peptide
VNFFRMVISNPAATHQD

SEQ ID NO: 249 - protein, 21 aa, Artificial sequence
  Autoantigenic peptide
DKVNFFRMVISNPAATHQDID

SEQ ID NO: 250 - protein, 10 aa, Artificial sequence
  Autoantigenic peptide
FFRMVISNPA

SEQ ID NO: 251 - protein, 14 aa, Artificial sequence
  Autoantigenic peptide
LTIQIESAADQDPS

SEQ ID NO: 252 - protein, 14 aa, Artificial sequence
  Autoantigenic peptide
RTGIAQALSSFDLH

SEQ ID NO: 253 - protein, 14 aa, Artificial sequence
  Autoantigenic peptide
LYPDYQIQAGIMIT

SEQ ID NO: 254 - protein, 14 aa, Artificial sequence
  Autoantigenic peptide
ILSVHVATAASQDS

SEQ ID NO: 255 - protein, 14 aa, Artificial sequence
  Autoantigenic peptide
SKRLTFGWYRAEIL

SEQ ID NO: 256 - protein, 14 aa, Artificial sequence
  Autoantigenic peptide
AILTDAAHLLIDLT

SEQ ID NO: 257 - protein, 14 aa, Artificial sequence
  Autoantigenic peptide
KATGNRSSKQAHAK

SEQ ID NO: 258 - protein, 14 aa, Artificial sequence
  Autoantigenic peptide
AVDGVISVHSLHIW

SEQ ID NO: 259 - protein, 21 aa, Artificial sequence
  Autoantigenic peptide
VSSVSSQFSDAAQASPSSFSD

SEQ ID NO: 260 - protein, 24 aa, Artificial sequence
  Autoantigenic peptide
LAKEWQALCAYQAEPNTCATAQGE

SEQ ID NO: 261 - protein, 24 aa, Artificial sequence
  Autoantigenic peptide
KLKVESSPSRSDYINASPIIEHDP

SEQ ID NO: 262 - protein, 20 aa, Artificial sequence
  Autoantigenic peptide
IKLKVESSPSRSDYINASPI

SEQ ID NO: 263 - protein, 21 aa, Artificial sequence
  Autoantigenic peptide
MVWESGCTVIVMLTPLVEDGV

SEQ ID NO: 264 - protein, 18 aa, Artificial sequence
  Autoantigenic peptide
RQHARQQDKERLAALGPE

SEQ ID NO: 265 - protein, 18 aa, Artificial sequence
  Autoantigenic peptide
GPEGAHGDTTFEYQDLCR

SEQ ID NO: 266 - protein, 18 aa, Artificial sequence
  Autoantigenic peptide
EGPPEPSRVSSVSSQFSD

SEQ ID NO: 267 - protein, 18 aa, Artificial sequence
  Autoantigenic peptide
FSDAAQASPSSHSSTPSW

SEQ ID NO: 268 - protein, 18 aa, Artificial sequence
  Autoantigenic peptide
AEPNTCATAQGEGNIKKN

SEQ ID NO: 269 - protein, 18 aa, Artificial sequence
  Autoantigenic peptide
NASPIIEHDPRMPAYIAT

SEQ ID NO: 270 - protein, 18 aa, Artificial sequence
  Autoantigenic peptide
DEGSALYHVYEVNLVSEH

SEQ ID NO: 271 - protein, 17 aa, Artificial sequence
  Autoantigenic peptide
KGVKEIDIAATLEHVRD

SEQ ID NO: 272 - protein, 19 aa, Artificial sequence
  Autoantigenic peptide
FALTAVAEEVNAILKALPQ

SEQ ID NO: 273 - protein, 15 aa, Artificial sequence
  Autoantigenic peptide
KNRSLAVLTYDHSRI

SEQ ID NO: 274 - protein, 15 aa, Artificial sequence
  Autoantigenic peptide
GADPSADATEAYQEL

SEQ ID NO: 275 - protein, 9 aa, Artificial sequence
  Autoantigenic peptide
EIDIAATLE

SEQ ID NO: 276 - protein, 9 aa, Artificial sequence
  Autoantigenic peptide
NTCATAQGE

SEQ ID NO: 277 - protein, 9 aa, Artificial sequence
  Autoantigenic peptide
EPNTCATAQ

SEQ ID NO: 278 - protein, 9 aa, Artificial sequence
  Autoantigenic peptide
ERLAALGPE

SEQ ID NO: 279 - protein, 9 aa, Artificial sequence
  Autoantigenic peptide
QHARQQDKE

SEQ ID NO: 280 - protein, 9 aa, Artificial sequence
  Autoantigenic peptide
YEVNLVSEH

SEQ ID NO: 281 - protein, 9 aa, Artificial sequence
  Autoantigenic peptide
GASLYHVYE

SEQ ID NO: 282 - protein, 9 aa, Artificial sequence
  Autoantigenic peptide
FALTAVAEE

SEQ ID NO: 283 - protein, 9 aa, Artificial sequence
  Autoantigenic peptide
GAHGDTTFE

SEQ ID NO: 284 - protein, 9 aa, Artificial sequence
  Autoantigenic peptide
GDTTFEYQD

SEQ ID NO: 285 - protein, 9 aa, Artificial sequence
  Autoantigenic peptide
AAQASPSSH

SEQ ID NO: 286 - protein, 9 aa, Artificial sequence
  Autoantigenic peptide
SRVSSVSSQ

SEQ ID NO: 287 - protein, 9 aa, Artificial sequence
  Autoantigenic peptide
TQFHFLSWP

SEQ ID NO: 288 - protein, 9 aa, Artificial sequence
  Autoantigenic peptide
EEPAQANMD

SEQ ID NO: 289 - protein, 9 aa, Artificial sequence
  Autoantigenic peptide
GHMILAYME

SEQ ID NO: 290 - protein, 9 aa, Artificial sequence
  Autoantigenic peptide
MILAYMEDH

SEQ ID NO: 291 - protein, 9 aa, Artificial sequence
  Autoantigenic peptide
QALCAYQAE

SEQ ID NO: 292 - protein, 9 aa, Artificial sequence
  Autoantigenic peptide
EWQALCAYQ

SEQ ID NO: 293 - protein, 9 aa, Artificial sequence
  Autoantigenic peptide
LVRSKDQFE

SEQ ID NO: 294 - protein, 9 aa, Artificial sequence
  Autoantigenic peptide
VEDGVKQCD

SEQ ID NO: 295 - protein, 9 aa, Artificial sequence
  Autoantigenic peptide
YILIDMVLN

SEQ ID NO: 296 - protein, 9 aa, Artificial sequence
  Autoantigenic peptide
ESGCTVIVM

SEQ ID NO: 297 - protein, 9 aa, Artificial sequence
  Autoantigenic peptide
LCAYQAEPN

SEQ ID NO: 298 - protein, 9 aa, Artificial sequence
  Autoantigenic peptide
ETRTLTQFH

SEQ ID NO: 299 - protein, 9 aa, Artificial sequence
  Autoantigenic peptide
VESSPSRSD

SEQ ID NO: 300 - protein, 9 aa, Artificial sequence
  Autoantigenic peptide
GPLSHTIAD

SEQ ID NO: 301 - protein, 9 aa, Artificial sequence
  Autoantigenic peptide
SLFNRAEGP

SEQ ID NO: 302 - protein, 9 aa, Artificial sequence
  Autoantigenic peptide
HPDFLPYDH

SEQ ID NO: 303 - protein, 9 aa, Artificial sequence
  Autoantigenic peptide
HFLSWPAEG

SEQ ID NO: 304 - protein, 9 aa, Artificial sequence
  Autoantigenic peptide
DFRRKVNKC

SEQ ID NO: 305 - protein, 9 aa, Artificial sequence
  Autoantigenic peptide
HCSDGAGRT

SEQ ID NO: 306 - protein, 9 aa, Artificial sequence
  Autoantigenic peptide
LVRSFYLKN

SEQ ID NO: 307 - protein, 15 aa, Artificial sequence
  Autoantigenic peptide
KNRSLAVLTYDHSRI

SEQ ID NO: 308 - protein, 15 aa, Artificial sequence
  Autoantigenic peptide
GADPSADATEAYQEL

SEQ ID NO: 309 - protein, 16 aa, Artificial sequence
  Autoantigenic peptide
ANMDISTGHMILAYME

SEQ ID NO: 310 - protein, 16 aa, Artificial sequence
  Autoantigenic peptide
WQALCAYQAEPNTCAT

SEQ ID NO: 311 - protein, 16 aa, Artificial sequence
  Autoantigenic peptide
LSHTIADFWQMVWESG

SEQ ID NO: 312 - protein, 16 aa, Artificial sequence
  Autoantigenic peptide
DFWQMVWESGCTVIVM

SEQ ID NO: 313 - protein, 16 aa, Artificial sequence
  Autoantigenic peptide
WESGCTVIVMLTPLVE

SEQ ID NO: 314 - protein, 16 aa, Artificial sequence
  Autoantigenic peptide
VIVMLTPLVEDGVKQC

SEQ ID NO: 315 - protein, 16 aa, Artificial sequence
  Autoantigenic peptide
SEHIWCEDFLVRSFYL

SEQ ID NO: 316 - protein, 16 aa, Artificial sequence
  Autoantigenic peptide
WCEDFLVRSFYLKNVQ

SEQ ID NO: 317 - protein, 16 aa, Artificial sequence
  Autoantigenic peptide
EDFLVRSFYLKNVQTQ

SEQ ID NO: 318 - protein, 16 aa, Artificial sequence
  Autoantigenic peptide
DFRRKVNKCYRGRSCP

SEQ ID NO: 319 - protein, 16 aa, Artificial sequence
  Autoantigenic peptide
YILIDMVLNRMAKGVK

SEQ ID NO: 320 - protein, 16 aa, Artificial sequence
  Autoantigenic peptide
FEFALTAVAEEVNAIL

SEQ ID NO: 321 - protein, 9 aa, Artificial sequence
  Autoantigenic peptide
EALYLVCGE

SEQ ID NO: 322 - protein, 9 aa, Artificial sequence
  Autoantigenic peptide
SICSLYQLE

SEQ ID NO: 323 - protein, 9 aa, Artificial sequence
  Autoantigenic peptide
ALLALWGPD

SEQ ID NO: 324 - protein, 9 aa, Artificial sequence
  Autoantigenic peptide
GSLQPLALE

SEQ ID NO: 325 - protein, 9 aa, Artificial sequence
  Autoantigenic peptide
TPKTRREAE

SEQ ID NO: 326 - protein, 9 aa, Artificial sequence
  Autoantigenic peptide
PAAAFVNQH

SEQ ID NO: 327 - protein, 9 aa, Artificial sequence
  Autoantigenic peptide
DPAAAFVNQ

SEQ ID NO: 328 - protein, 9 aa, Artificial sequence
  Autoantigenic peptide
PDPAAAFVN

SEQ ID NO: 329 - protein, 9 aa, Artificial sequence
  Autoantigenic peptide
QKRGIVEQC

SEQ ID NO: 330 - protein, 9 aa, Artificial sequence
  Autoantigenic peptide
ELGGGPGAG

SEQ ID NO: 331 - protein, 9 aa, Artificial sequence
  Autoantigenic peptide
EAEDLQVGQ

SEQ ID NO: 332 - protein, 9 aa, Artificial sequence
  Autoantigenic peptide
LQVGQVELG

SEQ ID NO: 333 - protein, 9 aa, Artificial sequence
  Autoantigenic peptide
HLCGSHLVE

SEQ ID NO: 334 - protein, 12 aa, Artificial sequence
  Autoantigenic peptide
GIVEQCCTSICS

SEQ ID NO: 335 - protein, 14 aa, Artificial sequence
  Autoantigenic peptide
KRGIVEQCCTSICS

SEQ ID NO: 336 - protein, 16 aa, Artificial sequence
  Autoantigenic peptide
LALLALWGPDPAAAFV

SEQ ID NO: 337 - protein, 16 aa, Artificial sequence
  Autoantigenic peptide
PAAAFVNQHLCGSHLV

SEQ ID NO: 338 - protein, 15 aa, Artificial sequence
  Autoantigenic peptide
SHLVEALYLVCGERG

SEQ ID NO: 339 - protein, 13 aa, Artificial sequence
  Autoantigenic peptide
FFYTPKTRREAED

SEQ ID NO: 340 - protein, 18 aa, Artificial sequence
  Autoantigenic peptide
GAGSLQPLALEGSLQKRG

SEQ ID NO: 341 - protein, 17 aa, Artificial sequence
  Autoantigenic peptide
SLQKRGIVEQCCTSICS

SEQ ID NO: 342 - protein, 20 aa, Artificial sequence
  Autoantigenic peptide
KFGADARALMLQGVDLLADA

SEQ ID NO: 343 - protein, 20 aa, Artificial sequence
  Autoantigenic peptide
NPVEIRRGVMLAVDAVIAEL

SEQ ID NO: 344 - protein, 21 aa, Artificial sequence
  Autoantigenic peptide
QSIVPALEIANAHRKPLVIIA

SEQ ID NO: 345 - protein, 20 aa, Artificial sequence
  Autoantigenic peptide
LVLNRLKVGLQVVAVKAPGF

SEQ ID NO: 346 - protein, 20 aa, Artificial sequence
  Autoantigenic peptide
IVLGGGCALLRCIPALDSLT

SEQ ID NO: 347 - protein, 24 aa, Artificial sequence
  Autoantigenic peptide
VLGGGCALLRCIPALDSLTPANED

SEQ ID NO: 348 - protein, 20 aa, Artificial sequence
  Autoantigenic peptide
EIIKRTLKIPAMTIAKNAGV

SEQ ID NO: 349 - protein, 20 aa, Artificial sequence
  Autoantigenic peptide
VNMVEKGIIDPTKVVRTALL

SEQ ID NO: 350 - protein, 20 aa, Artificial sequence
  Autoantigenic peptide
MAKAAAVGIDLGTTYSCVGV

SEQ ID NO: 351 - protein, 20 aa, Artificial sequence
  Autoantigenic peptide
GLNVLRIINEPTAAAIAYGL

SEQ ID NO: 352 - protein, 20 aa, Artificial sequence
  Autoantigenic peptide
TIDDGIFEVKATAGDTHLGG

SEQ ID NO: 353 - protein, 20 aa, Artificial sequence
  Autoantigenic peptide
THLGGEDFDNRLVNHFVEEF

SEQ ID NO: 354 - protein, 20 aa, Artificial sequence
  Autoantigenic peptide
KRTLSSSTQASLEIDSLFEG

SEQ ID NO: 355 - protein, 20 aa, Artificial sequence
  Autoantigenic peptide
LLLLDVAPLSLGLETAGGVM

SEQ ID NO: 356 - protein, 20 aa, Artificial sequence
  Autoantigenic peptide
PTKQTQIFTTYSDNQPGVLI

SEQ ID NO: 357 - protein, 20 aa, Artificial sequence
  Autoantigenic peptide
KANKITITNDKGRLSKEEIE

SEQ ID NO: 358 - protein, 20 aa, Artificial sequence
  Autoantigenic peptide
KEEIERMVQEAEKYKAEDEV

SEQ ID NO: 359 - protein, 13 aa, Artificial sequence
  Autoantigenic peptide
QHLQKDYRAYYTF

SEQ ID NO: 360 - protein, 13 aa, Artificial sequence
  Autoantigenic peptide
RVLNIDLLWSVPI

SEQ ID NO: 361 - protein, 13 aa, Artificial sequence
  Autoantigenic peptide
YTFLNFMSNVGDP

SEQ ID NO: 362 - protein, 13 aa, Artificial sequence
  Autoantigenic peptide
DWIHIDTTPFAGL

SEQ ID NO: 363 - protein, 20 aa, Artificial sequence
  Autoantigenic peptide
RSQPGLCNMYKDSHHPARTA

SEQ ID NO: 364 - protein, 21 aa, Artificial sequence
  Autoantigenic peptide
FKGVDAQGTLSKIFKLGGRDS

SEQ ID NO: 365 - protein, 21 aa, Artificial sequence
  Autoantigenic peptide
GDRGAPKRGSGKVPWLKPGRS

SEQ ID NO: 366 - protein, 20 aa, Artificial sequence
  Autoantigenic peptide
RSQPGLCNMYKDSHHPARTA

SEQ ID NO: 367 - protein, 21 aa, Artificial sequence
  Autoantigenic peptide
SDYKSAHKGFKGVDAQGTLSK

SEQ ID NO: 368 - protein, 21 aa, Artificial sequence
  Autoantigenic peptide
FKGVDAQGTLSKIFKLGGRDS

SEQ ID NO: 369 - protein, 21 aa, Artificial sequence
  Autoantigenic peptide
SDYKSAHKGFKGVDAQGTLSK

SEQ ID NO: 370 - protein, 15 aa, Artificial sequence
  Autoantigenic peptide
ENPVVHFFKNIVTPR

SEQ ID NO: 371 - protein, 21 aa, Artificial sequence
  Autoantigenic peptide
VFACSAVPVYIYFNTWTTCQS

SEQ ID NO: 372 - protein, 21 aa, Artificial sequence
  Autoantigenic peptide
YIYFNTWTTCQSIAFPSKTSA

SEQ ID NO: 373 - protein, 21 aa, Artificial sequence
  Autoantigenic peptide
AHSLERVCHCLGKWLGHPDKF

SEQ ID NO: 374 - protein, 21 aa, Artificial sequence
  Autoantigenic peptide
AVRQIFGDYKTTICGKGLSAT

SEQ ID NO: 375 - protein, 21 aa, Artificial sequence
  Autoantigenic peptide
FMIAATYNFAVLKLMGRGTKF

SEQ ID NO: 376 - protein, 21 aa, Artificial sequence
  Autoantigenic peptide
AHSLERVCHCLGKWLGHPDKF

SEQ ID NO: 377 - protein, 21 aa, Artificial sequence
  Autoantigenic peptide
AVRQIFGDYKTTICGKGLSAT

SEQ ID NO: 378 - protein, 20 aa, Artificial sequence
  Autoantigenic peptide
CQSIAFPSKTSASIGSLCAD

SEQ ID NO: 379 - protein, 20 aa, Artificial sequence
  Autoantigenic peptide
SKTSASIGSLCADARMYGVL

SEQ ID NO: 380 - protein, 20 aa, Artificial sequence
  Autoantigenic peptide
GVLPWNAFPGKVCGSNLLSI

SEQ ID NO: 381 - protein, 21 aa, Artificial sequence
  Autoantigenic peptide
FMIAATYNFAVLKLMGRGTKF

SEQ ID NO: 382 - protein, 21 aa, Artificial sequence
  Autoantigenic peptide
ELKVEDPFYWVSPGVLVLLAV

SEQ ID NO: 383 - protein, 20 aa, Artificial sequence
  Autoantigenic peptide
TFDPHFLRVPCWKITLFVIV

SEQ ID NO: 384 - protein, 21 aa, Artificial sequence
  Autoantigenic peptide
VIVPVLGPLVALIICYNWLHR

SEQ ID NO: 385 - protein, 21 aa, Artificial sequence
  Autoantigenic peptide
VALIICYNWLHRRLAGQFLEE

SEQ ID NO: 386 - protein, 21 aa, Artificial sequence
  Autoantigenic peptide
GFTCFFRDHSYQEEAAMELKV

SEQ ID NO: 387 - protein, 21 aa, Artificial sequence
  Autoantigenic peptide
ITVGLVFLCLQYRLRGKLRAE

SEQ ID NO: 388 - protein, 21 aa, Artificial sequence
  Autoantigenic peptide
VALIICYNWLHRRLAGQFLEE

SEQ ID NO: 389 - protein, 21 aa, Artificial sequence
  Autoantigenic peptide
LQYRLRGKLRAEIENLHRTFD

SEQ ID NO: 390 - protein, 21 aa, Artificial sequence
  Autoantigenic peptide
ELKVEDPFYWVSPGVLVLLAV

SEQ ID NO: 391 - protein, 21 aa, Artificial sequence
  Autoantigenic peptide
LQYRLRGKLRAEIENLHRTFD

SEQ ID NO: 392 - protein, 14 aa, Artificial sequence
  BirA recognition motif
LGGIFEAMKMELRD

SEQ ID NO: 393 - protein, 25 aa, Artificial sequence
  Autoantigenic peptide
PERYGRTELLKDAIGEGKVTLRIRN

SEQ ID NO: 394 - protein, 12 aa, Artificial sequence
  Autoantigenic peptide
TCFFRDHSYQEE

SEQ ID NO: 395 - protein, 9 aa, Artificial sequence
  Autoantigenic peptide
FVIVPVLGP

SEQ ID NO: 396 - protein, 13 aa, Artificial sequence
  Autoantigenic peptide
KITLFVIVPVLGP

SEQ ID NO: 397 - protein, 13 aa, Artificial sequence
  Autoantigenic peptide
AGFKGEQGPKGEP

SEQ ID NO: 398 - protein, 18 aa, Artificial sequence
  Autoantigenic peptide
EPGIAGFKGEQGPKGEPG

SEQ ID NO: 399 - protein, 15 aa, Artificial sequence
  Autoantigenic peptide
GVVSHSFPATLETQE

SEQ ID NO: 400 - protein, 15 aa, Artificial sequence
  Autoantigenic peptide
LSGLPSGGEVLEISV

SEQ ID NO: 401 - protein, 15 aa, Artificial sequence
  Autoantigenic peptide
ISGLPSGGDDLETST

SEQ ID NO: 402 - protein, 15 aa, Artificial sequence
  Autoantigenic peptide
FFYFFTGSSQLEFDP

SEQ ID NO: 403 - protein, 15 aa, Artificial sequence
  Autoantigenic peptide
HAYSVTGAEEVESNG

SEQ ID NO: 404 - protein, 15 aa, Artificial sequence
  Autoantigenic peptide
SAFWPSLPSGLDAAY

SEQ ID NO: 405 - protein, 15 aa, Artificial sequence
  Autoantigenic peptide
CVDTRSGNCYLDIRP

SEQ ID NO: 406 - protein, 15 aa, Artificial sequence
  Autoantigenic peptide
VKEGHSPPDDVDIVI

SEQ ID NO: 407 - protein, 15 aa, Artificial sequence
  Autoantigenic peptide
EPVSGSFTTALDGPS

SEQ ID NO: 408 - protein, 17 aa, Artificial sequence
  Autoantigenic peptide
RDYFEEYGKIDTIEIIT

SEQ ID NO: 409 - protein, 13 aa, Artificial sequence
  Autoantigenic peptide
LGQQQPFPPQQPY

SEQ ID NO: 410 - protein, 12 aa, Artificial sequence
  Autoantigenic peptide
QLQPFPQPQLPY

SEQ ID NO: 411 - protein, 14 aa, Artificial sequence
  Autoantigenic peptide
PQPQLPYPQPQLPY

SEQ ID NO: 412 - protein, 13 aa, Artificial sequence
  Autoantigenic peptide
PGQQQPFPPQQPY

SEQ ID NO: 413 - protein, 11 aa, Artificial sequence
  Autoantigenic peptide
GIIQPQQPAQL

SEQ ID NO: 414 - protein, 13 aa, Artificial sequence
  Autoantigenic peptide
FPQQPQQPYPQQP

SEQ ID NO: 415 - protein, 12 aa, Artificial sequence
  Autoantigenic peptide
FSQPQQQFPQPQ

SEQ ID NO: 416 - protein, 13 aa, Artificial sequence
  Autoantigenic peptide
PQQPFPQQPQQPY

SEQ ID NO: 417 - protein, 26 aa, Artificial sequence
  Autoantigenic peptide
FLQPQQPFPQQPQQPYPQQPQQPFPQ

SEQ ID NO: 418 - protein, 11 aa, Artificial sequence
  Autoantigenic peptide
ALPTAQVPTDP

SEQ ID NO: 419 - protein, 11 aa, Artificial sequence
  Autoantigenic peptide
GRLFDQRFGEG

SEQ ID NO: 420 - protein, 238 aa, Artificial sequence
  Green Fluorescent protein
MSKGEELFTGIVPVLIELDGDVHGHKFSVRGEGEGDADYGKLEIKFICTTGKLPVPWPTLVTTLGYGIQCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSLQTAISKDRNETRDHMVFLEFFSACGHTHGMDELYK

SEQ ID NO: 421 - protein, 489 aa, Artificial sequence
  Alkaline phosphatase
MKQSTIALALLPLLFTPVTKARTPEMPLQGTAVDGGGGSMHASLEVLENRAAQGDITAPGGARRLTGDQTAALRDSLSDKPAKNIILLIGDGMGDSEITAARNYAEGAGGFFKGIDALPLTGQYTHYALNKKTGKPDYVTDSAASATAWSTGVKTYNGALGVDIHEKDHPTILEMAKAAGLATGNVSTAELQDATPAALVAHVTSRKCYGPSATSEKCPGNALEKGGKGSITEQLLNARADVTLGGGAKTFAETATAGEWQGKTLREQAQARGYQLVSDAASLNSVTEANQQKPLLGLFADGNMPVRWLGPKATYHGNIDKPAVTCTPNPQRNDSVPTLAQMTDKAIELLSKNEKGFFLQVEGASIDKQDHAANPCGQIGETVDLDEAVQRALEFAKKEGNTLVIVTADHAHASQIVAPDTKAPGLTQALNTKDGAVMVMSYGNSEEDSQEHTGSQLRIAAYGPHAANVVGLTDQTDLFYTMKAALGLK

SEQ ID NO: 422 - protein, 309 aa, Artificial sequence
  Peroxidase
MQLTPTFYDNSCPNVSNIVRDTIVNELRSDPRIAASILRLHFHDCFVNGCDASILLDNTTSFRTEKDAFGNANSARGFPVIDRMKAAVESACPRTVSCADLLTIAAQQSVTLAGGPSWRVPLGRRDSLQAFLDLANANLPAPFFTLPQLKDSFRNVGLNRSSDLVALSGGHTFGKNQCRFIMDRLYNFSNTGLPDPTLNTTYLQTLRGLCPLNGNLSALVDFDLRTPTIFDNKYYVNLEEQKGLIQSDQELFSSPNATDTIPLVRSFANSTQTFFNAFVEAMDRMGNITPLTGTQGQIRLNCRVVNSNS

SEQ ID NO: 423 - protein, 286 aa, Artificial sequence
  His and myc tag containing construct
QSVLTQPPSVSGSPGQSITISCTGTSSDIGTYKIVSWYQQHPGKAPKLMIYDVNQRPSGVSDRFSGSKSGNTASLTISGLQAEDEADYYCSSYTSGSTLVFGGGTKLTVLGSSGGGGSGGGGSGGSALQVQLQQSGAEVKKPGESLKISCKGSGYSFTSYWIGWVRQMPGKGLEWMGIIYPGDSDTRYSPSFQGQVTISADKSISTAYLQWSSLKASDTAMYYCARHRAASGSPDACDYWGQGTLVTVSSGSASAPTLFPAAAHHHHHHGAAEQKLISEEDLNGAA

SEQ ID NO: 424 - protein, 238 aa, Artificial sequence
  orange fluorescent protein
MSKGEELFTGVVPILVELDGDVHGHKFSVRGEGEGDADYGKLEIKFICTTGKLPVPWPTLVTTLGYGILCFARYPEHMKMNDFFKSAMPEGYIQERTIFFQDDGKYKTRGEVKFEGDTLVNRIELKGMDFKEDGNILGHKLEYNFNSHNVYIMPDKANNGLKVNFKIRHNIEGGGVQLADHYQTNVPLGDGPVLIPINHYLSYQTAISKDRNETRDHMVFLEFFSACGHTHGMDELYK

SEQ ID NO: 425 - protein, 1019 aa, Artificial sequence
  Beta galactosidase
MADPVVLQRRDWENPGVTQLNRLAAHPPFASWRNSEEARTDRPSQQLRSLNGEWRFAWFPAPEAVPESWLECDLPEADTVVVPSNWQMHGYDAPIYTNVTYPITVNPPFVPTENPTGCYSLTFNVDESWLQEGQTRIIFDGVNSAFHLWCNGRWVGYGQDSRLPSEFDLSAFLRAGENRLAVMVLRWSDGSYLEDQDMWRMSGIFRDVSLLHKPTTQISDFHVATRFNDDFSRAVLEAEVQMCGELRDYLRVTVSLWQGETQVASGTAPFGGEIIDERGGYADRVTLRLNVENPKLWSAEIPNLYRAVVELHTADGTLIEAEACDVGFREVRIENGLLLLNGKPLLIRGVNRHEHHPLHGQVMDEQTMVQDILLMKQNNFNAVRCSHYPNHPLWYTLCDRYGLYVVDEANIETHGMVPMNRLTDDPRWLPAMSERVTRMVQRDRNHPSVIIWSLGNESGHGANHDALYRWIKSVDPSRPVQYEGGGADTTATDIICPMYARVDEDQPFPAVPKWSIKKWLSLPGETRPLILCEYAHAMGNSLGGFAKYWQAFRQYPRLQGGFVWDWVDQSLIKYDENGNPWSAYGGDFGDTPNDRQFCMNGLVFADRTPHPALTEAKHQQQFFQFRLSGQTIEVTSEYLFRHSDNELLHWMVALDGKPLASGEVPLDVAPQGKQLIELPELPQPESAGQLWLTVRVVQPNATAWSEAGHISAWQQWRLAENLSVTLPAASHAIPHLTTSEMDFCIELGNKRWQFNRQSGFLSQMWIGDKKQLLTPLRDQFTRAPLDNDIGVSEATRIDPNAWVERWKAAGHYQAEAALLQCTADTLADAVLITTAHAWQHQGKTLFISRKTYRIDGSGQMAITVDVEVASDTPHPARIGLNCQLAQVAERVNWLGLGPQENYPDRLTAACFDRWDLPLSDMYTPYVFPSENGLRCGTRELNYGPHQWRGDFQFNISRYSQQQLMETSHRHLLHAEEGTWLNIDGFHMGIGGDDSWSPSVSADFQLSAGRYHYQLVWCQK

SEQ ID NO: 426 - protein, 159 aa, Artificial sequence
  Streptavidin
DPSKDSKAQVSAAEAGITGTWYNQLGSTFIVTAGADGALTGTYESAVGNAESRYVLTGRYDSAPATDGSGTALGWTVAWKNNYRNAHSATTWSGQYVGGAEARINTQWLLTSGTTEANAWKSTLVGHDTFTKVKPSAASIDAAKKAGVNNGNPLDAVQQ

SEQ ID NO: 427 - DNA, 717 bp, Artificial sequence
  Green Fluorescent protein coding sequence
atgagtaaaggagaagaacttttcactgggattgtcccagttctcattgagttagacggtgatgtccatggacataaattctctgtcagaggagaaggggaaggcgatgcagattatggaaaacttgaaatcaaattcatttgcactactggaaagctaccagttccatggccaacacttgttactacactgggctacggcatccaatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaattcacataatgtatacattatgccggacaaagccaataatggactcaaagtcaatttcaaaattagacacaatatcgaaggtggtggtgtccaacttgctgatcattaccaaacaaatgttccccttggagacggtcctgtccttataccaatcaatcactacctatccttgcaaacagccatttcaaaagatcgaaatgagacgagagatcatatggtgtttctggaatttttctcagcttgtggacatacacatggcatggatgaactatacaaataa

SEQ ID NO: 428 - DNA, 7151 bp, Artificial sequence
  Alkaline phosphatase coding sequence
gaattccggatgagcattcatcaggcgggcaagaatgtgaataaaggccggataaaacttgtgcttatttttctttacggtctttaaaaaggccgtaatatccagctgaacggtctggttataggtacattgagcaactgactgaaatgcctcaaaatgttctttacgatgccattgggatatatcaacggtggtatatccagtgatttttttctccattttagcttccttagctcctgaaaatctcgataactcaaaaaatacgcccggtagtgatcttatttcattatggtgaaagttggaacctcttacgtgccgatcaacgtctcattttcgccaaaagttggcccagggcttcccggtatcaacagggacaccaggatttatttattctgcgaagtgatcttccgtcacaggtatttattcggcgcaaagtgcgtcgggtgatgctgccaacttactgatttagtgtatgatggtgtttttgaggtgctccagtggcttctgtttctatcagctgtccctcctgttcagctactgacggggtggtgcgtaacggcaaaagcaccgccggacatcagcgctagcggagtgtatactggcttactatgttggcactgatgagggtgtcagtgaagtgcttcatgtggcaggagaaaaaaggctgcaccggtgcgtcagcagaatatgtgatacaggatatattccgcttcctcgctcactgactcgctacgctcggtcgttcgactgcggcgagcggaaatggcttacgaacggggcggagatttcctggaagatgccaggaagatacttaacagggaagtgagagggccgcggcaaagccgtttttccataggctccgcccccctgacaagcatcacgaaatctgacgctcaaatcagtggtggcgaaacccgacaggactataaagataccaggcgtttccccctggcggctccctcgtgcgctctcctgttcctgcctttcggtttaccggtgtcattccgctgttatggccgcgtttgtctcattccacgcctgacactcagttccgggtaggcagttcgctccaagctggactgtatgcacgaaccccccgttcagtccgaccgctgcgccttatccggtaactatcgtcttgagtccaacccggaaagacatgcaaaagcaccactggcagcagccactggtaattgatttagaggagttagtcttgaagtcatgcgccggttaaggctaaactgaaaggacaagttttggtgactgcgctcctccaagccagttacctcggttcaaagagttggtagctcagagaaccttcgaaaaaccgccctgcaaggcggttttttcgttttcagagcaagagattacgcgcagaccaaaacgatctcaagaagatcatcttattaatcagataaaatatttctagatttcagtgcaatttatctcttcaaatgtagcacctgaagtcagccccatacgatataagttgtaattctcatgtttgacagcttatcatcgataagcttttttcgccaggcgcagacttgctgttcttcaggcaatcactcatgtaggtcttacgagcatcccctttcaacgcctgcgccgtcgcctgctgattacaggaggtcatacgctgttgttgtggggttaaagttctctcggcagcgccgacggtggttaaaaaaaccagaccgaaaagcaaggtaaccagtaatgttattttcatagcaccatccctcttcatgttttaaccatgagcgtatgcgcccgtgatctgccattaagtctggttgctaacagcaaaaaaaccacccggcagcgaaaattcactgccgggcgcggttttatttcagccccagagcggctttcatggtgtagaagagatcggtctggtcggtcagtccaacaacattggcggcatgcgggccatacgccgcaatacgcaactgactgccggtatgttcttgtgaatcctcttcggagttcccgtaactcatcaccatcactgcgccatctttggtatttagcgcctgggtgaggcccggagctttggtatccggcgcaacaatctggctggcgtgggcgtgatcagcggtgactatgaccagcgtgttaccctcctttttagcgaattccagcgcccgttgtacggcttcatcgagatcgaccgtctcgccaatttgcccacaaggattcgcagcatgatcctgtttatcgattgacgcaccttcaacttgcaggaaaaagcctttctcatttttactcaacaattcaatggctttgtcggtcatctgcgccagggttggtacactgtcattacgttgcggatttggcgtacaggtgactgcgggcttatcgatattgccatggtacgttgctttcggtcctagccagcgcactggcatattgccgtcagcaaacaggccaagcaggggtttttgctgattcgcttccgtcaccgaattcagtgaggcagcatcgctcaccaactgataaccacgcgcctgtgcctgttcacgcagcgtttttccctgccattcaccagcggttgccgtttcagcaaaggtttttgcgccgccgccaagcgtaacgtcggcacgagcgttaagcagctgttcggtaatcgatccttttccgcctttttccagagcgttacccggacatttttcactggtcgcgctcggaccgtagcatttgcgcgaggtcacatgtgccaccagcgcagcgggcgtggcatcctgcaactctgcggtagaaacgttaccggtcgccagacctgcggcttttgccatttccagaatcgttgggtgatctttttcgtgaatatcgacgcccagcgcgccgttataggttttgacaccggttgaccaggcggttgctgatgcagccgagtcggtgacgtagtccggtttgccggtttttttattcagcgcatagtgagtgtattgcccggtaagcggtaaggcatctatacctttaaaaaagccgcccgcaccttcggcataattacgtgcggcagtaatttccgagtcccccatcccatcgccaatcagcaaaataatattttttgcaggtttatcgctaagagaatcacgcagagcggcagtctgatcacccgttaaacggcgagcaccgccgggtgcagtaatatcgccctgagcagcccggttttccagaacctcgaggctagcatgcatagaaccgccaccaccgtcgacagcggtaccctgcagaggcatttctggtgtccgggcttttgtcacaggggtaaacagtaacggtaagagtgccagtgcaatagtgctttgtttcactttattttctccatgtcgcgtcttatcagggggaattctgtttcctgtgtgaaattgttatccgctcacaattccacacattatacgagccgatgattaattgtcaacagctcatttcagaatatttgccagaaccgttatgatgtcggcgcaaaaaacattatctagaggggaattgttatccgctcacaattcccctatagtgagtcgtattaatttcgcgggatcgagatctcgatcctctacgccggacgcatcgtggccggcatcaccggcgccacaggtgcggttgctggcgcctatatcgccgacatcaccgatggggaagatcgggctcgccacttcgggctcatgagcgcttgtttcggcgtgggtatggtggcaggccccgtggccgggggactgttgggcgccatctccttgcatgcaccattccttgcggcggcggtgctcaacggcctcaacctactactgggctgcttcctaatgcaggagtcgcataagggagagcgtcgagatcccggacaccatcgaatggcgcaaaacctttcgcggtatggcatgatagcgcccggaagagagtcaattcagggtggtgaatgtgaaaccagtaacgttatacgatgtcgcagagtatgccggtgtctcttatcagaccgtttcccgcgtggtgaaccaggccagccacgtttctgcgaaaacgcgggaaaaagtggaagcggcgatggcggagctgaattacattcccaaccgcgtggcacaacaactggcgggcaaacagtcgttgctgattggcgttgccacctccagtctggccctgcacgcgccgtcgcaaattgtcgcggcgattaaatctcgcgccgatcaactgggtgccagcgtggtggtgtcgatggtagaacgaagcggcgtcgaagcctgtaaagcggcggtgcacaatcttctcgcgcaacgcgtcagtgggctgatcattaactatccgctggatgaccaggatgccattgctgtggaagctgcctgcactaatgttccggcgttatttcttgatgtctctgaccagacacccatcaacagtattattttctcccatgaagacggtacgcgactgggcgtggagcatctggtcgcattgggtcaccagcaaatcgcgctgttagcgggcccattaagttctgtctcggcgcgtctgcgtctggctggctggcataaatatctcactcgcaatcaaattcagccgatagcggaacgggaaggcgactggagtgccatgtccggttttcaacaaaccatgcaaatgctgaatgagggcatcgttcccactgcgatgctggttgccaacgatcagatggcgctgggcgcaatgcgcgccattaccgagtccgggctgcgcgttggtgcggatatctcggtagtgggatacgacgataccgaagacagctcatgttatatcccgccgttaaccaccatcaaacaggattttcgcctgctggggcaaaccagcgtggaccgcttgctgcaactctctcagggccaggcggtgaagggcaatcagctgttgcccgtctcactggtgaaaagaaaaaccaccctggcgcccaatacgcaaaccgcctctccccgcgcgttggccgattcattaatgcagctggcacgacaggtttcccgactggaaagcgggcagtgagcgcaacgcaattaatgtaagttagctcactcattaggcaccgggatctcgaccgatgcccttgagagccttcaacccagtcagctccttccggtgggcgcggggcatgactatcgtcgccgcacttatgactgtcttctttatcatgcaactcgtaggacaggtgccggcagcgctctgggtcattttcggcgaggaccgctttcgctggagcgcgacgatgatcggcctgtcgcttgcggtattcggaatcttgcacgccctcgctcaagccttcgtcactggtcccgccaccaaacgtttcggcgagaagcaggccattatcgccggcatggcggccgacgcgctgggctacgtcttgctggcgttcgcgacgcgaggctggatggccttccccattatgattcttctcgcttccggcggcatcgggatgcccgcgttgcaggccatgctgtccaggcaggtagatgacgaccatcagggacagcttcaaggatcgctcgcggctcttaccagcctaacttcgatcactggaccgctgatcgtcacggcgatttatgccgcctcggcgagcacatggaacgggttggcatggattgtaggcgccgccctataccttgtctgcctccccgcgttgcgtcgcggtgcatggagccgggccacctcgacctgaatggaagccggcggcacctcgctaacggattcaccactccaagaattggagccaatcaattcttgcggagaactgtgaatgcgcaaaccaacccttggcagaacatatccatcgcgtccgccatctccagcagccgcacgcggcgcatctcgggcagcgttgggtcctggccacgggtgcgcatgatcgtgctcctgtcgttgaggacccggctaggctggcggggttgccttactggttagcagaatgaatcaccgatacgcgagcgaacgtgaagcgactgctgctgcaaaacgtctgcgacctgagcaacaacatgaatggtcttcggtttccgtgtttcgtaaagtctggaaacgcggaagtcccctacgtgctgctgaagttgcccgcaacagagagtggaaccaaccggtgataccacgatactatgactgagagtcaacgccatgagcggcctcatttcttattctgagttacaacagtccgcaccgctgtccggtagctccttccggtgggcgcggggcatgactatcgtcgccgcacttatgactgtcttctttatcatgcaactcgtaggacaggtgccggcagcgcccaacagtcccccggccacggggcctgccaccatacccacgccgaaacaagcgccctgcaccattatgttccggatctgcatcgcaggatgctgctggctaccctgtggaacacctacatctgtattaacgaagcgctaaccgtttttatcaggctctgggaggcagaataaatgatcatatcgtcaattattacctccacggggagagcctgagcaaactggcctcaggcatttgagaagcacacggtcacactgcttccggtagtcaataaaccggtaaaccagcaatagacataagcggctatttaacgaccctgccctgaaccgacgaccgggtcgaatttgctttcgaatttctgccattcatccgcttattatcacttattcaggcgtagcaccaggcgtttaagggcaccaataactgccttaaaaaaattacgccccgccctgccactcatcgcagtactgttgtaattcattaagcattctgccgacatggaagccatcacagacggcatgatgaacctgaatcgccagcggcatcagcaccttgtcgccttgcgtataatatttgcccatggtgaaaacgggggcgaagaagttgtccatattggccacgtttaaatcaaaactggtgaaactcacccagggattggctgagacgaaaaacatattctcaataaaccctttagggaaataggccaggttttcaccgtaacacgccacatcttgcgaatatatgtgtagaaactgccggaaatcgtcgtggtattcactccagagcgatgaaaacgtttcagtttgctcatggaaaacggtgtaacaagggtgaacactatcccatatcaccagctcaccgtctttcattgccatacg

SEQ ID NO: 429 - DNA, 955 bp, Artificial sequence
  peroxidase coding sequence
aagcttaaccatgcagttaacccctacattctacgacaatagctgtcccaacgtgtccaacatcgttcgcgacacaatcgtcaacgagctcagatccgatcccaggatcgctgcttcaatattacgtctgcacttccatgactgcttcgtgaatggttgcgacgctagcatattactggacaacaccaccagtttccgcactgaaaaggatgcattcgggaacgctaacagcgccaggggctttccagtgatcgatcgcatgaaggctgccgttgagtcagcatgcccacgaacagtcagttgtgcagacctgctgactatagctgcgcaacagagcgtgactcttgcaggcggaccgtcctggagagtgccgctcggtcgacgtgactccctacaggcattcctagatctggccaacgccaacttgcctgctccattcttcaccctgccccagctgaaggatagctttagaaacgtgggtctgaatcgctcgagtgaccttgtggctctgtccggaggacacacatttggaaagaaccagtgtaggttcatcatggataggctctacaatttcagcaacactgggttacctgaccccacgctgaacactacgtatctccagacactgagaggcttgtgcccactgaatggcaacctcagtgcactagtggactttgatctgcggaccccaaccatcttcgataacaagtactatgtgaatctagaggagcagaaaggcctgatacagagtgatcaagaactgtttagcagtccaaacgccactgacaccatcccactggtgagaagttttgctaactctactcaaaccttctttaacgccttcgtggaagccatggaccgtatgggtaacattacccctctgacgggtacccaaggccagattcgtctgaactgcagagtggtcaacagcaactcttaataaggatccgaattc

SEQ ID NO: 430 - DNA, 861 bp, Artificial sequence
  His and myc tag containing construct
cagtctgtgttgacgcagccgccctcagtgtctgggtctcctggacagtcgatcaccatctcctgcactggaaccagcagtgatattgggacttataaaattgtctcctggtaccaacagcaccctggcaaagcccccaaactcatgatttatgacgtcaatcagcggccctcaggggtttctgatcgcttctctggctccaagtctggcaacacggcctccctgacaatctctgggctccaggctgaggacgaggctgattattactgcagctcatatacaagcggcagcactctggtattcggcggggggaccaagctgaccgtcctaggctcgagtggtggaggcggttcaggcggaggtggctctggcggtagtgcacttcaggtacagctgcagcagtcaggagcagaggtgaaaaagcccggggagtctctgaagatctcctgtaagggttctggatacagctttaccagctactggatcggctgggtgcgccagatgcccgggaaaggcctggagtggatggggatcatctatcctggtgactctgataccagatacagcccgtccttccaaggccaggtcaccatctcagccgacaagtccatcagcaccgcctacctgcagtggagcagcctgaaggcctcggacaccgccatgtattactgtgcgagacatcgggccgctagtgggagcccggacgcgtgtgactactggggccagggaaccctggtcaccgtctcctcagggagtgcatccgccccaacccttttccccgcggccgcacatcatcatcaccatcacggggccgcagaacaaaaactcatctcagaagaggatctgaatggggccgcatag

SEQ ID NO: 431 - DNA, 717 bp, Artificial sequence
  orange fluorescent protein coding sequence
atgagtaaaggagaagaacttttcactggagttgtcccaattcttgttgaattagatggtgatgtccatggacataaattctctgtcagaggagaaggggaaggcgatgcagattatggaaaacttgaaatcaaattcatttgcactactggaaagctaccagttccatggccaacacttgttactacactgggctatggcatcctatgtttcgcaagatacccagaacacatgaaaatgaatgacttcttcaagagtgccatgcctgagggttacattcaagaaagaaccatctttttccaagatgatggaaaatacaagacacgtggtgaagtcaagtttgaaggtgatactcttgttaacagaattgagctcaaaggtatggactttaaagaagatggcaatatccttggacacaagttggagtacaattttaactcacataatgtatacattatgccggacaaagccaataatggactcaaagtcaatttcaaaattagacacaatatcgaaggtggtggtgtccaactcgctgatcattaccaaacaaatgttccccttggagacggtcctgtccttataccaatcaatcactacctatcctatcaaacagccatttcaaaagatcgaaatgagacgagagatcatatggtgtttctggaatttttctcagcttgtggacatacacatggcatggatgaactatacaaataa

SEQ ID NO: 432 - DNA, 8388 bp, Artificial sequence
  Beta galactosidase coding sequence
acgttaagggattttggtcatggacggccagcaggtaggccgacaggctcatgccggccgccgccgccttttcctcaatcgctcttcgttcgtctggaaggcagtacaccttgataggtgggctgcccttcctggttggcttggtttcatcagccatccgcttgccctcatctgttacgccggcggtagccggccagcctcgcagagcaggattcccgttgagcaccgccaggtgcgaataagggacagtgaagaaggaacacccgctcgcgggtgggcctacttcacctatcctgcccggctgacgccgttggatacaccaaggaaagtctacacgaaccctttggcaaaatcctgtatatcgtgcgaaaaaggatggatataccgaaaaaatcgctataatgaccccgaagcagggttatgcagcggaaaagcgctgcttccctgctgttttgtggaatatctaccgactggaaacaggcaaatgcaggaaattactgaactgaggggacaggcgagagacgatgccaaagagctacaccgacgagctggccgagtgggttgaatcccgcgcggccaagaagcgccggcgtgatgaggctgcggttgcgttcctggcggtgagggcggatgtcgatatgcgtaaggagaaaataccgcatcaggcgcatatttgaatgtatttagaaaaataaacaaaaagagtttgtagaaacgcaaaaaggccatccgtcaggatggccttctgcttaatttgatgcctggcagtttatggcgggcgtcctgcccgccaccctccgggccgttgcttcgcaacgttcaaatccgctcccggcggatttgtcctactcaggagagcgttcaccgacaaacaacagataaaacgaaaggcccagtctttcgactgagcctttcgttttatttgatgcctggcagttccctactctcgcatggggagaccccacactaccatcggcgctacggcgtttcacttctgagttcggcatggggtcaggtgggaccaccgcgctactgccgccaggcaaattctgttttatcagaccgcttctgcgttctgatttaatctgtatcaggctgaaaattaaggaatcccccaggacccaacgctgcccgagtttgtcagaaagcagaccaaacagcggttggaataatagcgagaacagagaaatagcggcaaaaataatacccgtatcacttttgctgatatggttgatgtcatgtagccaaatcgggaaaaacgggaagtaggctcccatgataaaaaagtaaaagaaaaagaataaaccgaacatccaaaagtttgtgttttttaaatagtacataatggatttccttacgcgaaatacgggcagacatggcctgcccggttattattatttttgacaccagaccaactggtaatggtagcgaccggcgctcagctggaaatccgccgatactgacgggctccaggagtcgtcgccaccaatccccatatggaaaccgtcgatattcagccatgtgccttcttccgcgtgcagcagatggcgatggctggtttccatcagttgctgttgactgtagcggctgatgttgaactggaagtcgccgcgccactggtgtgggccataattcaattcgcgcgtcccgcagcgcagaccgttttcgctcgggaagacgtacggggtatacatgtctgacaatggcagatcccagcggtcaaaacaggcggcagtaaggcggtcgggatagttttcttgcggccctaatccgagccagtttacccgctctgctacctgcgccagctggcagttcaggccaatccgcgccggatgcggtgtatcgctcgccacttcaacatcaacggtaatcgccatttgaccactaccatcaatccggtaggttttccggctgataaataaggttttcccctgatgctgccacgcgtgagcggtcgtaatcagcaccgcatcagcaagtgtatctgccgtgcactgcaacaacgctgcttcggcctggtaatggcccgccgccttccagcgttcgacccaggcgttagggtcaatgcgggtcgcttcacttacgccaatgtcgttatccagcggtgcacgggtgaactgatcgcgcagcggcgtcagcagttgttttttatcgccaatccacatctgtgaaagaaagcctgactggcggttaaattgccaacgcttattacccagctcgatgcaaaaatccatttcgctggtggtcagatgcgggatggcgtgggacgcggcggggagcgtcacactgaggttttccgccagacgccactgctgccaggcgctgatgtgcccggcttctgaccatgcggtcgcgttcggttgcactacgcgtactgtgagccagagttgcccggcgctctccggctgcggtagttcaggcagttcaatcaactgtttaccttgtggagcgacatccagaggcacttcaccgcttgccagcggcttaccatccagcgccaccatccagtgcaggagctcgttatcgctatgacggaacaggtattcgctggtcacttcgatggtttgcccggataaacggaactggaaaaactgctgctggtgttttgcttccgtcagcgctggatgcggcgtgcggtcggcaaagaccagaccgttcatacagaactggcgatcgttcggcgtatcgccaaaatcaccgccgtaagccgaccacgggttgccgttttcatcatatttaatcagcgactgatccacccagtcccagacgaagccgccctgtaaacggggatactgacgaaacgcctgccagtatttagcgaaaccgccaagactgttacccatcgcgtgggcgtattcgcaaaggatcagcgggcgcgtctctccaggtagcgaaagccattttttgatggaccatttcggcacagccgggaagggctggtcttcatccacgcgcgcgtacatcgggcaaataatatcggtggccgtggtgtcggctccgccgccttcatactgcaccgggcgggaaggatcgacagatttgatccagcgatacagcgcgtcgtgattagcgccgtggcctgattcattccccagcgaccagatgatcacactcgggtgattacgatcgcgctgcaccattcgcgttacgcgttcgctcatcgccggtagccagcgcggatcatcggtcagacgattcattggcaccatgccgtgggtttcaatattggcttcatccaccacatacaggccgtagcggtcgcacagcgtgtaccacagcggatggttcggataatgcgaacagcgcacggcgttaaagttgttctgcttcatcagcaggatatcctgcaccatcgtctgctcatccatgacctgaccatgcagaggatgatgctcgtgacggttaacgcctcgaatcagcaacggcttgccgttcagcagcagcagaccattttcaatccgcacctcgcggaaaccgacatcgcaggcttctgcttcaatcagcgtgccgtcggcggtgtgcagttcaaccaccgcacgatagagattcgggatttcggcgctccacagtttcgggttttcgacgttcagacgtagtgtgacgcgatcggcataaccaccacgctcatcgataatttcaccgccgaaaggcgcggtgccgctggcgacctgcgtttcaccctgccataaagaaactgttacccgtaggtagtcacgcaactcgccgcacatctgaacttcagcctccagtacagcgcggctgaaatcatcattaaagcgagtggcaacatggaaatcgctgatttgtgtagtcggtttatgcagcaacgagacgtcacggaaaatgccgctcatccgccacatatcctgatcttccagataactgccgtcactccaacgcagcaccatcaccgcgaggcggttttctccggcgcgtaaaaatgcgctcaggtcaaattcagacggcaaacgactgtcctggccgtaaccgacccagcgcccgttgcaccacagatgaaacgccgagttaacgccatcaaaaataattcgcgtctggccttcctgtagccagctttcatcaacattaaatgtgagcgagtaacaacccgtcggattctccgtgggaacaaacggcggattgaccgtaatgggataggttacgttggtgtagatgggcgcatcgtaaccgtgcatctgccagtttgaggggacgacgacagtatcggcctcaggaagatcgcactccagccagctttccggcaccgcttctggtgccggaaaccaggcaaagcgccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacgggatcagccatttttttctccttacttacttaggatccccgggtaccgagctcgaattggggatcttgaagttcctattccgaagttcctattctctagaaagtataggaacttcagagcgcttttgaagctaattcgagctcggtacccggggatcccccgggctcgactgcattaatgaatcggccaacgcgcggggagaggcggtttgcgtattgggcgctcttccgctcgaattgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtactcgagcagaaagtcaaaagcctccgaccggaggcttttgacttgagggggatcgatcccttatggctctgcacccggctccatcaccaacaggtcgcgcacgcgcttcactcggttgcggatcgacactgccagcccaacaaagccggttgccgccgccgccaggatcgcgccgatgatgccggccacaccggccatcgcccaccaggtcgccgccttccggttccattcctgctggtactgcttcgcaatgctggacctcggctcaccataggctgaccgctcgatggcgtatgccgcttctccccttggcgtaaaacccagcgccgcaggcggcattgccatgctgcccgccgctttcccgaccacgacgcgcgcaccaggcttgcggtccagaccttcggccacggcgagctgcgcaaggacataatcagccgccgacttggctccacgcgcctcgatcagctcttgcactcgcgcgaaatccttggcctccacggccgccatgaatcgcgcacgcggcgaaggctccgcagggccggcgtcgtgatcgccgccgagaatgcccttcaccaagttcgacgacacgaaaatcatgctgacggctatcaccatcatgcagacggatcgcacgaacccgcagaactcacccccgaacacgagcacggcacccgcgaccactatgccaagaatgcccaaggtaaaaattgccggccccgccatgaagtccgtgaatgccccgacggccgaagtgaagggcaggccgccacccaggccgccgccctcactgcccggcacctggtcgctgaatgtcgatgccagcacctgcggcacgtcaatgcttccgggcgtcgcgctcgggctgatcgcccatcccgttactgccccgatcccggcaatggcaaggactgccagcgccgcgatgaggaagcgggtgccccgcttcttcatcttcgcgcctcgggcctccaggccgcctacctgggcgaaaacatcggtgtttgtggcattcatacggactcctgttgggccagctcgcgcacgggctggcgggtcagcttggcttgaagatcgccacgcattgcggcgatctgcttctcggcatccttgcgcttctgcacgccttcctgctggatgcgaataacgtcctcgacggtcttgatgagcgtcgtctgaacctgcttgagcgtgtccacgtcgatcaccaggcgttggttctccttcgccgtctcgacggacgtgcgatgcagcagggccgcattgcgcttcatcaggtcgttggtggtgtcgtcgatggccgtggccagttcgacggcgttcttctgctcgttgaggctcaaggccagcatgaattgccgcttccacgccggcacggtgatttcgcggatggtgtggaatttatcgaccagcatctggttgttggcctggatcatgcggatggtcggcaggctctgcatggccgaatgttgcaaggcgatcaggtcgccgatgcgcttgtccaggttggcaaccatcgcatcgaggtcggccagctcctgcacgcggccagggtcgttcccgacattgccgcgcagaccctcggcctgctcgcgcagctcggcaaggcggaccttgccggccgcgatgtggacgccaagaaggcggtgttcctcgcgcacggctgcgaacatttcgtcgagcgaggcattgcgctgcgcgatgccttgctgggtggtctgcacttcgctgaccaggtgttcgatctgctcgcgggtcgtgtcgaagcgcgccatgaagcccgtcgaacggacgcggaagcggtcgatcagcgggccaatcaggggcaggcgggaacggttgtcggacaaagggccgacgttcagggaacgggccttggcgacaacctgggtcagtttctcgcctgcttcgtccaggtcgctgttgcgcacctggtccagcaggctatcggcgtagcgggacgtgtgctcggccacgtcgcggccgaactcggcaacggtctgcggactgccgacctcgatccgctgcgcgaccgcatggacttccggcacgtcgctttcctgcaagcccagctcgcgcagggttgccggggtcatgtcgaaggcgacgataggggccttggcgtcgtgcgtcgttttcagtgcgttcatagggttctcccgccgtgttattggttgatgccttccaggctctgcgaaaggctccgcatgagcgcctggtgagctttggccgcctcggcgaccattgccggattcatgttcttggtggtgatgagcgcgagggtgtgctgacgccagacgggcaccaggacggatgccgtttcagagaagcggtccagcatgtccacggcctgcgcccgcgtgagcttcatctgagtgacgctcatttcatgggacgccatgagggttgccaggttggcgagcttgcgcgcgaagcgttcgcgcggcttgtcgaactcgatcacgccggccttggccgcgccggcctcggggttctcgtccaggaactcgcgcccggcttgaatgtaggctctgagccggtctacctcggcctcatgcgtattgagcatgtcatccaaggcgcgcaacgtgtcccgcacgcgctgcgctacgccctcggcttcgtccagcaactggtcgagcgtcttgcgggcgacctgatacctcacctggcgttcaacctcacggccaagcatcttctcgaaccaggtaggcttttccgcgatcttgcgggggtccgcgtcggccagcttcgccacgatctggctgattttgtcggccagcgcggcaactgcgccgtgctccatcagattcgacagctcgttgagggaatccgccccgtcgatgccggccccgtactcgccaatcgtcgccggcgacgcgaagagggcgggcaaaacctcccccttcaatcgcgccatgttcacgctttgttcttccatggtatatctccttcttaaagttaaacaaaattattcggaacccagcatgatattccggaaataccaactaagtcaacggctgatggccaattcggcttcctcgctcactgactcgctgcgctcggtcgttcggctgcggcgagcggtatcagctcactcaaaggcggtaatacggttatccacagaatcaggggataacgcaggaaagaacatgtgagatcgatcagcagttcaacctgttgatagtacgtactaagctctcatgtttcacgtactaagctctcatgtttaacgtactaagctctcatgtttaacgaactaaaccctcatggctaacgtactaagctctcatggctaacgtactaagctctcatgtttcacgtactaagctctcatgtttgaacaataaaattaatataaatcagcaacttaaatagcctctaaggttttaagttttataagaaaaaaaagaatatataaggcttttaaagctagcttttaaggtttaacggttgtggacaacaagccagggatgtaacgcactgagaagcccttagagcctctcaaagcaattttcagtgacacaggaacacttaacggctgacatgacgctcagtggaacgaaaactc

SEQ ID NO: 433 - DNA, 483 bp, Artificial sequence
  Streptavidin coding sequence
gacccgagcaaagattctaaagcacaagtatctgctgcagaagcaggaattacaggcacatggtataatcagctgggatctacatttattgttacagccggcgcagatggagctcttacaggaacatatgaatctgctgttggaaatgcagaatctagatacgtgcttacaggaagatatgattctgcacctgcaacagatggatccggaacagcacttggatggacagttgcatggaaaaacaattatagaaacgcacatagcgctacaacatggtctggccaatatgtgggaggtgcagaagcaagaattaacacacaatggcttttaacatctggaacaacagaagcaaatgcatggaaaagtactcttgttggacatgatacatttacaaaagttaaacctagcgcagcatctatcgatgcagcgaaaaaagcaggagttaacaatggcaatcctttagatgcagttcaacaataatga

SEQ ID NO: 434 - protein, 216 aa, Artificial sequence
  Pseudomonas exotoxin
AEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTGLTLAAPEAAGEVERLIGHPLPLRLDAITGPEEEGGRLETILGWPLAERTVVIPSAIPTDPRNVGGDLAPSSIPDQEQAISALPDYASQPGKPSREDLK

SEQ ID NO: 435 - protein, 536 aa, Artificial sequence
  Diphtheria toxin
MGADDVVDSSKSFVMENFSSYHGTKPGYVDSIQKGIQKPKSGTQGNYDDDWKGFYSTDNKYDAAGYSVDNENPLSGKAGGVVKVTYPGLTKVLALKVDNAETIKKELGLSLTEPLMEQVGTEEFIKRFGDGASRVVLSLPFAEGSSSVEYINNWEQAKALSVELEINFETRGKRGQDAMYEYMAQACAGNRVRRSVGSSLSCINLDWDVIRDKTKTKIESLKEHGPIKNKMSESPNKTVSEEKAKQYLEEFHQTALEHPELSELKTVTGTNPVFAGANYAAWAVNVAQVIDSETADNLEKTTAALSILPGIGSVMGIADGAVHHNTEEIVAQSIALSSLMVAQAIPLVGELVDIGFAAYNFVESIINLFQVVHNSYNRPAYSPGHKTQPFLHDGYAVSWNTVEDSIIRTGFQGESGHDIKITAENTPLPIAGVLLPTIPGKLDVNKSKTHISVNGRKIRMRCRAIDGDVTFCRPKSPVYVGNGVHANLHVAFHRSSSEKIHSNEISSDSIGVLGYQKTVDHTKVNSKLSLFFEIKS

SEQ ID NO: 436 - protein, 127 aa, Artificial sequence
  Interleukin 2
TKKTQLQLEHLLLDLQMILNGINNYKNPKLTRMLTFKFYMPKKATELKHLQCLEEELKPLEEVLNLAQSKNFHLRPRDLISNINVIVLELKGSETTFMCEYADETATIVEFLNRWITFCQSIISTLT

SEQ ID NO: 437 - protein, 207 aa, Artificial sequence
  CD3
MQSGTHWRVLGLCLLSVGVWGQDGNEEMGGITQTPYKVSISGTTVILTCPQYPGSEILWQHNDKNIGGDEDDKNIGSDEDHLSLKEFSELEQSGYYVCYPRGSKPEDANFYLYLRARVCENCMEMDVMSVATIVIVDICITGGLLLLVYYWSKNRKAKAKPVTRGAGAGGRQRGQNKERPPPVPNPDYEPIRKGQRDLYSGLNQRRI

SEQ ID NO: 438 - protein, 290 aa, Artificial sequence
  CD16
MGGGAGERLFTSSCLVGLVPLGLRISLVTCPLQCGIMWQLLLPTALLLLVSAGMRTEDLPKAVVFLEPQWYRVLEKDSVTLKCQGAYSPEDNSTQWFHNESLISSQASSYFIDAATVDDSGEYRCQTNLSTLSDPVQLEVHIGWLLLQAPRWVFKEEDPIHLRCHSWKNTALHKVTYLQNGKGRKYFHHNSDFYIPKATLKDSGSYFCRGLFGSKNVSSETVNITITQGLAVSTISSFFPPGYQVSFCLVMVLLFAVDTGLYFSVKTNIRSSTRDWKDHKFKWRKDPQDK

SEQ ID NO: 439 - protein, 153 aa, Artificial sequence [sequence omitted]

SEQ ID NO: 440 - protein, 365 aa, Artificial sequence [sequence omitted]

SEQ ID NO: 441 - protein, 178 aa, Artificial sequence
  interleukin 10
MHSSALLCCLVLLTGVRASPGQGTQSENSCTHFPGNLPNMLRDLRDAFSRVKTFFQMKDQLDNLLLKESLLEDFKGYLGCQALSEMIQFYLEEVMPQAENQDPDIKAHVNSLGENLKTLRLRLRRCHRFLPCENKSKAVEQVKNAFNKLQEKGIYKAMSEFDIFINYIEAYMTMKIRN

SEQ ID NO: 442 - protein, 576 aa, Artificial sequence
  Ricin toxin
MKPGGNTIVIWMYAVATWLCFGSTSGWSFTLEDNNIFPKQYPIINFTTAGATVQSYTNFIRAVRGRLTTGADVRHEIPVLPNRVGLPINQRFILVELSNHAELSVTLALDVTNAYVVGYRAGNSAYFFHPDNQEDAEAITHLFTDVQNRYTFAFGGNYDRLEQLAGNLRENIELGNGPLEEAISALYYYSTGGTQLPTLARSFIICIQMISEAARFQYIEGEMRTRIRYNRRSAPDPSVITLENSWGRLSTAIQESNQGAFASPIQLQRRNGSKFSVYDVSILIPIIALMVYRCAPPPSSQFSLLIRPVVPNFNADVCMDPEPIVRIVGRNGLCVDVRDGRFHNGNAIQLWPCKSNTDANQLWTLKRDNTIRSNGKCLTTYGYSPGVYVMIYDCNTAATDATRWQIWDNGTIINPRSSLVLAATSGNSGTTLTVQTNIYAVSQGWLPTNNTQPFVTTIVGLYGLCLQANSGQVWIEDCSSEKAEQQWALYADGSIRPQQNRDNCLTSDSNIRETVVKILSCGPASSGQRWMFKNDGTILNLYSGLVLDVRASDPSLKQIILYPLHGDPNQIWLPLF

SEQ ID NO: 443 - DNA, 651 bp, Artificial sequence
  Pseudomonas exotoxin coding sequence
gcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggaccttgacgcgatctggcgcggtttctatatcgccggcgatccggcgctggcctacggctacgcccaggaccaggaacccgacgcgcgcggccggatccgcaacggtgccctgctgcgggtctatgtgccgcgctcgagtctgccgggcttctaccgcaccggcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccacgcaacgtcggcggcgacctcgccccgtccagcatccccgaccaggaacaggcgatcagcgccctgccggactacgccagccagcccggcaaaccgtcgcgcgaggacctgaagtaa

SEQ ID NO: 444 - DNA, 1611 bp, Artificial sequence
  Diphtheria toxin coding sequence
atgggcgctgatgatgttgttgattcttctaaatcttttgtgatggaaaacttttcttcgtaccacgggactaaacctggttatgtagattccattcaaaaaggtatacaaaagccaaaatctggtacacaaggaaattatgacgatgattggaaagggttttatagtaccgacaataaatacgacgctgcgggatactctgtagataatgaaaacccgctctctggaaaagctggaggcgtggtcaaagtgacgtatccaggactgacgaaggttctcgcactaaaagtggataatgccgaaactattaagaaagagttaggtttaagtctcactgaaccgttgatggagcaagtcggaacggaagagtttatcaaaaggttcggtgatggtgcttcgcgtgtagtgctcagccttcccttcgctgaggggagttctagcgttgaatatattaataactgggaacaggcgaaagcgttaagcgtagaacttgagattaattttgaaacccgtggaaaacgtggccaagatgcgatgtatgagtatatggctcaagcctgtgcaggaaatcgtgtcaggcgatcagtaggtagctcattgtcatgcataaatcttgattgggatgtcataagggataaaactaagacaaagatagagtctttgaaagagcatggccctatcaaaaataaaatgagcgaaagtcccaataaaacagtatctgaggaaaaagctaaacaatacctagaagaatttcatcaaacggcattagagcatcctgaattgtcagaacttaaaaccgttactgggaccaatcctgtattcgctggggctaactatgcggcgtgggcagtaaacgttgcgcaagttatcgatagcgaaacagctgataatttggaaaagacaactgctgctctttcgatacttcctggtatcggtagcgtaatgggcattgcagacggtgccgttcaccacaatacagaagagatagtggcacaatcaatagctttatcatctttaatggttgctcaagctattccattggtaggagagctagttgatattggtttcgctgcatataattttgtagagagtattatcaatttatttcaagtagttcataattcgtataatcgtcccgcgtattctccggggcataaaacgcaaccatttcttcatgacgggtatgctgtcagttggaacactgttgaagattcgataatccgaactggttttcaaggggagagtgggcacgacataaaaattactgctgaaaataccccgcttccaatcgcgggtgtcctactaccgactattcctggaaagctggacgttaataagtccaagactcatatttccgtaaatggtcggaaaataaggatgcgttgcagagctatagacggtgatgtaactttttgtcgccctaaatctcctgtttatgttggtaatggtgtgcatgcgaatcttcacgtggcatttcacagaagcagctcggagaaaattcattctaatgaaatttcatcggattccataggcgttcttgggtaccagaaaacagtagatcacaccaaggttaattctaagctatcgctattttttgaaatcaaaagctga

SEQ ID NO: 445 - DNA, 698 bp, Artificial sequence
  Interleukin 2 coding sequence
gggngggggacaaagaaaacacagctacaactggagcatttacttctggatttacagatgattttgaatggaattaataattacaagaatcccaaactcaccaggatgctcacatttaagttttacatgcccaagaaggccacagaactgaaacatcttcagtgtctagaagaagaactcaaacctctggaggaagtgctaaatttagctcaaagcaaaaactttcacttaagacccagggacttaatcagcaatatcaacgtaatagttctggaactaaagggatctgaaacaacattcatgtgtgaatatgctgatgagacagcaaccattgtagaatttctgaacagatggattaccttttgtcaaagcatcatctcaacactgacttgataattaagtgcttcccacttaaaacatatcaggccttctatttatttaaatatttaaattttatatttattgttgaatgtatggtttgctacctattgtaactattattcttaatcttaaaactataaatatggatcttttatgattctttttgtaagccctaggggctctaaaatggtttcacttatttatcccaaaatatttattattatgttgaatgttaaatatagtatctatgtagattggttagtaaaactatttaataaatttgataaatataaacaaaaaaaaaaaaaccccccccc

SEQ ID NO: 446 - DNA, 1311 bp, Artificial sequence
  CD3 coding sequence
gtaagtctgctggcctccgccatcttagtaaagtaacagtcccatgaaacaaagatgcagtcgggcactcactggagagttctgggcctctgcctcttatcagttggcgtttgggggcaagatggtaatgaagaaatgggtggtattacacagacaccatataaagtctccatctctggaaccacagtaatattgacatgccctcagtatcctggatctgaaatactatggcaacacaatgataaaaacataggcggtgatgaggatgataaaaacataggcagtgatgaggatcacctgtcactgaaggaattttcagaattggagcaaagtggttattatgtctgctaccccagaggaagcaaaccagaagatgcgaacttttatctctacctgagggcaagagtgtgtgagaactgcatggagatggatgtgatgtcggtggccacaattgtcatagtggacatctgcatcactgggggcttgctgctgctggtttactactggagcaagaatagaaaggccaaggccaagcctgtgacacgaggagcgggtgctggcggcaggcaaaggggacaaaacaaggagaggccaccacctgttcccaacccagactatgagcccatccggaaaggccagcgggacctgtattctggcctgaatcagagacgcatctgaccctctggagaacactgcctcccgctggcccaggtctcctctccagtccccctgcgactccctgtttcctgggctagtcttggaccccacgagagagaatcgttcctcagcctcatggtgaactcgcgccctccagcctgatcccccgctccctcctccctgccttctctgctggtacccagtcctaaaatattgctgcttcctcttcctttgaagcatcatcagtagtcacaccctcacagctggcctgccctcttgccaggatatttatttgtgctattcactcccttccctttggatgtaacttctccgttcagttccctccttttcttgcatgtaagttgtcccccatcccaaagtattccatctacttttctatcgccgtccccttttgcagccctctctggggatggactgggtaaatgttgacagaggccctgccccgttcacagatcctggccctgagccagccctgtgctcctccctcccccaacactccctaccaaccccctaatcccctactccctccaaccccccctcccactgtaggccactggatggtcatttggcatctccgtatatgtgctctggctcctcagctgagagagaaaaaaataaactgtatttggctgc

SEQ ID NO: 447 - DNA, 2406 bp, Artificial sequence
  CD16 coding sequence
gattctgtgtgtgtcctcagatgctcagccacagacctttgagggagtaaagggggcagacccacccaccttgcctccaggctctttccttcctggtcctgttctatggtggggctcccttgccagacttcagactgagaagtcagatgaagtttcaagaaaaggaaattggtgggtgacagagatgggtggaggggctggggaaaggctgtttacttcctcctgtctagtcggtttggtccctttagggctccggatatctttggtgacttgtccactccagtgtggcatcatgtggcagctgctcctcccaactgctctgctacttctagtttcagctggcatgcggactgaagatctcccaaaggctgtggtgttcctggagcctcaatggtacagggtgctcgagaaggacagtgtgactctgaagtgccagggagcctactcccctgaggacaattccacacagtggtttcacaatgagagcctcatctcaagccaggcctcgagctacttcattgacgctgccacagtcgacgacagtggagagtacaggtgccagacaaacctctccaccctcagtgacccggtgcagctagaagtccatatcggctggctgttgctccaggcccctcggtgggtgttcaaggaggaagaccctattcacctgaggtgtcacagctggaagaacactgctctgcataaggtcacatatttacagaatggcaaaggcaggaagtattttcatcataattctgacttctacattccaaaagccacactcaaagacagcggctcctacttctgcagggggctttttgggagtaaaaatgtgtcttcagagactgtgaacatcaccatcactcaaggtttggcagtgtcaaccatctcatcattctttccacctgggtaccaagtctctttctgcttggtgatggtactcctttttgcagtggacacaggactatatttctctgtgaagacaaacattcgaagctcaacaagagactggaaggaccataaatttaaatggagaaaggaccctcaagacaaatgacccccatcccatgggggtaataagagcagtagcagcagcatctctgaacatttctctggatttgcaaccccatcatcctcaggcctctctacaagcagcaggaaacatagaactcagagccagatcccttatccaactctcgacttttccttggtctccagtggaagggaaaagcccatgatcttcaagcagggaagccccagtgagtagctgcattcctagaaattgaagtttcagagctacacaaacactttttctgtcccaaccgttccctcacagcaaagcaacaatacaggctagggatggtaatcctttaaacatacaaaaattgctcgtgttataaattacccagtttagaggggaaaaaaaaacaattattcctaaataaatggataagtagaattaatggttgaggcaggaccatacagagtgtgggaactgctggggatctagggaattcagtgggaccaatgaaagcatggctgagaaatagcaggtagtccaggatagtctaagggaggtgttcccatctgagcccagagataagggtgtcttcctagaacattagccgtagtggaattaacaggaaatcatgagggtgacgtagaattgagtcttccaggggactctatcagaactggaccatctccaagtatataacgatgagtcctcttaatgctaggagtagaaaatggtcctaggaaggggactgaggattgcggtggggggtggggtggaaaagaaagtacagaacaaaccctgtgtcactgtcccaagttgctaagtgaacagaactatctcagcatcagaatgagaaagcctgagaagaaagaaccaaccacaagcacacaggaaggaaagcgcaggaggtgaaaatgctttcttggccagggtagtaagaattagaggttaatgcagggactgtaaaaccaccttttctgcttcaatatctaattcctgtgtagctttgttcattgcatttattaaacaaatgttgtataaccaatactaaatgtactactgagcttcgctgagttaagttatgaaactttcaaatccttcatcatgtcagttccaatgaggtggggatggagaagacaattgttgcttatgaaagaaagctttagctgtctctgttttgtaagctttaagcgcaacatttcttggttccaataaagcattttacaagatcttgcatgctactcttagatagaagatgggaaaaccatggtaataaaatatgaatgataaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaaa

SEQ ID NO: 448 - DNA, 921 bp, Artificial sequence
  interleukin 4 coding sequence
ttctatgcaaagcaaaaagccagcagcagccccaagctgataagattaatctaaagagcaaattatggtgtaatttcctatgctgaaactttgtagttaattttttaaaaaggtttcattttcctattggtctgatttcacaggaacattttacctgtttgtgaggcattttttctcctggaagagaggtgctgattggccccaagtgactgacaatctggtgtaacgaaaatttccaatgtaaactcattttccctcggtttcagcaattttaaatctatatatagagatatctttgtcagcattgcatcgttagcttctcctgataaactaattgcctcacattgtcactgcaaatcgacacctattaatgggtctcacctcccaactgcttccccctctgttcttcctgctagcatgtgccggcaactttgtccacggacacaagtgcgatatcaccttacaggagatcatcaaaactttgaacagcctcacagagcagaagactctgtgcaccgagttgaccgtaacagacatctttgctgcctccaagaacacaactgagaaggaaaccttctgcagggctgcgactgtgctccggcagttctacagccaccatgagaaggacactcgctgcctgggtgcgactgcacagcagttccacaggcacaagcagctgatccgattcctgaaacggctcgacaggaacctctggggcctggcgggcttgaattcctgtcctgtgaaggaagccaaccagagtacgttggaaaacttcttggaaaggctaaagacgatcatgagagagaaatattcaaagtgttcgagctgaatattttaatttatgagtttttgatagctttattttttaagtatttatatatttataactcatcataaaataaagtatatatagaatct

SEQ ID NO: 449 - DNA, 4000 bp, Artificial sequence
  HLA-A2  coding sequence
aagcttactctctggcaccaaactccatgggatgatttttccttcctagaagagtccaggtggacaggtaaggagtgggagtcagggagtccagttccagggacagagattacgggataaaaagtgaaaggagagggacggggcccatgccgagggtttctcccttgtttctcagacagctcttgggccaagactcagggagacattgagacagagcgcttggcacagaagcagaggggtcagggcgaagtccagggccccaggcgttggctctcagggtctcaggccccgaaggcggtgtatggattggggagtcccagccttggggattccccaactccgcagtttcttttctccctctcccaacctatgtagggtccttcttcctggatactcacgacgcggacccagttctcactcccattgggtgtcgggtttccagagaagccaatcagtgtcgtcgcggtcgcggttctaaagtccgcacgcacccaccgggactcagattctccccagacgccgaggatggccgtcatggcgccccgaaccctcgtcctgctactctcgggggctctggccctgacccagacctgggcgggtgagtgcggggtcgggagggaaacggcctctgtggggagaagcaacgggccgcctggcgggggcgcaggacccgggaagccgcgccgggaggagggtcgggcgggtctcagccactcctcgtccccaggctctcactccatgaggtatttcttcacatccgtgtcccggcccggccgcggggagccccgcttcatcgcagtgggctacgtggacgacacgcagttcgtgcggttcgacagcgacgccgcgagccagaggatggagccgcgggcgccgtggatagagcaggagggtccggagtattgggacggggagacacggaaagtgaaggcccactcacagactcaccgagtggacctggggaccctgcgcggctactacaaccagagcgaggccggtgagtgaccccggcccggggcgcaggtcacgacctctcatcccccacggacgggccaggtcgcccacagtctccgggtccgagatccgccccgaagccgcgggaccccgagacccttgccccgggagaggcccaggcgcctttacccggtttcattttcagtttaggccaaaaatccccccaggttggtcggggcggggcggggctcgggggaccgggctgaccgcggggtccgggccaggttctcacaccgtccagaggatgtatggctgcgacgtggggtcggactggcgcttcctccgcgggtaccaccagtacgcctacgacggcaaggattacatcgccctgaaagaggacctgcgctcttggaccgcggcggacatggcagctcagaccaccaagcacaagtgggaggcggcccatgtggcggagcagttgagagcctacctggagggcacgtgcgtggagtggctccgcagatacctggagaacgggaaggagacgctgcagcgcacgggtaccaggggccacggggcgcctccctgatcgcctgtagatctcccgggctggcctcccacaaggaggggagacaattgggaccaacactagaatatcgccctccctctggtcctgagggagaggaatcctcctgggtttccagatcctgtaccagagagtgactctgaggttccgccctgctctctgacacaattaagggataaaatctctgaaggaatgacgggaagacgatccctcgaatactgatgagtggttccctttgacacacacaggcagcagccttgggcccgtgacttttcctctcaggccttgttctctgcttcacactcaatgtgtgtgggggtctgagtccagcacttctgagtccttcagcctccactcaggtcaggaccagaagtcgctgttccctcttcagggactagaatttccacggaataggagattatcccaggtgcctgtgtccaggctggtgtctgggttctgtgctcccttccccatcccaggtgtcctgtccattctcaagatagccacatgtgtgctggaggagtgtcccatgacagatcgaaaatgcctgaatgatctgactcttcctgacagacgcccccaaaacgcatatgactcaccacgctgtctctgaccatgaagccaccctgaggtgctgggccctgagcttctaccctgcggagatcacactgacctggcagcgggatggggaggaccagacccaggacacggagctcgtggagaccaggcctgcaggggatggaaccttccagaagtgggcggctgtggtggtgccttctggacaggagcagagatacacctgccatgtgcagcatgagggtttgcccaagcccctcaccctgagatggggtaaggagggagacgggggtgtcatgtcttttagggaaagcaggagcctctctgacctttagcagggtcagggcccctcaccttcccctcttttcccagagccgtcttcccagcccaccatccccatcgtgggcatcattgctggcctggttctctttggagctgtgatcactggagctgtggtcgctgctgtgatgtggaggaggaagagctcaggtggggaaggggtgaagggtgggtctgagatttcttgtctcactgagggttccaagacccaggtagaagtgtgccctgcctcgttactgggaagcaccacccacaattatgggcctacccagcctgggccctgtgtgccagcacttactcttttgtaaagcacctgttaaaatgaaggacagatttatcaccttgattacagcggtgatgggacctgatcccagcagtcacaagtcacaggggaaggtccctgaggaccttcaggagggcggttggtccaggacccacacctgctttcttcatgtttcctgatcccgccctgggtctgcagtcacacatttctggaaacttctctgaggtccaagacttggaggttcctctaggaccttaaggccctgactcttttctggtatctcacaggacattttcttcccacagatagaaaaggagggagctactctcaggctgcaagtaagtatgaaggaggctgatgcctgaggtccttgggatattgtgtttgggagcccatgggggagctcacccaccccacaattcctcctctagccacatcttctgtgggatctgaccaggttctgtttttgttctaccccaggcagtgacagtgcccagggctctgatgtgtctctcacagcttgtaaaggtgagagcctggagggcctgatgtgtgttgggtgttgggcggaacagtggacacagctgtgctatggggtttctttccattggatgtattgagcatgcgatgggctgtttaaagtgtgacccctcactgtgacagatacgaatttgttcatgaatatttttttctatagtgtgagacagctgccttgtgtgggactgagaggcaagagttgttcctgcccttccctttgtgacttgaagaaccctgactttgtttctgcaaaggcacctgcatgtgtctgtgttcgtgtaggcataatgtgaggaggtggggagaccaccccacccccatgtccaccatgaccctcttcccacgctgacctgtgctccctccccaatcatctttcctgttccagagaggtggggctgaggtgtctccatctctgtctcaacttcatggtgcactgagctgtaacttcttccttccctattaaaattagaacctgagtataaatttactttctcaaattcttgccatgagaggttgatgagttaattaaaggagaagattcctaaaatttgagagacaaaataaatggaacacatgagaaccttccagagtccacgtgttgcttatgctgatttgttgcaggggaggagagtagatggggctgtgcccagtttctgttccggccactatgggctttatgtggtcactgcttggctgggtcatctttgctgctccattgtccttgg

SEQ ID NO: 450 - DNA, 1601 bp, Artificial sequence
  interleukin 10 coding sequence
aaaccacaagacagacttgcaaaagaaggcatgcacagctcagcactgctctgttgcctggtcctcctgactggggtgagggccagcccaggccagggcacccagtctgagaacagctgcacccacttcccaggcaacctgcctaacatgcttcgagatctccgagatgccttcagcagagtgaagactttctttcaaatgaaggatcagctggacaacttgttgttaaaggagtccttgctggaggactttaagggttacctgggttgccaagccttgtctgagatgatccagttttacctggaggaggtgatgccccaagctgagaaccaagacccagacatcaaggcgcatgtgaactccctgggggagaacctgaagaccctcaggctgaggctacggcgctgtcatcgatttcttccctgtgaaaacaagagcaaggccgtggagcaggtgaagaatgcctttaataagctccaagagaaaggcatctacaaagccatgagtgagtttgacatcttcatcaactacatagaagcctacatgacaatgaagatacgaaactgagacatcagggtggcgactctatagactctaggacataaattagaggtctccaaaatcggatctggggctctgggatagctgacccagccccttgagaaaccttattgtacctctcttatagaatatttattacctctgatacctcaacccccatttctatttatttactgagcttctctgtgaacgatttagaaagaagcccaatattataatttttttcaatatttattattttcacctgtttttaagctgtttccatagggtgacacactatggtatttgagtgttttaagataaattataagttacataagggaggaaaaaaaatgttctttggggagccaacagaagcttccattccaagcctgaccacgctttctagctgttgagctgttttccctgacctccctctaatttatcttgtctctgggcttggggcttcctaactgctacaaatactcttaggaagagaaaccagggagcccctttgatgattaattcaccttccagtgtctcggagggattcccctaacctcattccccaaccacttcattcttgaaagctgtggccagcttgttatttataacaacctaaatttggttctaggccgggcgcggtggctcacgcctgtaatcccagcactttgggaggctgaggcgggtggatcacttgaggtcaggagttcctaaccagcctggtcaacatggtgaaaccccgtctctactaaaaatacaaaaattagccgggcatggtggcgcgcacctgtaatcccagctacttgggaggctgaggcaagagaattgcttgaacccaggagatggaagttgcagtgagctgatatcatgcccctgtactccagcctgggtgacagagcaagactctgtctcaaaaaaataaaaataaaaataaatttggttctaatagaactcagttttaactagaatttattcaattcctctgggaatgttacattgtttgtctgtcttcatagcagattttaattttgaataaataaatgtatcttattcacatc

SEQ ID NO: 451 - DNA, 12150 bp, Artificial sequence
  Ricin toxin coding sequence
atatttcacgaactgatacaatatagcaagagattgaaaaaactcaaattcttacaaaactgaactgaaataaaacaagagatgcataataaaaaaatacaaatcttgatacttattacaactggaatatgcgactaagctgaaataaactgacattaaagacatgacagaaatacatgttttggtctataacaacctaagtagaaatcgcagcctgttgatgcacagatacatgtattcttatatatttgtattaatattttaatttatgtacatatcgagatgaatgaaaataagctaatatttgtcatttagaaattattccaaaactgaatcatcttacttgagttatttaattaataaaattaaatatatttcattaaacaatgcattttctctttaaaacaatccatttgaatcttactttataggaagaccttgatagataaacaatgtattggatatacgttttttttagatgctctaaagattgcattagaatgaaataaaactttttacttttaagatttttgctctttaaaataaaaagtacaaactttgataagttttatataacctaaaaagaaattgtaacatgtgagtttgagatgtcttatataatctacaaagatatttaaagagtataaacagtaaagcaataatacttaggtgatgaaaatacctcacttagtactaatacaacaaagcaagatatcgaacaaatcttaaatctttgcaaaactgaactgaagtaaaacaagaaatgcataataataagacaaatcttggaacttattatagctggaatatgccagtaagctgaaataaattgatactaaggacacaacagaaacacatgcgtctgcaacctaagaagaagttgcaacctgtcgatgcataaatatacatatgcgacgtatattgctatgtatgaaaataagcgaatacttatcatttggaatttattccaaaattggatcatcttgctgagttattcaattaataataatgaatatattttattaagaaataaaaaatttaccttttcctctttaaaacgatttatttgaatattactttatgagaagactttttataatcagaaaacttgatatataaagaatgtatttgataatgtttctttagagatgctctaaagattgtattgcacttaaataaaaccttttactcttaatagtttagtatttctcaaaattattactctttaaagtaaatagctcaaactttgagaagtcttatgtaaactaaacaaaaattgtaacctacgagtttgagatgtcttatacaacctaaaataaaatttaaagaatataaactttaagatgtctttttctatataatcaattttattttgaaaaaccaccaaagcaaaaaaaatttaggtgatggaaatatcctacctagaactaataaaatatagcaaaagattaaacaaatctcaaattcttataaaattgaactgaagtaaaacaagagatgtataataacaaattttggtacttaatacaaaaatatgtcattaagctaaagtaaagtgataataaggacacaatataaacacatgctttggcctccagcaatctaataagaaattgcaacctatcgatgcacaaatacacgtatctttatatgcttatataagtattttaatttgtgtacgtgtattcaaatgactgaaaataaactaaaacttgtcatataaaattttttcaaaattaaatgatcttgctaagttattcactaataataataaatatatttcattaaaaataaaaatttgtcctttctctataaaagagtccattagaatcttactatatatgaaggctttttataattaaaaaacttgatatataaagaatgtattggaggtgcattttttttttgagatgttctgaagattgcattggactacaataaaagtttttacttttacttcttagaattcttgctctttaaaataataagcacaaactttgagatgtcttgtagaacctaagaaaaaaattacaatttgtgatttttagatgttttatacaatctaaaaagagatttgaagagtataaattttaagatgttttatacaacctaaaacaaattgtaatctgtcgacgcacagacacgtatttttatatatttataaagatcattttaatttatgtacttatatttagataaataaaaataagctaatatttgtcatatataaattatttcataactgaattgtctgatttgttagtgttattaaattaataataatagttatattttattaaataataaaagatttaccttttctctataaaaaggggaagaaatgtggttactaaaaccatctcatattcctccgctttcttcctcagctgctcactttgtaagtattacgactttctcaaacttcctacttgttttcaattaatgttattctacctgacttcatatatatctcttttcttttgggatcattattactgctactgtaatgatcgagaaaaatctcttcttcttatcattatgacttcatatattatcatcttctatcacttcatataatgtttttgattttatgattcaatatatttataattaagttctatctttaaacgagagcaaccaattaaagaaacaagagtaaatatatataaattgtaaggtattatttgtttgatttaaatcattaaatagctagatcttctttttcttttctttttttctttctcatccaaagtttttatgaattgcaggctattagtaatatagatggtagaaagaaaattaaaattaaaacttcttcaatcatcacaaatgagagtaccaactaaactatgtgattttggtaaaaaatgcaaaacaagtacttatatatatatatatatatatatatatatatatatatatatatatattcttttataaatcagtatatacattttactctgattaccataatattatagattttactaaggtgacactaatgttatatattttggttagaatggtagtgttttcttttcttaaagatgctctagaggattcttaacaaaagaatataatatataaataatatattaaagatgccctagaaaatgcatttactgtacttaaataacctgttttgctcttaatattttattatttttatttctataataaaaaatattttaagaaatatttaagtataaaaaataaagtattttattgatgtccactgtactttttatattttatttcttattttacttttgtttccaagggcatcaatatcttcttcttttttctgtttaatttttattaataaaaaaaataattacaaatattaattaatcaaatacatagaaatttatttttataaaaaaaatccttcaaattcttttaaaatgtcattttgaccctaaatttcttttaatagttagtgttctaataaaaaaaatttacccaataatttttctaatatttcattattcttttataagacaaactcttagcctctagaattattttaaggatatatataatttgtctctctttctctttaacatagccttagtttccaataaataaataatgaaatatatttcactcttcatttctttaaacttcttacatttttttttgtagcattctttgtaagtggaatgacaaaaccgttaatgatgttcttttaaaagtgaaagatgtttatatattgcagtacagataatgatatatctactgcactacataaaacaatttaaatctccctgtttattttaagaagttatattttctttctttctcatcctaagaaagttaaattactgtaatcgacattatatgaattttaactaattccgtttctaatttataattatttcgttaaaccaatcaattccctttaaacactgcttatgcatattctgtctcaatttatatatggcatgcatcttccgtattaatttataagttcatttttattgatcaagtatttgtggttttctttatataaaaaaatgtattagtgtttttctgtattaattttataagttcatctttatgagaatgctaatgtatttggacagccaataaaattccaagaattgctgcaatcaaagatgaaaccgggaggaaatactattgtaatatggatgtatgcagtggcaacatggctttgttttggatccacctcagggtggtctttcacattagaggataacaacatattccccaaacaatacccaattataaactttaccacagcgggtgccactgtgcaaagctacacaaactttatcagagctgttcgcggtcgtttaacaactggagctgatgtgagacatgaaataccagtgttgccaaacagagttggtttgcctataaaccaacggtttattttagttgaactctcaaatcatgcagagctttctgttacattagcgctggatgtcaccaatgcatatgtggtcggctaccgtgctggaaatagcgcatatttctttcatcctgacaatcaggaagatgcagaagcaatcactcatcttttcactgatgttcaaaatcgatatacattcgcctttggtggtaattatgatagacttgaacaacttgctggtaatctgagagaaaatatcgagttgggaaatggtccactagaggaggctatctcagcgctttattattacagtactggtggcactcagcttccaactctggctcgttcctttataatttgcatccaaatgatttcagaagcagcaagattccaatatattgagggagaaatgcgcacgagaattaggtacaaccggagatctgcaccagatcctagcgtaattacacttgagaatagttgggggagactttccactgcaattcaagagtctaaccaaggagcctttgctagtccaattcaactgcaaagacgtaatggttccaaattcagtgtgtacgatgtgagtatattaatccctatcatagctctcatggtgtatagatgcgcacctccaccatcgtcacagttttctttgcttataaggccagtggtaccaaattttaatgctgatgtttgtatggatcctgagcccatagtgcgtatcgtaggtcgaaatggtctatgtgttgatgttagggatggaagattccacaacggaaacgcaatacagttgtggccatgcaagtctaatacagatgcaaatcagctctggactttgaaaagagacaatactattcgatctaatggaaagtgtttaactacttacgggtacagtccgggagtctatgtgatgatctatgattgcaatactgctgcaactgatgccacccgctggcaaatatgggataatggaaccatcataaatcccagatctagtctagttttagcagcgacatcagggaacagtggtaccacacttacagtgcaaaccaacatttatgccgttagtcaaggttggcttcctactaataatacacaaccttttgtgacaaccattgttgggctatatggtctgtgcttgcaagcaaatagtggacaagtatggatagaggactgtagcagtgaaaaggctgaacaacagtgggctctttatgcagatggttcaatacgtcctcagcaaaaccgagataattgccttacaagtgattctaatatacgggaaacagttgtcaagatcctctcttgtggccctgcatcctctggccaacgatggatgttcaagaatgatggaaccattttaaatttgtatagtgggttggtgttagatgtgagggcatcggatccgagccttaaacaaatcattctttaccctctccatggtgacccaaaccaaatatggttaccattattttgatagacagattactctcttgcagtgtgtatgtcctgccatgaaaatagatggcttaaataaaaaggacattgtaaattttgtaactgaaaggacagcaagttattgcagtccagtatctaataagagcacaactattgtcttgtgcattctaaatttatggatgaattgtatgaattaagctaattattttggtcatcagacttgatatctttttgaataaaataaataatatgttttttcaaacttataaaactatgaatgatatgaatataatgcggagactagtcaatcttttatgtaattctatgatgataaaagcttgtctcttaacttagtgaatttgtatccaagtaaaaaacagcctactaagtcatggattccttcaaatttacgctcttattataagcttaattttcatccacgatcatccctattcatgtgatgcacaagaacttaaggtatatagatttgaagtaattccttaattataatttttaagtttatcactttctttactttctaatttctttttctcaaattgtactaattaactcattgaagaatttaacaacttgttcatcagttccatagtaattctcaataaatttatggtctcacaactcaagcatccatgggttcataagttgtgataatcaggcagcttcatatatattgaaattaacataaggagtaaagtgggttgagcaatgccaggtggtacatccaggtggtacatccatatatacttcttcctccaaatcaccatgtaaaaaagtattttttacatcaaattgtcttaatgcccatcctttagttgctgcaatagaaatcagtattcaaatcgtaccaagtttcgccacaggtgcaaacgtttcctggtagtcaattccatacttctgagtaaatcctttcgctaccagtctcgcttttcgcctgtctagactcccatctgccttaagcttcactgtatagatccatttacagccaatcgtattcttcccctctggtaatttcacaaactcccatgttgcattcttctctagggctttcacctcttcctccatcgcctcttttcatttcaagtcttgcattgcctcctgcacactattaggaatcgcaatcccggataattcactagtgctaagtgcataataatctgttaaatgattatgtcgaataccattcttagttaaaaaattacttaaatgactatccataaattcctttgcattatcagtttgccaaactaaaacccatgtacgatattgatttctaataaaatttcaaaaatcacaaaaagctttacttacctcacttttgttccgaagtaaggttatccaagtcatacgagtacattcgtctataaaagttccgaaatatctagcacctgataatgaaggaatttttgcaggaccccaaacatcagaatgtataacagcaaatggttcagtacttttattaaaactaggcaaataagtagcacggtggctcttaccaagttcacacacatcacaattaaacacggaatcatccaaattaacaaacaattcaggttttaattttttcaaataactaaatgacaaatgtccaaggcgtcgatgccatagccaaattctttccattgtattacttaagctttcagtcgtgtaaacctcacttattttcttctctcctatctcagtcaagtctagataataaagtttgcctcgtttaacaccataaccaagagtctcccgagtcaggatgtcctgaaaaacacaatatgaaggccagaaagttacagtacagttaagagacgaggttatttgtccaaccgagagtaagttacaagacagtgtaagaacaatcaatacagattttaaattcagattttttgaaagagtaatggatccttctccactaacaatacatttggatccatttgttattgacacggtaaaatgagaggatggaatgaaagaataaattcgatcagaattgcataccatatgatcagatgcaccagggtctattgtccacaatgttcacacagcaagttacatttttttccagctccatttttcctgttttttccataattctggcaaacctgaaatacagtaggctccacagttgcatttgttcctcccattgtcttacattgttgatcttctcttcgaacataagcatatgtttgttctaaatcaagtttagggtctttgcgcaaaatctcacctcggacttgatcaaagtttgaatcaagcccactgaggaagatatgcatgcgtagccttgccatggcagaatgcaacgctactactccatccactgtaccttcatgtgactgagtacgatgatctatctcttgaaagatttccatcaactcagagtaatacgtaggcaatggtctgccctcttgtttgattgcgaaagacttctgattcaattcgaataacctcgtttcatcagttccatcataaaatgtttttgctgctgcctcccaaacatctttggcagtaggaagtctgatgaaatgttgcatcagtgaaggtatcatcgaatcaatcagccagctctttaccttttgattctcagttatccaggtagagtatctcgtatctgtagttgcaggcttcacttccgatccggttaaatacccagttttatttcgtgctccaatacgcatctccatgagctgtgaccatagagaataattcgattcatccagtataacactggtagggaatgatacattatcttgttgaatgataatttgattcgaagacggattcatgatttgagtatgaggtggatcttctgtactcataattctgtttagggattcagttttgatttgaaaaaaatttgttttcagtatggtgagcggctgttgtgataccatgtgagtaatgaaactctaataattgattaattttcatcctttttctctattcattcaacctgaatatatacaaaagtttgttacaaggataacttctaactacttatgtaacaagcaaccttatcaaaaatacaattagaataacatctaactactacagcttatacatacttatctctatactaacaaattaactaaatatagttaacaagttctgaatcatagtaattttagtctctataatattaacaaatgagggactaaaatacttttcttttcttaaatatgattctattttaaaaagcgggagatagaaaaatttatataatttcttactaaaaaaaagtaaggataattttatttaattctaagaaacaaagcaaaggaaaatgggaaagtttataatttgatctaatttctttgcatgatagacaggtaactgattctcaatacgattctaagatgatccaatataaattatttgtgggaaataggttcacgaaaaagccatttgttggggattcaatgtttattgatttgttttgtgtagtatgttttgcatcaattaattagatcagctagtcatttacatattcttgcaaatttgtgccatttacaaatgaattatctgaatttagtgtaatttgattaatttaattagacgggttttccttcttcagtaatcaacatacacaaagtgaaaggcaaacgagttctagcaatttaattgattaggttcaagttaattaattaatttgataatttatggttcagtccgttactgaaaatttaaaaagaagtagcatgcatctttagattctttctctattttatgcatgccattttctgattaattgtatatgttcgttcttcttgattaagtatttatatttattttttatttgattagtgagctatatatatttttaagagaatcttgatgtatttggacaactaaaaaaattccaagaattgctacaattggagatgaaactgaaaggaaacactatggtgtggatatacgcagtggcaacgtggctctgcttgggatcagccttggggtggtaattcaatgccactgtaagctacacattcccaactgtaagctttaccactgttgatgccacggagcaaatgtacagagagtgttgtagaaaataagaataagtatatatacttatcggcttagtagccaagaagtcttttggtaacctacaactcttaaacatcatatacttaatacataagttacacgcttaatgaatatatatgtaacttacatatttaataggtgactctttaatttcataacttaagcattaaaaactatacataatgattattaaattaaatttagtttttcaacactccttaaatttaatttcagatcaaaccgagtcttgctcttaatctggcaaaatcttcaaactttaatgccttcgtcaaaatatcagcaagttgatcttgcgacttgacatacttcagctctatttccttcttacggatactttctctgatgaaatgatatcttctattaatgtatttactcttaacatgtaaaataggattcttgctcaatgctatagacgatttattatcaataaaaatatcaattggttcttcgtttgaaaccaatagcacctctaacacctttcttaaccatgtggcatgacaaacacaagcagcagcaacaacatacttaacttcacaagttgaaagaattacaatagattacttctttgaactccatgtaaaagcagtctctccaatagaaaacacaaaacccattgtactctttcgatcatcaatatcacctcctcaatcactatcactatagcctactagactgaaactttttgaaggtgagtaaaacaaaccataatctaaagttccttcgacatatctcaaaattcttttaccagccttcatgtgcgaatctgttggtgcctccatgtagcgactaataagtcctactccataaagaatatcaggtctagtacaagtcaagtacctcaagtttccaacaaatctttgaaacaaaattggattcaccttctcagtattatcatatcttgacagctttactccacattcaactagtgtgctgacaagctgacattgctccatgccaaactcctttaagatctttcttacatagctcttgtgagaaataaaaatgccatcattagtttgtttcactttgattcccagataataagacatcaaccctatatctgtcatctcaaattcagcagccatctcctttttgaattattcaatcattgctgaattatttttctgtaaagatcaaatcatcaacatacaagcacaccacaagaacatcaccattctctttcttcttagaataaagagaatattcatgaggatatttcagaaaatttttctttttgaaataatcatcaatcctgttgtaccatgcccttagtacttgcttcaatccatacaaaggtaccttcagttttaaaactttgttttcttcccctctaacaacatatctgaaaggttgttttagatatccttcttcttctaagtatccattcaagaacgccgatttcacgtccatttgaaaaattcttcatctcttttgtgcagctaaagcaatgattaaacaaattatttccaatcgagcaactactacaaagacttcatcatagtcaattccatgttgttggctaaacccttttgcaacaagtcgagccttgtatttctcgacatctccgttttcattctttttaactttttataaatccatttcacaccaacaacttgcttgccttaggaaagattgctaattcccaagtgtcattcttttgaattgattttatttcgtcatctatggctactctccatttttcatttttcatcgcttcttcaaaattcattggttcagtatctgcagaaatacaataatgaacaaaatctaaaacttcatcaatgttagcatatatgtcctacagatttctcatctttgtaggtttttcagatgatcttgaacgacttgaatctccttgaatacttgctggagttcttggaagtgtcaagttctgtacatcaaaattaaaatgttgaacatcttgatctgaataatcaaaaattggaaaaaaattataattttctgattgattctcccaatttcatccatcagcttcctcaaacttgacatctcgactcaggataatctttttgcttgtcggattgtacaacttgtagactttagactttgcatcatatccaacaaaaatgaattttccacttttatcatccagcttaattctggtttcatctgcaacatgaacataagcaacataaccaaaaactctaagatgagaaagagttggctttcttccactccatgcttcctgtggtatagacttctgcaaactctttataggacatatg

SEQ ID NO: 452 - protein, 345 aa, Artificial sequence
  PE38KDEL
EGGSLAALTAHQACHLPLETFTRHRQPRGWEQLEQCGYPVQRLVALYLAARLSWNQVDQVIRNALASPGSGGDLGEAIREQPEQARLALTLAAAESERFVRQGTGNDEAGAANGPADSGDALLERNYPTGAEFLGDGGDVSFSTRGTQNWTVERLLQAHRQLEERGYVFVGYHGTFLEAAQSIVFGGVRARSQDLDAIWRGFYIAGDPALAYGYAQDQEPDARGRIRNGALLRVYVPRSSLPGFYRTSLTLAAPEAAGEVERLIGHPLPLRLDAITGPEEEGGRLETILGWPLAERTVVIPSAIPTDPRNVGGDLDPSSIPDKEQAISALPDYASQPGKPPKDEL

SEQ ID NO: 453 - DNA, 1035 bp, Artificial sequence
  PE38KDEL coding sequence
gagggcggcagcctggccgcgctgaccgcgcaccaggcttgccacctgccgctggagactttcacccgtcatcgccagccgcgcggctgggaacaactggagcagtgcggctatccggtgcagcggctggtcgccctctacctggcggcgcggctgtcgtggaaccaggtcgaccaggtgatccgcaacgccctggccagccccggcagcggcggcgacctgggcgaagcgatccgcgagcagccggagcaggcccgtctggccctgaccctggccgccgccgagagcgagcgcttcgtccggcagggcaccggcaacgacgaggccggcgcggccaacggcccggcggacagcggcgacgccctgctggagcgcaactatcccactggcgcggagttcctcggcgacggcggcgacgtcagcttcagcacccgcggcacgcagaactggacggtggagcggctgctccaggcgcaccgccaactggaggagcgcggctatgtgttcgtcggctaccacggcaccttcctcgaagcggcgcaaagcatcgtcttcggcggggtgcgcgcgcgcagccaggacctcgacgcgatctggcgcggtttctatatcgccggcgatccggcgctggcctacggctacgcccaggaccaggaacccgacgcacgcggccggatccgcaacggtgccctgctgcgggtctatgtgccgcgctcgagcctgccgggcttctaccgcaccagcctgaccctggccgcgccggaggcggcgggcgaggtcgaacggctgatcggccatccgctgccgctgcgcctggacgccatcaccggccccgaggaggaaggcgggcgcctggagaccattctcggctggccgctggccgagcgcaccgtggtgattccctcggcgatccccaccgacccgcgcaacgtcggcggcgacctcgacccgtccagcatccccgacaaggaacaggcgatcagcgccctgccggactacgccagccagcccggcaaaccgccgaaagacgagctc

SEQ ID NO: 454 - DNA, 31 bp, Artificial sequence
  Single strand DNA oligonucleotide
ttaagcgttggcgcatatggaagttggttgg

SEQ ID NO: 455 - DNA, 98 bp, Artificial sequence; identical to SEQ ID NO: 194
  Single strand DNA oligonucleotide

SEQ ID NO: 456 - DNA, 32 bp, Artificial sequence
  Single strand DNA oligonucleotide
ttaagcgttggcgcatatgggggacacccgag